SEQUENCE LISTING (6 sequences)

SEQ ID NO: 1 - DNA, 22 bp, synthetic construct
  source 1..22: mol_type = other DNA
acccgttgaaccccattcgtga

SEQ ID NO: 2 - DNA, 23 bp, synthetic construct
  source 1..23: mol_type = other DNA
gcctcactaaaccatccaatcgg

SEQ ID NO: 3 - DNA, 21 bp, synthetic construct
  source 1..21: mol_type = other DNA
cgcagatcacccagaagatcg

SEQ ID NO: 4 - DNA, 20 bp, synthetic construct
  source 1..20: mol_type = other DNA
ttcgtcgcatttgtccacca

SEQ ID NO: 5 - DNA, 21 bp, synthetic construct
  source 1..21: mol_type = other DNA
agcacttcctccagaggtttg

SEQ ID NO: 6 - DNA, 20 bp, synthetic construct
  source 1..20: mol_type = other DNA
actcaccaggatgctcacat

(19) United States
(12) Patent Application Publication
(10) Pub. No.: US 2026/0158068 A1
(43) Pub. Date: Jun. 11, 2026

(54) HUMAN IFN-ALPHA2 AND PD-L1 OVEREXPRESSING T CELLS FOR IMMUNE MODULATION OF AUTOIMMUNE AND ALLOIMMUNE DISORDERS

(71) Applicants: Hackensack Meridian Health, Inc., Edison, NJ (US); Hackensack Meridian Health Center for Discovery and Innovation, Nutley, NJ (US)

(72) Inventors: Yi ZHANG, Wallingford, PA (US); Yuanyuan Tian, Edison, NJ (US); David Perlin, New York, NY (US)

(73) Assignees: Hackensack Meridian Health, Inc., Edison, NJ (US); Hackensack Meridian Health Center for Discovery and Innovation, Nutley, NJ (US)

(21) Appl. No.: 19/405,840

(22) Filed: Dec. 2, 2025

Related U.S. Application Data
(60) Provisional application No. 63/729,145, filed on Dec. 6, 2024.

(51) Int. Cl. A61K 35/17; A61K 9/00; A61K 40/11; A61K 40/42; A61K 45/06; A61P 35/02; A61P 37/04; C07K 14/56; C07K 14/705; C07K 16/28; C12N 5/0783; C12N 9/12; C12N 15/86

(52) U.S. Cl. CPC A61K 35/17; A61K 9/0019; A61K 40/11; A61K 40/421; A61K 40/4235; A61K 45/06; A61P 35/02; A61P 37/04; C07K 14/56; C07K 14/70532; C07K 16/2818; C12N 5/0637; C12N 9/12; C12N 15/86; A61K 2239/31; A61K 2239/48; C12N 2510/00; C12N 2740/15043; C12Y 207/10002

(57) ABSTRACT

The present disclosure relates to cell therapy methods comprising administering a hematopoietic cell transplant comprising a population of genetically engineered T cells that overexpress PD-L1 and IFN-α2 (αρ-T cells) to a recipient subject in need thereof, wherein the recipient in need thereof is afflicted with an autoimmune or alloimmune disorder. The genetically engineered population of αρ-T cells is characterized by expansion in vitro, persistence in vivo, capacity to infiltrate target tissues; and sustained immune modulatory function. In one application, an administered allogeneic or autologous hematopoietic cell transplant suppresses graft versus host reactivity in a recipient subject with a hematologic cancer while preserving graft versus tumor immunity. In a second application, the administered hematopoietic cell transplant suppresses autoimmune disorders or disorders with an autoimmune component.

### CROSS-REFERENCE TO RELATED APPLICATIONS

This application claims the benefit of priority to U.S. provisional application 63/729,145 (filed Dec. 6, 2024) entitled “Human IFN-alpha2 and PD-L1 overexpressing T cells (alpha p-T cells) for graft-versus-host disease prevention,” the contents of which are incorporated herein by reference in their entirety.

### SEQUENCE LISTING

The application contains a Sequence Listing which has been submitted electronically in .XML format and is hereby incorporated by reference in its entirety. Said .XML copy, created on Dec. 1, 2025, is named “2798-003US-SeqList.xml” and is 6,468 bytes in size. The sequence listing contained in this .XML file is part of the specification and is hereby incorporated by reference herein in its entirety.

### FIELD OF THE INVENTION

The present disclosure relates to cell therapy methods comprising delivery of genetically engineered T cells for immune modulation of autoimmune and alloimmune disorders.

### BACKGROUND OF THE INVENTION

### Cell Therapy

Cellular therapy as a modality for successful treatment of disease has existed for decades, for example, in the form of blood stem cell transplants to treat a variety of hematological malignancies and immunological disorders. Patient-specific blood stem cells derived from bone marrow, mobilized peripheral blood, and cord blood have been successfully used as sources for blood stem cells to treat these diseases. With proven utility for treating various diseases, blood stem cell transplants still face many challenges in making the therapy more broadly applicable, including insufficient quantities of blood stem and progenitor cells available for transplant, less intense conditioning regimens that support long-term efficacy with less toxicity, the risk of chronic graft versus host disease (GVHD), and relapse [Granat et al. Haematologica, 2020; 105 (12): 2716-2729]. In an effort to address the insufficient cell supply for transplant, some companies have focused development on novel mobilizing agents and ex vivo expansion protocols.

In 2017, the first chimeric antigen receptor (CAR)-T cell therapy, KYMRIAH®, was approved by the United States Food and Drug Administration for use in pediatric and young adult patients with B cell Acute Lymphoblastic Leukemia (ALL). Since then, several additional therapies and indications have been approved utilizing this autologous cell treatment approach. The process for manufacturing these therapies is intensive and expensive, involving the isolation of donor T cells, genetic engineering of T cells for targeting the cancer and persistence of T cell population, and T cell activation and expansion [Vormittag et al., Curr Opin Biotechnol. 2018; 53:164-181]. While development of allogenic/off the shelf versions of CAR-T cell therapies should reduce the cost of treatment, the manufacturing process will still rely on the core steps of isolation, engineering, activation, and expansion. Both CAR-T and blood stem cell transplants have proven successful in treating disorders originating in the blood [Goldsmith et al., Frontiers in Oncology, 2020; 10:2904] but are limited in treating diseases that do not originate from or exist within blood as their developmental potential is generally restricted to blood cell types.

In 1998, the first human embryonic stem cells (hESCs) were isolated in the lab of James Thomson at the University of Wisconsin-Madison [Thomson et al. Science, 1998; 282:861-872; Takahashi et al. Cell, 2007; 131:861-872]. These cells, derived from a day 5 preimplantation blastocyst stage during embryogenesis, were not restricted in their potential to generate the variety of cell types that exist in the human body. This state of developmental potential, known as pluripotency, in combination with the hESCs unique ability to self-renew (e.g., the persistent ability to generate additional cells of identical genetic makeup and pluripotentiality), opened the door to treating diseases in all cell types and tissues in the human body.

hESCs, while harboring this unlimited therapeutic potential, also came with moral and ethical objections as their isolation resulted in the destruction of human embryos. These objections led to limited financial support, thus slowing their development towards therapeutic utility. In 2007, these objections were obviated as the labs of Shinya Yamanaka and James Thomson independently developed the ability to convert human adult/somatic skin cells to pluripotent stem cells by exogenously delivering a combination of genes associated with the embryonic cell state [Takahashi et al. Cell, 2007; 131:861-872; Yu et al. Science, 2007; 318:1917-1920]. This process, known as cellular reprogramming, not only obviated the moral and ethical issues associated with using hESCs to develop new treatments, but also made it possible to create pluripotent stem cells (PSCs) from each individual. This opened the door to autologous treatment of diseases affecting all tissues of the human body, not just blood.

With this capability in hand, the field focused efforts on the development of best systems and practices to ensure compatibility with intended future uses in the development of iPSC-derived cell therapies. As such, primary somatic cell materials, cellular reprogramming systems, cell culture/expansion systems and environments, gene editing technologies, and differentiation protocols were evaluated and developed towards consistency/reproducibility, integrity, quality, and scalability in manufacturing processes. Cellular reprogramming protocol development focused on the use of blood derived cell types as a primary somatic cell starting material given the ease of access in a clinical setting and the pre-existence of biobanked blood materials. Of additional benefit, blood derived cell types are better protected from environmental mutagens such as UV light, which can lead to accumulation of genetic variations/mutations in DNA sequences and chromosome structure.

Surgery and chemotherapy remain the most common treatments for cancer. Cell therapy requires the administration of cells to a subject for the purposes of treating a disease or disorder, such as cancer. Chemotherapies are designed to kill rapidly dividing cells, including cancer cells, but it also effects healthy cells, including those of the immune system. Thus, chemotherapy has the potential to damage or kill immune cells, which potentially hinders the effectiveness of future cell therapies and leading to unwanted side effects.

### Cells of the Immune System

There are a large number of cellular interactions that comprise the immune system. These interactions occur through specific receptor-ligand pairs that signal in both directions so that each cell receives instructions based on the temporal and spatial distribution of those signals.

Murine models have been highly useful in discovering immunomodulatory pathways, but clinical utility of these pathways does not always translate from an inbred homogeneous mouse strain to an outbred heterogeneous human population, since an outbred human population may have individuals that rely to varying extents on individual immunomodulatory pathways.

Cells of the immune system include lymphocytes, monocytes/macrophages, dendritic cells, the closely related Langerhans cells, natural killer (NK) cells, mast cells, basophils, and other members of the myeloid lineage of cells. In addition, a series of specialized epithelial and stromal cells provide the anatomic environment in which immunity occurs, often by secreting critical factors that regulate growth and/or gene activation in cells of the immune system, which also play direct roles in the induction and effector phases of the immune response [Paul, W. E., “Chapter 1: The immune system: an introduction,” Fundamental Immunology, 4th Edition, Ed. Paul, W. E., Lippincott-Raven Publishers, Philadelphia, (1999), at p. 102].

The cells of the immune system are found in peripheral organized tissues, such as the spleen, lymph nodes, Peyer's patches of the intestine and tonsils. Lymphocytes also are found in the central lymphoid organs, the thymus, and bone marrow, where they undergo developmental steps that equip them to mediate the myriad responses of the mature immune system. A substantial portion of lymphocytes and macrophages comprise a recirculating pool of cells found in the blood and lymph, providing the means to deliver immunocompetent cells to sites where they are needed and to allow immunity that is generated locally to become generalized [Paul, W. E., “Chapter 1: The immune system: an introduction,” Fundamental Immunology, 4th Edition, Ed. Paul, W. E., Lippincott-Raven Publishers, Philadelphia, (1999), at p. 102].

Two broad classes of lymphocytes are recognized: the B lymphocytes (B cells), which are precursors of antibody-secreting cells, and T lymphocytes (T cells).

### T Lymphocytes

T lymphocytes, or T cells, derived from precursors in hematopoietic tissue, undergo differentiation in the thymus, and are then seeded to peripheral lymphoid tissue and to a recirculating pool of lymphocytes. T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines [Paul, W. E., “Chapter 1: The immune system: an introduction”, Fundamental Immunology, 4th Edition, Ed. Paul, W. E., Lippincott-Raven Publishers, Philadelphia, (1999)].

T cells differ from B lymphocytes or B cells in their mechanism of antigen recognition. Immunoglobulin, the B cell's receptor, binds to individual epitopes on soluble molecules or on particulate surfaces. B cell receptors see epitopes expressed on the surface of native molecules. While antibody and B cell receptors evolved to bind to and to protect against microorganisms in extracellular fluids, T cells recognize antigens on the surface of antigen-presenting cells (APCs) and mediate their functions by interacting with, and altering, their behavior.

There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When encountering invading microbes at these sites, they endocytose the pathogens and their products and carry them via the lymph to local lymph nodes or to gut-associated lymphoid organs. The encounter with a pathogen induces the dendritic cell to mature from an antigen-capturing cell to an APC that can activate T cells.

APCs display three types of protein molecules on their surface that have a role in activating a T cell to become an effector cell: (1) major histocompatibility complex (MHC) proteins, which present foreign antigen to the T cell receptor; (2) costimulatory proteins which bind to complementary receptors on the T cell surface; and (3) cell-cell adhesion molecules, which enable a T cell to bind to the APC for long enough to become activated [“Chapter 24: The adaptive immune system,” Molecular Biology of the Cell, Alberts, B. et al., Garland Science, NY, (2002)].

T cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. Cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs [Paul, W. E., “Chapter 1: The immune system: an introduction,” Fundamental Immunology, 4th Edition, Ed. Paul, W. E., Lippincott-Raven Publishers, Philadelphia, (1999)].

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes [Paul, W. E., “Chapter 1: The immune system: an introduction,” Fundamental Immunology, 4th Edition, Ed. Paul, W. E., Lippincott-Raven Publishers, Philadelphia, (1999)].

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources [Paul, W. E., “Chapter 1: The immune system: an introduction,” Fundamental Immunology, 4th Edition, Ed. Paul, W. E., Lippincott-Raven Publishers, Philadelphia, (1999)].

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors.

This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted [Paul, W. E., “Chapter 1: The immune system: an introduction,” Fundamental Immunology, 4th Edition, Ed. Paul, W. E., Lippincott-Raven Publishers, Philadelphia, (1999)]. T cells can also be classified based on their function as helper T cells; T cells involved in inducing cellular immunity; suppressor T cells; and cytotoxic T cells.

### T Cell Development

Lymphopoiesis takes place in specialized lymphoid tissues known as the central or primary lymphoid tissues, which are the bone marrow for most B cells and the thymus for most T cells. Precursors for both populations originate in the bone marrow; B cells complete most of their development there, while the precursors of most T cells migrate to the thymus where they develop into mature T cells. In the fetus and the juvenile, the central lymphoid tissues are the sources of large numbers of new lymphocytes, which migrate to populate the peripheral lymphoid tissues (also called secondary lymphoid tissues), such as lymph node, spleen and mucosal lymphoid tissue. In mature individuals, the development of new T cells in the thymus slows down, and peripheral T cell numbers are maintained by the division of mature T cells outside the central lymphoid organs. New B cells are continually produced from the bone marrow, even in adults [Janeway's Immunobiology, 9^{th} Ed., Murphy K. and Weaver, C. Eds. Garland Science (2017) at p. 295].

The cells of the lymphoid lineage (B cells, T cells, and innate lymphoid cells, including NK cells) are all derived from common lymphoid progenitor cells, which themselves derive from the multipotent hematopoietic stem cells (HSCs) that give rise to all blood cells. Development from the precursor stem cell into cells that are committed to becoming B cells or T cells follows the basic principles of cell differentiation, i.e., properties that are essential for the function of the mature cell are gradually acquired, and properties that are more characteristic of the immature cell are lost. In the case of lymphoid development, cells become committed first to the lymphoid lineage, as opposed to the myeloid, and then to either the B cell or the T cell lineage. Notch signaling in thymic progenitor cells is essential to initiate the T cell specific gene expression program and commitment to the T cell lineage. Together T cell factor 1 (TCF1) and GATA2 initiate expression of several T lineage specific genes. A third transcription factor, Bcl11b, is required to induce T-lineage commitment by restricting progenitor cells from adopting alternative fates [Janeway's Immunobiology, 9th Ed., Murphy K. and Weaver, C. Eds. Garland Science (2017) at p. 297-317].

T cell development parallels that of B cells in many ways, including the orderly and stepwise rearrangement of antigen-receptor genes, the sequential testing for successful gene rearrangement, and the eventual assembly of a heterodimeric antigen receptor. T cell development has some features not seen for B cells, such as the generation of two distinct lineages of T cells expressing antigen receptors encoded by distinct genes, the γ:δ lineage and the α:β lineage. Developing T cells also undergo rigorous selection that depends on interactions with thymic cells and that shapes the mature repertoire of T cells to ensure self-MHC restriction as well as self-tolerance [Janeway's Immunobiology, 9th Ed., Murphy K. and Weaver, C. Eds. Garland Science (2017) at p. 315].

Phenotypic and functional analysis of CD4+ and CD8+ T cells from blood, lymphoid and mucosal sites employed to study T cells isolated from human tissues obtained from organ donors to gain insights into how T cell subsets are distributed and functionally maintained in humans revealed that the organization, differentiation and maintenance of human T cells was strikingly tissue intrinsic [Sathaliyawalla, T. et al. Immunity (2013) 38:187-97]. This multidimensional analysis revealed distinct compartmentalization of naïve, effector and memory CD4+ and CD8+ T cell subsets intrinsic to the tissue site that was remarkably consistent in diverse individuals. Memory CD4+ T cells represent the majority subset in mucosal tissues and accumulate in lymphoid tissue throughout life. CD8+ T cell subsets, by contrast, are maintained as naïve cells in lymphoid compartments over decades, with memory CD8+ T cells mainly in mucosal sites and terminal effector cells confined to circulation. Memory T cells in all tissues specifically upregulate CD69 expression, a marker of T cell receptor (TCR)-mediated signaling, which distinguishes tissue-resident from circulating populations. Functionally, the majority of tissue-resident T cells were quiescent or IL-2-producing memory CD4+ T cells, followed by IFN-γ-producing memory CD8+ T cells, with IL-17 production confined to memory CD4+ T cells in mucosal compartments.

### T Cell Differentiation

T cells acquire their functional properties in two main phases. The first occurs in the thymus, as T cells transit through successive stages that install the gene expression programs that will run at steady state. The second phase of differentiation occurs in the periphery after exposure to signals that occur during an immune response. These signals activate accessible but latent sub-routines that are kept in check prior to the initiation of the immune response. Both processes depend on the activity of E protein transcription factors and their antagonists, the Id factors. In many different T cell lineages, E proteins have a context-dependent use; T cell receptor (TCR) signaling and Id3 activity, in collaboration with other extracellular signals, creates those contexts. While TCR signaling is required for peripheral CD4 T cell differentiation, the specific functional pathways accessed in the periphery are very sensitive to the cytokine milieu. In contrast, the progression of T cell precursors into different pathways in the thymus appears to be driven more by TCR signal strength. In both cases, TCR-dependent upregulation of Id3 is important for allowing changes in chromatin remodeling and gene expression that are needed to restrict E protein activity to the appropriate targets [Anderson, MK. Front. Immunol. (2022) 13: doi.org/10.3389/fimmu.2022.956156].

Conventional CD4 T cells emerge from the thymus as “naïve” cells ready for activation. The functional T helper cell differentiation pathways they take upon antigen encounter depends on the types of inflammatory molecules produced during the innate immune response [Id., citing Martinez-Sosa, P. and Mendoza, L. Biosystems (2013) 113 (2): 96-103]. Each T helper cell (T_{H}) subset is dependent on a specific “master regulator” transcription factor that directly induces the effector genes of each program [Id., citing Hirahara, K. et al. J. Allergy Clin. Immunol. (2013) 131 (5): 1276-87]. The T_{H}17 lineage, characterized by secretion of IL-17A, IL-17F, and IL-22, is triggered by the innate response to bacteria and fungi. RORγt (Rorc) is the T_{H}17 master regulator. Viruses and other intracellular pathogens induce differentiation into the T-bet (Tbx21) dependent T_{H}1 pathway, leading to IL-2, TFNα, and IFNγ production. Helminth infection induces the T_{H}2 fate, leading to secretion IL-4, IL-5, and IL-13, under the control of GATA3 [Id., citing Butcher, M J and Zhu, J. Fac. Rev. (2021) 10:30].

Other T_{H} subsets generated in the periphery include Bcl6-driven T-follicular helper cells (T_{FH}) [Id., citing Hatzi, K., et al. J. Exp. Med. (2015) 212 (4): 539-53], specialized for B cell help in the germinal center, and induced T-reg cells, which, like thymic-derived T-regs, depend on FoxP3 [Id., citing Georgiev, P. et al. J. Clin. Immunol. (2019) 39 (7): 623-40]. In addition to playing unique roles in immunity, T_{H} subsets also have pathogenic impacts when dysregulated [Id., citing Nakayama, T. et al. Annu. Rev. Immunol. (2017) 35:53-84]. In general, T_{H}1 and T_{H}17 cells contribute to autoimmune pathology, T_{H}2 cells are largely responsible for allergic reactions, and T-regs inhibit anti-cancer immunity [Id., citing Dobrzanski, MJ. Front. Oncol. (2013) 3:63; Sarkar, T. et al. Curr. Res. Immunol. (2021) 2:132-41]. Most T_{H} subsets retain plasticity after activation, and some can transdifferentiate from one type to another [Id., citing Hirahara, K. et al. J. Allergy Clin. Immunol. (2013) 131 (5): 1276-87]. Additional T_{H} subsets continue to be identified, including T_{H}22, T_{H}9, T_{FH}13, and T_{R}1 cells, suggesting that the networks controlling these effector functions are dynamic, and represent more of a physiological state than a committed fate [Id., citing Plank, M W, et al. J. Immunol. (2017) 198 (5): 2182-90; Schmitt, E. et al. Trends Immunol. (2014) 35 (2): 61-6; Gowthaman, U. et al. Science (2019) 365 (6456): 1-22].

### T Cell Activation

T cell activation is dependent on the interaction of the T cell receptor (TCR)/CD3 complex with its cognate ligand, a peptide bound in the groove of a class I or class II MHC molecule. Full responsiveness of a T cell requires, in addition to receptor engagement, an accessory cell-delivered costimulatory activity, e.g., engagement of CD28 on the T cell by CD80 and/or CD86 on an antigen presenting cell (APC). The soluble product of an activated T lymphocyte is lymphokines (meaning cytokines produced by lymphocytes).

Naïve T cells must initially be activated by dendritic cells (DCs) [Yewdell, J W and B P Dolan. Nature (2011) 471 (73340): 581-82]. During T cell priming, the clonal selection of antigen-specific naïve T lymphocytes is initiated by the engagement of the TCR by its cognate ligand on the surface of antigen-presenting mature dendritic cells. Engagement of the TCR occurs in secondary lymphoid organs during a physical interaction between the naïve T cells and DCs. The stability of these contacts determined the duration of the engagement of the TCR and thereby the intensity of TCR signaling. In naive T lymphocytes, signaling through the TCR occurs during the interaction with DCs presenting the cognate TCR ligand at their surface. Stable DC-T cell interactions are antigen dependent, influenced by chemokines [Scholer, A. et al. Al. Immunity (2008) 28:258-70, citing Molon, B. et al (2005) Nature Immunol. (2005) 6:465-71] and require an active actin DC cytoskeleton [Id., citing Benvenuti, F. Science (2004) 305:1150-3]. In addition, expression of Intercellular Adhesion Molecule-1 (ICAM-1) by mature DCs is required to establish long-lasting DC-T cell contacts [Id.]. The genetic ablation of ICAM-1 prevents the establishment of long-lasting interactions and fails to induce effective memory. T cell priming in the absence of ICAM-1 expression resulted in normal activation, proliferation, and effector cytotoxic T lymphocyte (CTL) generation, but the effector CD8+ T cells produced low amounts of IFN-gamma. CD8+ T cells then were deleted, and the mice became unresponsive to antigenic rechallenge. Furthermore, in ICAM−/− mice, DC-T cell contacts were not stabilized. The authors conclude that ICAM-1 dependent long-lasting DC-T cell interactions during priming are required for the survival of activated CD8+ T cells and the establishment of effective memory [Id.]. Whitmire, J K et al. (J. Immunol. (2007) 179:1190-7 reported that direct IFN-gamma signaling enhances the development of CD8+ T cell memory, suggesting that impaired CD8+ T cell immunity in ICAM−/− mice may be due to reduced IFN-gamma production by CD8+ T cells during the primary response.

### Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments of B cells, the antigen is fragmented into peptides by proteolytic enzymes, and one or more of the generated peptides are loaded into class II MHC molecules, which traffic through this vesicular compartment. The resulting peptide/class II MHC complex is then exported to the B cell surface membrane. T cells with receptors specific for the peptide/class II molecular complex recognize this complex on the B cell surface [Paul, W. E., “Chapter 1: The immune system: an introduction,” Fundamental Immunology, 4th Edition, Ed. Paul, W. E., Lippincott-Raven Publishers, Philadelphia (1999)].

B cell activation depends both on the binding of the T cell through its TCR and on the interaction of the T cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B cell activation. The subsequent events in the B cell response, including proliferation, Ig secretion, and class switching of the Ig class being expressed, either depend on or are enhanced by the actions of T cell-derived cytokines [Paul, W. E., “Chapter 1: The immune system: an introduction,” Fundamental Immunology, 4th Edition, Ed. Paul, W. E., Lippincott-Raven Publishers, Philadelphia, (1999)].

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B cells develop into antibody-producing cells, whereas the T_{H}1 cells are effective inducers of cellular immune responses, involving enhancement of microbicidal activity of monocytes and macrophages, and consequently increased efficiency in lysing microorganisms in intracellular vesicular compartments. Although CD4⁺ T cells with the phenotype of T_{H}2 cells (i.e., IL-4, IL-5, IL-6 and IL-10) are efficient helper cells, T_{H}1 cells also have the capacity to be helpers [Paul, W. E., “Chapter 1: The immune system: an introduction, “Fundamental Immunology, 4th Edition, Ed. Paul, W. E., Lippincott-Raven Publishers, Philadelphia, (1999)].

### T-Memory Cells

Following the recognition and eradication of pathogens through adaptive immune responses, the vast majority (90-95%) of T cells undergo apoptosis with the remaining cells forming a pool of memory T cells, designated central memory T cells (T_{CM}), effector memory T cells (T_{EM}), and resident memory T cells (T_{RM}) [Clark, R. A., “Resident memory T cells in human health and disease”, Sci. Transl. Med., 7, 269rv1, (2015)].

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases [Clark, R. A., “Resident memory T cells in human health and disease”, Sci. Transl. Med., Vol. 7, 269rv1, (2015)].

### Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter [Scwartz, R. H., “T cell anergy”, Annu. Rev. Immunol., Vol. 21:305-334 (2003)] contribute to the downregulation of the immune response. A third mechanism is provided by active suppression of activated T cells by suppressor or regulatory CD4⁺ T (Treg) cells [Reviewed in Kronenberg, M. et al., “Regulation of immunity by self-reactive T cells”, Nature, Vol. 435:598-604 (2005)]. CD4⁺ Tregs that constitutively express the IL-2 receptor alpha (IL-2Rα) chain (CD4⁺CD25⁺) are a naturally occurring T cell subset that are anergic and suppressive [Taams, L. S. et al., “Human anergic/suppressive CD4⁺CD25⁺ T cells: a highly differentiated and apoptosis-prone population”, Eur. J. Immunol. Vol. 31:1122-1131 (2001)]. Depletion of CD4⁺CD25⁺ Tregs results in systemic autoimmune disease in mice. Furthermore, transfer of these Tregs prevents development of autoimmune disease. Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and non-suppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development [Battaglia, M. et al., “Rapamycin promotes expansion of functional CD4⁺CD25⁺Foxp3⁺ regulator T cells of both healthy subjects and type 1 diabetic patients”, J. Immunol., Vol. 177:8338-8347, (2006)].

### Cytotoxic T Lymphocytes (CTLs)

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Innate Lymphoid Cells (ILCs)

Innate lymphoid cells (ILCs) are the innate counterparts of T lymphocytes. They lack adaptive antigen receptors generated by the recombination of genetic elements [Vivier, E. et al. Cell (2018) 174:1054-66, citing Spits, et al. Nat. Rev. Immunol. (2013) 13:145-49; Eberl, G et al. Science (2015) 348: aaa6566; Artis, D. and Spits, H. Nature (2015) 517:293-301]. All ILCs express interleukin-7 receptor-α (IL-7Rα; CD127). ILCs may be activated by signals from other cells around them upon exposure to foreign antigens (including microbes), rather than by being directly activated by foreign antigens. Some ILCs express toll-like receptors (TLRs) that recognize microbes, and the cells may be directly activated by the PAMPs of microbes. However, there have been some reports showing that ILCs express various kinds of receptors for cytokines, danger signals, neuropeptides and lipid mediators that are more dominant than TLRs [Id.].

ILCs are generally thought to be tissue-resident cells that differentiate into mature effector cells in tissues and show minimal movement between organs. Instead, they have functional plasticity that enables them to respond promptly to microenvironmental changes, thereby precluding any need for differentiation and/or migration of new ILC subsets adapted to a new environment. For example, trans-differentiation has been shown between ILC1s and ILC3s [Id., citing Bernink J H, et al. Immunity (2015) 43:146-160, Bernink J H, et al. Nat Immunol (2013) 14:221-229), between ILCs and ILC2 (Id., citing Bal S M, et al. Nat Immunol (2016) 17:636-645; Silver J S, et al. Nat Immunol 2016; 17:626-635; Ohne Y, et al. Nat Immunol (2016) 17:646-655), and between ILC2s and ILC3s (Id. citing Bernink J H, et al. Nat Immunol (2019) 20:992-1003, Golebski K, et al. Nat Commun (2019) 10:2162].

ILCs currently are divided into 3 different subtypes, according to their expression of cytokines and transcription factors: group 1 ILCs (ILC1s), group 2 ILCs (ILC2s), and group 3 ILCs (ILC3s).

ILCs are defined as ILCs that express T box-expressed in T cells (T-bet) and produce interferon (IFN-γ); they include conventional natural killer cells (cNK) and are considered to be involved in anti-viral immunity, like T_{H}1 cells [Orimo, K. et al., Allergy Asthma Immunol. Res. (2020) 19 (3): 381-98].

ICL2s are defined as ILCs that express GATA-binding protein 3 and produce such cytokines as IL-4, IL-5, IL-9, and IL-13, as well as the epidermal growth factor, amphiregulin; like T_{H}2 cells, they are considered to be involved in anti-helminth immunity. (Id.) ILC3s are defined as ILCs that express retinoic acid receptor-related orphan receptor-γt and produce cytokines, such as IL-17A, IL-22 and GM-CSF; they include both natural cytotoxicity receptor (NCR)-ILC3s and NCR+ILC3s, and are considered to be involved in antibacterial immunity, like T_{H}17 cells. (Id.)

In humans, ILC3s are the predominant population in mucosal tissues, including the lung and gut, whereas the proportion of ILC2s is a little higher in the skin compared to mucosal tissues [Id., citing Bal S M, et al. Nat Immunol (2016) 17:636-645]. The proportion of the ILC subsets is influenced by age; although ILC3s are the predominant population in the fetal human lung, their proportion decreases while the proportions of ILC1s and ILC2s increase with age in the adult human lung [Id., citing Bal S M, et al. Nat Immunol (2016) 17:636-645]. There is substantial heterogeneity in each subset of ILCs. Moreover, ILCs show different phenotypes depending on the organ [Id., citing Ricardo-Gonzalez R R, et al. (2018) 19:1093-1099]. For example, although ILC2s from different organs share canonical markers such as GATA3 and IL-7R, expression of IL-33R, IL-25R, and IL-18R1 differs depending on the organ [Id., citing Ricardo-Gonzalez R R, et al. Nat Immunol (2018) 19:1093-1099].

Another ILC subset, called regulatory ILCs (ILCregs), which resemble regulatory T cells (Tregs) and have regulatory functions, has been reported [Id., citing Morita H, et al. Allergy Clin Immunol (2019) 143:2190-2201.e9; Wang S, et al. Cell (2017) 171:201-216.e18; Seehus C R, et al. Nat Commun (2017) 8:1900]. ILCregs produce regulatory cytokines such as IL-10 and/or TGFβ, but they do not express FOXP3, the canonical transcription factor of Tregs. It remains controversial wither ILCregs represents an independent effector subset, or just a temporary state of ILCs.

There is increasing evidence to suggest that like T helper cell subsets, ILC subsets also display a certain degree of plasticity, which enables them to adjust to their microenvironment. Thus, ILC subsets can change their phenotype and functional capacities. For example, although ILC2s from different organs share canonical markers such as GATA3 and IL-7R, expression of IL-33R, IL-25R, and IL-18R1 differs depending on the organ [Id., citing Ricardo-Gonzalez R R, et al. Nat Immunol (2018) 19:1093-1099]. This requires accessible polarizing signals in the tissue in which conversion occurs, together with the expression of cognate cytokine receptors and key transcription factors in the responding ILCs [Vivier, E. et al. Cell (2018) 174:1054-66].

### Natural Killer (NK) Cells

Natural Killer (NK) cells are cytolytic granular lymphocytes found in humans and other mammals. They are characterized by their innate capacity for lytic activity even in the absence of prior immunization to targets [Seaman (2000) “Natural Killer Cells and Natural Killer T Cells.” Arthritis & Rheumatism 43 (6): 1204-1217]. NKs have the morphology of activated cytotoxic T cells, in that they are typically large with an expanded cytoplasm containing granules used in cytotoxicity.

A particular NK cell will typically express two to four inhibitory receptors in addition to an array of activation receptors, and the varied combinations of inhibitory and activating receptors results in a sizeable heterogeneity within an NK population. It is for this reason that NKs are considered to have the ability to respond to a variety of stimuli and to participate in various immune responses under different pathological conditions [Mandal and Viswanathan (2015). “Natural killer cells: In health and disease.” Hematol. Oncol. Stem Cell The. 8 (2): 47-55].

NK cells primarily develop in the bone-marrow, similar to B cells and myeloid origin cells. They have also been found to develop in lymph nodes and the liver. They can be generated from hematopoietic stem cells (HSCs) that show a commitment towards NK lineage, thus generating NK precursors (NKPs), which eventually mature into NKs under the influence of certain transcription factors. Transcription, soluble, and membrane factors involved in the development of NKs include, in the generation phase, Ets-1, Id2, Ikaros, and PU.1; in the maturation of immature NKs, Gata-3, and IRF-2; and in the functional differentiation of matured NKs, CEBP-γ, MEF, and MITF. The cytokine interleukin 15 (IL-15) has been shown to be essential for NK development homeostasis and survival. The cytokine interleukin-2 (IL-2), a peptide derived from T cells, has been implicated in the cytolytic functional maturation of NK cells [Id.].

NKs are typically found circulating in peripheral blood until activated, when they infiltrate into most tissues that contain pathogen-infected or malignant cells. They represent 10% of all cells in the total peripheral blood mononuclear cells (PBMC) population of circulating human lymphocytes. NKs found in secondary lymphoid tissues, such as tonsils, lymph nodes, and the spleen, differ from NKs in peripheral blood in that lymphoid NKs are activated by DCs and secrete certain cytokines such as interferon, which stimulate a more efficient killing response by T cells [Id.].

Invariant Natural Killer T Cells (iNKTs)

Invariant Natural Killer T Cells are an innate-like T cell subset that expresses an invariant T cell receptor (TCR) α-chain and recognizes lipids. iNKT cells secrete a diverse array of cytokines and can influence many types of immune responses. The invariant TCRs of iNKT cells recognize lipids presented by CD1d, a non-polymorphic MHC class I-like antigen-presenting molecule. iNKT cells are self-reactive, their specificity is highly conserved, they have a different developmental pathway in the thymus that includes positive selection by thymocytes instead of by cortical epithelial cells, they respond very rapidly to TCR and/or cytokine signals with an immediate and copious production of cytokines, and the cells are tissue homing and tissue resident [Crosby et al. Nat Rev Immunol. (2018) 18 (9): 559-574].

### Dendritic Cells (DCs)

Dendritic cells are specialized antigen-presenting cells (APCs) that represent the interface between innate and adaptive immunity; they are able to present endogenous and exogenous antigens to T cells in the context of MHC molecules. Four different lineages can be classified as DCs: classical DCs (cDCs) [Briseno, C G et al. Curr Opin. Immunol. (2014) 29:69-781), citing Steinman, R M and Cohn, ZA. J. Ex. Med. (1973) 137:1142-62): plasmacytoid DCs (pDCs), (Id., citing Siegal, F P et al. Science (1999) 284L 1835037; Cella, M. et al. Nature Medicine (1999) 5:919-23), monocyte derived DCs (moDCs) (Randolph, G J et al. Immunity (1999) 11:753-61; Serbina, N V et al. Immunity (2003) 19:59-70; Geissmann, F. et al. Immunity (2003) 19:71-82) and Langerhans cells (Id., citing Schuler, G. and Steinman, RM. J. Exp. Med. (1985) 161:526-46].

### Immune Response

Responses in the immune system may generally be divided into two arms, referred to as “innate immunity” and “adaptive immunity.”

The innate arm of the immune system is a nonspecific fast response to pathogens that are predominantly responsible for an initial inflammatory response via a number of soluble factors, including the complement system and the chemokine/cytokine system; and a number of specialized cell types, including mast cells, macrophages, dendritic cells (DCs), and natural killer cells (NKs).

The adaptive immune arm involves a specific, delayed and longer-lasting response by various types of cells that create long-term immunological memory against a specific antigen. It can be further subdivided into cellular and humoral branches, the former largely mediated by T cells and the latter by B cells. T cells further can be categorized by the expression of CD4+ molecules or the expression of CD8+ molecules, the latter of which allows for the identification of CD8+ cytotoxic T lymphocytes (CTLs).

A third arm of the immune system involves lineage members of the adaptive arm that have effector functions in the innate arm, therefore bridging the gap between the innate and adaptive immune response. These include cells such as γδ T cells and T cells with limited T cell receptor repertoires, such as natural killer T (NKT) cells and mucosal-associated invariant T (MAIT) cells. The third arm will be referred to herein as “innate-like immunity.”

The three arms of immunity do not operate independently of each other but rather work together to elicit effective immune responses. Because the initiation of an adaptive immune response requires some time, innate immunity and innate-like immunity provide the first line of defense during the critical period just after the host's exposure to a pathogen.

Generally speaking, immune responses are initiated by an encounter between an individual and a foreign substance, e.g., an infectious microorganism. The infected individual rapidly responds with both a humoral immune response with the production of antibody molecules specific for the antigenic determinants/epitopes of the immunogen, and a cell mediated immune response with the expansion and differentiation of antigen-specific regulatory and effector T-lymphocytes, including cells that produce cytokines and killer T cells, capable of lysing infected cells. Primary immunization with a given microorganism evokes antibodies and T cells that are specific for the antigenic determinants/epitopes found on that microorganism, but that usually fail to recognize or recognize only poorly antigenic determinants expressed by unrelated microbes [Paul, W. E., “Chapter 1: The immune system: an introduction,” Fundamental Immunology, 4th Edition, Ed. Paul, W. E., Lippincott-Raven Publishers, Philadelphia, (1999), at p. 102].

For an effective immune response to an antigen, antigen presenting cells (APCs) must process and display the antigen in a proper major histocompatibility complex (MHC) context to a T cell, which then will result in T cell stimulation of cytotoxic and helper T cells. Following antigen presentation, successful interaction of co-stimulatory molecules on both APCs and T cells must occur or activation will be aborted.

As a consequence of this initial response, the immunized individual develops a state of immunologic memory. If the same or a closely related microorganism is encountered again, a secondary response ensues. This secondary response generally consists of an antibody response that is more rapid, greater in magnitude and composed of antibodies that bind to the antigen with greater affinity and that are more effective in clearing the microbe from the body, and a similarly enhanced and often more effective T cell response. However, immune responses against infectious agents do not always lead to elimination of the pathogen [Paul, W. E., “Chapter 1: The immune system: an introduction,” Fundamental Immunology, 4th Edition, Ed. Paul, W. E., Lippincott-Raven Publishers, Philadelphia, (1999), at p. 102].

### Immune Tolerance

The immune system is tolerant of self-antigens, i.e., it can discriminate between antigenic determinants expressed on foreign substances, and antigenic determinants expressed by tissues of the host. The capacity of the system to ignore host antigens, referred to as immune tolerance or immunological tolerance, is an active process involving the elimination or inactivation of cells that could recognize self-antigens through immunologic tolerance [Fundamental immunology, 4th Edn, William E. Paul, Ed. Lippincott-Raven Publishers, Philadelphia, (1999), at p. 2].

Immune tolerance is classified into 1) central tolerance or 2) peripheral tolerance depending on where the state is originally induced, i.e., whether it is in the thymus and bone marrow (central) or in other tissues and lymph nodes (peripheral). The biological mechanisms whereby these forms of tolerance are established are distinct, but the resulting effect is similar [Raker V. K. et al., “Tolerogenic Dendritic Cells for Regulatory T Cell Induction in Man”, Front Immunol, Vol., 6 (569): 1-11, (2015)].

Central tolerance, the principal way in which the immune system is educated to discriminate self-molecules from non-self-molecules, is established by deleting autoreactive lymphocyte clones at a point before they mature into fully immunocompetent cells. It occurs during lymphocyte development in the thymus and bone marrow for T and B lymphocytes, respectively [Sprent J. et al., “The thymus and central tolerance”, Philos Trans R Soc Lond B Biol Sci, Vol. 356 (1409): 609-616, (2001)]. In these tissues, maturing lymphocytes are exposed to self-antigens presented by thymic epithelial cells and thymic dendritic cells, or bone marrow cells. Self-antigens are present due to endogenous expression, importation of antigen from peripheral sites via circulating blood, and in the case of thymic stromal cells, expression of proteins of other non-thymic tissues by the action of the transcription factor AIRE [Murphy, Kenneth. Janeway's Immunobiology: 8th ed. Chapter 15: Garland Science. (2012), pp. 611-668]; [Klein L., “Aire gets company for immune tolerance”, Cell, Vol. 163 (4): 794-795, (2015)].

Those lymphocytes that have receptors that bind strongly to self-antigens are removed by means of apoptosis of the autoreactive cells, or by induction of anergy, meaning a state of non-reactivity [Id. at pp. 275-334]. Weakly autoreactive B cells may also remain in a state of immunological inactivity where they do not respond to stimulation of their B cell receptor. Some weakly self-recognizing T cells are alternatively differentiated into natural regulatory T cells (nTreg cells), which act as sentinels in the periphery to lower potential instances of T cell autoreactivity [Id. at pp. 611-668].

The deletion threshold is more stringent for T cells than for B cells since T cells are the main populations of cells that can cause direct tissue damage. Furthermore, it is more advantageous for the organism to let its B cells recognize a wider variety of antigens, so that they can elicit antibodies against a greater diversity of pathogens. Since B cells can only be fully activated after confirmation by more self-restricted T cells that recognize the same antigen, autoreactivity is held greatly in check [Murphy, Kenneth. Janeway's Immunobiology: 8th ed. Chapter 8: Garland Sciences. pp. 275-334].

This process of negative selection ensures that T and B cells that potentially may initiate a potent immune response to the individual's own tissues are destroyed while preserving the ability to recognize foreign antigens. This step in lymphocyte education is detrimental to preventing autoimmunity. Lymphocyte development and education is most active in fetal development, but continues throughout life as immature lymphocytes are generated, slowing as the thymus degenerates and the bone marrow shrinks in the adult life [Murphy, Kenneth. Janeway's Immunobiology: 8th ed. Chapter 8: Garland Sciences. (2012), pp. 275-334]; [Jiang T. T., “Regulatory T cells: new keys for further unlocking the enigma of fetal tolerance and pregnancy complications”, J Immunol., Vol. 192 (11): 4949-4956, (2014)].

Peripheral tolerance develops after T and B cells mature and enter the peripheral tissues and lymph nodes [Murphy, Kenneth. Janeway's Immunobiology: 8th ed. Chapter 8: Garland Sciences. pp. 275-334]. It is set forth by a number of overlapping mechanisms that predominantly involve control at the level of T cells, especially CD4⁺ helper T cells, which orchestrate immune responses and give B cells the confirmatory signals that the B cells need in order to progress to produce antibodies. Inappropriate reactivity toward a normal self-antigen that was not eliminated in the thymus can occur, since the T cells that leave the thymus are relatively but not completely safe. Some will have receptors (TCRs) that can respond to self-antigens that the T cell did not encounter in the thymus [Murphy, Kenneth. Janeway's Immunobiology: 8th ed. Chapter 8: Garland Sciences. (2012), pp. 275-334]. Those self-reactive T cells that escape intra-thymic negative selection in the thymus can inflict cell injury unless they are deleted in the peripheral tissue chiefly by nTreg cells.

CCR7 is a homing receptor important in T, B and dendritic cell migration into secondary lymphoid organs [Forster R. et al., “CCR7 coordinates the primary immune response by establishing functional microenvironments in secondary lymphoid organs”, Cell, Vol. 99:23-33, (1999)]. Multiple roles for CCR7 have been described, [Hopken U. E. et al., “The chemokine receptor CCR7 controls lymph node-dependent cytotoxic T cell priming in alloimmune responses”, Eur J Immunol., Vol. 34:461-470, (2004)], including induction and maintenance of central and peripheral tolerance [Hugues S. et al., “Immunity, 16:169-181, (2002)]. Based on the expression of the two isoforms of CD45 leucocyte, T cells are often characterized as naive and/or effector CD45RA⁺ T cells or memory CD45RO⁺ T cells.

Autoimmune regulator (Aire), usually expressed in thymic medullary epithelial cells, plays a role in immune tolerance by mediating ectopic expression of peripheral self-antigens and mediating the deletion of auto-reactive T cells [Metzger T. C. et al., “Control of central and peripheral tolerance by Aire”, Immunol. Rev. 2011, Vol. 241:89-103, (2011)].

Appropriate reactivity towards certain antigens can also be suppressed by induction of tolerance after repeated exposure. Naïve CD4+ helper T cells differentiate into induced Treg cells (iTreg cells) in the peripheral tissue, or accordingly, in nearby lymphoid tissue (lymph nodes, mucosal-associated lymphoid tissue, etc.). This differentiation is mediated by IL-2 produced upon T cell-activation, and TGF-β from any of a variety of sources, including tolerizing dendritic cells (DCs) or other antigen presenting cells [Curotto de Lafaille et al., “Effective recruitment and retention of older adults in physical activity research: PALS study”, Immunity, Vol. 30 (6): 626-635, (2009)].

### Autoimmunity

Failure to establish immunologic tolerance or unusual presentations of self-antigens that give rise to tissue-damaging immune responses directed against antigenic determinants/epitopes on host molecules often results in autoimmune disease, meaning an illness that occurs when the body's tissues are attacked by its own immune system [Round J. L. et al., “Coordination of tolerogenic immune responses by the commensal microbiota”, J Autoimmun, Vol. 34 (3): 220-225, (2010)]; [Choi J. et al., “The pathogenesis of systemic lupus erythematosus—an update”, Curr Opin Immunol, Vol. 24 (6): 651-657, (2012)].

### Graft-Versus-Host Disease (GVHD) in Adoptive Cell Therapy

Allogeneic hematopoietic stem cell transplantation (alloSCT), a robust form of adoptive cell therapy (ACT) that has been tremendously effective in the treatment of leukemia, is a potentially curative therapy for malignant and nonmalignant disorders of hematologic cells. Δβ T cells in the donor graft that recognize recipients as “non-self” (alloreactive) promote engraftment by attacking host hematopoietic and immune cells, and in the application of alloSCT for neoplastic disease, such T cells can kill malignant blood-lineage cells, mediating a graft-versus-leukemia (GVL) effect. However, alloreactive T cells also can attack normal host tissues, causing graft-versus-host disease (GVHD).

Graft-versus-host disease (GVHD) is caused by host-reactive donor T cells that infiltrate and damage peripheral tissues in recipients after allogeneic hematopoietic stem cell transplantation (allo-HSCT). These organ-infiltrating donor T cells produce a plethora of inflammatory cytokines and cytotoxic molecules to mediate tissue injury. Tissue inflammation frequently emerges despite the concurrent use of immunosuppressive agents targeting systemic T cells; early treatment resistance is common. GVHD syndromes therefore pose a significant threat of morbidity, escalated and prolonged immunosuppressive therapy, organ dysfunction, impaired quality of life, and ultimately an increased risk for mortality.

Acute GVHD (aGVHD) results from an immune-mediated attack on recipient tissue by donor T cells contained in or developed from a graft that recognize and bind their cognate ligands on recipient APCs in combination with essential second signals on the APC surface. The resulting cytolytic T cell response is executed via perforin, granzymes and Fas ligand, with inflammatory cytokines augmenting this response [Sonntag, K. et al. J. Autoimmunity 62 (2015): 55-66].

Graft content in T-cell subsets has been correlated to the risk of aGvHD, in particular naïve CD45RA+CD4+ T cells [Duti, S. et al. J. Immunology (2007) 179:6547-54], CD4-iNKT cells [Chaidos, A. et al. Blood (2012) 119 (21): 5030-36; Coman, T. et al. Oncoimmunology (2018) 7 (1): e1470735; Rubio, M-T et al., Leukemia (2017) 31:903-12, citing Chang, Y J, et al. J. Clin. Immunol. (2009) 29:696-704], naïve and central memory CCR7+CD4+ T cells [Rubio, M-T et al., Leukemia (2017) 31:903-12, citing Yakoub-Agha, I. et al. Leukemia (2006) 20:1557-65], or CD 8 effector memory T cells (CD45RA−CD62L−) [Rubio, M-T et al., Leukemia (2017) 31:903-12, citing Loschi, M. et al. Biol. Blood Marrow Transplant (2015) 21:569-74].

The pathophysiology of chronic GVHD (cGVHD) is less well understood. It is typically an autoimmune-like syndrome developing gradually involving donor-derived T cells primed on APCs. It is thought to involve three main pathological mechanisms: autoantibody production, systemic fibrosis, and defects in thymic function. For example, autoimmune systemic sclerodermatous GVHD was reported in NSG mouse cohorts that were humanized using exclusively CD34+-selected, CD3+-depleted stem cell grafts (hhCD34+NSG) [Sonntag, K. et al. J. Autoimmunity 62 (2015): 55-66].

Although the initiation of the alloreactive T cell response has been well studied, much less is known about how GVHD is established and maintained after early alloreactive T cell activation.

T cell infiltration into secondary lymphoid organs and non-lymphoid tissues is central to T cell function and occurs during homeostatic tissue surveillance, as well as during T cell-driven immunopathology (including auto- and allo-immune disorders) and T cell-mediated anti-tumor immune attack. Organ infiltration by donor T cells is critical to the development of acute graft-versus-host disease (aGVHD) in recipients after allogeneic hematopoietic stem cell transplant (allo-HCT). During aGVHD, donor T cells first populate secondary lymphoid organs, where they undergo allo-antigen priming, and then home towards and infiltrate non-lymphoid GVHD-target organs. Upon infiltration, these cells induce the immunologic and clinical manifestations of aGVHD, including wide-spread organ damage.

Persisting alloreactive donor T cells in target tissues are a determinant of graft-versus-host disease (GVHD). While tissue-infiltrating donor T cells are initially activated in the secondary lymphoid organs, they are expanded and maintained locally within the resident tissues during the effector phase.

Once established, GVHD is largely maintained locally in tissues by a TCF-1+ progenitor-like T cell population. These tissue-infiltrating donor T cells express a tissue-resident memory-like phenotype, and may be locally maintained by TCF-1+ progenitor-like T cells. The transcriptional regulators that control the persistence and function of tissue-infiltrating T cells are not understood.

Allogeneic hematopoietic cell transplantation (allo-HCT) is a potentially curative therapy for malignant hematological disease. However, its application is impeded by complications including graft-versus-host disease (GVHD) and long-term anti-GVHD treatment-associated aggressive immunosuppression and drug adverse effects [Blazar, B. R., et al., (2020). Nat Rev Clin Oncol 17, 475-492, Zeiser, R., and Blazar, B. R. (2017). N Engl J Med 377, 2565-2579, Zeiser, R., and Blazar, B. R. (2017). 377, 2167-2179]. Live cell therapy for immune regulation, such as infusion of regulatory T cells (Tregs) and mesenchymal stem cells (MSCs), can preferentially target inflammatory cells and provide a more nuanced regulatory activity.

While these traits make MSCs attractive candidates for the treatment of immune-related disorders like autoimmune diseases, acute graft-versus-host disease (aGvHD), and sepsis, their modulatory action strongly depends on the environmental stimuli [Muller, L. et al. Front. Cell Dev. Biol. (2021) 9:637725, citing (Wang, L-T. et al. J. Biomed Sci. (2016) 23:76]. It has been shown that under certain conditions, MSCs can promote immune responses by secreting proinflammatory cytokines and acting as antigen-presenting cells. Their immunostimulatory capabilities can be converted into an immunosuppressive phenotype by a process termed “licensing.” This phenotypic and functional shift is mediated by inflammatory cytokines such as interferon (IFN)-γ or tumor necrosis factor (TNF)-α [Id., citing (Krampera, M. Leukemia (2011) 25:1408-1414].

Novel approaches to produce immune suppressive cells that can persist and sustain immune modulatory function in vivo after infusion remain an unmet need.

Inadequate purification and ex vivo expansion of Tregs and MSCs could reduce their suppressive effects. Moreover, the limited capacity of Tregs and MSCs to persist in vivo after infusion has been another major limitation for their application [Doglio, M., et al., (2022). Front Immunol 13, 1045168, Hefazi, M., Bolivar-Wagers, S., and Blazar, B. R. (2021). Int J Mol Sci 22, Voermans, C., and Hazenberg, M. D. (2020). Blood 136, 410-417, Blazar, B. R., et al., (2018). Blood 131, 2651-2660, Mamo, T., et al., (2022). Transfusion 62, 904-915].

Plasmacytoid dendritic cells (pDCs) could be used for reducing GVHD [Li, B., et al., (2021). Transplant Cell Ther 27, 611 e611-611 e612, Tian, Y., et al. (2021). J Clin Invest 131, Waller, E. K., et al. (2014). J Clin Oncol 32, 2365-2372]. pDCs specialize in rapidly producing IFN-α, protecting against pathogenic infection. They also play important roles in regulating both immune protection and tolerance and can suppress antigen-driven T cell responses and allo-immunity [Fearnley, D. B., et al., (1999). Blood 93, 728-736, Vakkila, J., et al., (2005). Bone Marrow Transplant 35, 501-507, Tel, J., et al. (2013) Cancer Res 73, 1063-1075, Devi, K. S., and Anandasabapathy, N. (2017). Semin Immunopathol 39, 137-152, Audiger, C., et al. (2017). Journal of immunology 198, 2223-2231, Ganguly, D., et al., (2013). Nat Rev Immunol 13, 566-577].

In allogeneic hematopoietic stem cell transplant (allo-HCT) mice, transfer of donor pDCs early, after allo-HCT, reduces GVHD and retains potent graft-versus-leukemia (GVL) activity [Tian, Y., et al. (2021). J Clin Invest 131). This suppressive effect of murine pDCs is associated with immune modulatory effects of IFN-α and PD-L1 [Tian, Y., et al. (2021). J Clin Invest 131, Reizis, B. (2019). Immunity 50, 37-50, Alculumbre, S. G., et al. (2018). Nat Immunol 19, 63-75].

However, whether human pDCs may directly suppress proliferation and survival of activated human T cells has yet to be determined. Furthermore, human pDCs are a rare population for enrichment, presenting a major challenge in meeting the therapeutic needs of patients. It is reasoned that if pDC-derived immune suppressive molecules, specifically IFN-α and PD-L1, can be delivered by genome modified human T cells, a new live cell therapy can be developed to inhibit GVHD.

The present disclosure addresses cell therapy approaches to modulation of auto- and allo-immune disorders including the problem of reducing risk of GVHD while retaining GVT activity in allogeneic cell therapies without relying on pDCs. It provides IFN-α2 and PD-L1 engineered human T cells, termed αρ-T cells, that can sustain immune modulatory function in vivo and that suppress GVHD while retaining GVT activity and reduce autoimmune reactivity in mouse models.

### SUMMARY OF THE INVENTION

According to one aspect, the present disclosure provides a cell therapy method comprising: (a) genetically engineering a population of T cells derived from a healthy donor to overexpress PD-L1 and IFN-α2, thereby generating a population of αρ-T cells, wherein the genetically engineered population of αρ-T cells is characterized by expansion in vitro, persistence in vivo, capacity to infiltrate target tissues, and sustained immune modulatory function; and administering a hematopoietic cell transplant comprising the genetically engineered population of αρ-T cells to a recipient subject in need thereof, wherein the recipient subject in need thereof is afflicted with an autoimmune or alloimmune disorder; wherein the administering of the population of T cells derived from a healthy donor genetically engineered to overexpress PD-L1 and IFN-α2 (αρ-T cells) is by intravenous infusion.

According to some embodiments, the genetic engineering comprises: (a) transducing T cells from a healthy donor with a lentivirus vector encoding PD-L1 and IFN-α2; (b) isolating PD-L1 and IFN-α2-expressing T cells using fluorescence activated cell sorting or magnetic beads to form a purified population of PD-L1, IFN-α2 expressing αρ-T cells; (c) expanding the purified population of αρ-T cells by culturing; and (d) activating the expanded purified population of αρ-T cells.

According to some embodiments, the population of αρ-T cells is allogeneic or autologous to the recipient patient.

According to some embodiments, the recipient subject is a mammal. According to some embodiments, the mammal is a human. According to some embodiments, when the mammal is a human, the human is a child, a young adult, or an adult.

According to some embodiments, the genetically engineered T cell population comprises genetically engineered CD4+ T cells, genetically engineered CD8+ T cells, or both genetically engineered CD4+ T cells and genetically engineered CD8+ T cells.

According to some embodiments, upregulating expression of IFN-α induced gene programs includes increased expression of IFN-stimulated genes, genes involved in cell migration, genes involved in tissue infiltration, genes encoding inhibitory receptors, genes encoding transcription factors, genes involved in inflammatory response, genes involved in INFα response, genes involved in IFN-γ response, or a combination thereof.

According to some embodiments, the IFN stimulated genes comprise IFI27, IFITM1, and ISG15. According to some embodiments, the genes involved in cell migration and tissue infiltration comprise ITGA1, ITGA3, CCR5, CCR9, α4β7, CXCR3, and CCR7. According to some embodiments, the genes encoding inhibitory receptors comprise HAVCR2, LAG3, ENTPD1, and TIGIT. According to some embodiments, the genes encoding transcription factors comprise PRDM1, TOX, STAT1, and CEBPB. According to some embodiments, the genes involved in inflammatory response, INFα response, and IFN-γ response comprise LAG3, HAVCR2, and TOX.

According to some embodiments, the transplantation of the allogeneic or autologous hematopoietic cells comprising the population of genetically engineered T cells results in increased expression of Src homology 2-containing protein tyrosine phosphatase 2 (pSHP2) compared to a control subject.

According to some embodiments, the administered hematopoietic cell transplant comprising the population of genetically engineered T cells increases frequency of PD-1+CX3CR1+CD4+ conventional T cells in the recipient subject compared to a control subject. According to some embodiments, the increased frequency of PD-1+CX3CR1+CD4+ conventional T cells in the recipient subject is detectable in one or more of peripheral blood, bone marrow, liver, or spleen.

According to some embodiments, the PD-1+CX3CR1+CD4+ conventional T cells are TOX+TCF1− T cells.

According to some embodiments, the recipient subject in need thereof is afflicted with a hematologic cancer. According to some embodiments, the method suppresses graft versus host disease (GVHD), thereby decreasing risk of a graft versus host reaction in the recipient subject while preserving graft versus tumor (GVT) immunity compared to a non-genetically engineered control.

According to some embodiments, the hematologic cancer is a leukemia, a myeloma, or a lymphoma.

According to some embodiments, the leukemia is an acute myeloid leukemia, acute lymphoid leukemia, chronic myelomonocytic leukemia, chronic myeloid leukemia, myelodysplastic syndrome (MDS), or a myeloproliferative neoplasm. According to some embodiments, the lymphoma is a non-Hodgkin's lymphoma.

According to some embodiments, the population of genetically engineered αρ-T cells suppresses GVHD by: (a) upregulating expression of IFN-α induced gene programs in alloreactive non-genetically engineered CD4+ T cells in the administered hematopoietic cell transplant; (b) impairing expansion and survival of alloreactive T cell populations in the administered hematopoietic cell transplant; (c) inducing apoptosis, T cell exhaustion, and T cell anergy of the non-genetically engineered alloreactive T cells in the transplant by PD-L1 ligation of PD-1; (d) promoting differentiation of host-reactive T cells into IFN-γ producing effector cells; or (e) a combination thereof.

According to some embodiments, the exhaustion-like T cells are characterized by a PD-1+TIM3+ phenotype.

According to some embodiments, the administered hematopoietic cell transplant comprising the population of genetically engineered αρ-T cells: (i) decreases clinical signs of ongoing graft-versus-host disease in the recipient subject compared to a control subject; (ii) increases survival of the recipient subject compared to a control subject; (iii) reduces inflammation compared to a control subject; or (iv) a combination thereof.

According to some embodiments, preserving GVT immunity in the recipient subject comprises maintaining the production of cytotoxic effector T cells in the recipient subject.

According to some embodiments, the cell therapy method further comprises administering an additional therapeutic agent.

According to some embodiments, the additional therapeutic agent comprises an immunomodulator, a chemotherapy agent, or a combination thereof.

According to some embodiments, the immunomodulator is ipilimumab (YERVOY®).

According to some embodiments, the recipient subject in need thereof is afflicted with or at risk for an autoimmune disorder or disorder with an autoimmune component; and wherein the administering of hematopoietic cells comprising the population of genetically engineered T cells: (i) suppresses expansion of autoreactive conventional T cells in the recipient subject compared to a control subject; (ii) decreases survival of autoreactive conventional T cells in the recipient subject compared to a control subject; (iii) increases autoreactive conventional T cell differentiation into exhaustion-like T cells in the recipient subject compared to a control subject; or (v) a combination thereof.

According to some embodiments, the method reduces expansion of autoreactive populations of T cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% compared to a control subject.

According to some embodiments, the survival of the autoreactive populations of conventional T cells in the recipient subject is decreased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% compared to a control subject.

According to some embodiments, the cell therapy method further comprises administering an additional therapeutic agent.

According to some embodiments, the additional therapeutic agent comprises alpha-1-antitrypsin, prednisolone, dexamethasone, azathioprine, cyclosporine, tacrolimus, sirolimus, methotrexate, mycophenolate mofetil, adalimumab, infliximab, anakinra, tocilizumab, baricitinib, ruxolitinib, certolizumab, etanercept, golimumab, canakinumab, sarilumab, secukinumab, ustekinumab, abatacept, rituximab, fludarabine, busulfan, melphalan, or belimumab.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustrating that engineering human T cells to co-express IFN-α2 and PD-L1 (i.e., αρ-T cells) renders them effective to inhibit xenogeneic graft-versus-host disease (x-GVHD) without impairing their anti-leukemia effect in immunodeficient mice. The αρ-T cells maintained stable expression of PD-L1 and IFN-α and potent immunosuppressive effects in peripheral tissues during GVHD control. CD4⁺ αρ-T cells activated transcriptional programs that promoted effector differentiation, exhaustion, and proliferation inhibition in both themselves and their treated conventional T (conv-T) cells, ultimately reducing GVHD. The CD4⁺ αρ-T cells created a synergistic regulatory loop that modulated conv-T cell responses in which IFN-α suppressed expansion and survival of activated conv-T cells promoted their differentiation into PD-1⁺TIM3⁺ exhaustion-like T cells and sensitized them to PD-L1-mediated suppression. These regulatory effects of murine CD4⁺ αρ-T cells also reduced GVHD of allogeneic hematopoietic cell transplantation (allo-HCT) in immunocompetent mice.

FIG. 2A, FIG. 2B, FIG. 2C, FIG. 2D, FIG. 2E, FIG. 2F, FIG. 2G, FIG. 2H, FIG. 2I, and FIG. 2K show immune modulatory roles of human pDCs. FIG. 2A shows pDCs that were FACS-sorted from healthy donors (HD) with anti-CD304 microbeads. Flow plots show cell purity before and after cell sorting. FIG. 2B shows sorted pDCs that were activated with a TLR agonist cocktail (TLR4 agonist LPS, 100.0 ng/ml; TLR7/8 agonist R848, 100.0 ng/ml; and TLR9 agonist CpG ODN2216, 5.0 μM) overnight. Dot plots show PD-L1, IFN-α2 and LILRB4 expression on pDCs. The graph of FIG. 2C shows expansion of CD4⁺ T cells 7 days after culture of human CD4⁺ T cells that were cocultured with autologous pDCs in the presence of anti-CD3/28 antibody (Ab) (1.0 μg/ml for each) as indicated. The graph of FIG. 2D shows expansion of CD4⁺ T cells 7 days after culture of human CD4⁺ T cells that were cocultured with autologous pDCs at 1:1 ratio in the presence of anti-CD3/28 Ab, with or without anti-PD-L1, anti-LILRB4 or anti-IFNAR1 neutralizing Ab (10.0 μg/ml). FIG. 2E shows CD4+ T cells that were activated with anti-CD3/28 Ab cultured in the presence of IFN-α2a for 7 days to test their expansion. FIG. 2F shows CD4⁺ T cells that were activated with anti-CD3/28 Ab cultured in the presence of IFN-α2a for 7 days to test their cell death (flow cytometric analysis). FIG. 2G shows histograms displaying expression of PD-L1 and LILRB4 on. K562 cells that were transduced with lentivirus encoding human PD-L1 or LILRB4. FIG. 2H shows graphs showing expansion of CD4⁺ T cells 7 days after coculture with vector control or PD-L1- and LILRB4-overexpressing K562 cells at 1:1 ratio. FIG. 2I shows flow cytometry plots showing Lineage (Lin)⁻HLA-DR⁺ cells DC, CD11c⁺CD123⁻cDCs and CD11c⁻CD123⁺ pDCs and their expression of PD-L1, LILRB4 and IFN-α2. PBMCs from G-CSF mobilized HD (G-CSF HD, n=24) were stimulated with the TLR agonist cocktail overnight. Cells were harvested for flow cytometry analysis. FIG. 2J shows graphs showing the fraction of PD-L1- and IFN-α2-expressing pDCs and cDCs PBMCs from G-CSF mobilized HD (G-CSF HD, n=24) were stimulated with the TLR agonist cocktail overnight. Cells were harvested for flow cytometry analysis. FIG. 2K shows graphs showing pDC and cDC frequencies in grafts for patients grade 0-I GVHD (n=11) and patients with grade II-IV GVHD (n=13). *, P<0.05; **, P<0.01; ***, P<0.001. PBMCs from G-CSF mobilized HD (G-CSF HD, n=24) were stimulated with the TLR agonist cocktail overnight. Cells were harvested for flow cytometry analysis.

FIG. 3A and FIG. 3B show person correlation analysis of PD-L1 and IFN-α expression in human pDCs. FIG. 3A shows the frequency of IFN-α2 on pDCs obtained from PB of healthy donors. FIG. 3B shows the frequence of IFN-α2 on pDCs obtained from G-CSF mobilized PBSC allo-grafts.

FIG. 4A, FIG. 4B, FIG. 4C, FIG. 4D, and FIG. 4E show generation of human αρ-T cells that can inhibit x-GVHD. FIG. 4A shows a schematic structure of the lentiviral vector encoding PD-L1 and IFN-α2a. FIG. 4B shows plots showing PD-L1 and IFN-α2 expression on CD4 v-T or αρ-T cells. Empty lentiviral vector transduced T cells (v-T) and αρ-pLU virus transduced human T cells (both CD4⁺ and CD8⁺) (αρ-T cells) were expanded 7 days and stimulated with PMA and Ionomycin for 6 hours. FIG. 4C is a bar graph showing IFN-α2 level in the supernatant on day 7 from 4 healthy donors. v-T or αρ-T cells that were cultured at 0.5×10⁶/ml overnight and supernatants were harvest for ELISA. FIG. 4D is a bar graph showing the percentage of PD-L1 expression in cultured v-T and αρ-T cells from 4 healthy donors. FIG. 4E is a graph showing the expansion of v-T and αρ-T cells cultured in medium containing human IL-2 (10.0 ng/ml) over time. *, P<0.05; **, P<0.01; ***, P<0.001.

FIG. 5A, FIG. 5B, FIG. 5C, FIG. 5D and FIG. 5E show purified PD-L1+αρ-T cell expansion in vitro and PD-1 signaling in cell subsets. PD-L1+αρ-T cells were sorted on day 5 and continued culturing until day 11 with v-T cells as a control. FIG. 5A shows a histogram depicting purity of PD-L1+αρ-T cells on day 5 after culture. FIG. 5B shows PD-L1+αρ-T cell expansion rate in vitro from day 0 to day 11. FIG. 5C shows a histogram depicting PD-1 expression on v-T cells or PD-L1 positive or negative components from αρ-T cells on day 7 after culture. FIG. 5D shows a histogram depicting pSHP2 expression on v-T cells or PD-L1 positive or negative components from αρ-T cells on day 7 after culture. FIG. 5E shows bar graphs of MFI of pSHP2 expression on T cell subsets. ***, P<0.001.

FIG. 6A, FIG. 6B, FIG. 6C, FIG. 6E, and FIG. 6F show results from NSG mice administered with 10 million TCD-PBMCs at day −7 followed by injection of 10 million v-T or αρ-T cell products at day 0. FIG. 6A shows the PD-L1 expression in v-T and αρ-T cells (mean±SD) before transplantation. FIG. 6B shows the schematic experimental procedure. FIG. 6C shows the overall survival. FIG. 6D shows the clinical score of the animals. FIG. 6E shows tissues that were harvested at study endpoint. Images (H&E stained) were obtained at ×100 magnification. FIG. 6F show graphs that depict the histological score of inflammation in the liver and lung. *, P<0.05; **, P<0.01; ***, P<0.001.

FIG. 7A, FIG. 7B, FIG. 7C, FIG. 7D, FIG. 7E, FIG. 7F, FIG. 7G, FIG. 7H, FIG. 7I, FIG. 7J, FIG. 7K, FIG. 7L, FIG. 7M, FIG. 7N, FIG. 7O, and FIG. 7P show that αρ-T cell treatment reduces xenoreactive T cell responses in x-GVHD mice. Circulating peripheral blood (PB) was collected on day 46 after transplantation for flow cytometric analysis. FIG. 7A shows dot plots of circulating PB that was collected on day 46 after transplantation for flow cytometric analysis, depicting human CD45⁺ cells in the PB (upper panel) and frequencies of CD4⁺ and CD8⁺ T cells within gated CD45⁺ cells (lower panel). FIG. 7B shows cell number of human CD45⁺ cells in the PB. FIG. 7C shows graphs depicting the frequencies of donor cells. FIG. 7D shows total cell number of circulating PB. FIG. 7E shows IFN-γ⁺CD4⁺ T cells of circulating PB. FIG. 7F shows the frequency of IFN-γ⁺CD4⁺ T cells in circulating PB. FIG. 7G shows the viability of donor CD4⁺ T cells. FIG. 7H shows the total number of CD4⁺ T cells in bone marrow (BM), liver and spleen tissues that were harvested at study end time point. FIG. 7I shows IFN-γ⁺CD4⁺ T cells in BM, liver, and spleen tissues that were harvested at study end time point. FIG. 7J shows plots depicting PD-1⁺TIM3⁺ expression of CD4 T cells in the PB, BM, liver and spleen. FIG. 7K shows bar graphs depicting PD-1⁺TIM3⁺ expression of CD4 T cells in the PB, BM, liver and spleen. FIG. 7L shows bar graphs depicting percent cell of adhesion molecular expression on CD4⁺ T cells in vitro prior to transplantation. FIG. 7M shows bar graphs show frequency of adhesion molecule-expressing CD4⁺ T cells derived from GVHD livers of mice received v-T or αρ-T cells 26 days after transplantation. FIG. 7N shows bar graphs show frequency of adhesion molecule-expressing CD4⁺ T cells derived from GVHD spleens of mice received v-T or αρ-T cells 26 days after transplantation. FIG. 7O shows histograms depicting PD-1 (upper panel) and p-SHP2 (lower panel) expression on CD4⁺ T cell subsets. FIG. 7P shows bar graphs depicting pSHP2 expression on CD4⁺ T cell subset derived from liver. *, P<0.05; **, P<0.01; ***, P<0.001.

FIG. 8A, FIG. 8B, FIG. 8C, FIG. 8D and FIG. 8E show the characterization of human αρ-T cells in x-GVHD mice. Blood was collected on day 46. Tissues were collected at study endpoints. FIG. 8A shows representative dot plots showing IFN-g production in CD4+ T cells derived from v-T or αρ-T cell recipients. FIG. 8B shows graphs depicting percentage of Foxp3+CD4+ Tregs in total CD4+ T cells derived from v-T or αρ-T cell recipients. FIG. 8C shows graphs depicting PD-L1 percentage in CD4+ T cells derived from v-T or αρ-T cell recipients. FIG. 8D shows mononuclear cells that were collected and stimulated with PMA and ionomycin for 8 hours from liver and spleen on day 85 after transplantation. FIG. 8E shows a graph depicting IFN-α2 expression in serum on day 85 after transplantation. *, P<0.05; **, P<0.01; ***, P<0.001.

FIG. 9A, FIG. 9B, FIG. 9C, FIG. 9D and FIG. 9D show PD-L1+αρ-T cells displaying increased dividing capacity in vivo. NSG mice were administered with 10 million TCD PBMCs on day −7, followed by injection of 10 million or αρ-T cell products on day 0. Mice were sacrificed for analysis on day 26 after transplantation. FIG. 9A shows representative plots depicting PD-L1 expression from liver. FIG. 9B shows bar graphs depicting percent cell of PD-L1 positive CD4+ T cells in tissues. FIG. 9C shows representative plots depicting BrdU+ cells in CD4+ T cell subsets from liver. FIG. 9D shows bar graphs depicting percent cell of BrdU incorporation in CD4+ T cell subsets. *, P<0.05; **, P<0.01; ***, P<0.001.

FIG. 10A, FIG. 10B, FIG. 10C, FIG. 10D, FIG. 10E, and FIG. 10F show the characteristics of CD4⁺αρ-T cells suppressive function. CD4+PD-L1⁺αρ-T cells and v-T cells were produced and FACS-sorted at day 12 of culture for bulk RNA-seq analysis after stimulated with PMA and ionomycin for 2 hours. FIG. 10A shows bar plots depicting GSEA enrichment analysis. FIG. 10B shows GSEA analysis depicting featured pathways. FIG. 10C shows a heatmap depicting the expression of featured differential expressed genes (DEGs). FIG. 10D shows a heatmap depicting the expression of featured differential expressed genes (DEGs). FIG. 10E shows FACS-sorted day 12 PD-L1⁺αρ-T cells and v-T cells that were re-cultured in the presence of anti-CD3/28 Ab for an additional 10 days. Plots and graphs show the percentage of PD1⁺TIM3⁺ cells. FIG. 10F shows FACS-sorted day 12 PD-L1⁺αρ-T cells and v-T cells that were re-cultured in the presence of anti-CD3/28 Ab for an additional 10 days. Graph shows cell viability. *, P<0.05; **, P<0.01; ***, P<0.001.

FIG. 11A, FIG. 11B, FIG. 11C, and FIG. 11D show GFP-expressing CD4⁺αρ-T cells or GFP-expressing CD4+v-T cells that were FACS-sorted 12 days after production, stimulated with PMA+ionomycin for additional 2 hrs, added to autologous non-transduced CD4⁺ conv-T cells labeled with CellTrace™ violet (CTV) at varying ratios, activated with anti-CD3 Ab (1.0 μg/ml) and anti-CD28 Ab (50.0 ng/ml) and cultured for additional 5 days. FIG. 11A shows a graph depicting the expansion of CD4⁺ conv-T cells. FIG. 11B shows plots depicting CTV dilution. FIG. 11C shows graphs depicting CTV dilution. FIG. 11D shows CD4⁺ conv-T cells that were collected 8 days after cultured with CD4⁺αρ-T cells or GFP-expressing CD4⁺v-T cells, restimulated with anti-CD3 (1.0 μg/ml) for additional 6 hrs to measure their viability. *, P<0.05; **, P<0.01; ***, P<0.001.

FIG. 12A, FIG. 12B, FIG. 12C, and FIG. 12D show αρ-T cells suppress autologous primary CD8+ conv-T cells. GFP-expressing CD4+αρ-T cells or GFP-expressing CD8+v-T cells were FACS-sorted 12 days after production, stimulated with PMA+Ionomycin for additional 2 hours, added to autologous CD8+ conv-T cell labeled with CellTrace™ violet (CTV) at varying ratios, activated with anti-CD3 Ab (1 μg/mL) and anti-CD28 Ab (50 ng/ml) and cultured for an additional 5 days. FIG. 12A shows the recovery rates of cultured Cd8+ conv-T cells on day 5 of culture. FIG. 12B shows a plot depicting CellTrace™ dilution on day 5 of culture. FIG. 12C shows a graph depicting CellTrace™ dilution on day 5 of culture. FIG. 12D shows the fraction of PD-1+TIM3+ cells in CD8+ conv-T cells. **, P<0.01; ***, P<0.001.

FIG. 13A, FIG. 13B, and FIG. 13C show PD-L1⁻CD4⁺ conv-T cells that were FACS-sorted 5 days after cultured with CD4⁺αρ-T cells and CD4⁺v-T cells, respectively, and stimulated with PMA and ionomycin for 2 hours for bulk RNA-seq analysis. FIG. 13A shows a Gene Set Enrichment Analysis (GSEA) bar plot for Hallmark pathways. FIG. 13B shows a heatmap depicting expression of representative differentially expressed genes (DEGs). FIG. 13C shows a heatmap depicting expression of additional representative DEGs. *, P<0.05; **, P<0.01; ***, P<0.001.

FIG. 14A, FIG. 14B, FIG. 14C, and FIG. 14D show the effects of a neutralizing Ab against PD-1 or IFNAR1 that was added into the CD4⁺αρ-T cells and CVT labeled CD4⁺ conv-T cell coculture system from day 0-day 5. FIG. 14A shows recovery rate of conv-T cells in each group. FIG. 14B shows cell death of conv-T cells in each group. FIG. 14C shows bar graphs depicting RNA levels of ISG15 expression on conv-T cells. Dividing conv-T cells were sorted on day 5 after coculture. FIG. 14D shows IL-2 expression on conv-T cells. Dividing conv-T cells were sorted on day 5 after coculture. *, P<0.05; **, P<0.01; ***, P<0.001.

FIG. 15A, FIG. 15B, FIG. 15C, FIG. 15D, and FIG. 15E show that αρ-T cells produce a synergistic reinforcing regulatory loop to repress activated conv-T cell responses. FIG. 15A is a schematic of the experimental design showing that equal numbers of CD4+PD-L1⁺αρ-T cells plus CD4+ conv-T cells were injected into hu-NSG mice to induce GVH reaction, with transfer of CD4+ conv-T cells as control. FIG. 15B shows flow cytometric plots of donor T cells in the PB from NSG mice 46 days after transplantation. FIG. 15C shows flow cytometric graphs of donor T cells in the PB from NSG mice 46 days after transplantation. FIG. 15D shows expression of PD-1 and TIM3 on Donor T cells that were isolated from BM, liver and spleen. FIG. 15E shows expression of TOX and EOMES in T cell subsets. *, P<0.05; **, P<0.01; *** P<0.001.

FIG. 16A, FIG. 16B, FIG. 16C, FIG. 16D, FIG. 16E, FIG. 16F, and FIG. 16H show characteristics of PD-1⁻TIM3⁻ cells and PD-1⁺TIM3⁺ cells that were FACS-sorted from v-T cells at day 12 of culture, and re-cultured in the presence of anti-CD3 Ab with or without adding IFN-α2a (10.0 ng/ml), K562 or PD-L1⁺K562 for additional 7 days. FIG. 16A shows the graphic summary of the experiment design. FIG. 16B shows frequencies of PD-1⁺TIM3⁺ cells. FIG. 16C shows the recovery rate of PD-1⁺TIM3⁺ cells. FIG. 16D shows IFN-γ expression percentage of PD-1⁺TIM3⁺ cells. FIG. 16E shows frequencies of cell death in IFN-α2a treated cell subsets generated from PD-1⁻TIM3⁻ cells. FIG. 16F shows the recovery rate of PD-1⁺TIM3⁺ cells. FIG. 16G shows the frequency of cell death of PD-1⁺TIM3⁺ cells. FIG. 16H shows the IFN-γ expression percentage of PD1+TIM3+CD4 and the recovery rate of IFN-γ⁺PD-1⁺TIM3⁺. *, P<0.05; **, P<0.01; ***, P<0.001.

FIG. 17A and FIG. 17B show that murine CD4⁺αρ-T cells reduced GVHD and T cell response in vivo. FIG. 17A shows the retroviral vector structure encoding murine PD-L1 and IFN-α (αρ-MigR1, upper panel). Murine CD4⁺ T cells were activated with anti-CD3/28 Ab and infected with αρ-MigR1 retrovirus. PD-L1⁺αρ-T cells were sorted by FACS (lower panel). FIG. 17B shows a schematic of the experimental procedure. *, P<0.05; **, P<0.01; ***, P<0.001.

FIG. 18A and FIG. 18B show survival and clinical score of Balb/c recipients that were irradiated with 8.5Gy and transplanted with 5×10⁶ B6xB6/SJL F1 (CD45.1⁺CD45.2⁺) TCD BM (TCD-BM, n=7), together with 0.5×10⁶ FACS-sorted B6/SJL (CD45.1⁺) PD-L1⁺CD4⁺αρ-T cells (αρ-T, n=12), 0.2×10⁶ B6 (CD45.2⁺) CD4⁺ T_{N} cells (T_{N}, n=14), or 0.2×10⁶ CD4⁺ T_{N} cells+0.5×10⁶ PDL1⁺CD4⁺αρ-T cells (αρ-T+T_{N}, n=15). FIG. 18A shows survival. FIG. 18B shows clinical score. *, P<0.05; **, P<0.01; ***, P<0.001.

FIG. 19A, FIG. 19B, FIG. 19C, FIG. 19D, FIG. 19E, FIG. 19F, and FIG. 19G show an analysis of blood collected on day 14 and tissues collected on day 21 after transplantation. FIG. 19A shows the number of donor CD4+ T cells in the peripheral blood (PB). FIG. 19B shows GVHD target tissues. FIG. 19C shows IFN-γ+ cytokine production in donor CD4+ T cells. FIG. 19D shows IL2+ cytokine production in donor CD4+ T cells. FIG. 19E depicts a plot showing the frequency of PD-1⁺CX3CR1⁺CD4⁺ conv-T cells and frequency of TOX⁺TCF1⁻cells within the PD-1⁺CX3CR1⁺CD4⁺ conv-T cell population. FIG. 19F shows a graph showing the frequency of PD-1⁺CX3CR1⁺CD4⁺ conv-T cells within the PD-1⁺CX3CR1⁺ CD4⁺ conv-T cell population. FIG. 19G shows a graph showing the frequency of TOX⁺TCF1⁻cells within the PD-1⁺CX3CR1⁺CD4⁺ conv-T cell population. *, P<0.05; **, P<0.01; ***, P<0.001.

FIG. 20A, FIG. 20B, FIG. 20C, and FIG. 20D show murine CD4+αρ-T cells reduced GVHD and T cell response in vivo. Balb/c recipients were irradiated with 8.5Gy and transplanted with 5×10⁶ B6xB6/SJL F1 (CD45.1+CD45.2+) TCD BM (TCD-BM, n=7), together with 0.5×10⁶ FACS-sorted B6/SJL (CD45.1+) PD-L1+CD4+αρ-T cells plus 0.2×10⁶ B6 (CD45.2+) CD4+ T_{N} cells (αρ-T+T_{N}, n=6), or (0.2×10⁶ CD4+ T_{N} cells (T_{N}, n=6). FIG. 20A shows cytokine expression of blood collected on day 14. FIG. 20B shows cytokine expression of blood collected on day 14. FIG. 20C shows PD-1+CX3CR1+ cell percentage on CD45.2+ T_{N} derived conv-T cells. FIG. 20D shows histograms depicting the fraction of molecules critical for T cell migration on CD4+αρ-T cells derived from spleen. *, P<0.05; **, P<0.01; ***, P<0.001.

FIG. 21A and FIG. 21D show αρ-T cell administration suppressed the onset of GVHD. FIG. 21A shows a schematic depicting the experimental procedure. NSG mice were administered with 10 million TCD PBMCs at day −7, followed by injection of 10 million v-T cell products on day 0. Sorted PD-L1+αρ-T cells were administered on day 30. FIG. 21B shows photos of mice with or without αρ-T cell treatment. FIG. 21C shows clinical scores over time after transplantation. FIG. 21D shows overall survival of animals. *, P<0.05; **, P<0.01.

FIG. 22A, FIG. 22B, and FIG. 22C show survival and clinical score of Balb/c recipients that were irradiated with 8.5 Gy and transplanted with 5×10⁶ B6 TCD-BM together with 0.2×10⁶ B6 CD4+ T_{N} cells to induce GVHD. FACS sorted 0.5×10⁶ PD-L1+CD4+αρ-T cells were administered on day 7. FIG. 22A shows a schematic depicting the experimental procedure. FIG. 22B shows percent survival over time after transplantation. FIG. 22C shows clinical score over time after transplantation. *, P<0.05; **, P<0.01.

FIGS. 23A, FIG. 23B, FIG. 23C, FIG. 23D, FIG. 23E, and FIG. 23F show that human αρ-T cell treatment preserves GVT effects in human xenograft leukemia-bearing NSG mice. FIG. 23A shows the schematic experimental procedure. NSG mice were administrated of 10×10⁶ TCD-PBMCs at day −7, followed by injection of 1.0×10⁵ Raji^{TGL} cells together with 10×10⁶ autologous v-T cells, or 10×10⁶ autologous v-T cells+8×10⁶ αρ-T cells at day 0. FIG. 23B shows representative in vivo imaging at different time points. FIG. 23C shows quantification of photon flux over time. FIG. 23D shows Kaplan-Meier survival analysis. FIG. 23E shows GVHD clinical scores. FIG. 23F shows a summary table of leukemia- or GVHD-related mortality. *, P<0.05.

FIG. 24A, FIG. 24B, FIG. 24C, and FIG. 24D show results when NSG mice were administered 10×10⁶ TCD-PBMCs at day −7, followed by injection of 10⁶ MV4-11^{TGL} cells together with 10×10⁶ autologous v-T cells, or 10×10⁶ autologous v-T cells+8×10⁶ αρ-T cells at day 0. FIG. 24A shows a schematic of the experimental procedure. FIG. 24B shows Kaplan-Meier survival analysis. FIG. 24C shows representative in vivo imaging at different time points. FIG. 24D shows quantification of photon flux over time. *, P<0.05.

FIG. 25A, FIG. 25B, FIG. 25C, FIG. 25D, FIG. 25E, FIG. 25F, and FIG. 25G show that αρ-T cells are not affected by immunosuppressive agents and retain antiviral function in vitro. FIG. 25A shows conv-T cells that were activated with plate-bound anti-CD3 (1.0 μg/mL) and anti-CD28 (1.0 μg/mL). Bar graphs show recovery rate of conv-T cells treated with various doses of Tacrolimus (Tac). FIG. 25B shows PD-L1+αρ-T cells that were sorted on day 5 and treated with Tac (25 ng/ml) from day 5 to day 10. Bar graphs show recovery of αρ-T cells. FIG. 25C shows αρ-T cell that were treated with or without Tac and were co-cultured with conv-T cells, activated with anti-CD3 (1 μg/mL) and anti-CD28 (50 ng/ml), and cultured for 5 additional days. Bar graphs show recovery of conv-T cells. FIG. 25D shows representative plots depicting IFN-γ expression in CD8 T cells. FIG. 25E shows representative graphs depicting IFN-γ expression in CD8 T cells. FIG. 25F shows representative plots showing CD62L+CD27+ memory subsets among IFN-γ⁺CD8 T cells. FIG. 25G shows representative graphs showing CD62L+CD27+ memory subsets among IFN-γ+CD8 T cells. *, P<0.05; **, P<0.01; ***, P<0.001.

FIG. 26 is an illustration depicting the Ras-MAPK signaling pathway. Activation of the pathway begins when a signal binds to a protein tyrosine kinase receptor. The epidermic growth factor receptor (EGFR) and the platelet-derived growth factor receptor (PDGFR) are the best-known receptors in the pathway. However, multiple upstream receptors including other receptor tyrosine kinases, integrins, serpentine receptors, heterotrimeric G-proteins and cytokine receptors are able to activate K-ras. Binding of a ligand to EGF receptor induces oligomerization of the receptor, a process that results in juxtaposition of the cytoplasmic, catalytic domains in a manner that allows activation of the kinase activity and transphosphorylation. Adaptor proteins such as Grb2 can recognize sequence homology 2 (SH2) domains such as Shc, which in turn, recruit guanine nucleotide exchange factors (GEFs) like SOS-1 or CDC25 to the cell membrane [taken from The Ras/Raf/MAPK Pathway. Molina, Julian R. et al. Journal of Thoracic Oncology (2006) Volume 1, Issue 1, 7-9].

FIG. 27A and FIG. 27B are illustrations that show the JAK-STAT signaling pathway. FIG. 27A is schematic of the signaling mechanism of the JAK/STAT pathway. When cytokines bind to transmembrane receptors, receptor-associated JAKs are activated, which then phosphorylate STAT proteins. Activation of STAT proteins forms homo- or heterodimers that are transferred to the nucleus and regulate gene transcription. The JAK/STAT pathway is negatively regulated through SOCS and PIAS [taken from Hu et al., (2023) Front. Bioeng. Biotechnol. Vol. 11-2023]. FIG. 27B is a schematic of the intracellular signaling crosstalk of the JAK/STAT pathway. Different signaling proteins activate different STATs and induce the transcription and expression of genes for different cellular functions, including cell cycle, apoptosis, cell proliferation, epithelial-mesenchymal transition (EMT), angiogenesis, inflammatory factor production, etc, which in turn are involved in the development of various diseases [taken from Hu et al., (2023) Front. Bioeng. Biotechnol. Vol. 11-2023].

### DETAILED DESCRIPTION OF THE INVENTION

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

The term “about” is used herein to mean within the typical ranges of tolerances in the art. For example, “about” can be understood as about 2 standard deviations from the mean. According to certain embodiments, about means plus or minus 10% of the numerical value of the number with which it is being used. According to certain embodiments, about means plus or minus 5%. When about is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues (e.g. tag and targeting peptides as mentioned herein). “Consisting essentially of” means excluding other components or steps of any essential significance. Thus, a composition consisting essentially of the recited components would not exclude trace contaminants and pharmaceutically acceptable carriers. A polypeptide “consists essentially of” an amino acid sequence when such an amino acid sequence is present with eventually only a few additional amino acid residues. “Consisting of” means excluding more than trace elements of other components or steps. For example, a polypeptide “consists of” an amino acid sequence when the polypeptide does not contain any amino acids but the recited amino acid sequence.

The terms “activate,” “stimulate,” “enhance,” “increase,” and/or “induce” (and like terms) are used interchangeably to refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

The term “acute myeloid leukemia” or “AML” as used herein refers to a malignant disease of the bone marrow in which hematopoietic precursors are arrested in an early stage of development. Most AML subtypes are distinguished from other related blood disorders by the presence of more than 20% blasts in the bone marrow. The underlying pathophysiology in AML consists of a maturational arrest of bone marrow cells in the earliest stages of development. The mechanism of this arrest is under study, but in many cases, it involves the activation or inactivation of genes through chromosomal translocations and other genetic and/or epigenetic abnormalities [Seiter, K., medscape.com/article/197802, updated Dec. 6, 2022, citing Arber, D A et al. Blood (2016) 127 (20): 2391-405; Smith M T et al. IARC Sci. Publ. (2004) 373-92; Ghiaur, G. et al. Semin. Hematol. (2015) 52 (3): 200-6]. This developmental arrest results in two disease processes. First, the production of normal blood cells markedly decreases, which results in varying degrees of anemia, thrombocytopenia, and neutropenia. Second, the rapid proliferation of the abnormal myeloblasts, along with a reduction in their ability to undergo programmed cell death (apoptosis), results in their accumulation in the bone marrow, the blood, and, frequently, the spleen and liver.

The term “adaptive immune response” as used herein refers to an immune response mediated by uniquely specific recognition of a non-self entity by T and B lymphocytes whose activation leads to elimination of the entity and the production of specific memory lymphocytes.

The term “adoptive immunotherapy” also known as “cellular immunotherapy” is a type of immunotherapy in which T cells are given to a recipient patient to help the body fight diseases.

The term “administer” and its various grammatical forms as used herein refers to apply, provide, or give a treatment (e.g., a drug, a medication, a transplantation, a therapy) to a subject. To administer can refer to the act of transplanting cells to a subject.

The term “affliction” as used herein refers to something that causes pain or suffering, such as a disease or illness.

The term “alloantigen” as used herein refers to an antigen from a genetically different individual of the same species.

The term “allogeneic” as used herein refers to a donor and a recipient of different genetic makeup but of the same species.

As used herein, the term an “allogeneic cell” refers to a cell that is not derived from the individual to which the cell is to be administered, that is, it has a genetic constitution different from the recipient individual. An allogeneic cell is generally obtained from the same species as the recipient individual to which the cell is to be administered. For example, the allogeneic cell can be a human cell, as disclosed herein, for administering to a human patient.

The term “allograft” as used herein refers to a transplant of tissue from an allogeneic donor of the same species. Such grafts are invariably rejected unless the recipient is immunosuppressed.

The term “allograft rejection” as used herein refers to the immunologically mediated rejection of grafted tissues or organs from a genetically nonidentical donor. It is due chiefly to recognition of non-self MHC molecules on the graft.

The term “alloreactive” as used herein refers to a strong primary T cell response against allelic variants of major histocompatibility complex (MHC) molecules in a species.

The term “antigen presentation” as used herein, generally refers to the display to a T cell of antigen on the surface of a cell, e.g., in the form of peptide fragments bound to MHC molecules.

The term “anergy” as used herein refers to a state of lymphocyte non-responsiveness to a specific antigen induced by an encounter of the lymphocyte with a cognate antigen under less than optimal conditions, such as in the absence of costimulation.

As used herein, the term “antibody” is used in the broadest sense and encompasses various antibody structures, including but not limited to monoclonal antibodies, polyclonal antibodies, antibody fragments, chimeric antibodies and wholly synthetic antibodies as long as they exhibit the desired antigen-binding activity. In nature, antibodies are serum proteins, the molecules of which possess small areas of their surface that are complementary to small chemical groupings on their targets. These complementary regions (referred to as the antibody combining sites or antigen binding sites) of which there are at least two per whole antibody molecule, and in some types of antibody molecules ten, eight, or in some species as many as 12, may react with their corresponding complementary region on an antigen (the antigenic determinant or epitope) to link several molecules of multivalent antigen together to form a lattice. The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, K and 2; individual molecules of immunoglobulin generally are only one or the other. In mammals, there are five classes of antibodies, IgA, IgD, IgE, IgG, and IgM, each with its own class of heavy chain. All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The term “antigen” as used herein refers to a molecule containing one or more epitopes (either linear, conformational or both) that will stimulate a host's immune-system to make a humoral and/or cellular antigen-specific response. The term is used interchangeably with the terms “immunogen” or “epitope.” Normally, a B cell epitope will include at least about 5 amino acids but can be as small as 3-4 amino acids. A T cell epitope, such as a CTL epitope, will include at least about 7-9 amino acids, and a helper T cell epitope will include at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

As used herein, the term “antigen presenting cell (APC)” refers to a class of cells capable of displaying on its surface (“presenting”) one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen. An APC can be an “artificial APC,” meaning a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

The terms “apoptosis” or “programmed cell death” refer to a highly regulated and active process that contributes to biologic homeostasis comprising a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism. Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways.

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to death domain (DD) containing receptors like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system, and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits inhibitor of apoptosis (IAP) proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome c is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation, can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. [Loberg, R D, et al., J. Biol. Chem. (2002) 277 (44): 41667-673]. It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the anti-apoptotic effects of Akt.

The term “autoimmune disease” as used herein refers to a disease in which the pathology is caused by adaptive immune responses to self-antigens. Exemplary autoimmune disorders include, without limitation: Addison disease; celiac disease, dermatomyositis; graft versus host disease, Graves disease; Hashimoto thyroiditis; inflammatory bowel disease (Crohn disease, ulcerative colitis); multiple sclerosis; myasthenia gravis; pernicious anemia; reactive arthritis; rheumatoid arthritis; Sjogren syndrome; systemic lupus erythematosus; and Type 1 diabetes (TID).

The term “autoimmune reactivity” or “autoreactivity” refers to a process in which the immune system mistakenly attacks the body's own healthy cells and tissues. Under normal conditions, the immune system distinguishes between itself and invaders. However, in autoimmune reactivity, this process fails, leading to the production of self-reactive antibodies and immune cells that cause inflammation and tissue damage. Autoimmune reactivity can lead to autoimmune disorders.

The term “autologous” as used herein refers to being present in or derived from an individual's own tissues.

As used herein, the term an “autologous cell” refers to a cell that is derived from the same individual to which the cell is to be administered, that is, it has a genetic constitution that is the same as the recipient individual.

The terms “B lymphocyte” or “B cell” are used interchangeably to refer to a broad class of lymphocytes, which are precursors of antibody-secreting cells, that express clonally diverse cell surface immunoglobulin (Ig) receptors (BCRs) recognizing specific antigenic epitopes. Mammalian B-cell development encompasses a continuum of stages that begin in primary lymphoid tissue (e.g., human fetal liver and fetal/adult marrow), with subsequent functional maturation in secondary lymphoid tissue (e.g., human lymph nodes and spleen). The functional/protective end point is antibody production by terminally differentiated plasma cells. A mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”) [LeBien, T W & T F Tedder, Blood (2008) 112 (5): 1570-80].

The term “binding” and its various grammatical forms means a lasting attraction between chemical substances.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

As used herein, the term “biomarker” (or “biosignature”) refers to a peptide, protein, nucleic acid, antibody, gene, metabolite, or any other substance used as an indicator of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of treatment or administration regimes). In evaluating potential therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The terms “central lymphoid organs”, “central lymphoid tissues,” and “primary lymphoid organs” as used herein refers to the sites of lymphocyte development. In humans, these are the bone marrow and the thymus. B lymphocytes develop in the bone marrow. T lymphocytes develop within the thymus from bone marrow-derived progenitors.

The term “CD28” as used herein refers to an activating receptor expressed constitutively by T cells that binds to the B7 co-stimulatory molecules present on specialized antigen-presenting cells such as dendritic cells. CD28 is the major co-stimulatory receptor on naïve T cells. It is the founding member of a subfamily of costimulatory molecules characterized by an extracellular variable immunoglobulin-like domain. Other members of the subfamily include ICOS, CD152, PD1, PD1H, and BTLA [Chen, L. and Flies, D. B., Nat Rev Immunol. (2013) 13:227-242]. CD28 is expressed on roughly 80% of human CD4+ T cells and 50% CD8+ T cells. The proportion of CD28 positive T cells in humans declines with age. Although CD28 expression has been identified on other cell lineages, including bone marrow stromal cells, plasma cells, neutrophils, and eosinophils, the functional importance of CD28 on these cells is not completely understood [Gray Parkin, K., et al., J Immunol. (2002) 169:2292-2302; Rozanski, C H et al., J Exp Med. (2011) 208:1435-1446; Venuprasad, K., et al., Eur J Immunol. (2001) 31:1536-1543; Woerly, G. et al., Clin Exp Allergy. (2004) 34:1379-1387].

Ligation of the CD28 receptor on T cells provides a critical second signal alongside T cell receptor (TCR) ligation for naive T cell activation [Esenstein, J H et al, Immunity (2016) 44 (5): 973-988]. CD28 drives critical intracellular biochemical events including unique phosphorylation and transcriptional signaling, metabolism, and the production of key cytokines, chemokines, and survival signals that are essential for long-term expansion and differentiation of T cells [Bluestone, J A et al., Immunity. (2006) 24:233-238; Bour-Jordan, H. et al., Immunol Rev. (2011) 241:180-205; Martin, P J et al., J Immunol. (1986) 136:3282-3287; Weiss, A. et al., J Immunol. (1986) 137:819-825].

The CD28 ligands CD80 and CD86 diverge in their expression patterns, multimeric states, and functionality, adding another layer of complexity to the regulation of CD28 signaling. CD80 is present in predominantly dimeric form on the cell surface whereas CD86 is monomeric [Bhatia, S. et al., Proc. Nat'l Acad. Sci. USA. (2005) 102:15569-155742005]. CD86 is expressed constitutively on antigen presenting cells (APCs) and is rapidly upregulated by innate stimuli of APCs [Lenschow, D J et al., J Immunol. (1994) 153:1990-1997], whereas CD80 is upregulated at later time points [Sharpe, A J and Freeman, G J, Nat Rev Immunol. (2002) 2:116-126]. CD86 may therefore be more important in the initiation of immune responses. CD80 and CD86 are induced by different stimuli in different cell types and they are not interchangeable in function.

The term “CD62L” or “L-selectin” as used herein refers to a marker found on naïve T cells that distinguishes central memory (T_{CM}, CD62L+) from effector memory (T_{EM}, CD62L−) T cells. The regulation of CD62L plays a pivotal role in controlling the traffic of T lymphocytes to and from peripheral lymph nodes. [Yang, S. et al. PLoS One (2011) 6 (7): e22560].

As used herein, the term “cell growth” refers to the process by which cells accumulate mass and increase in physical size. There are many different examples in nature of how cells can grow. In some cases, cell size is proportional to DNA content. For instance, continued DNA replication in the absence of cell division (called “endoreplication”) results in increased cell size. Megakaryoblasts, which mature into granular megakaryocytes, the platelet-producing cells of bone marrow, typically grow this way. By a different strategy, adipocytes can grow to approximately 85 μm to 120 μm, inclusive, by accumulating intracellular lipids. In contrast to endoreplication or lipid accumulation, some terminally differentiated cells, such as neurons and cardiac muscle cells, cease dividing and grow without increasing their DNA content. These cells proportionately increase their macromolecule content (largely protein) to a point necessary to perform their specialized functions. This involves coordination between extracellular cues from nutrients and growth factors and intracellular signaling networks responsible for controlling cellular energy availability and macromolecular synthesis. Perhaps the most tightly regulated cell growth occurs in dividing cells, where cell growth and cell division are clearly separable processes. Dividing cells generally must increase in size with each passage through the cell division cycle to ensure that a consistent average cell size is maintained. For a typical dividing mammalian cell, growth occurs in the G1 phase of the cell cycle and is tightly coordinated with S phase (DNA synthesis) and M phase (mitosis). The combined influence of growth factors, hormones, and nutrient availability provides the external cues for cells to grow [Guertin, D. A., Sabatini, D. M., “Cell Growth,” in The Molecular Basis of Cancer (4^{th} Edn) Mendelsohn, J. et al Eds, Saunders (2015), 179-190].

As used herein, the term “cell proliferation” refers to the process that results in an increase of the number of cells and is defined by the balance between cell divisions and cell loss through cell death or differentiation.

The term “cell sorting” as used herein refers to a process of cell identification and cell selection and subsequent separation of the different cell species. Cells can be sorted by different characteristics such as morphology but also based on markers. For many cell sorting methods, fluorescently labeled antibodies, which only bind to specific cell types or cells in certain stages of cellular development, are applied to identify the cells of interest and thus to distinguish target cells from unwanted cells. Cell sorting is widely used to obtain a homogeneous cell population from mixed cell samples. One of the most commonly used methods in cell sorting is FACS (fluorescence activated cell sorting). This method relies on cell suspensions which contain target cells that have been specifically labeled with a fluorescent dye via antibodies or that express fluorescent proteins that can be detected by a FACS cell sorter. A similar method to FACS is Magnetic Activated Cell Sorting (MACS), where antibody-coated magnetic beads specifically bind to the target cells to be able to separate them with a strong magnet from non-labeled cells. FACS allows use of several markers at once to identify rare cell subsets but is not efficient to collect large absolute numbers of cells. MACS enables the collection of large amounts of cells but typically uses a small number of cell surface markers to do so.

The term “chemokine” as used herein refers to a class of chemotactic cytokines that orchestrate migration and positioning of immune cells within the tissues. Chemokines bind to seven transmembrane G protein-coupled receptors that trigger intracellular signaling that drives cell polarization, adhesion, and migration [Vilgelm, A E and Richmond, A. Front. Immunol. (2019), citing Griffith, J W., et al. Annu. Rev. Immunol. (2014) 32:659-702; Nagarsheth, N., et al. Nat. Rev. Immunol. (2017) 17:559-72]. They are divided into four families based upon structure: CXC, CC, CX3C, and C chemokines. The receptors follow a similar nomenclature system, based upon the family of chemokines to which they bind. In addition, there is a family of atypical chemokine receptors that do not directly couple to G proteins but are reported to have a variety of roles in development, homeostasis, inflammatory disease, infection, and cancer [Id., citing Nibbs, R J, Graham, G J. Nat. Rev. Immunol. (2013) 13:815-29]. Chemokines play an essential role in guiding the migration of both activating and suppressive immune cell types. The continuous migration of immune cells between lymphoid and nonlymphoid organs is a key feature of the immune system, facilitating the distribution of effector cells within nearly all compartments of the body. Reaching their correct position within primary, secondary, or tertiary lymphoid organs is a prerequisite to ensure immune cells' unimpaired differentiation, maturation, and selection, as well as their activation or functional silencing. The superfamilies of chemokines and chemokine receptors are of major importance in guiding immune cells to and within lymphoid and nonlymphoid tissues [Schulz, O., et al. Annu. Rev. Immunol. (2016) 34:203-42]. Most chemokine receptors are transmembrane-spanning heterotrimeric G-protein-coupled receptors [Kohli, K., et al. Cancer Gene Therapy (2022) 29:10-21].

The term “cognate antigen” as used herein refers to an antigen known to be recognized by a given lymphocyte antigen receptor because it was used for the original activation of that lymphocyte.

As used herein, the term “conventional T cell” or “conv-T cell” refers to non-genetically engineered T cells that comprise an alpha-beta T cell receptor (TCR) and are marked by either CD4 or CD8 co-receptors.

The term “costimulation” as used herein refers to the second signal required for completion of lymphocyte activation and prevention of anergy, which is supplied by engagement of CD28 by CD80 and CD86 (T cells) and of CD40 by CD40 Ligand (B cells).

The term “costimulatory molecule” as used herein refers to molecules that are displayed on the cell surface of antigen presenting cells that enhance the activation of a T cell already being stimulated through its TCR. For example, HLA proteins, which present foreign antigen to the T cell receptor, require costimulatory proteins which bind to complementary receptors on the T cell's surface to result in enhanced activation of the T cell. Costimulatory molecules are highly active immunomodulatory proteins that play a critical role in the development and maintenance of an adaptive immune response [Kaufman and Wolchok eds., General Principles of Tumor Immunotherapy, Chpt 5, 67-121 (2007)]. The two signal hypothesis of T cell response involves the interaction between an antigen bound to an HLA molecule and with its cognate T cell receptor (TCR), and an interaction of a co-stimulatory molecule and its ligand. Specialized APCs, which are carriers of a co-stimulatory second signal, are able to activate T cell responses following binding of the HLA molecule with TCR. By contrast, somatic tissues do not express the second signal and thereby induce T cell non-responsiveness [Id.]. Many of the co-stimulatory molecules involved in the two-signal model can be blocked by co-inhibitory molecules that are expressed by normal tissue [Id.].

The term “costimulatory receptor” as used herein refers to a cell surface receptor on naïve lymphocytes through which they receive signals additional to those received through the antigen receptor, and which are necessary for the full activation of the lymphocyte. Examples are CD30 and CD40 on B cells, and CD27 and CD28 on T cells.

The term “CRISPR” (meaning “clustered regularly interspaced short palindromic repeats”)—“Cas” (meaning “CRISPR-associated”) refers to a class of enzymes derived from bacteria used to selectively modify the DNA of living organisms. In brief, CRISPR “spacer” sequences are transcribed into short RNA sequences (“CRISPR RNAs” or “crRNAs”) capable of guiding the system to matching sequences of DNA. When a target DNA is found, Cas9—one of the enzymes produced by the CRISPR system-binds to the DNA and cuts it, shutting the targeted gene off. Since the CRISPR-Cas9 system itself is capable of cutting DNA strands, CRISPRs do not need to be paired with separate cleaving enzymes as other tools do. They can also be matched with tailor-made “guide” RNA (gRNA) sequences designed to lead them to their DNA targets.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines. Non-limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL15-RA, IL-17, IL-18, IL-21, IL-23, TGF-β, IFNγ, GM-CSF, Groα, MCP-1 and TNF-α.

The term “cytotoxic T lymphocytes” (CTLs) as used herein refers to effector CD8+ T cells. Cytotoxic T cells kill by inducing their targets to undergo apoptosis/programmed cell death via extrinsic and intrinsic pathways.

The term “CTLA-4” or “cytotoxic T-lymphocyte-associated antigen 4” or “CD152” as used herein refers to an inhibitory receptor and immune checkpoint that aids in maintaining self-antigen immunity by dampening T cell responses. It is a counter-receptor to costimulatory molecule CD28.

The terms “decrease” and “reduce” and each of their various grammatical forms are used herein to refer to a diminution, a reduction, an attenuation or abatement of the degree, intensity, extent, size, amount, density or number of occurrences, events or characteristics.

The term “dendritic cells” as used herein refers to specialized antigen-presenting cells (APCs) that represent the interface between innate and adaptive immunity; they are able to present endogenous and exogenous antigens to T cells in the context of MHC molecules. Four different lineages can be classified as DCs: classical DCs (cDCs) [Briseno, C G et al. Curr Opin. Immunol. (2014) 29:69-781], citing Steinman, R M and Cohn, ZA. J. Ex. Med. (1973) 137:1142-62]; plasmacytoid DCs (pDCs), [Id., citing Siegal, F P et al. Science (1999) 284L 1835037; Cella, M. et al. Nature Medicine (1999) 5:919-23], monocyte derived DCs (moDCs) [Randolph, G J et al. Immunity (1999) 11:753-61; Serbina, N V et al. Immunity (2003) 19:59-70; Geissmann, F. et al. Immunity (2003) 19:71-82] and Langerhans cells [Id., citing Schuler, G. and Steinman, R M. J. Exp. Med. (1985) 161:526-46].

Myeloid, classical, or conventional DCs (cDCs): The original markers, CD141 and CD1c have limitations as both are induced on cDC and monocyte-derived cells in tissues and in culture. Expression profiling has now provided a suite of more consistent markers that perform well across species, such as CLEC9A, CADM1, BTLA and CD26 for CD141+ myeloid cDC1, and CD2, FcER1 and SIRPA for CD1c+ myeloid cDC2 [Collin, M. and Bigley, V. Immunology (2018) 154 (1): 3-20, citing Guilliams, M. et al. Immunity (2016) 45:669-84; Heidkamp, G F et al. Sci. Immunol. (2016) 1: eaai76777; Granot, T. et al. Immunity (2017) 46:504-15]. The emergence of subsets of cDC2 and the realization that conventional pDC gates include myeloid cDC precursors that also express CD123 and CD303 add additional complexity [Id., citing Yin, X. et al. J. Immunol. (2017) 198:1553-64; Villani, A C et al. Science (2017) 356: eaah4573; See, P. et al. Science (2017) 356: eaag3009].

Plasmacytoid DCs: Plasmacytoid dendritic cells have an eccentric nucleus and prominent endoplasmic reticulum and golgi (resembling a plasma cell) for the production of type I interferon. Unlike myeloid cDC, they do not express the myeloid antigens CD11c, CD33, CD11b or CD13 [Id., citing Dzionek, A. et al. J. Immunol. (2000) 165:6037-46; MacDonald, K P et al. Blood (2002) 100:4512-20; Dzionek, A. et al. J. Exp. Med. (2001) 194:1823-34]. They retain expression of the GMDP markers CD123 (IL-3R) and CD45RA, which are down-regulated when DC progenitors differentiate into myeloid cDC. Like all human DC they express CD4, at a higher level than myeloid cDC [Id., citing Jardine, L. et al. Front. Immunol. (2013) 4:495]. In addition, pDC have an array of surface receptors that are intimately involved in the regulation of their major physiological function, the production of type I interferon. These include the well-known human pDC markers CD303 (CLEC4C; BDCA-2), CD304 (neuropilin; BDCA-4) CD85k (ILT3) and CD85g (ILT7) together with more recently characterized antigens FcER1, BTLA, DR6 (TNFRSF21/CD358) and CD300A [Id., citing Ju, X. et al. Blood (2008) 112:1184-94; Bao, M. et al. Protein Cell (2013) 4:40-52]. Transcriptional profiling has also added the markers FAM129C, CUX2 and GZMB [Id., citing Heidkamp, G F et al. Sci. Immunol. 2016]1: eaai7677]. Several recent papers have described a small subset of CD123+ pDC that express CD2+ [Id., citing Matsui, T. et al. J. Immunol. (2009) 182:6815-23; Bryant, C. et al. Immunol. Cell Biol. (2016) 94:447-57], CD56+ [Id., citing Osaki, Y. et al. PLoS One (2013) 8: e81722; Yu, H. et al. Protein Cell (2015) 6:297-306] or CD5 [Id., citing Zhang, H. et al. Proc. Natl Acad Sci. USA (2017) 114:1988-93]. These cells have a distinct gene expression pattern that overlaps with myeloid cDC and are now known to contain AXL+SIGLEC 6+ myeloid pre-cDC as described above. The two populations do not completely overlap; some AXL-negative pDC appear to express CD2 or CD5 [Id., citing Bryant, C. et al. Immunol. Cell Biol. (2016) 94:447-57; Zhang, H. et al. Proc. Natl Acad. Sci. USA (2017) 114:1988-93] and CD56 cannot be evaluated in the single-cell studies because it was excluded in lineage.

As used herein, the term “derived from” refers to any method of receiving, obtaining, or modifying something from a source origin.

The term “differential expression” as used herein refers a statistically significant difference in expression levels of a gene between two samples or populations of samples. The statistically significant difference in expression can refer to increased gene expression or reduced gene expression. The expression levels can differ, for example, by at least a factor of 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or greater. Differential expression also includes expression of genes in a given sample or population of samples and not in other samples or populations of samples, i.e., the other samples or populations of samples are substantially free of expression in those genes that are expressed in the given sample or population of samples.

The terms “disease” or “disorder” as used herein refer to an impairment of health or a condition of abnormal functioning.

The term “effector cell” as used herein refers to a cell that carries out a final response or function. The main effector cells of the immune system, for example, are activated lymphocytes and phagocytes.

The term “effector functions” as used herein refers to the actions taken by effector cells and antibodies to eliminate foreign entities, and includes, without limitation, cytokine secretion, cytotoxicity, and antibody-mediated clearance.

The term “enzyme linked immunospot” or ELISpot, as used herein refers to a technique that was developed for the detection of secreted proteins, such as cytokines and growth factors. It is performed using a PVDF or nitrocellulose membrane 96-well plate pre-coated with an antibody specific to the secreted protein. Cells are added to the plate and attach to the coated membrane. Cells are then stimulated and the secreted protein binds to the antibody. Next, a detection antibody is added that binds specifically to the bound protein. The resulting antibody complex can be detected either through enzymatic action to produce a colored substrate or with fluorescent tags. The membrane can be analyzed by manually counting the spots or with an automated reader designed for this purpose. Each secreting cell appears as a spot of color or fluorescence.

The term “enrich” as used herein refers to increasing the proportion of a desired substance, for example, to increase the relative frequency of a subtype of cell compared to its natural frequency in a cell population. Positive selection, negative selection, or both are generally considered necessary to any enrichment scheme. Selection methods include, without limitation, magnetic separation and FACS. Regardless of the specific technology used for enrichment, the specific markers used in the selection process are critical, since developmental stages and activation-specific responses can change a cell's antigenic profile.

As used herein, the term “expression” and its other grammatical forms refers to production of an observable phenotype by a gene, usually by directing the synthesis of a protein. It includes the biosynthesis of mRNA, polypeptide biosynthesis, polypeptide activation, e.g., by post-translational modification, or an activation of expression by changing the subcellular location or by recruitment to chromatin.

The term “expression vector” as used herein refers to a cloning vector that encodes functions for the transcription/translation of an inserted fragment of DNA. In addition to an origin or replication and a marker gene, an exemplary expression vector includes a promoter; a ribosome binding site to ensure that following transcription of the insert, the mRNA contains a Shine-Dalgarno sequence needed for binding to the ribosome; a multiple cloning site that permits flexibility in the preparation of the insert; and a transcription terminator to inhibit unwanted readthrough.

The term “eomesodermin” or EOMES” as used herein refers to a transcription factor involved in development and function of certain types of NK cells, ILCs and CD8 T cells.

The term “expand” or “amplify” as used herein with respect to cells refers to increasing in cell number.

The term “flow cytometry” as used herein, refers to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow analysis and differentiation of the cells is based on size, granularity, and whether the cell is carrying fluorescent molecules in the form of either antibodies or dyes. As the cell passes through the laser beam, light is scattered in all directions, and the light scattered in the forward direction at low angles (0.5°-10°, inclusive) from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population [Marion G. Macey, Flow cytometry: principles and applications, Humana Press, 2007]. Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

The terms “formulation” and “composition” are used interchangeably herein to refer to a product of the present disclosure that comprises all active and inert ingredients. The terms “pharmaceutical formulation” or “pharmaceutical composition” as used herein refer to a formulation or composition that is employed to prevent, reduce in intensity, cure or otherwise treat a target condition or disease.

The term “gene set enrichment analysis” or “GSEA” refers to a powerful analytic method for interpreting gene expression data. The method focuses on gene sets, i.e., groups of genes that share a biological function, chromosomal location, or regulation. There are three key elements of the GSEA method: (1) calculation of an enrichment score: an enrichment score reflects the degree to which a gene set is overrepresented at the extremes of the entire ranked list; (2) estimation of significance level of the enrichment score: the estimation of statistical significance is performed using an empirical phenotype-based permutation test procedure that preserves the complex correlation structure of the gene expression data; and (3) adjustment for multiple hypothesis testing: when an entire database of gene sets is evaluation, the estimated significance level is adjusted to account for the size of the set, yielding a normalized enrichment score [A. Subramanian, et al., Proc. Natl. Acad. Sci. U.S.A. 102 (43) 15545-15550].

The term “gene signature” refers to global changes in a group of genes between normal and cancer cells; for example, between healthy and autoimmune subjects. A standard scheme for gene signature construction includes selection of an extended list of candidate genes that can be potentially utilized in a gene signature whose expression can reliably be measured using the selected technology, optionally satisfying additional conditions, e.g., association with a particular pathology or positive expression in a significant fraction of samples. Then a learning set of samples with known clinical annotation is taken, genes are ranked according to their individual informative power and the top genes are taken into further account with possible additional supervised filtration. Finally, a classification algorithm based on the identified gene set is selected and fine-tuned.

The term “healthy subject” or “healthy donor” or “healthy adult donor” or “healthy control” as used herein refers to an individual having no signs or symptoms of a disease, e.g., an autoimmune disease or a cancer.

The term “hematopoietic stem cell” (HSC) as used herein refers to a cell isolated from the blood or from the bone marrow that can renew itself, can differentiate to a variety of specialized cells, can mobilize out of the bone marrow into the circulating blood, and can undergo apoptosis. In nature, HSCs continuously replenish all classes of blood cells through a series of lineage restriction steps that results in the progressive loss of differentiation potential to other lineages. Hematopoietic stem cells derived from human subjects express at least one type of cell surface marker including, without limitation, CD34, CD38, HLA-DR, c-kit, CD59, Sca-1, Thy-1 or a combination thereof. The terms “myeloid” and “lymphoid” refer to the two major branches of hematopoietic stem cells involved in the immune system.

The term “hematopoietic stem cell transplantation” as used herein refers to the process whereby hematopoietic stem and progenitor cells derived from a donor are delivered to a recipient by intravenous infusion in order to restore normal hematopoiesis and/or to treat malignancy.

The term “graft versus host disease” or “GVHD” as used herein refers to an attack on the tissues of a recipient by mature T cells from a nonidentical donor, which can cause a variety of symptoms, sometimes severe.

Classic acute GVHD occurs before day 100 and is staged according to the percentage of body surface area with rash, total bilirubin elevation, and volume of diarrhea. Late acute GVHD occurs after day 100 and is defined as signs and symptoms of acute GVHD without chronic GVHD. Late acute GVHD is further subdivided into “persistent” if it is a continuation of classic acute GVHD, “recurrent” if classic acute GVHD resolves then recurs after day 100, or “de novo” if initial onset is after day 100 without any prior acute GVHD [Lee, S J. Blood (2017) 129 (1): 30-37]. Chronic GVHD is a pleiotropic, multiorgan syndrome involving tissue inflammation and fibrosis that often results in permanent organ dysfunction. [Id.]

Several systems for grading acute GVHD have been developed. The two most popular are the Glucksberg grade (I-IV) and the International Bone Marrow Transplant Registry (IBMTR) grading system (A-D) [Glucksberg, H. et al. Transplantation (194) 18:295; Rowlings, P A et al. Br. J. Haematol. (1977) 97:855]. The severity of acute GVHD is determined by an assessment of the degree of involvement of the skin, liver, and gastrointestinal tract. The stages of individual organ involvement are combined with (Glucksberg) or without (IBMTR) the patient's performance status to produce an overall grade, which has prognostic significance. Grade I (A) GVHD is characterized as mild disease, grade II (B) GVHD as moderate, grade III (C) as severe, and grade IV (D) life-threatening [Przepiorka, D. et al. Bone Marrow Transplant (1995) 15:825; Cahn, J Y et al. Blood (2005) 106:1495].

The IBMTR grading system defines the severity of acute GVHD as follows [Rowlings, P A et al. Br. J. Haematol. (1977) 97:855]:
Grade A—Stage 1 skin involvement alone (maculopapular rash over <25 percent of the body) with no liver or gastrointestinal involvement
Grade B—Stage 2 skin involvement; Stage 1 to 2 gut or liver involvement
Grade C—Stage 3 involvement of any organ system (generalized erythroderma; bilirubin 6.1 to 15.0 mg/dL; diarrhea 1500 to 2000 mL/day)
Grade D—Stage 4 involvement of any organ system (generalized erythroderma with bullous formation; bilirubin >15 mg/dL; diarrhea >2000 mL/day or pain or ileus)

The term “graft versus tumor” or “GVT” effect as used herein refers to immune reactivity mediated by donor T cells against the recipient's tumor cells because the donor's immune system sees the recipient's tumor cells as foreign, a process that relies on genetic differences, specifically the major histocompatibility complex (MHC) system. The GVT effect is a beneficial outcome of allogeneic hematopoietic cell transplantation, although it is often balanced against the risk of graft-versus-host disease. In GVT, donor immune cells, primarily T cells, recognize and attack the recipient's tumor cells, viewing them as “non-self”. The term “graft versus leukemia” or “GVL” as used herein refers to a specific type of GVT effect, wherein the donor immune cells target the recipient's leukemic cells.

As used herein, the term “immune checkpoints” refers to the array of inhibitory pathways necessary for maintaining self-tolerance and that modulate the duration and extent of immune responses to minimize damage to normal tissue. In T cells, the ultimate amplitude and quality of the immune response, which is initiated through antigen recognition by the TCR, is regulated by a balance between co-stimulatory and inhibitory signals (immune checkpoints) [Pardoll, DM. Nat. Rev. Cancer (2012) 12 (4): 252-64]. Immune checkpoint molecules such as PD-1, PD-L1, CTLA-4 are cell surface signaling receptors that play a role in modulating the T-cell response in the tumor microenvironment. Tumor cells have been shown to utilize these checkpoints to their benefit by up-regulating their expression and activity. With the tumor cell's ability to commandeer some immune checkpoint pathways as a mechanism of immune resistance, it has been hypothesized that checkpoint inhibitors that bind to molecules of immune cells to activate or inactivate them may relieve their inhibition of an immune response. Immune checkpoint inhibitors have been reported to block discrete checkpoints in an active host immune response allowing an endogenous anti-cancer immune response to be sustained. Recent discoveries have identified immune checkpoints or targets, like PD-1, PD-L1, PD-L2, CTLA-4, TIGIT, TIM-3, LAG-3, CCR4, OX40, OX40L, IDO, and A2AR, as proteins responsible for immune evasion.

The terms “immune response” and “immune-mediated” are used interchangeably herein to refer to any functional expression of a subject's immune system, against either foreign or self-antigens, whether the consequences of these reactions are beneficial or harmful to the subject. A “primary immune response” is the adaptive immune response that follows the first exposure to a particular antigen. When B and T cells replicate during the primary immune response, they produce T and B effector cells and long-lived antigen-specific memory cells. A “secondary immune response” or “memory recall response” is the immune response that occurs in response to a subsequent exposure to an antigen generated by the reactivation of memory lymphocytes. In comparison with the primary response, it starts sooner after exposure, produces greater levels of antibody, and produces class-switched antibodies. Memory T cells are antigen-experienced cells that mediate a faster and more potent response upon repeat encounter with antigen.

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunological synapse” (“IS”) as used herein refers to a highly structured body that functions to concentrate T cell signaling in a defined area. It is associated with the selective recruitment of signaling molecules and exclusion of negative regulators. The synapse is stabilized by a ring of adhesion molecules, including, for example, LFA1, which binds to ICAM1 on the APC. The immune synapse modulates TCR signaling by several mechanisms. In the earliest stages of immune synapse formation, TCR-containing microclusters are recruited to the central molecular cluster. The TCR responds to two distinct pMHC ligands (agonist and self-peptide-MHC) in co-agonism rather than nonspecific TCR-MHC interactions, which adds to the overall binding strength of the TCR-p-MHC complex. The strength of the TCR-p-MHC interactions has a role in determining the influence of coreceptors that are recruited to the immune synapse (e.g., CD8). The immune synapse also modulates TCR signaling by regulating interactions between kinases in the TCR pathway and their substrates [Morris, G. and Allen, P M. Nature Immunol. (2012) doi: 10.1038/nm.2190]. The T cell signaling pathway includes proximate signaling (including phosphorylation of the invariant signaling protein CD3 and early signaling molecules, calcium-mediated signaling (release of intracellular Ca2+ stores and influx of extracellular Ca2+), and GTPase Ras-MAPK signaling [Id.].

The term “immunological response” to an antigen or composition as used herein is meant to refer to the development in a subject of a humoral and/or a cellular immune response to an antigen. For purposes of the present disclosure, a “humoral immune response” refers to an immune response mediated by antibody molecules, while a “cellular immune response” is one mediated by T-lymphocytes and/or other white blood cells. One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of T cells, suppressor T-cells and/or γδ T-cells directed specifically to an antigen or antigens present in the composition of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immunomodulatory or immune modulatory therapy” as used herein refers to treatments that seek to modify an immune response in a beneficial way, for example to reduce or prevent an undesirable response.

The term “immunosuppression” and its various grammatical forms as used herein refers to a state of temporary or permanent dysfunction of the immune response resulting from insults to the immune system and leading to increased susceptibility to disease and often a suboptimal antibody response. Immunosuppression may be deliberately induced with drugs, as in preparation for bone marrow or other organ transplantation, to prevent rejection of the donor tissue. It may also result from certain diseases.

As used herein, the term “increase” and its various grammatical forms refers to becoming or making greater in size, amount, intensity, or degree, such as a n increase in the number of occurrences, events or characteristics.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

Multiple signaling pathways, such as nuclear factor kappa B (NF-κB), Janus kinase/signal transducers and activators of transcription (JAK-STAT), toll-like receptor (TLR) pathways, cGAS/STING, and mitogen-activated protein kinase (MAPK); inflammatory factors, including cytokines (e.g., interleukin (IL), interferon (IFN), and tumor necrosis factor (TNF)-α), chemokines (e.g., C-C motif chemokine ligands (CCLs) and C-X-C motif chemokine ligands (CXCLs)), growth factors (e.g., vascular endothelial growth factor (VEGF), transforming growth factor (TGF)-β), and inflammasome; as well as inflammatory metabolites including prostaglandins, leukotrienes, thromboxane, and specialized proresolving mediators (SPM), have been identified as regulators of the initiation and resolution of inflammation [Zhao, H. et al. Signal Transduction and Targeted Therapy (2021) 6:263].

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents. The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.′

The term “inflammatory cell infiltrate” as used herein refers to a collection of immune ells, primarily mononuclear cells such as lymphocytes, plasma cells and macrophages, that accumulate in tissue during inflammation.

The term “inhibitor receptor lymphocyte activation gene-3” or “LAG-3” as used herein refers to a member of the immunoglobulin superfamily (IgSF), which binds to major histocompatibility complex (MHC) class II. LAG-3 expression on tumor infiltrating lymphocytes (TILs) is associated with tumor-mediated immune suppression.

The term “innate immune response” as used herein refers to the various mechanisms encountered by a pathogen or transformed cell before adaptive immunity is induced, such as anatomical barriers, antimicrobial peptides, the complement system, and macrophages and neutrophils carrying nonspecific pattern-recognition receptors. Innate immunity is present in all individuals at all times, does not increase with repeated exposure, and discriminates between groups of similar pathogens, rather than responding to a particular pathogen.

The term “innate lymphoid cells” or “ILCs” as used herein refers to a class of innate immune cells having overlapping characteristics with T cells but lacking an antigen receptor. They arise in several groups: ILC1, ILC2, ILC3, and NK cells, which exhibit properties roughly similar to TH1, TH2, TH17 and CD8 T cells. ILC1 express the transcription factor T-BET and produce IFN-γ upon stimulation with IL-12 and/or IL-18. ILC2 are defined by expression of the transcription factor GATA3, express the IL-33 receptor and, in humans, the chemokine receptor of Th2 cells (CRTH2), and produce type II cytokines (mainly IL-5 and IL-13). ILC3 can be discriminated by the expression of the transcription factor retinoic acid orphan receptor (ROR)γt. ILC3 reside in epithelial tissues where they produce their main effector cytokines IL-17A and IL-22. [Bar-Ephraim, Y E et al. J. Immunol. (2019) 202 (1): 171-182].

The term “INKT” as used herein refers to invariant NKT cells, which are a CD1d-restricted T cell population that can respond to lipid antigenic stimulation within minutes by secreting a wide variety of cytokines.

The term “interferon-α receptor (IFNAR) as used herein refers to a receptor that recognizes IFN-α and IFN-β to activate STAT1 and STAT2 and induce expression of many interferon stimulated genes (ISGs).

The term “Interferon regulatory factors” or “IRFs” are a family of master transcription factors, and although IRFs are recognized as transcriptional regulators of type I IFNs (IFN-I) and IFN-inducible genes, this family is now characterized as key factors in the regulation of many different processes, such as immunity, oncogenesis, metabolism, cell differentiation and apoptosis [Ma, W. et al. Front. Immunol. (2023) 14:1236923].

The IRF family has a conserved N-terminal region, and all members possess a helix-turn-helix DNA-binding domain (DBD), which contains five tryptophan repeats and recognizes the core DNA sequence of the 5′-GAAA-3′ tetranucleotide contained within the IFN-stimulated response elements (ISREs). The C-terminal regions of IRFs are diverse and related to distinct functions and contain two types of IRF-associated domains (IADs). The IAD mediates homo and heteromeric interactions with other IRF members, transcription factors, or cofactors to recognize DNA sequences and regulate gene transcription [Id.].

The IFN-regulatory factor (IRF) family of transcription factors includes nine members in mammals that bind to related target-gene sequences [Bollig, N., et al. Proc. Natl Acad. Sci. USA (2012) 109 (22): 8664-9, citing Lohoff, M. and Mak, TW. Nat. Rev. Immunol. (2005) 5:125-35].

In the T lineage, IRF1 is decisive for T_{H}1 cell generation because it is ubiquitously expressed and redundantly addresses many genes with independent T_{H}1-supporting function [Id., citing Lohoff, M. and Mak, TW, Nat. Rev. Immunol. (2005) 5:125-35; Lohoff, M. et al. Immunity (1997) 6:681-9; Taki, S. et al. Immunity (1997) 6:673-9].

IRF4 has been characterized as an important transcription factor for differentiation of T_{H}2, T_{H}9 and T_{H}17 cells [Id., citing Brustle, A. et al. Nat. Immunol. (2007) 8:958-66; Huber, M., et al. Proc. Natl Acad. Sci. USA (2008) 105:20846-51; Lohoff, M., et al. Proc. Natl Acad. Sci. USA (2002) 99:11808-12; Rengarajan, J., et al. J. Exp. Med. (2002) 195:1003-12]. In addition, aspects of Treg cell function entirely depend on IR4 [citing, Zheng, Y., et al. Nature (2009) 458:351-6; Chen, Q., et al. Immunity (2008) 29:899-911; Staudt, V., et al. Immunity (2010) 33:192-202]. Treg-specific IRF4 deficiency or lack of IRF4 binding protein leads to a generalized autoimmune syndrome [Id., citing Zheng, Y., et al. Nature (2009) 458:351-6; Chen, Q., et al. Immunity (2008) 29:899-911]. IRF4 also plays a role in the development of T_{FH} cells, which are mainly responsible for the intricate organization of T-B interactions and antibody maturation in vivo. Irf4−/− Mice fail to generate germinal centers and fail to generate T_{FH} dells. In the B-cell lineage, IRF4 is important for plasma cell differentiation and isotype switching [Id., citing Klein, U., et al. Nat. Immunol. (2006) 7:773-82; Sciammas, R. et al. Immunity (2006) 25:225-36].

IRF7 is a lymphoid-specific factor that is predominantly expressed in the cytoplasm of the spleen, thymus, and peripheral blood lymphocytes, such as B cells, plasmacytoid dendritic cells (pDCs) and monocytes. Although IRF7 was originally identified in Epstein-Barr virus (EBV) infection and characterized as a transcriptional regulator of IFN-I and IFN-stimulated gene (ISG), recent studies have revealed that IRF7 exerts a broad range of activities in different biological processes [Ma, W. et al. Front. Immunol. (2023) 14:1236923].

The regulation of IRF7 is complex and involves many positive and negative feedback mechanisms. The stability of IRF7 can be regulated by various mechanisms at the transcriptional, translational, posttranslational, epigenetic levels, such as phosphorylation, ubiquitination, SUMOylation, acetylation, et al., and the dimerization and nuclear translocation of IRF7. The transcription of IRF7 has two distinct pathways: IFN-triggered and IFN-independent signaling pathways. IFN signals through its receptor to induce the phosphorylation of STAT1 and STAT2, which results in the formation of ISGF3 and then promotes the transcription of IRF7 by binding directly to the IRF7 interferon (IFN)-stimulated response element (ISRE) and IRF-binding element (IRFE), which is IFN-triggered signaling [Id., citing Marie, I. et al. EMBO J. (1998) 17 (220:6660-6669; ato, M. et al. FEBS Lett. (1998) 441 (1): 106-110]. Virus-induced formation of a virus-activated factor complex (formed by IRF7, IRF3 and p300/CREB-binding protein) directly binds to the IRF7 ISRE and IRFE and stimulates the intrinsic transcriptional activity of IRF7, and this induction is independent of the IFN-triggered pathway [Id.]. Some factors can regulate IRF7 transcription positively or negatively.

Phosphorylated RelA and bromodomain containing (BRD) 4 induced by respiratory syncytial virus (RSV) can bind to the IRF7 promoter, triggering the IRF7-RIG-1 amplification loop for IFN-I/III expression [Id.]. Transcription factor nuclear factor of activated T cells (NFATC) 3 selectively binds to the autoinhibitory domain (373-443 aa) of IRF7, promotes transcription and nuclear translocation, and enhances CpG DNA-induced IFN-α production in pDCs [Id.].

FOXO3 is a negative regulator of IRF7 transcription, and the ternary complex consisting of FOXO3, nuclear corepressor 2 (NCOR2) and histone deacetylase 3 (HDAC3) on the Irf7 promoter enhances the closed chromatin structure and represses Irf7 expression and Ifnb 1 production [Id.]. B-cell lymphoma (Bcl), interacting with NCOR2 and HDAC3, binds to IRF7 loci and restrains its transcription [Id.]. Myc forms a repressor complex together with NOCR2 and HDAC3 to reduce the expression of IRF7 through histone deacetylation [Id.]. Activating transcription factor (ATF) 4 inhibits the transcription of IRF7 by regulating its promoter. However, IRF7 increases the expression and function of ATF4 directly, and cross-regulation between IFN and integrated stress responses is mediated by the reverse correlation between ATF4 and IRF7 [Id.].

Upregulation of IFN-I is a hallmark of systemic autoimmune disease (SAD), and the continuous increase in IFN-I/III may be accompanied by clinical manifestations and disease activity [Id.]. As the master regulator of IFN-I/III, IRF7 has a dual role as a protector and cause of autoimmune diseases. In published articles, decreased expression was observed in multiple sclerosis (MS)/experimental autoimmune encephalomyelitis (EAE) and rheumatoid arthritis (RA), while increased expression of IRF7 was observed in patients with systemic lupus erythematosus (SLE), systemic sclerosis (SSc), autoimmune pancreatitis (AIP), autoimmune thyroid diseases (AITD) and diabetes compared to healthy controls [Id., Table 1 and sections 3.2.1 through 3.2.7].

The term “interferon stimulated genes” or “ISGs” as used herein refers to a category of gene induced by interferons, which include many that promote innate defense against pathogens.

The term “interferons” or “IFNs” as used herein refers to several related families of cytokines originally named for their interference of vial replication. IFN-α and IFN-β are antiviral in their effects; IFN-γ has other roles in the immune system.

The term “isolated” is used herein to refer to material, such as, but not limited to, a nucleic acid, peptide, polypeptide, protein, or cell which is: (1) substantially or essentially free from components that normally accompany or interact with it as found in its naturally occurring environment. For example, a naturally occurring polynucleotide or polypeptide present in a living animal is not isolated, but the same polynucleotide or polypeptide, separated from some or all of the coexisting materials in the natural system, is isolated. The terms “substantially free” or “essentially free” are used herein to refer to considerably or significantly free of, or more than about 95% free of, or more than about 99% free of such components, or 100% free of such components. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment.

The term “JAK-STAT pathway”, as used herein refers to the Janus kinase/signal transduction and transcription activation pathway. Janus kinase/signal transduction and transcription activation (JAK/STAT) pathways were originally thought to be intracellular signaling pathways that mediate cytokine signals in mammals. Existing studies show that the JAK/STAT pathway regulates the downstream signaling of numerous membrane proteins such as G-protein-associated receptors, integrins and so on. Mounting evidence shows that the JAK/STAT pathways play an important role in human disease pathology and pharmacological mechanism. The JAK/STAT pathways are related to aspects of all aspects of the immune system function, such as fighting infection, maintaining immune tolerance, strengthening barrier function, and cancer prevention, which are all important factors involved in immune response. In addition, the JAK/STAT pathways play an important role in extracellular mechanistic signaling and might be an important mediator of mechanistic signals that influence disease progression and immune environment. [Hu et al., (2023) Front. Bioeng. Biotechnol. Vol. 11-2023].

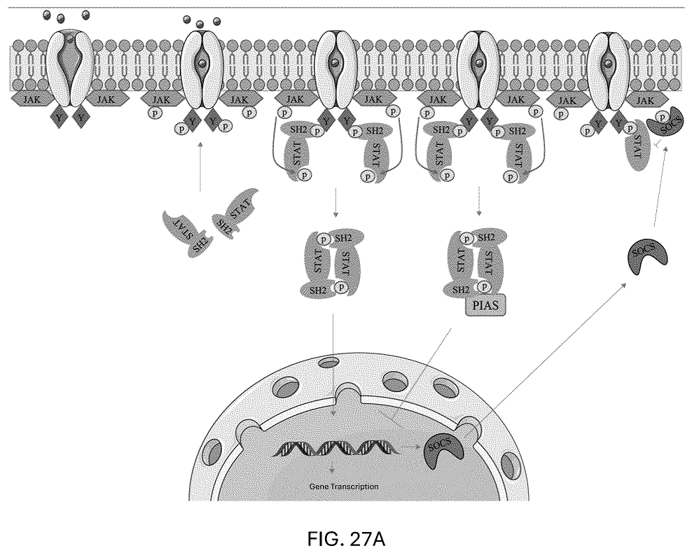

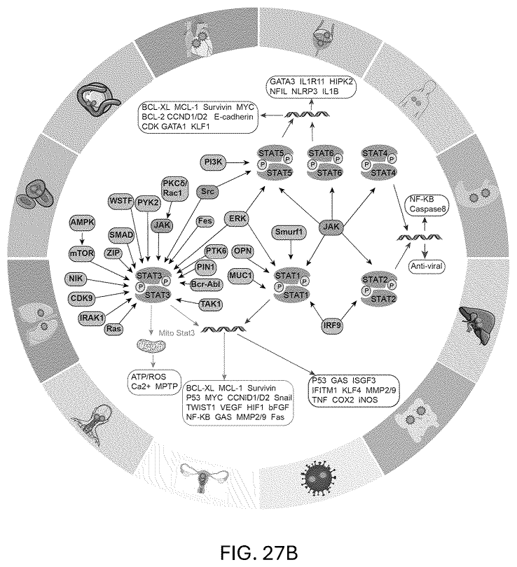

The JAK/STAT pathway has three components: cellular receptors, JAK protein, and STAT protein. The JAK family is a group of non-transmembrane tyrosine kinases, which is mainly composed of four members: JAK1, JAK2, JAK3, and TYK2 with molecular weights ranging from 120 to 140 kDa. JAK1, JAK2, and TYK2 are ubiquitous, while JAK3 is mainly expressed in hematopoietic cells. There are seven members of the STAT family in a mammal: STAT1, STAT2, STAT3, STAT4, STAT5A, STAT5B, and STAT6. Each member of the STAT family can be activated by a variety of cytokines and associated JAKs. First, cytokines bind to the corresponding transmembrane receptors and induce dimerization then activating JAK kinases couple to and phosphorylate the receptors. Second, the tyrosine residues on the catalytic domain of the receptor are phosphorylated to form a docking site to which STAT proteins with SH2 domains are recruited and STATs are phosphorylated and form homodimers or heterodimers. Finally, dimerized STATs dissociate from receptors and translocate into the nucleus, where they bind to DNA-binding sites and regulate gene transcription. Therefore, the activation of the JAK/STAT signaling cascade pathway is necessarily influenced by upstream extracellular cytokines and downstream JAK/STAT family protein types. For example, IFN-α/β activate STAT1, STAT2, and STAT4 via JAK1 and TYK2, whereas IFN-γ activates STAT1 or STAT5 via JAK1 and JAK2. IL-6 and IL-11 activate STAT1, STAT3 via JAK1, JAK2, and TYK2, but IL-12 and IL-23 activate STAT3 and STAT4 via JAK2 and TYK2. At the same time, STAT can be directly activated independently of JAK pathways, such as by epidermal growth factor (EGF), platelet-derived growth factor (PDGF), and mitogen-activated protein kinase (MAPK). In addition, the JAK/STAT pathway receives regulation by multiple mechanisms, including that PIAS inhibits gene transcription by directly binding to STAT dimers and thereby blocking STAT binding to DNA, and SOCS protein can negatively regulate the JAK/STAT signaling cascade by inhibiting JAK activity, competing with STAT to bind phosphorylation sites on cytokine receptors, and inducing STAT proteasomal degradation (see, FIG. 27A) [Hu et al., (2023) Front. Bioeng. Biotechnol. Vol. 11-2023].

The JAK/STAT signaling axis is a central pathway that mediates the cellular inflammation response and carcinogenesis and participates in the transduction of cellular physiological signals, such as renin-angiotensin signaling, insulin-like growth factor (IGF-IR) signaling (see, FIG. 27B). STAT promotes the transcriptional activation of target genes in response to specific extracellular stimuli (including cytokines, growth factors, and other agents) through tyrosine phosphorylation-mediated activation, most of which is mediated by JAKs, but with the interaction of multiple intracellular signaling proteins then affecting key cellular processes, including differentiation, proliferation, survival and functional activation, which in turn are involved in the development of various diseases [Hu et al., (2023) Front. Bioeng. Biotechnol. Vol. 11-2023].

The term “Kaplan-Meier survival curve” or “survival curve” as used herein refers to the probability of surviving in a given length of time while considering time in many small intervals. It is commonly used to analyze time-to-event (survival) data, such as the time until death or the time until a specific event occurs. Time is plotted on the x-axis and the survival rate is plotted on the y-axis. Each subject is characterized by three variables: (1) their serial time; (2) their status at the end of their serial time (occurrence of an event of interest or censored); and (3) the study group they are in. “Serial time” refers to the clinical course duration for each subject having a beginning and an end along the timeline of the complete study. An “interval”, which is graphed as a horizontal line, is the serial time duration of known survival. An interval therefore is terminated only by the event of interest. “Censoring” means the total survival time for that subject cannot be accurately determined; this can happen when something negative for the study occurs, such as the subject drops out, is lost to follow-up, or required data is not available, or, conversely, something good happens, such as the study ends before the subject had the event of interest occur. Censoring can occur within the study or terminally at the end. Censored subjects are indicated as tick marks; these do not terminate the interval [Rich, J T., et al. Otolaryngol. Head Neck Surg. (2010) 143 (3): 331-36]. The Kaplan Meier plot assumes that: (i) at any time subjects who are censored (i.e., lost) have the same survival prospects as subjects who continue to be followed; (ii) the survival probabilities are the same for subjects recruited early and late in the study; and (iii) the event (e.g., death) happens at the time specified. Probabilities of occurrence of an event are computed at a certain point of time with successive probabilities multiplied by any earlier computed probabilities to get a final estimate. The survival probability at any particular time is calculated as the number of subjects surviving divided by the number of subjects at risk. Subjects who have died, dropped out, or have been censored from the study are not counted as at risk.

The term “leukemia” as used herein refers to the clonal expansion of leukemic cells in the bone marrow, classically resulting in elevated numbers of cells of the affected lineage in circulating blood and, with certain lymphoid malignancies, abnormal cellular proliferation in lymphatic tissue [Bispo, J A B., et al. Cold Spring Harb. Perspect. Med. (2020) 10 (6): a034819]. Leukemias are generally classified into subtypes defined by cell lineage (lymphocytic or myeloid) and stage of maturation arrest (acute or chronic). Lymphocytic leukemias start in lymphoid cells while myelogenous leukemias start in myeloid cells. The types of leukemia include: (1) acute lymphocytic leukemia (ALL), the most common leukemia, which starts in the lymphoid cells of the bone marrow; (2) acute myelogenous leukemia (AML), also sometimes called acute granulocytic leukemia, acute myelocytic leukemia, acute myeloid leukemia and acute non-lymphocytic leukemia, which starts in the myeloid cells of the bone marrow; (3) chronic lymphocytic leukemia (CLL), a slow-growing leukemia that starts in the lymphoid cells of the bone marrow; (4) chronic myelogenous leukemia (CML), also called chronic myeloid leukemia, which starts in the myeloid cells of the bone marrow; (5) myeloproliferative neoplasms, which are a group of blood disorders, and (6) chronic myelomonocytic leukemia (MPNs), which starts in the myeloid cells of the bone marrow, which can lead to AML. The causes of leukemia in pediatric patients remain elusive. Several genetic syndromes and immune disorders are associated with both ALL and AML risk, although most cases are not familial. These include Down syndrome (DS), Li-Fraumeni syndrome, neurofibromatosis, DNA repair deficiency syndromes like Fanconi anemia and Bloom syndrome, and rare inherited bone marrow failure syndromes like Kostmann syndrome, Diamond-Blackfan anemia, dyskeratosis congenita, and Schwachman-Diamond syndrome [Bispo, J A B., et al. Cold Spring Harb. Perspect. Med. (2020) 10 (6): a034819].

Current treatments for leukemia include chemotherapy, radiation, and bone marrow transplantation; chemotherapy still remains the most important intervening strategy in treating different types of hematological malignancies [Sak, K. & Evaraus, H., Curr. Genomics (2017) 18 (1): 3-26, citing Zhang, D. et al. Oxid. Med. Cell Longev. (2012) 209843; Liu, X. et al. Nutr. Cancer (2015) 67 (2): 238-249; Mahbub, A A et al. Anticencer. Agents Med. Chem. (2013) 13 (10:1601-13; Davis, A S, et al. Am. Fam. Physician (2014) 89 (9): 731-38; Lu, H F, et al. Anticancer Res. (2007) 17 (1A): 117-25; Li, R F et al. Exp. Ther. Med. (2015) 9 (3): 697-706; Lin, C C, et al. In vivo (2012) 26 (4): 665-70; Davenport, A. et al. Int. J. Mol. Med. (2010) 25 (3): 465-70; Lee, C Y et al. Oncol. Rep. (2012) 28 (5): 1883-88].

However, standard chemotherapy agents are usually expensive and often associated with toxicity towards normal cells resulting in serious side effects and limiting the overall efficacy of drugs [Id., citing Zu, Y. et al., Planta Med. (2009) 75 (10): 1134-40, Liu, X. et al. Nutr. Cancer (2015) 67 (2): 238-49, Mahbub, A A et al. Anticancer. Agents Med. Chem. (2013) 13 (10): 1601-13, Davis, A S et al. Am. Fam. Physician (2014) 89 (9): 731-8, Li, R F et al. Exp. Ther. Med. (2015) 9 (3): 697-706; Davenport, A. et al. Int. J. Mol. Med. (2010) 25 (30): 465-70; Li, D. et al. Free Radic. Biol. Med. (2009) 46 (6): 731-36; Lugli, E. et al, Leuk Res. (2009) 33 (1): 140-50; Lee, R. et al. Toxicol. (2004) 195 (2-3): 87-95].

In addition, drug resistance represents a major problem in the current treatment of leukemia [Id., citing Zheng, J. et al. Asian Pac. J. Cancer Prev. (2012) 13 (4): 1119-24, Liu, X. et al. Nutr. Cancer (2015) 67 (2): 238-49, De Martino, L. et al. Mini Rev. Med. Chem. (2011) 11 (6): 492-502, Davenport, A. et al. Int. J. Mol. Med. (2010) 25 (30): 465-70, Kilani-Jaziri, S. et al. Chem. Biol. Interact. (2009) 181 (1): 85-94; Chen, F Y et al. Mol. Med. Rep. (2015) 11 (1): 341-48; Shen, J. et al. Int. J. Hyperthermia (2008) 24 (2): 151-9; Cheng, J. et al. Int. J. Nanomedicine (2012) 7:2843-52]. Such chemoresistance can be either intrinsic or acquired after initial therapy, thus being a main reason for treatment failure [Id., citing De Martino, L. et al. Mini Rev. Med. Chem. (2011) 11 (6): 492-502].

Introduction of targeted therapies has brought about considerable improvement in survival of patients with certain types of leukemia; all-trans retinoic acid (ATRA) for AML and Imatinib against CML represent two examples of the success of target-based therapies [Id., citing Liu, X. et al. Nutr. Cancer (2015) 67 (2): 238-49; Winter, E. et al. Toxicol. In vitro (2014) 28 (5): 769-77; Noori-Daloii, M R et al. Leuk Res. (2014) 38 (5): 575-80; Kimura, S. et al. Int. J. Oncol. (2014) 19 (1): 3-9; Park, J. et al. Ther. Adv. Hematol. (2011) 2 (5): 335-52].

Agents that force immature leukemia cells to undergo terminal differentiation (“differentiation therapy”) may be a less toxic alternative to treat hematopoietic neoplasms [Id., citing De Martino, L. et al. Mini Rev. Med. Chem. (2011) 11 (6): 492-502, Chen, H. et al. Molecules (2012) 17 (11): 13424-38; Isoda, H. et al. Chem. Biol. Interact. (2014) 220:269-77; Tsolmon, S. et al. Mol. Nutr. Food Res. (2011) 55 (Suppl. 1): S93-S102; Chen, Y. et al. Blood (2013) 121 (18): 3682-91; Lee, C Y et al. Oncol. Rep. (2012) 28 (5): 1883-88, Li, D. et al. Free Radic. Biol. Med. (2009) 46 (6): 731-6, Chen, H. et al. Cell Biol. Int. (2013) 37 (11): 1215-1224; Wang, M. et al. Arch. Pharm Res. (2012) 35 (1): 129-135; Yang, H. et al. Oncotarget (2014) 5 (18): 8188-8201].

Indeed, granulocytic differentiation induced by ATRA plus arsenic trioxide (ATO) has proven to be a highly curative standard of care for treatment of acute promyelocytic leukemia (APL), an AML subtype [Kantarjian, H M et al. Cancer (2018) 124:4301-13]. However, application of differentiation therapy can be effective only in certain forms of leukemia and the treatment might be accompanied by severe side effects as well as development of resistance [Sak, K. & Evaraus, H., Curr. Genomics (2017) 18 (1): 3-26, citing Qin, Y. et al., Eur. J. Pharm. Sci. (2012) 45 (5): 648-56; Zhang, K. et al. Cancer Sci. (20008) 99 (4): 689-95; Hui, H. et al. Gene (2014) 551 (20:230-35; Nakazato, T. et al. Haematologica (2005) 990 (3): 317-25; Philchenkov, A A et al. Ukr. Biokhim. Zh. (2010) 82 (2): 104-110; Nakazaki, E. et al. Eur. J. Nutr. (2013) 52 (1): 25-35].

Management strategies for CML have made significant progress after the discovery of Imatinib as a selective protein tyrosine kinase inhibitor against BCR-ABL, which is formed as a consequence of the reciprocal translocation between chromosomes 9 and 22; its constitutive tyrosine kinase activity contributes to antiapoptotic mechanisms, uncontrolled cell proliferation and survival advantage in CML [Id., citing Zhu, J F et al. PLoS One (2011) 6 (8): e23720; Kim, J H et al., Leuk Res. (2012) 36 (9): 1157-64; Noori-Daloii, M R et al. Leuk Res. (2014) 38 (5): 575-80; Yang, H. et al. Oncotarget (2014) 5 (18): 8188-8201; Iwasaki, R. et al. Cancer Sci. (2009) 100 (2): 349-56; Tolomeo, M. et al. Cancer let. (2008) 265 (2): 289-97; Solmaz, S. et al. Nutr. Cancer (2014) 66 (4): 599-612; Lust, S. et al. Mol. Nutr. Food Res. (2010) 54 (6): 823-32; Monteghirfo, S. et al. Mol Cancer Ther. (2008) 7 (9): 2692-2702; Valdes, A. et al. Electrophoresis (2012) 33 (15): 2314-27].

However, despite the initial therapeutic efficiency of Imatinib, development of resistance and disease relapse are still serious problems for most patients [Tolomeo, M. et al. Cancer let. (2008) 265 (2): 289-97; Solmaz, S. et al. Nutr. Cancer (2014) 66 (4): 599-612; Lust, S. et al. Mol. Nutr. Food Res. (2010) 54 (6): 823-32; Monteghirfo, S. et al. Mol Cancer Ther. (2008) 7 (9): 2692-2702]. The use of new generation inhibitors specifically targeting the tyrosine kinase domain of Bcr-Abl, such as Dasatinib and Nilotinib, can also be limited due to emergence of resistance and adverse effects of these agents [Kim, J H et al., Leuk Res. (2012) 36 (9): 1157-64, Iwasaki, R. et al. Cancer Sci. (2009) 100 (2): 349-56, Tolomeo, M. et al. Cancer let. (2008) 265 (2): 289-97; Solmaz, S. et al. Nutr. Cancer (2014) 66 (4): 599-612].

Emerging therapies in AML include FLT3 inhibitors, IDH1/IDH2 inhibitors, gemtuzumab oxogamicin (GO) and novel antibodies targeting CD33 and CD123, venetoclax as a BCL3 inhibitor and the introduction of checkpoint inhibitors [Antarjian, H M et al. Cancer (2018) 124:4301-13].

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for antigenic determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Lymphocytes are much more common in the lymphatic system, and include B cells, T cells, killer T-cells, and natural killer (NK) cells. There are two broad categories of lymphocytes, namely T cells and B cells. T-cells are responsible for cell-mediated immunity whereas B-cells are responsible for humoral immunity (relating to antibodies). T-cells are so-named because these lymphocytes mature in the thymus; B-cells mature in bone marrow. B cells make antibodies that bind to pathogens to enable their destruction. CD4+ (helper) T cells coordinate the immune response. CD8+ (cytotoxic) T cells and Natural Killer (NK) cells are able to kill cells of the body that are, e.g., infected by a virus or display an antigenic sequence.

The term “lymphocyte activation” or “activation” refers to stimulation of lymphocytes by specific antigens, nonspecific mitogens, or allogeneic cells resulting in synthesis of RNA, protein and DNA and production of lymphokines, the soluble product of lymphocytes; it is followed by proliferation and differentiation of various effector and memory cells. For example, a mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “lymphoma”, as used herein refers to solid cancers of the lymphatic system that initiate from the malignant transformation of a single lymphocyte. The affected lymphocyte usually is located in a lymph node but may be resident in another organized lymphoid tissue outside the bone marrow, such as the spleen or thymus. When the transformed lymphocyte is positioned in a diffuse lymphoid tissue, such as the gut associated lymphoid tissue (GALT), the lymphoma that develops is said to be extranodal. Lymphomas almost always depend on surrounding stromal cells for survival and growth factors as well as vital intercellular contacts, and so are generally restricted to sites within tissues. From its initiation site, a lymphoma tends to spread to additional secondary lymphoid tissues and eventually to non-lymphoid organs. Occasionally, a lymphoma cell undergoes additional mutations that allow it to survive and circulate in the blood, i.e., it becomes a leukemic cell. The disease then may be called a “leukemia/lymphoma” [Mak, T W et al. Ch. 20 Hematopoietic cancers, in Primer to the Immune response (2014) Elsevier, Inc., pp. 573-574].

The progression of any lymphoma can be described in four stages:

In stage I, one or more diseased lymph nodes are present in a single group of lymph nodes in one particular lymphoid tissue of the body.

In stage II, diseased lymph nodes are present in more than one group of lymph nodes, but all diseased nodes are contained either above or below the diaphragm. Tumor cells may also be present in a single organ near an affected node.

In stage III, diseased lymph nodes are present in two or more groups on both sides of the diaphragm. Tumor cells also may be present in the spleen and/or another organ near an affected node.

In stage IV. There is wide dissemination of tumor cells into multiple lymph nodes, bone marrow, liver and multiple organs. [Mak, T W et al. Ch. 20 Hematopoietic cancers, in Primer to the Immune response (2014) Elsevier, Inc., pp. 573-574]

Lymphomas display a tumor microenvironment (TME), with huge differences amongst the various forms. Hodgkin lymphoma (HL), both classic HL and nodular lymphocyte predominant HL, as well as several T cell lymphoma entities, such as angioimmunoblastic T-cell lymphomas (AITL), predominantly (>80% of the tumor mass) consist of TME cells. In indolent B-cell lymphomas, such as follicular lymphoma (FL) or marginal zone lymphomas, the TME constitutes about 50% of the cellular mass. In aggressive lymphomas, such as diffuse large B-cell lymphomas (DLBCL), the proportion of the TME varies and is generally lower. In Burkitt lymphoma, plasmablastic lymphoma, and lymphoblastic T cell and B cell lymphomas, the TME is barely existent. [Menter, T. & Tzankov, A. Pathobiology (2019) 86:225-36].

Hodgkin lymphoma is a lymphoma in which the tumor mass is made up of a reactive infiltrate of non-transformed lymphocytes, macrophages and fibroblasts plus scattered, malignant Reed-Sternberg cells. Reed-Sternberg cells are large, abnormal lymphocytes that may contain more than one nucleus, are clonal in their growth and are the tumor cells of the malignant mass. While of the B-cell lineage, they lack common B-cell specific surface markers such as CD19 and CD79a as well as Ig gene transcripts. [Hertel, C B et al. Oncogene (2002) 21:4908-20]. Hodgkin lymphoma most commonly affects lymph nodes in the neck or in the area between the lungs and behind the breastbone. It can also begin in groups of lymph nodes under an arm, in the groin, or in the abdomen or pelvis. If it spreads, it may spread to the lung, spleen liver, bone marrow or bone.

There are two major categories of Hodgkin lymphoma: classical Hodgkin lymphoma, which is divided into 4 subtypes based on the appearance of lymph node structure and cells, and nodular lymphocyte-predominant Hodgkin lymphoma.

Classical Hodgkin lymphoma (cHL) represents about 95% of cases of Hodgkin lymphoma. It is diagnosed when characteristic abnormal lymphocytes, (Reed-Sternberg cells) are found. There are 4 subtypes of cHL.

Nodular sclerosis Hodgkin lymphoma, the most common subtype of cHL, affects up to 80% of people diagnosed with cHL. It is most common in young adults, especially women. In addition to Reed-Sternberg cells, there are bands of connective tissue (called fibrosis) found in the lymph node. This type of lymphoma often affects the lymph nodes in the mediastinum.

Lymphocyte-rich classic Hodgkin lymphoma affects up to 6% of individuals with cHL. It is more common in men and usually affects areas other than the mediastinum. In addition to Reed-Sternberg cells, the lymph node tissue contains many normal lymphocytes.

Mixed cellularity Hodgkin lymphoma occurs most often in older adults. It sometimes develops in the abdomen and carries many different cell types, including large numbers of Reed-Sternberg cells.

Lymphocyte-depleted Hodgkin lymphoma is the least common subtype of cHL, and represents only about 1% of patients with cHL. It is most common in older adults; people with HIC; and people in non-industrial countries. The lymph node contains almost all Reed-Sternberg cells.

Nodular lymphocyte-predominant Hodgkin lymphoma (also called LPHL, lymphocyte-predominant Hodgkin lymphoma, and NLPHL) is a rare type of Hodgkin lymphoma marked by the presence of lymphocyte-predominant cells, which used to be called popcorn cells. These cells are different from the typical Reed-Sternberg cells found in classic Hodgkin lymphoma. Nodular lymphocyte-predominant Hodgkin lymphoma may change into diffuse large B-cell lymphoma.

Non-Hodgkin lymphoma (NHL) is a heterogeneous group of lymphomas in which the solid tumor mass consists almost entirely of malignant lymphocytes. [Mak, T W et al. Ch. 20 Hematopoietic cancers, in Primer to the Immune response (2014) Elsevier, Inc., pp. 573-574] NHL may be described by how quickly the cancer is growing. An “indolent or low grade lymphoma’ is a type of lymphoma that tends to grow and spread slowly, and has few symptoms. When indolent lymphoma is in stages 1 and 2, it is called localized disease. An “aggressive lymphoma” “high-grade lymphoma” or intermediate-grade lymphoma” is a type of lymphoma that grows and spreads quickly and has severe symptoms. In children, aggressive non-Hodgkin lymphoma is more common. Some types of lymphoma cannot be easily classified as indolent or aggressive.

There are more than 60 NHL subtypes of NHL that tend to mimic stages of normal B cell differentiation [Mancuso, S. et al. Immunity & Aging (2018) 15:22].

T cell lymphomas: T cell lymphomas make up approximately 10%-15% of lymphoid malignancies. [Armitage, J. O. Am. J. Hematol. (2017) 92 (7): 706-15, citing 1]. The frequency of these lymphomas varies geographically, with the highest incidence in parts of Asia. [Id., citing Anderson, J R et al. Ann. Oncol. (1998) 9:717-20] T-cell lymphomas can be divided into those of precursor T-cells (i.e., precursor T-lymphoblastic lymphoma) and those arising in more mature T-cells, with the latter termed peripheral T-cell lymphomas (PTCLs). As is true for B-cell lymphomas, a subset of PTCLs such as mycosis fungoides and the CD30-positive cutaneous lymphoproliferative disorders (i.e., cutaneous anaplastic large-cell lymphoma and lymphomatoid papulosis) have a prolonged natural history. The aggressive PTCLs are associated with a short survival. These can be subdivided into those of primarily nodal origin and those that are typically present in specific extranodal sites and are often associated with characteristic clinical syndromes [Armitage, J. O. Am. J. Hematol. (2017) 92 (7): 706-15]

Less than 1% of individuals with lymphoma have NK-cell lymphoma. The most common subtypes of T and NK-cell lymphoma are:

Anaplastic large cell lymphoma, primary cutaneous type, which only involves the skin. It is often indolent, although aggressive subtypes are possible.

Anaplastic large cell lymphoma, systemic type is an aggressive form of lymphoma that makes up about 2% of all lymphomas and about 10% of all childhood lymphomas. An increased amount of the ALK-1 protein may be found in the cancer cells, which leads to a better prognosis.

Breast implant-associated anaplastic large cell lymphoma arises in areas near breast implants. It is usually less aggressive than the systemic type of anaplastic large cell lymphoma.

Peripheral T-cell lymphoma not otherwise specified (NOS) is an aggressive form of lymphoma most common in individuals older than 60 and makes up about 6% of all lymphomas in the United States and Europe. The cells of this lymphoma vary in size and have CD4 or CD8 on their surface.

Angioimmunoblastic T-cell lymphoma (AITL) is an aggressive form of lymphoma characterized by enlarged often tender lymph nodes, fever, weight loss, rash and high levels of immunoglobulins (Igs) in the blood.

Adult T-cell lymphoma/leukemia (human T cell lymphotropic virus type I positive) is caused by the human T-cell lymphotropic virus type 1. It is an aggressive disease that often involves the bone and skin. Lymphoma cells are often found in the blood.

Extranodal NK/T cell lymphoma nasal type is an aggressive type of lymphoma that is very rare in the United States and Europe in general, but more common in Asian and Hispanic communities. While most often involving the nasal area and sinuses, it also can involve the gastrointestinal tract, skin, testicles or other areas in the body.

Enteropathy-associated T cell lymphoma is rare in the United States but is more common in Europe. It is an aggressive form of T-cell lymphoma that involves the intestines. Some with this subtype have celiac disease or a history of gluten intolerance.

Hepatosplenic T-cell lymphoma is an aggressive form of peripheral T cell lymphoma that involves the liver and spleen. It occurs most often in teenaged and young men.

Subcutaneous panniculitis-like T cell lymphoma is a high risk aggressive lymphoma. It is a form of peripheral T cell lymphoma that is similar to hepatosplenic T cell lymphoma. It involves the tissue under the skin, and is often first diagnosed as panniculitis (inflammation of fatty tissues).

Mycosis fungoides is a rare T cell lymphoma that primarily involves the skin. It often has a very long and indolent course but may become more aggressive and spread to lymph nodes or internal organs.

There are three major types of NHL in children. These include aggressive mature B cell non-Hodgkin lymphoma; lymphoblastic lymphoma; and anaplastic large cell lymphoma. Aggressive mature B-cell non-Hodgkin lymphomas include Burkitt lymphoma/leukemia; diffuse large B cell lymphoma; and primary mediastinal B-cell lymphoma. Lymphoblastic lymphoma is a type of lymphoma that mainly affects T-cell lymphocytes. Anaplastic large cell lymphoma is a type of lymphoma that mainly affects T-cell lymphocytes. It usually forms in the lymph nodes, skin, or bone, and sometimes forms in the gastrointestinal tract, lung, tissue that covers the lungs, and muscle. Patients with anaplastic large cell lymphoma have a receptor, called CD30, on the surface of their T cells. In many children, anaplastic large cell lymphoma is marked by changes in the ALK gene that makes a protein called anaplastic lymphoma kinase.

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups-class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAP1: TAP2 peptide transporter, the PSMB (or LMP) genes that encode proteasome subunits, the genes encoding the DMα and BMβ chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyzes peptide binding to MHC class II molecules are related to the MHC class II genes, as are the DOA and DOB genes that encode the subunits of the regulatory HLA-DO molecule. [Janeway's Immunobiology. 9th ed., GS, Garland Science, Taylor & Francis Group, 2017. pps. 232-233]. In humans, there are three MHC class II isotypes: HLA-DR, HLA-DP, and HLA-DQ, encoded by α and β chain genes within the Human Leukocyte Antigen (HLA) locus on chromosome 6 [Wosen, J E., et al. Front. Immunol. (2018) doi.10.3389/fimmu.2018.02144].

MHC-like molecules, while not encoded by the same gene group as true MHCs, have the same folding and overall structure of MHCs, and specifically MHC class I molecules, and thus possesses similar biological functions such as antigen presentation. The CD1 family of molecules is an example of an MHC-like molecule. It consists of two groups based on amino acid homology: group 1, which includes CD1a, b, and c; and group 2, which consists of CD1d. Group 1 CDIs can present antigens to a wide variety of T cells, whereas CD1d presents antigens mostly to NKT cells [Brutkiewicz. “CD1d Ligands: The Good, the Bad, and the Ugly.” The Journal of Immunology (2006) 177 (2) 769-775]. While CD1d structurally resembles MHC Class I molecules, it traffics through the endosome of the exogenous antigen presentation pathway. The binding groove of the CD1d molecules tethers the lipid tail of a glycolipid antigen, while the carbohydrate head group of the antigen projects out of the groove for recognition by the TCR of the NKT cell [Wah, MakTak, et al. “Chapter 11: NK, γδ T and NKT Cells.” Primer to the Immune Response. Elsevier, 2014].

CD1d presents lipid antigens and requires the presence of particular mechanisms to induce uptake of these molecules by APCs and subsequent loading onto CD1d molecules. Lipid transfer protein such as apolipoprotein E and fatty acid amide hydrolase (FAAH) have been shown to enhance the presentation of certain antigens by CD1d. Loading efficiency can be enhanced by specific proteins, such as saposins and microsomal triglyceride transfer protein, present in the endosomal and lysosomal compartments of cells by promoting lipid antigen exchange. Similar to MHC antigens, lipid antigens can also be processed by lysosomal enzymes to yield active compounds, as demonstrated in the case of CD1d for synthetic antigens, microbial antigens, and self-antigens [Giradi and Zajonc (2012). Immunol Rev. 250 (1): 167-179].

MHC Class I-like molecules are nonclassical MHC type molecules, while including CD1d also include CD1a, CD1b, CD1c, CD1e, and MR1 are also expressed on APCs and can activate various subsets of T cells [Kumar and Delovitch (2014) Immunology 142:321-336]. Other non-classical histocompatibility molecules include MR1, which activate MAIT cells.

The term “MHC restriction” as used herein refers to the requirement that APCs or target cells express MHC molecules that a T cell recognizes as self in order for T cell to respond to the antigen presented by that APC or target cell (T cells will only recognize antigens presented by their own MHC molecules). For example, CD8 T cells bind class I MHC which are expressed on most cells in the body, and CD4 T cells bind class II MHC which are only expressed on specialized APCs.

The term “mediate” and its various grammatical forms as used herein refers to depending on, acting by or connected through some intervening agency.

The term “memory cells” as used herein refers to B and T lymphocytes generated during a primary immune response that remain in a quiescent state until fully activated by a subsequent exposure to specific antigen (secondary immune response). Memory cells generally are more sensitive than naïve lymphocytes to antigen and respond rapidly on re-exposure to the antigen that originally induced them. During an immune response, naïve T cells (T_{N}) are primed by antigen-presenting cells (APCs). Depending on the strength and quality of stimulatory signals, proliferating T cells progress along a differentiation pathway that culminates in the generation of terminally differentiated short-lived effector T (T_{EFF}) cells. When antigenic and inflammatory stimuli cease, primed T cells become quiescent and enter into the memory stem cell (T_{SCM}), central memory (T_{CM}) cell or effector memory (T_{EM}) cell pools, depending on the signal strength received. T_{SCM} cells possess stem cell-like attributes to a greater extent than any other memory lymphocyte population. Although both T_{CM} and T_{EM} cells can also undergo self-renewal, the capacity to form diverse progeny is progressively restricted, so that only T_{SCM} cells are capable of generating all three memory subsets and T_{EFF} cells; T_{CM} cells can give rise to T_{CM}, T_{EM} and T_{EFF} cells, and T_{EM} cells can only produce themselves and T_{EFF} cells [Gattinoni, L. et al. Nature Revs. Cancer (2012) 12:671-84].

The term “modulate” as used herein means to regulate, alter, adapt, or adjust to a certain measure or proportion.

The term “myeloid” as used herein means of or pertaining to bone marrow. The term “myeloid cell” as used herein refers to any white blood cell other than lymphocytes. Granulocytes and monocytes, collectively called “myeloid cells”, are differentiated descendants from common progenitors derived from hematopoietic stem cells in the bone marrow. Commitment to either lineage of myeloid cells is controlled by distinct transcription factors followed by terminal differentiation in response to specific colony-stimulating factors and release into the circulation. Upon pathogen invasion, myeloid cells are rapidly recruited into local tissues via various chemokine receptors, where they are activated for phagocytosis as well as secretion of inflammatory cytokines, thereby playing major roles in innate immunity [Kawamoto, H., Minato, N. Intl J. Biochem. Cell Biol. (2004) 36 (8): 1374-9].

The term “myeloid-derived suppressor cells” as used herein refers to a heterogeneous population of cells that represent a pathologic state of activation of monocytes and relatively immature neutrophils. MDSCs are characterized by a distinct set of genomic and biochemical features, and can, on the basis of recent findings, be distinguished by specific surface molecules. The salient feature of these cells is their ability to inhibit T cell function and thus contribute to the pathogenesis of various diseases [Veglia, F. et al. Nature Immunol. (2018) 19:108-19].

The term “myeloma” as used herein refers to a plasma cell tumor that secretes large quantities of an Ig protein of usually unknown specificity. Myelomas are tumors of fully differentiated plasma cells that are present either as solid masses or as dispersed clones in the bone marrow, blood or tissues. Unlike normal plasma cells, which do not divide after they differentiate, myeloma cells continue to proliferate in an uncontrolled way and synthesize large amounts of Ig chains. When tumors are present in multiple body sites, the disease is referred to as multiple myeloma (MM).

Normal plasma cells cannot divide and so die soon after secreting antigen-specific antibody. In contrast, cancerous plasma cells divide uncontrollably and express huge quantities of antibodies or single Ig chains of unknown antigenic specificity.

The progression of multiple myeloma (MM), a B cell malignancy, begins with the precursor pathogenic state of monoclonal gammopathy of undetermined significance (“MGUS”). In the absence of clinical symptoms, MGUS is diagnosed by quantifying the amount of immunoglobulin present in both the bloodstream and BM, specifically with a plasma cell population of <10% in the BM. The Ig protein produced by the malignant plasma cell is called a paraprotein, IgM paraprotein, para-IgM or M protein. Evidence indicates that MGUS, previously characterized by myeloma cell growth without bone destruction or other organ involvement, is in fact associated with alterations in the bone. Epidemiologic evidence has shown that patients with MGUS suffer from a significantly increased fracture risk, and that the prevalence of MGUS is increased in patients with osteoporosis. [Fairfield, H. et al., Ann. NY Acad. Sci. (2016) 1364 (1): 32-51, citing Drake, M T. J. Bone Miner. Res. (2014) 29:2529-33] It has been demonstrated that the onset of MGUS is concurrent with the deterioration of both auxiliary and appendicular microarchitecture leading to skeletal fragility [Id., citing Drake, M T. J. Bone Miner. Res. (2014) 29:2529-33].

The disease state transitions into either smoldering MM (SMM) or MM (if clinically manifested), with a plasma cell content exceeding 10% [Fairfield, H. et al., Ann. NY Acad. Sci. (2016) 1364 (1): 32-51, citing Berenson, J R et al. Br. J. Haematol. (2010) 150:28-38].

Smoldering multiple myeloma (SMM) is an intermediate clinical stage between MGUS and MM. It is classified as having high serum or urinary monoclonal protein as well as clonal BM plasma cells in the range of 10-60%, in the absence of additional myeloma-defining events [Id., citing Glavey, S V & Ghobrial, I M. Expert Rev. Hematol. (2015) 8:273-5], such as hypercalcaemia, renal insufficiency, anemia, or bone lesions. [Id., citing Rajkumar, S V et al. Lancet Oncol. (2014) 15: e538-3548]. MGUS progresses to MM at a rate of 1-2% of patients per year; this transition is likely influenced by the presence of mutational diversity or clonality of MM cell populations as well as changes in the local bone marrow (BM) and other systemic factors [Id., citing Jemal, A. et al. C A Cancer J. Clin. (2009) 59:1-25; Pawlyn, C. et al. Blood (2015) 125 (5): 831-40; Barlogie, B. et al. Blood (2004) 103:20-32; Rollig, C. et al. Lancet (2014) 385]. MM cells are thought to initially create a plasmacytoma, a single tumor, and then develop into multiple lesions to form the disease of multiple myeloma. [Id., citing Lorsbach, R B et al. Am. J. Clin. Pathol. (2011) 136:168-182].

MM begins as monoclonal gammopathy of undetermined significance (MGUS), progresses to smoldering (asymptomatic) myeloma and finally becomes overt (symptomatic) myeloma, resulting in BM infiltration and osteolytic lesions. [Fairfield, H. et al., Ann. NY Acad. Sci. (2016) 1364 (1): 32-51].

The term “myelodysplastic syndrome” or “MDS” is a diverse collection of hematologic neoplasms characterized as a clonal disorder of hematopoietic stem cells, resulting in dysplasia and ineffective hematopoiesis within the bone marrow. This condition often leads to various degrees of cytopenias, which can manifest as anemia, leukopenia, or thrombocytopenia. Some individuals with MDS may progress to acute myeloid leukemia (AML), further complicating their clinical course. The prognosis for patients with MDS is highly variable and is influenced by factors such as cytogenetic abnormalities and the severity of cytopenias.

The term “myeloproliferative neoplasms” refers to a group of blood cancers where the bone marrow produces too many red blood cells, white blood cells, or platelets. This overproduction leads to build up of excess cells in the blood and bone marrow. The abnormal proliferation of one or more terminal myeloid cell lines in the peripheral blood gives rise to myeloproliferative neoplasms. Chronic myeloid leukemia (CML), polycythemia vera (PV), essential thrombocythemia (ET), and primary myelofibrosis (PMF) are four classic types of myeloproliferative neoplasms. WHO classification also included chronic neutrophilic leukemia (CNL), chronic eosinophilic leukemia (CEL), and MPN, unclassifiable. Out of the classic types of MPNs, CML is BCR-ABL1 positive, but PV, ET, and PMF are BCR-ABL1 negative.

The BCR-ABL1-negative myeloproliferative neoplasms (MPNs) are clonal disorders of the hematopoietic stem cell, mainly characterized by hyperproliferative bone marrow with varying degrees of reticulin/collagen fibrosis, extramedullary hematopoiesis, abnormal peripheral blood count, and constitutional symptoms [Iurlo, A. et al. Intl J. Mol. Sci. (2019) 20 (8): 1839]. The major causes of morbidity and mortality in these patients are most commonly represented by thrombo-hemorrhagic events and less frequently by infectious complications, and/or transformation to blast phase, often termed secondary acute myeloid leukemia (AML) or blast-phase MPN (MPN-BP).

The term “naïve T cell” as used herein refers to T cells that previously have not been exposed to an antigen. Naïve T cells are conventionally defined by co-expression of the RA isoform of the transmembrane phosphatase CD45, the lymph node homing molecules L-selectin (CD62L) and CCR7, and the costimulatory receptors CD27 and CD28 [De Rosa, S C et al. Nature Med. (2001) 7:245-48].

The term “natural killer (NK) cells” as used herein refers to lymphocytes in the same family as T and B cells, classified as group I innate lymphocytes. They have an ability to kill tumor cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell. Inhibitory receptors act as a check on NK cell killing. Most normal healthy cells express MHCI receptors, which mark them as “self.” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germline encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells. Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed. Consequently, those tumors that express low MHC Class I and which are thought to be capable of evading a T cell-mediated attack may be susceptible to an NK cell-mediated immune response instead. NK stimulation and effector function depends on the integration of signals derived from its various receptors. NK cells can recognize and kill virally infected and neoplastic cells through their cytotoxic function. NK cells further play an immunoregulatory role where NK cells stimulate the production of cytokines. In this manner, NK cells have the capacity to regulate the activity of other cells, particularly the cells of the immune system. The pattern of cytokines released by NK induction varies with stimulus. Thus, NK cells, like T cells, differentiate into discrete functional subsets with differing effectiveness on adaptive immunity. The presence of IFN-γ and other functional immunostimulatory factors, such as IL-2, and IL-12, on NK cells may lead to the activation and expansion of NK cells into lymphokine-activated killer (LAK) cells, which may give rise to cytokine induced killer cells (CIKs), which are CD3-, CD56-positive, non-major histocompatibility complex (MHC)-restricted, natural killer (NK)-like T lymphocytes. LAK cells upregulate effectors or adhesion molecules, such as perforin, NKp44, granzymes, FasL and TRAIL, and secrete IFN-γ to adhere to and lyse tumor cells [Nair and Dhodapkar (2017). Frontiers in Immunology 8:1178]. CIKs may enhance the cytolytic activity on tumor targets.

The term “natural killer T cell” or “NKT” as used herein, refers to invariant natural killer T (iNKT) cells, also known as type-I NKT cells, as well as all subsets of non-invariant (Vα24− and Vα24+) natural killer T cells, which express CD3 and an αβ T cell receptor (TCR) (herein termed “natural killer αβ T cells”) or γδ TCR (herein termed “natural killer γδ T cells”), all of which have demonstrated capacity to respond to non-protein antigens presented by CD1 antigens. The non-invariant NKT cells share in common with type-I NKT cells the expression of surface receptors commonly attributed to natural killer (NK) cells, as well as a TCR of either αβ or γδ TCR gene locus rearrangement/recombination. Accordingly, as used herein, the term “NKT cells” refers to a population of cells that includes CD3+Vα24+NKT cells, CD3+Vα24− NKT cells, CD3+Vα24−CD56+NKT cells, CD3+Vα24−CD161+NKT cells, CD3+γδ−TCR+ T cells, and mixtures thereof.

The term “invariant natural killer T cell” as used herein, is meant to be used interchangeably with the term “INKT,” and refers to a subset of T cell receptor (TCR) α-expressing cells that express a restricted TCR repertoire that, in humans, is composed of a Vα24− Ja18 TCRα chain, which is, for example, coupled with a Vβ11 TCRβ chain. iNKT is meant to encompass all subsets of CD3+Vα24+ type-I NKT cells (CD3+CD4+CD8−Vα24+, CD3+CD4−CD8+Vα24−+, and CD3+CD4−CD8−Vα24+) as well as those cells that can be confirmed to be type-I NKT cells by gene expression or other immune profiling but have down-regulated surface expression of Vα24 (CD3+Vα24−). This includes cells which either do or do not express the regulatory transcription factor FOXP3. Unlike conventional T cells, which mostly recognize peptide antigens presented by MHC molecules, iNKT cells recognize glycolipid antigens presented by the non-polymorphic MHC class 1-like CD1d.

The term “Next Generation Sequencing” or “NGS” as used herein refers to a method of parallel sequencing. For instance, a nucleic acid (e.g., DNA) sample is obtained and prepared into a library (meaning a collection of nucleic acid fragments from the sample). The library is prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. According to some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. Adapters include sequences, such as barcodes, restriction sites, and primer sequences. The term “NGS read length” as used herein refers to the number of base pairs (bp) sequenced from a DNA fragment. After sequencing, the regions of overlap between reads are used to assemble and align the reads to a reference genome, reconstructing the full DNA sequence.

The abbreviation “NFκB” as used herein refers to a proinflammatory transcription factor that switches on multiple inflammatory genes, including cytokines, chemokines, proteases, and inhibitors of apoptosis, resulting in amplification of the inflammatory response [Barnes, PJ, (2016) Pharmacol. Rev. 68:788-815]. The molecular pathways involved in NF-κB activation include several kinases. The classic (canonical) pathway for inflammatory stimuli and infections to activate NF-κB signaling involves the IKK (inhibitor of κB kinase) complex, which is composed of two catalytic subunits, IKK-α and IKK-β, and a regulatory subunit IKK-γ (or NFκB essential modulator [Id., citing Hayden, M S and Ghosh, S (2012) Genes Dev. 26:203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to κB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β [Id., citing Sun, S C. (2012) Immunol. Rev. 246:125-140]. This pathway switches on different gene sets and may mediate different immune functions from the canonical pathway. Dominant-negative IKK-β inhibits most of the proinflammatory functions of NF-κB, whereas inhibiting IKK-α has a role only in response to limited stimuli and in certain cells, such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151:179-89].

The term “non-expanded” as used herein, is meant to refer to a cell population that has not been grown in culture (in vitro) to increase the number of cells in the cell population.

The term “overall survival” as used herein refers to the length of time from either the date of diagnosis or the start of treatment for a disease, such as cancer, that subjects diagnosed with the disease are still alive.

The term “PAMPs” is an abbreviation for pathogen-associated molecular patterns. PAMPS are structural patterns present in components or products common to a wide variety of microbes, but not host cells. PAMPS are ligands for pattern recognition molecules (PRMs).

The term “pattern recognition molecules” or “PRMs” as used herein refer to proteins recognizing PAMPs. Soluble PRMs include the collectins, acute phase proteins and NOD proteins. Membrane-bound PRMs are pattern recognition receptors.

The term “pattern recognition receptors” or “PRRs” refers to widely distributed membrane bound PRMs fixed in either the plasma membrane of a cell or in the membranes of its endocytic vesicles. The term PRRs includes toll-like receptors (TLRs) and scavenger receptors. Engagement of PRRs induces pro-inflammatory cytokines.

The term “PD-1” or “programmed cell death protein 1” as used herein refers to an inhibitory receptor expressed on the surface of activated T cells. Its ligands, PD-L1 and PD-L2, are expressed on the surface of DCs or macrophages. PD-1 and its ligands PD-L1/PL-L2 act as co-inhibitory factors that can limit the development of the T cell response. PD-L1 is overexpressed on tumor cells or on non-transformed cells in the tumor microenvironment [Pardoll, D M. Nat. Rev. Cancer (2012) 12:252-64]. PD-L1 expressed on the tumor cells binds to PD-1 receptors on the activated T cells, which leads to the inhibition of the cytotoxic T cells. These deactivated T cells remain inhibited in the tumor microenvironment.

The term “Pearson correlation” as used herein refers to a statistical method that measures the strength of the linear relationship between wo variables with normally distributed data. It has a value between −1 and +1, with a value of −1 meaning a total negative linear correlation, 0 being no correlation, and +1 meaning a total positive correlation.

The term “persistence” as used herein refers to a stably maintained functionality in the body after infusion.

The terms “peripheral blood mononuclear cells” or “PBMCs” are used interchangeably herein to refer to blood cells having a single round nucleus. PBMCs include lymphocytes, monocytes, natural killer cells (NK cells) and dendritic cells. PBMCs can be separated from granulocytes and red blood cells in peripheral blood by density gradient centrifugation. For example, when Ficoll® is used to fractionate peripheral blood, PBMCs remain at the less dense, upper interface of the Ficoll® layer, often referred to as the buffy coat, and are the cells collected. These cells consist of lymphocytes (T cells, B cells, NK cells) and monocytes. In humans, lymphocytes make up the majority of the PBMC population, followed by monocytes, and only a small percentage of dendritic cells.

The term “phenotype” as used herein refers to qualitative and quantitative observable characteristics of cells. A cell's phenotype is the culmination of several cellular processes through a complex network of molecular interactions that ultimately result in a unique morphological signature. Clinical, biochemical and imaging methodologies can be used to refine and characterize a phenotype.

The terms “potent” and “potency” as used herein refer to the specific ability or capacity of the disclosed population of ap T cells, as indicated by appropriate laboratory tests or by adequately controlled clinical data obtained through administration in the manner intended, to effect a given result.

The term “progression” as used herein refers to the course of disease as it becomes worse or spreads in the body.

The term “progression-free survival” or PFS” as used herein refers to the length of time during and after the treatment of the disease that a patient lives with the disease but it does not get worse.

The term “purify” and its various grammatical forms as used herein refers to being free from extraneous or undesirable elements.

The term “Ras-MAPK pathway” as used herein, refers to a crucial signaling cascade in cells that regulates growth, proliferation, and survival. The function of this pathway is to transduce signals from the extracellular milieu to the cell nucleus where specific genes are activated for cell growth, division and differentiation. The Ras/Raf/MAPK pathway is also involved in cell cycle regulation, wound healing and tissue repair, integrin signaling and cell migration. Finally, the Ras/Raf/MAPK pathway is able to stimulate angiogenesis through changes in expression of genes directly involved in the formation of new blood vessels. Thus, signaling through the Ras/Raf/MAPK regulates a variety of cellular functions that are important for tumorigenesis [The Ras/Raf/MAPK Pathway. Molina, Julian R. et al. Journal of Thoracic Oncology, Volume 1, Issue 1, 7-9].

The ras superfamily of genes encodes small GTP-binding proteins that are responsible for regulation of many cellular processes, including differentiation, cytoskeletal organization, and protein trafficking. Oncogenic ras genes in human cells include H-ras, N-ras and K-ras. The 21-kd transforming proteins H- and K-ras genes were first identified as the counterparts of the oncogenes of the Harvey and Kirsten rat sarcoma viruses, whereas the N-ras oncogene was isolated from a neuroblastoma and has not been found in any retroviruses. K-ras is initially synthesized as an inactive cytosolic pro-peptide. The protein undergoes a series of post-translational modifications at its carboxyl terminus that increase its hydrophobicity allowing its localization to the lipid-rich cell membrane. An important post-translational modification is farnesylation in the hydrophobic tail of the carboxyl terminal group. This reaction is catalyzed by the enzyme farnesyltransferase which adds a 15-carbon hydrobobic farnesyl isoprenyl to the carboxyl terminus of Ras. Once in the cell membrane, K-ras cycles between inactive guanosine diphosphate-bound and active guanosine triphosphate (GTP)-bound states, thereby activating a series of effector kinases—such as Raf and MAPK—that phosphorylate a cascade of signaling proteins. The principal consequence of the mutated proteins is a marked decrease in interactions between Ras and its GTPase activator protein. Instead of reverting to its inactive guanosine diphosphate-bound state, the modified conformation of mutant Ras favors its active GTP-bound state, which has a higher propensity to activate downstream effectors even in the absence of growth factor stimulation, conferring a proliferative advantage to tumors [The Ras/Raf/MAPK Pathway. Molina, Julian R. et al. Journal of Thoracic Oncology, Volume 1, Issue 1, 7-9].

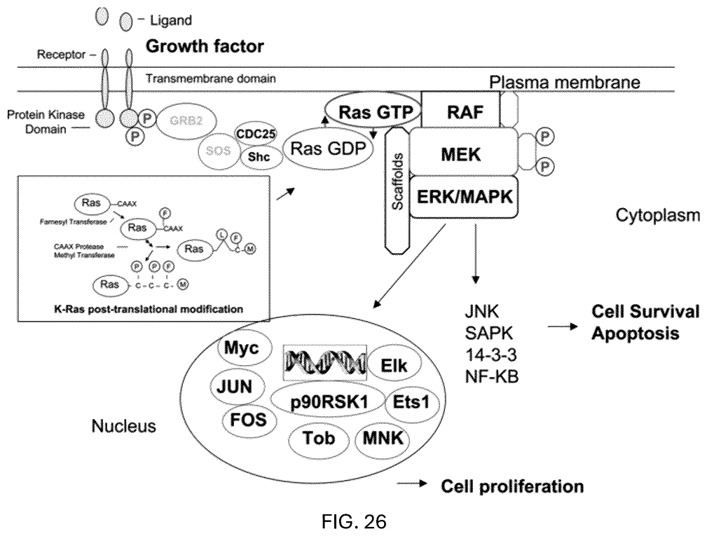

Activation of the pathway begins when a signal binds to a protein tyrosine kinase receptor. The epidermic growth factor receptor (EGFR) and the platelet-derived growth factor receptor (PDGFR) are the best-known receptors in the pathway. However, multiple upstream receptors including other receptor tyrosine kinases, integrins, serpentine receptors, heterotrimeric G-proteins and cytokine receptors are able to activate K-ras. Binding of a ligand to EGF receptor induces oligomerization of the receptor, a process that results in juxtaposition of the cytoplasmic, catalytic domains in a manner that allows activation of the kinase activity and transphosphorylation. Adaptor proteins such as Grb2 are now able to recognize sequence homology 2 (SH2) domains such as She, which in turn, recruit guanine nucleotide exchange factors (GEFs) like SOS-1 or CDC25 to the cell membrane (See, FIG. 26). The GEF becomes capable of interacting with Ras proteins at the cell membrane to promote a conformational change and the exchange of GDP for GTP. Following Ras activation, Raf is recruited to the cell membrane through binding to the switch I domain of Ras and also by lipid binding [The Ras/Raf/MAPK Pathway. Molina, Julian R. et al. Journal of Thoracic Oncology, Volume 1, Issue 1, 7-9].

Raf is the best characterized Ras effector and is a member of a family of serine/threonine kinases, that includes Raf-1, A-Raf and B-Raf. Raf activation stimulates a signaling cascade by phosphorylation of MAPK which successively phosphorylate and activate downstream proteins such as ERK1 and ERK2 (See, FIG. 26). Activation of ERK is critical for a large number of Ras-induced cellular responses. ERK1 and ERK2 phosphorylate and activate a variety of nuclear transcription factors and kinases, including Elk-1, c-Ets1, c-Ets2, p90RSK1, MNK1, MNK2, as well as other proteins such as the anti-proliferative protein Tob. Many of these MAPK/ERK targets have been implicated in Ras induced cell transformation. MAPK, which in mammalians is also called MEK, is a serine/threonine kinase activated in response to multiple signals including growth factors and cytokines to promote cell survival and apoptosis through a number of mediators such as JNK, SAPK, 14-3-3 and NF-KB. MAPK may also regulate both Raf and ERK, providing for cross talk between multiple signaling pathways. Indeed, MAPK appears to induce apoptosis by dysregulation of a number of pathways including ERK, JNK and p38. MAPK has been shown to directly interact with K-ras in a GTP-dependent manner. Raf and MAPK are not the only downstream targets of K-ras, other downstream effectors of K-ras include the PI3K cell survival pathway, the small GTP-binding proteins Rac and Rho, and the stress-activated protein kinase pathway (also referred to as the c-jun N-terminal kinase (JNK) pathway). In addition, in response to cellular stress and cytokine stimulation mediated through K-ras, the dual-specificity p38^{MAPK} kinases (MKK3 and MKK6) and the INK kinases (MKK4 and MKK7) phosphorylate p38^{MAPK} and JNK, respectively binding [The Ras/Raf/MAPK Pathway. Molina, Julian R. et al. Journal of Thoracic Oncology, Volume 1, Issue 1, 7-9].

The term “recurrent cancer” or “recurrence” means a cancer that has come back, usually after a period of time during which the cancer could not be detected. The cancer may come back to the same place as the primary tumor or to another place in the body.

The term “refractory cancer” or “resistant cancer” means a cancer that does not respond to treatment. The cancer may be resistant at the beginning of treatment or it may become resistant during treatment.

The term “relapse” refers to the return of a disease or the signs and symptoms of a disease after a period of improvement.

The terms “relapse-free survival” (RFS) or “disease-free survival” (DFS) mean the length of time after primary treatment for a cancer ends that the patient survives without any signs or symptoms of that cancer.

The term “relative frequency” with respect to a cell population as used herein refers to the proportion of a cell type compared to the total number of cells.

The term “RNA-seq” as used herein refers to a method that uses next-generation sequencing to analyze the transcriptome (i.e., the complete set of RNA transcripts in a cell or organism). RNA seq provides both a qualitative and a quantitative snapshot of gene expression, revealing which genes are active, their abundance, and other information such as genetic abnormalities and alternative splicing. The steps consist of first converting the RNA into cDNA; then (optionally) amplifying the cDNA by PCR; and finally fragmenting the cDNA into short pieces/fragments. After the sequencing library is prepared, the fragments are used as input for next-generation sequencing. The resulting sequence reads contained in FASTQ files are then aligned to a known reference sequence. [Deshpande, D., et al. Frontiers Genetics (2023) 14:997383].

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). According to some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. According to some embodiments, the sequence reads are of a mean, median or average length of about 1000 bp, 2000 bp, 5000 bp, 10,000 bp, or 50,000 bp or more. Nanopore® sequencing methods and associated devices provided by Oxford Nanopore Technology PLC of Oxford, UK, for example, can provide sequence reads that can vary in size from tens to hundreds to thousands of base pairs. Illumina® parallel sequencing methods and associated devices provided by Illumina Inc. of San Diego, CA, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

The term “STAT” or “signal transducers and activators of transcription” as used herein refers to a family of seven transcription factors activated by many cytokine and growth factor receptors.

As used herein, the term “stimulate” and its various grammatical forms as used herein refers to inducing activation or increasing activity.

The term “stimulate an immune cell” or “stimulating an immune cell” as used herein refers to a process (e.g., involving a signaling event or stimulus) causing or resulting in a cellular response, such as activation and/or expansion, of an immune cell, e.g. a CD8+ T cell.

The term “suppression” and its various grammatical forms as used herein refers to the act of reducing, preventing, inhibiting, stopping, or restraining something. As used herein, the term “suppression of expansion” and its various grammatical forms refers to the inhibition or reduction of cell proliferation.

The terms “T cell” or “T lymphocyte” or are used interchangeably to refer to cells that mediate a wide range of immunologic functions, including the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. T cells recognize antigens on the surface of antigen presenting cells (APCs) and mediate their functions by interacting with, and altering, the behavior of these APCs. T cells can also be classified based on their function as helper T cells; T cells involved in inducing cellular immunity; suppressor T cells; and cytotoxic T cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adaptor proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The ultimate amplitude and quality of the T cell immune response, which is initiated through antigen recognition by the TCR, is regulated by a balance between co-stimulatory and inhibitory signals (immune checkpoints).

Although the lineage relationship between T cell subsets remains controversial, T cells cluster in populations that can be arranged as a progressive continuum on the basis of phenotypic, functional and transcriptional attributes. T lymphocytes transition through progressive stages of differentiation that are characterized by a stepwise loss of functional and therapeutic potential in the order from naive T (T_{N}) cells to T memory stem cells (T_{SCM}) (the most immature antigen experienced T cells), to T central memory (T_{CM}) cells, which patrol central lymphoid organs, to T effector memory (T_{EM}) cells, which patrol peripheral tissues. In contrast to T_{N} cells, memory T cells are capable of rapidly releasing cytokines on restimulation. TCM cells more efficiently secrete IL-2 and T_{EM} have an increased capacity for IFNγ release and cytotoxicity. All antigen-experienced T cells upregulate the common IL-2 and IL-15β receptor (IL-2Rβ) conferring the ability to undergo homeostatic proliferation in response to IL-15, and also display high amounts of CD95 (also known as FAS), a receptor that provides either costimulatory or pro-apoptotic signals depending on the efficiency of CD95 signaling complex formation and on which particular intracellular signaling proteins are part of the complex. [Gattinoni, L. et al. Nature Revs. Cancer (2012) 12:671-684].

The term “T cell antigen” as used herein refers to a protein or fragment thereof which can be processed into a peptide that can bind to either Class I MHC, Class II MHC, non-classical MHC, or CD1 family molecules (collectively antigen presenting molecules), and in this combination can engage a T cell receptor on a T cell.

The term “T cell epitope” as used herein is meant to refer to a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

The term “T cell exhaustion” as used herein refers to a state of T cell hypo-responsiveness with reduced cytotoxic activity, decreased cytokine production, and high expression of inhibitory receptors under persistent Ag and chronic TCR stimulation. It is defined by poor effector function, sustained expression of inhibitory receptors and a transcriptional state distinct from that of functional effector or memory T cells. Exhausted T cells become hyporesponsive via a progressive loss of functionality that is mainly mediated by upregulation of multiple inhibitory receptors, including the inhibitory pathways mediated by PD1 in response to binding of PD1 ligand 1 (PDL1) and/or PDL2 [Wherry E J and Kurachi, M. Nature (2015) 15:486-99, citing Okazaki T., et al., Nature Immunol. (2013) 14:1212-1218, Odorizzi P M, Wherry E J. J. Immunol. (2012) 188:2957-2965, Araki K., et al. Cold Spring Harb. Symp. Quant. Biol. (2013) 78:239-247]. Exhausted T cells can co-express PD1 together with lymphocyte activation gene 3 protein (LAG3), 2B4 (also known as CD244), CD160, T cell immunoglobulin domain and mucin domain-containing protein 3 (TIM3; also known as HAVCR2), CTLA4 and many other inhibitory receptors [Id., citing Blackburn S D., et al. Nat. Immunol. (2009) 10:29-37]. Typically, the higher the number of inhibitory receptors co-expressed by exhausted T cells, the more severe the exhaustion. While long thought to be a dysfunctional state, T cell dysfunction is now appreciated as a protective mechanism to avoid damage to normal tissues rather than a complete loss of function [Wen, S. et al. J. Leuk. Biol. (2021) 110:585-90, citing Blank, C U et al. Nat. Rev. Immunol. (2019) 19:665-74]. The population of exhausted T cells is highly heterogeneous and includes such subpopulations as precursor exhausted T cells (T_{PEX} cells), which are defined above. Terminally exhausted T cells preferentially express markers such as CD39, CD244, and TIM3 [Jiang, W. et al. Front. Immunol. (2021) 11:622509].

The term “T cell receptor” (TCR) as used herein refers to a complex of integral membrane proteins that participate in the activation of T cells in response to an antigen. The TCR expressed by the majority of T cells consisting of a heterodimer of α and β chains. A small group of T cells express receptors made of γ and δ chains. Among the α/β T cells are two sublineages: those that express the coreceptor molecule CD4 (CD4+ cells), and those that express CD8 (CD8+ cells). These cells differ in how they recognize antigen and in their effector and regulatory functions. CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated. The cytokines can be directly toxic to target cells and can mobilize potent inflammatory mechanisms. CD8+ T cells can develop into cytotoxic T-lymphocytes (CTLs) capable of efficiently lysing target cells that express antigens recognized by the CTLs.

Naive conventional CD4 T cells can differentiate into four distinct T cell populations, a process that is determined by the pattern of signals they receive during their initial interaction with antigen. These four T cell populations are T_{H}1, T_{H}2, T_{H}17, and induced regulatory T (iTreg) cells. T_{H}1 cells, which are effective inducers of cellular immune responses, mediate immune responses against intracellular pathogens, and are responsible for the induction of some autoimmune diseases. Their principal cytokine products are IFNγ (which enhances several mechanisms important in activating macrophages to increase their microbiocidal activity), lymphotoxin α (LTα), and IL-2, which is important for CD4 T cell memory. T_{H}2 cells, which are effective in helping B cells develop into antibody producing cells, mediate host defense against extracellular parasites, are important in the induction and persistence of asthma and other allergic disease, and produce IL-4, IL-5, IL-9, IL-10 (which suppresses T_{H}1 cell proliferation and can suppress dendritic cell function), IL-13, IL-25 (signaling through IL-17RB, enhances the production of IL-4, IL-5, and IL-13 by a c-kit-FcεRI-nonlymphocyte population, serves as an initiation factor as well as an amplification factor for T_{H}2 responses) and amphiregulin. IL-4 and IL-10 produced by T_{H}2 cells block IFNγ production by T_{H}1 cells. T_{H}17 cells produce IL-17a, IL-17f, IL-21, and IL-22. IL-17a can induce many inflammatory cytokines, IL6 as well as chemokines such as IL-8 and plays an important role in inducing inflammatory responses. Treg cells play a critical role in maintaining self-tolerance and in regulating immune responses. They exert their suppressive function through several mechanisms, some of which require cell-cell contact. The molecular basis of suppression in some cases is through their production of cytokines, including TGFβ, IL-10, and IL-35. TGFβ produced by T reg cells may also result in the induction if iTreg cells from naïve CD4 T cells. CD4+ T cells bear receptors on their surface specific for the B cell's class II/peptide complex. B cell activation depends not only on the binding of the T cell through its T cell receptor (TCR), but this interaction also allows an activation ligand on the T cell (CD40 ligand) to bind to its receptor on the B cell (CD40) signaling B cell activation [Zhu, J. and Paul, W E, Blood (2008) 11:1557-69]. Resting naïve CD8+ T cells, when primed by antigen presenting cells that have acquired antigens from the infected macrophages through direct infection or cross-presentation in secondary lymphoid organs, such as lymph nodes and spleen, react to pathogens by massive expansion and differentiation into cytotoxic T lymphocyte effector cells that migrate to all corners of the body to clear the infection. In the majority of viral infections, however, CD8 T cell activation requires CD4 effector T cell help to activate dendritic cells for them to become able to stimulate a complete CD8 T cell response. CD4 T cells that recognize related antigens presented by the APC can amplify the activation of naïve CD8 T cells by further activating the APC. B7 expressed by the dendritic cell first activates the CD4 T cells to express IL-2 and CD40 ligand. CD40 ligand binds CD40 on the dendritic cell, delivering an additional signal that increases the expression of B7 and 4-1BBL by the dendritic cell, which in turn provides additional co-stimulation to the naïve CD8 T cell. The IL-2 produced by activated CD4 T cells also acts to promote effector CD T cell differentiation.

The term “target gene” as used herein refers to a specific gene of interest in the genome that is intended for genetic modification. Genetic modifications include, but are not limited to, knocking out a target gene (i.e., inactivating the gene), knocking down the target gene (i.e., reducing expression of the target gene), changing the target gene sequence (i.e., generating point mutations or single nucleotide variations), knocking in a gene (i.e., inserting a new gene into the genome), upregulating a target gene (i.e., overexpressing a target gene). A target gene can be inserted into an expression vector that is designed to ensure that the target gene is expressed within the host cell. A target gene can also be the specific gene sequence identified by a guide RNA for manipulation via CRISPR/Cas9.

The term “Tbet” as used herein refers to a T_{H}1 cell transcription factor. Differential expression of the T_{H}1 cell transcription factor Tbet and a closely related T-box family transcription factor particularly in CD8+ T cells, Eomesodermin (Eomes) facilitate the cooperative maintenance of the pool of antiviral CD8+ T cells during chronic viral infection [Paley, M A., et al., Science (2012) 338:1220-125]. During chronic infections, T-bet is reduced in virus-specific CD8+ T cells; this reduction correlates with T cell exhaustion. In contrast, Eomes mRNA expression is up-regulated in exhausted CD8+ T cells during chronic infection [Id.]

The term “helper T cells” or “T_{H}” cells as used herein refers to effector CD4 T cells that stimulate or “help” B cells to make antibody in response to antigenic challenge. T_{H}2, T_{H}1 and the T_{H}F subsets of effector CD4 T cells can perform this function.

The term “T follicular helper (THF) cells” as used herein refers to a distinct subset of CD4+ T lymphocytes, specialized in B cell help and in regulation of antibody responses. They develop within secondary lymphoid organs (SLO) and can be identified based on their unique surface phenotype, cytokine secretion profile, and signature transcription factor. They support B cells to produce high-affinity antibodies toward antigens in order to develop a robust humoral immune response and are crucial for the generation of B cell memory. They are essential for infectious disease control and optimal antibody responses after vaccination. Stringent control of their production and function is critically important, both for the induction of an optimal humoral response against thymus-dependent antigens but also for the prevention of self-reactivity [Gensous, N. et al. Front. Immunol. (2018) doi.org/10.3389/fimmu.2018.01637].

The term “T_{H}1 cells” as used herein refers to a lineage of CD4+ effector T cells that promotes cell-mediated immune responses and is required for host defense against intracellular viral and bacterial pathogens. They are mainly involved in activating macrophages but can also help stimulate B cells to produce antibody. T_{H}1 cells secrete IFN-gamma, IL-2, IL-10, and TNF-alpha/beta. IL-12 and IFN-γ make naive CD4+ T cells highly express T-bet and STAT4 and differentiate to T_{H}1 cells [Zhang, Y. et al. Adv. Exp. Med. Bio. (2014) 841:15-44].

The term “T_{H}2 cells” as used herein refers to a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naive CD4+ T cells highly express STAT6 and GATA3 and differentiate to T_{H}2 cells [Zhang, Y. et al. Adv. Exp. Med. Bio. (2014) 841:15-44].

The term “T_{H}17 cells” as used herein refers to a CD4+ T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity [Tesmer, L A, et al., Immunol. Rev. (2008) 223:87-113]. The key transcription factor in T_{H}17 cell development is RORγt.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3+CD4+CD25+FoxP3+ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated conventional T cell activation: CTLA-4 and LAG-3 outcompete CD28 and T cell receptor expressed on conventional T cells for binding to CD80/86 and MHC class II on DCs, and Nrp1 stabilizes DC-Treg contact, thereby preventing antigen presentation to conventional T cells [Ikebuchi, R. et al. Front. Immunol. (2019) doi.org/10.3389/fimmu.2019.01098].

The term “TIGIT” as used herein refers to a member of the Ig super family and an immune inhibitory receptor.

The term “TIM-3” as used herein refers to a transmembrane protein and immune checkpoint receptor. It is associated with tumor-mediated immune suppression.

The term “toll-like receptor (TLR)” as used herein refers to membrane bound pattern recognition receptors (PRR) on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The terms “TOX” or “thymocytes selection-associated HMG BOX” as used herein refers to a member of a family of transcriptional factors that contain the highly conserved high mobility group box (HMG-box) region. Increasing studies have shown that TOX is involved in maintaining tumors and promoting T cell exhaustion [Liang, C., et al. Biomark Res. (2021) 9:20].

The terms “transcription” or “DNA transcription” are used interchangeably herein to refer to copying of one strand of DNA into a complementary RNA sequence by the enzyme RNA polymerase.

The term “transcriptional control” as used herein refers to control of gene expression by controlling when and how often a gene is transcribed.

The term “transcription factor” as used herein refers to a protein required to initiate or regulate transcription in eukaryotes. This includes both gene regulatory proteins as well as the general transcription factors.

The term “transduction” as used herein refers to a process whereby foreign DNA is introduced into another cell via a viral vector.

The term “transfection” as used herein refers to the process of introducing a foreign DNA molecule into a eukaryotic cell by nonviral methods.

The terms “translation” and “RNA translation” are used interchangeably herein to refer to the process by which the sequence of nucleotides in a messenger RNA molecule directs the incorporation of amino acids into protein. Translation occurs on a ribosome.

The term “translational control” as used herein refers to control of gene expression by selection of which mRNAs in the cytoplasm are translated by ribosomes.

The term “transplant rejection” as used herein refers to an attack by a recipient immune system on transplanted donor tissue (a graft).

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “tumor necrosis factor-related apoptosis-inducing ligand” (“TRAIL”) as used herein refers to a ligand that has been shown to selectively induce apoptotic cell death in various tumor cells by engaging its death-inducing receptors (TRAIL-R1 and TRAIL-R2). Besides activating caspase-dependent apoptosis in several cancer cells, TRAIL may also activate nonapoptotic signal transduction pathways such as nuclear factor-kappa B, mitogen-activated protein kinases, AKT, and signal transducers and activators of transcription 3, which may contribute to TRAIL resistance in various cancers. TRAIL interacts with five distinct receptors that are encoded by separate genes but share high sequence homology in the extracellular domains. However, only DR4 and DR5, which contain an intracellular death domain, can produce apoptotic signals. [Dai, X et al. Exp. Biol. Med. (Maywood) (2015) 240 (6): 760-773, citing Gasparian, M E et al. Intl. J. Program Cell Death (2009) 14:778-787] The apoptotic signaling pathway of TRAIL is triggered by trimerized TRAIL binding to DR4 and DR5, which enables the receptors to homotrimerize, thereby driving formation of the death-inducing signaling complex (DISC). [Id., citing Wagner, K W et al. Nat. Med. (2007) 13:1070-1077] Upon ligand stimulation, DR4 and DR5 recruit Fas-associated death domain protein (FADD) through death domain interactions. FADD then recruits pro-caspase-8 and 10, and/or the cellular FLICE (caspase-8)-like inhibitory protein (c-FLIP) to the DISC. c-FLIP competes with caspase-8 for FADD binding in the DISC and inhibits the apoptosis signal [Id., citing Scaffidi, C. et al. J. Biol. Chem. (1999) 274:1541-1548]. Following recruitment, procaspase-8 comes into contact with the ubiquitin E3 ligase subunit (CUL3), which catalyzes polyubiquitylation of caspase-8 on its C-terminal region. Polyubiquitylated caspase-8 binds with the ubiquitin-binding protein p62, which promotes the translocation of caspase-8 from the DISC into intracellular ubiquitin-rich foci and subsequently leads to the cleavage and activation of caspase-8 [Id., citing Gonzalvaz, F. and Ashkenazi, A. Oncogene (2010) 29:4752-4765] Activation of caspase-8 at the DISC transfers the apoptosis signal to executioners of apoptosis either directly via the extrinsic or indirectly via the intrinsic-mitochondrial pathway.

The term “signal transducers and activators of transcription” or “STATs” refers to a family of transcription factors activated by many cytokine and growth factor receptors. There are seven STATs (1-4, 5a, 5b, and 6), which reside in the cytoplasm in an inactive form until activated by cytokine receptors. Before activation, most STATs form homodimers due to a specific homotypic interaction between domains present at the amino termini of the individual STAT proteins. The receptor specificity of each STAT is determined by the recognition of the distinctive phosphotyrosine sequence on each activated receptor by the different SH2 domains within the various STAT proteins. Recruitment of a STAT to the activated receptor brings the STAT close to an activated Janus kinase (JAK), which can then phosphorylate a conserved tyrosine residue in the carboxy terminus of the particular STAT. This leads to a rearrangement, in which the phosphotyrosine of each STAT protein binds to the SH2 domain of the other STAT, forming a configuration that can bind DNA with high affinity. Activated STATs predominantly form homodimers with cytokine typically activating one type of STAT. For example, IFN-gamma activates STAT1 and generates STAT 1 homodimers, while IL-4 activates STAT6, generating STAT1 homodimers. Other cytokine receptors can activate several STATS, and some STAT heterodimers can be formed. The phosphorylated STAT dimer enters the nucleus, where it acts as a transcription factor to initiate the expression of selected genes that can regulate growth and differentiation of particular subsets of lymphocytes [Janeway's Immunobiology, 9th Ed., Murphy K. & Weaver, C. Eds. Garland Science, New York (2017) at 110-111].

STAT1 is the major mediator of the cellular response to interferons (IFNs) [Tolomeo, M. et al. Int. J. Mol. Sci. (2022) 23 ((8): 4095, citing Dale, T C et al. Proc. Natl Acad. Sci. USA (1989) 86:1203-1207; Levy, D E et al. Genes Dev. (1989) 3:1362-1371]. It plays a key role in the immune response against viruses and mycobacteria by transducing in the nucleus the signal from type I IFNs (IFNα, IFNβ, IFNε, IFNκ, and IFNω), type II IFN (IFNγ) and type III IFN (IFNλ). As with the other members of the STAT family, STAT1 is activated by tyrosine phosphorylation within the cytoplasm by a class of non-receptor tyrosine kinases called Janus kinases (JAKs and TYK2) associated with IFN receptors [Id., citing Ihle, J N. Proc. Soc. Exp. Biol. Med. (1994) 206:268-272; Darnell, J E et al. Science (1994) 264:1415-1421; Simonovic, N. et al. J. Immunol. (2019) 202:1724-1734]. Upon IFN stimulation, phosphorylated STAT1 molecules dimerize through reciprocal phosphotyrosine (pTyr)-SH2 interactions. STAT1 dimers are transferred in the nucleus where they activate gene transcription [Id., citing Wang, X et al. Annu. Rev. Immunol. (2009) 27:29-60; Seidel, H M et al. Proc. Natl Acad. Sci. USA (1995) 92:3041-3045].

As used herein, the term “xenogeneic graft-versus-host disease” or “x-GVHD” refers to a life threatening reaction that occurs when immune cells from a xenograft attack the recipient's body, viewing it as foreign. This complication is seen particularly in the case of cell transplantations, where the transplanted cells recognize the host cells as foreign and trigger an immune response that attacks the recipient's tissue.

As used herein, the term “xenograft” refers to cells transplanted from one species to another species.

As used herein, the term “xeno-reactive” refers to an immune reaction in one species generated in response to cells, tissues, organs, etc. from a different species.

### Embodiments

### Cell Therapy Methods for Inhibiting Autoimmune Reactivity

The present disclosure provides cell therapy methods for inhibiting auto and allo-immune reactivity. According to some embodiments, the cell therapy method comprises administering a population of cells from a donor subject to a recipient subject. According to some embodiments, the donor cells are autologous to the recipient subject. According to some embodiments, the donor cells are allogeneic to the recipient subject. According to some embodiments, the donor cells are autologous to the recipient subject According to some embodiments, the donor cell population is a hematopoietic cell population. According to some embodiments, the donor hematopoietic cells are purified from peripheral blood (PB). According to some embodiments, the donor hematopoietic cells are purified from umbilical cord blood.

In one aspect, the recipient subject is afflicted with a disease or disorder comprising an autoimmune component. Exemplary diseases or disorders comprising an autoimmune component include, without limitation, Addison's disease, Alopecia Areata (AA), amyloidosis, celiac disease, Crohn's disease, glomerulonephritis, Hashimoto thyroiditis, multiple sclerosis, type 1 diabetes mellitus, type 2 diabetes mellitus, myasthenia gravis, polymyositis, psoriasis, rheumatoid arthritis, scleroderma, Sjogren syndrome, and systemic lupus erythematosus. The presence of one autoimmune disease is thought to increase the chance for developing another simultaneous autoimmune disease.

In one aspect, the cell therapy method comprises administering an allogeneic or autologous hematopoietic cell population derived from a healthy donor to a recipient subject, wherein the allogeneic or autologous hematopoietic cells comprise a population of genetically engineered T cells. According to some embodiments, the genetically engineered population of T cells comprises a genetically engineered subpopulation of CD4+ T cells. According to some embodiments, the genetically engineered population of T cells comprises a genetically engineered subpopulation of CD8+ T cells. According to some embodiments, the genetically engineered population of T cells comprises both a genetically engineered subpopulation of CD4+ T cells and a genetically engineered subpopulation of CD8+ T cells.

According to some embodiments, the genetically engineered population of T cells is derived from a healthy donor. According to some embodiments, the genetically engineered T cells are derived from peripheral blood. According to some embodiments, the genetically engineered T cells are derived from peripheral blood after mobilization of peripheral blood stem cells with an infusion of a colony-stimulating factor, e.g., G-CSF. According to some embodiments, the genetically engineered T cells are derived from umbilical cord blood.

G-CSF-mobilized peripheral blood stem cell (PBSC) allo-grafts involve using granulocyte colony stimulating factor (G-CSF) to stimulate a donor's bone marrow to produce and release a large number of stem cells into the donor's bloodstream, which are then collected for an allogeneic transplant [Fan, Q., et al. J Hematol Oncol 10, 135 (2017)]. The term “apheresis” as used herein refers to a medical technology in which the blood of a donor or patient is passed through an apparatus that separates out one particular constituent and returns the remainder back to the donor or patient's circulation. Leukapheresis is one type of apheresis where leukocytes (white blood cells) are selectively removed.

According to some embodiments, the genetically engineered subpopulation of CD4+ T cells and the genetically engineered subpopulation of CD8+ T cells are present in the genetically engineered population of T cells in equal cell-cell ratios. According to some embodiments, the genetically engineered subpopulation of CD4+ T cells and the genetically engineered subpopulation of CD8+ T cells are present in the genetically engineered population of T cells in unequal cell: cell ratios. According to some embodiments the ratio of genetically engineered CD4+ T cells to genetically engineered CD8+ T cells is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, inclusive.

According to some embodiments, the allogeneic or autologous hematopoietic cell population comprises a cell count of about 10 million-100 million cells, inclusive, about 100-500 million cells, inclusive, about 500 million-1 billion cells, inclusive, about 1-2 billion cells, inclusive, about 2-3 billion cells, inclusive, about 3-4 billion cells, inclusive, about 4-5 billion cells, inclusive, about 5-6 billion cells, inclusive, about 6-7 billion cells, inclusive, about 7-8 billion cells, inclusive, about 8-9 billion cells, inclusive, about 9-10 billion cells, inclusive, about 10-11 billion cells, inclusive, about 11-12 billion cells, inclusive, about 12-13 billion cells, inclusive, about 13-14 billion cells, inclusive, about 14-15 billion cells, inclusive, about 15-16 billion cells, inclusive, about 16-17 billion cells, inclusive, about 17-18 billon cells, inclusive, about 18-19 billion cells, inclusive, or about 19-20 billion cells, inclusive.

According to some embodiments, the genetically engineered population of T cells comprises a cell count of about 10 million-100 million cells, inclusive, about 100-500 million cells, inclusive, about 500 million-1 billion cells, inclusive, about 1-2 billion cells, inclusive, about 2-3 billion cells, inclusive, about 3-4 billion cells, inclusive, about 4-5 billion cells, inclusive, about 5-6 billion cells, inclusive, about 6-7 billion cells, inclusive, about 7-8 billion cells, inclusive, about 8-9 billion cells, inclusive, about 9-10 billion cells, inclusive, about 10-11 billion cells, inclusive, about 11-12 billion cells, inclusive, about 12-13 billion cells, inclusive, about 13-14 billion cells, inclusive, about 14-15 billion cells, inclusive, about 15-16 billion cells, inclusive, about 16-17 billion cells, inclusive, about 17-18 billon cells, inclusive, about 18-19 billion cells, inclusive, or about 19-20 billion cells, inclusive.

According to some embodiments, the genetically engineered subpopulation of T cells comprises about 1-100%, inclusive, of the total allogeneic or autologous hematopoietic population of T cells. According to some embodiments, the genetically engineered subpopulation of T cells comprises about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the total allogeneic or autologous hematopoietic population of T cells.

According to some embodiments, cell count can be assessed by any technique known to the skilled artisan. For example, According to some embodiments, the genetically engineered population of T cell is counted by flow cytometry. According to some embodiments, cell count can be assessed by a cell viability assay including, but not limited to, a dye exclusion assay or a metabolic assay, or a cell proliferation assay including, but not limited to, a DNA synthesis assay or a cell counting assay

According to some embodiments, the genetically engineered population of T cell is assessed for cell viability. According to some embodiments, cell viability can be assessed by a dye exclusion assay including, but not limited to, a 7-aminoactinomycin D (7-AAD) assay (where dead or dying cells allow 7-AAD to enter through the cell membrane, where the 7-AAD intercalates into double stranded DNA), a propidium iodide assay (where the propidium iodide fluorescence molecule cannot passively cross an intact cell plasma membrane and therefore is not permeant to live cells) or a Calcein-AM assay (where in live cells, the nonfluorescent calcein AM is converted to green fluorescent calcein by intracellular esterases), a metabolic assay including, but not limited to, a 3-[4,5-dimethylthiazol-2-yl]-2,5 diphenyl tetrazolium bromide (MTT) or 2,3-bis-(2-methoxy-4-nitro-5-sulphenyl)-(2H)-tetrazolium-5-carboxanilide (XTT) assay or a Resazurin (7-hydroxy-10-oxido-phenoxazin-10-ium-3-on) redox dye assay, a proliferation assay including, but not limited to, a 5-bromo-2′-deoxyuridine (BrdU) assay or a carboxyfluorescein (CFSE) assay, flow cytometry, or a combination thereof.

According to some embodiments, the genetically engineered population of T cells is genetically engineered to increase expression of at least one gene when compared to a nonengineered control. According to some embodiments, the genetic engineering process increases expression of the gene(s) by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a control. According to some embodiments, this increased expression is termed overexpression. According to some embodiments, the control is a non-genetically engineered population of cells, including, but not limited to immune cells, e.g., T cells, NK cells, NKT cells, B cells, dendritic cells, monocytes, macrophages.

According to some embodiments, the genetically engineered population of T cells is genetically engineered to reduce expression of at least one gene when compared to a nonengineered control. According to some embodiments, the genetic engineering reduces expression by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a control. According to some embodiments, the control is a non-genetically engineered population of cells, including, but not limited to immune cells, e.g., T cells, NK cells, NKT cells, B cells, dendritic cells, monocytes, macrophages.

According to some embodiments, the genetically engineered population of T cells overexpresses PD-L1. According to some embodiments, the genetically engineered population of T cells overexpresses IFN-α2. According to some embodiments, the genetically engineered CD4+ subpopulation of T cells overexpresses PD-L1. According to some embodiments, the genetically engineered CD4+ subpopulation of T cells overexpresses IFN-α2. According to some embodiments, the genetically engineered subpopulation of CD8+ T cells overexpresses PD-L1. According to some embodiments, the genetically engineered subpopulation of CD8+ T cells overexpresses IFN-α2. According to some embodiments, the genetically engineered population of T cells overexpresses PD-L1 and IFN-α2. According to some embodiments, the genetically engineered subpopulation of CD4+ T cells overexpresses PD-L1 and IFN-α2. According to some embodiments, the genetically engineered subpopulation of CD8+ T cells overexpresses PD-L1 and IFN-α2. According to some embodiments, both the genetically engineered subpopulation of CD4+ T cells population and the genetically engineered subpopulation of CD8+ T cells overexpress PD-L1 and IFN-α2.

According to some embodiments, the genetically engineered population of T cells is genetically engineered to increase expression of PD-L1. According to some embodiments, the expression of PD-L1 is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a non-genetically engineered control. According to some embodiments, the control is a non-genetically engineered population of cells, including, but not limited to immune cells, e.g., T cells, NK cells, NKT cells, B cells, dendritic cells, monocytes, macrophages.

According to some embodiments, the genetically engineered population of T cells is genetically engineered to increase expression of IFN-α2. According to some embodiments, the expression of IFN-α2 is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a non-genetically engineered control. According to some embodiments, the control is a non-genetically engineered population of cells, including, but not limited to immune cells, e.g., T cells, NK cells, NKT cells, B cells, dendritic cells, monocytes, macrophages.

According to some embodiments, expression vectors are employed to genetically engineer the population of T cells. According to some embodiments, the expression vectors comprise a target gene, wherein the target gene is cloned into the vector, wherein the vector comprises a promoter and regulatory elements. According to some embodiments, the expression vector is a viral vector. According to some embodiments, the viral vector is an adenoviral vector. According to some embodiments, the viral vector is an adeno-associated viral vector. According to some embodiments, the viral vector is a lentiviral vector. According to some embodiments, the viral vector is a retroviral vector.

According to some embodiments, the genetically engineered population of T cells is genetically engineered using a CRISPR/Cas9 system. According to some embodiments, the CRISPR/Cas9 system comprises a guide RNA (gRNA). According to some embodiments, the gRNA is about 15-150 nucleotides in length, inclusive. According to some embodiments, the gRNA is about 20 nucleotides in length. According to some embodiments, the gRNA is homologous (meaning the degree of similarity between base sequences in different DNA molecules or in different parts of the same DNA molecule; two DNA molecules that are 100% homologous have identical sequences of nucleotides) to a target gene. According to some embodiments, the gRNA forms a complex with a Cas9 enzyme, generating a functional ribonucleoprotein complex (i.e., a structure consisting of both RNA and protein). According to some embodiments, the gRNA guides the Cas9 enzyme to the target gene of interest. According to some embodiments, the gRNA interacts with the target gene of interest through complementary base-pairing (a base pair is a pair of bases, each in a separate nucleotide, in which each base in hydrogen bonded to the other. A classical Watson-Crick base pair always contains one purine and one pyrimidine: adenine pairs specifically with thymine (A-T), guanine with cytosine (G-C) and uracil with adenine (U-A); the two bases in a classical base pair are said to be complementary to each other. Classical base pairing is responsible for holding together the strands in dsDNA or dsRNA). According to some embodiments, the Cas9 enzyme cleaves the target gene based on the amount of complementary base pairing between the gRNA and the target gene. According to some embodiments, the Cas9 enzyme cleaves the target gene when 15 nucleotides of the gRNA base pairs with the target gene. According to some embodiments, the Cas9 enzyme cleaves the target gene when 16 nucleotides of the gRNA base pairs with the target gene. According to some embodiments, the Cas9 enzyme cleaves the target gene when 17 nucleotides of the gRNA base pairs with the target gene. According to some embodiments, the Cas9 enzyme cleaves the target gene when 18 nucleotides of the gRNA base pairs with the target gene. According to some embodiments, the Cas9 enzyme cleaves the target gene when 19 nucleotides of the gRNA base pairs with the target gene. According to some embodiments, the Cas9 enzyme cleaves the target gene when 20 nucleotides of the gRNA base pairs with the target gene. According to some embodiments, the Cas9 enzyme cleaves the target gene when 21 nucleotides of the gRNA base pairs with the target gene. According to some embodiments, the Cas9 enzyme cleaves the target gene when 22 nucleotides of the gRNA base pairs with the target gene.

According to some embodiments, the genetically engineered population of T cells comprising an overexpression of IFN-α2, PD-L1, or IFN-α2 and PD-L1 is activated and expanded. According to some embodiments, the genetically engineered population of T cells comprising an overexpression of IFN-α2, PD-L1, or IFN-α2 and PD-L1 is activated and expanded in the presence of one or more cytokines. According to some embodiments, the one or more cytokines comprise IL-2, IL7, IL15, IL-18, IL-21 or a combination thereof. According to some embodiments, the genetically engineered population of T cells comprising an overexpression of IFN-α2, PD-L1, or IFN-α2 and PD-L1 is activated and expanded in the presence of CD3/CD28 activating agents. CD3 CD28 T cell activation is achieved by treating T cells with monoclonal anti-CD3 and anti-CD28 antibodies, which provide a co-stimulatory signal essential for effective T cell stimulation. A non-limiting example of a CD3/CD28 activating agent is CD3/CD28 Dynabeads™, as described in thermofisher.com/order/catalog/product/11132D.

Activated T cells proliferate and express surface receptors, such as CD25 (the IL-2 receptor) and CD71 (the transferrin receptor), or costimulatory molecules, such as CD26, CD27, CD28, and CD154. Both surface marker expression and cell proliferation can be assessed by flow cytometry. In addition, T-cell surface proteins (e.g., CD30) are released by activated T cells into the circulation as soluble protein (e.g., sCD30) and can be measured in serum or plasma by enzyme-linked immunoassays. Alternatively, T-cell activation can be monitored by cytokine production and gene expression assessment (e.g., Elispot).

Another approach to assess the state of T cell activation is to examine the upregulation of cytokines. CD4+ T_{H}1 cells are the primary source of the pro-inflammatory cytokines IFN-γ, IL-2, and TNFβ, while IL-4, IL-5, IL-9, IL-10, IL-13 and IL-25 are produced by CD4+ T_{H}2 cells [Id., citing Zhu, J. and Paul, W E. Blood (2008) 112:1557-69]. Activated CD8+ effector and memory T cells also produce IL-2, IL-10, TNFα, and IFNγ [Id., citing Zhang, N. and Bevan, M J. Immunity (2011) 35:161-8]. Cytokines can be monitored inside activated T cells (e.g., by flow cytometry) or the release of cytokines from stimulated T cells can be followed (e.g., by ELISAs, antibody-coated beads and flow cytometry, ELISPOT).

Since T cell activation is accompanied by upregulation of genes, such as those coding for cell division, cytokines, adhesion molecules or cytotoxic peptides, another approach is to follow these genes individually. The term “gene signature” as used herein refers to global changes in a group of genes associated with a particular pathology. A standard scheme for gene signature construction includes the following steps:
(1) selecting an extended list of candidate genes that potentially can be utilized in a gene signature whose expression can reliably be measured using the selected technology that are associated with a particular pathology or positive expression in a significant fraction of samples;
(2) taking a learning set of samples with known clinical annotation and ranking genes in that learning sample; and
(3) selecting and fine-tuning a classification algorithm based on the identified gene set.

According to some embodiments, gene signatures can be described by quantitative polymerase chain reaction (qPCR). According to some embodiments, gene signatures can be described using DNA microarrays, a technology in which thousands of nucleic acids are bound to a surface and used to measure the relative concentration of nucleic acid sequences in a mixture by hybridization and subsequent detection of the hybridization events. The principle of hybridization analysis is that a single-stranded DNA or RNA molecule of defined sequence (the probe) can base pair to a second DNA or RNA sequence that contains a complementary sequence (the target), with the stability of the hybrid depending on the extent of base pairing that occurs. Compared to qPCR, DNA microarrays are less sensitive to detect small changes in gene expression due to background noise.

According to some embodiments, proliferation can be detected by incorporation of labeled DNA precursors. Alternatively, cell division and growth can be detected by incubating cells with a colorimetric substrate, such as the tetrazolium salt MTT (3-(4,5-dimethylthiazol-2-yl)-2,5-diphenyl-tetrazolium bromide; thazolyl blue), which is reduced by mitochondrial succinate dehydrogenase. The succimidyl ester of carboxy fluorescein diacetate (CFSE), which irreversibly binds to proteins both on the cell surface and intracellularly by reaction with lysine and other amine groups, also can be used to study cell proliferation. During cell division, CFSE labeling is distributed equally between daughter cells, thereby losing fluorescence with cycles of cell division, which can be followed by flow cytometry.

According to some embodiments, the administration of the allogeneic or autologous hematopoietic cell population comprising the subpopulation of genetically engineered T cells suppresses the expansion of autoreactive conventional T cells in the recipient subject compared to a control. According to some embodiments, suppression of autoreactive T cell expansion can be measured by flow cytometry. According to some embodiments, expansion of the autoreactive conventional T cell population(s) is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a control. According to some embodiments, the control is an allogeneic or autologous hematopoietic cell population substantially free of genetically engineered T cells.

According to some embodiments, the administration of the allogeneic or autologous hematopoietic cell population comprising the subpopulation of genetically engineered T cells causes a decline in/reduces survival of autoreactive conventional T cells in the recipient subject compared to a control. According to some embodiments, the decline/reduction of survival can be measured by flow cytometry. According to some embodiments, survival of conventional T cells is reduced by/the decline in autoreactive conventional T cells is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a control. According to some embodiments, the control is an allogeneic or autologous hematopoietic cell population substantially free of the genetically engineered T cells.

According to some embodiments, the administration of the allogeneic or autologous hematopoietic cell population comprising the population of genetically engineered T cells results in an increase in differentiation of autoreactive conventional T cells into exhaustion-like T cells in the recipient subject compared to a control. According to some embodiments, T cell exhaustion can be quantified using flow cytometry to identify exhaustion-linked surface markers (e.g., PD-1, CTLA-4, LAG-3).

According to some embodiments, autoreactive conventional T cell differentiation into exhaustion-like T cells increases by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% compared to a control, where the control contains allogeneic or autologous hematopoietic cells substantially free of the genetically engineered T cells. According to some embodiments, the exhaustion-like T cells are characterized by a PD-1+TIM3+ phenotype.

According to some embodiments, the administration of the allogeneic or autologous hematopoietic cell population comprising the subpopulation of genetically engineered T cells produces a decreased amount or decreased frequency of autoreactive T cells in the total T cell population in the recipient subject compared to a non-genetically engineered control.

According to some embodiments, the decreased frequency of autoreactive T cells in the total T cell population in the recipient subject comprises a decreased frequency of the autoreactive CD4+ T cell subpopulation compared to the control. According to some embodiments, the decreased frequency of autoreactive T cells in the total T cell population in the recipient subject comprises a decreased frequency of the autoreactive CD8+ T cell subpopulation compared to the control. According to some embodiments, the decrease in the total T cell population in the recipient subject comprises a decreased frequency of IFN-γ+CD4+ effector T cell subpopulation compared to the control. According to some embodiments, the decrease in the total T cell population in the recipient subject comprises a decreased frequency of the IFN-α producing effector CD4+ T cell subpopulation compared to the control. According to some embodiments, the decreased number of total T cells in the T cell population in the recipient subject comprises a decreased frequency of the alloreactive T cell subpopulation compared to the control. According to some embodiments, the decrease in the total T cell population in the recipient subject comprises a decreased frequency of the IL-2 producing T cell subpopulation compared to the control. According to some embodiments, the decrease in the total T cell population in the recipient subject comprises a decreased frequency of the alloreactive effector T cell subpopulation compared to the control. According to some embodiments, the decreased frequency of alloreactive T cells can be measured by sampling one or more of peripheral blood, bone marrow, liver, or spleen of the recipient subject.

According to some embodiments, the decreased in the amount of autoreactive T cells in the total T cell population is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or, 100% compared to a non-genetically engineered control.

According to some embodiments, the administration of the allogeneic or autologous hematopoietic cell population to the recipient subject is by infusion. According to some embodiments, the infusion is intravenous infusion. According to some embodiments, the recipient subject is a mammal. According to some embodiments, the recipient subject is a human.

According to some embodiments, the administration of the allogeneic or autologous hematopoietic cell population comprising the population of genetically engineered T cells elicits: (i) suppressed expansion of conventional T cells in the recipient subject compared to a control; and/or (ii) decreased survival of conventional T cells in the recipient subject compared to a control; and/or (iii) increased conventional T cell differentiation into exhaustion-like T cells in the recipient subject compared to a control; and/or (iv) a decrease in an amount of autoreactive T cells in the recipient subject compared to a control.

In another aspect, the recipient subject is afflicted with a hematologic cancer.

According to some embodiments, the hematologic cancer is a leukemia, a myelodysplastic neoplasm, a myeloproliferative neoplasm, a myeloma, or a lymphoma.

According to some embodiments, the hematologic cancer is a leukemia. Leukemias are tumors that arise from the transformation of a hematopoietic cell in the blood or a hematopoietic precursor in bone marrow; in the latter case, the cancerous progeny of the transformed cell usually make their way into the blood. Leukemias most often occur as “liquid tumors” that are manifested as greatly increased numbers of myeloid, lymphoid, or (more rarely) erythroid lineage cells in the blood and bone marrow.

Examples of leukemias include, without limitation, acute lymphoblastic leukemia (also called acute lymphocytic leukemia and ALL), acute myelogenous leukemia (also called acute myeloblastic leukemia, acute myeloid leukemia, acute nonlymphocytic leukemia, AML, and ANLL), acute myeloid leukemia with myelodysplasia, acute promyelocytic leukemia (also called APL), chronic eosinophilic leukemia; chronic granulocytic leukemia (also called chronic myelogenous leukemia, chronic myeloid leukemia, and CML), erythroleukemia; mast cell leukemia; and hairy cell leukemia.

According to some embodiments, the recipient subject is a child with cancer. According to some embodiments, the recipient subject is a young adult with cancer. According to some embodiments, the recipient subject is an adult with cancer.

Cancers that affect children from birth through age 14 are known as childhood cancers or pediatric cancers. Cancers that affect children ages 15 to 19 are known as adolescent cancers, (see, cancer.org/cancer/childhood-cancer). According to some embodiments, the subject with a hematologic cancer is an adult. Although there is no strict definition of what separates “childhood cancers” from cancers in young adults, or when exactly a person is no longer a young adult, cancers in young adults are often thought of as those that start in people between the ages of 20 and 39 (see, cancer.org/cancer/types/cancer-in-young-adults/cancers-in-young-adults).

According to some embodiments, the lymphoma is a Hodgkin lymphoma. According to some embodiments, the lymphoma is a non-Hodgkin lymphoma. According to some embodiments, the non-Hodgkin lymphoma is a B cell lymphoma. According to some embodiments, the non-Hodgkin lymphoma is a T cell lymphoma.

According to some embodiments, the hematologic cancer is a monoclonal gammopathy of undetermined significance (MGUS). According to some embodiments, the hematologic cancer is a smoldering myeloma. According to some embodiments, the hematologic cancer is a multiple myeloma.

According to some embodiments, the hematologic cancer is a leukemia, a myelodysplastic neoplasm, a myeloma, or a lymphoma. According to some embodiments, the hematologic cancer is acute myeloid leukemia, acute lymphoid leukemia, chronic myelomonocytic leukemia, myelodysplastic syndrome, non-Hodgkins lymphoma, or peripheral T cell lymphoma.

According to some embodiments, the administration of the allogeneic or autologous hematopoietic cell population comprising the population of genetically engineered T cells results in: (i) decreased risk of graft-versus host disease of the recipient subject; (ii) decreased clinical signs of graft-versus-host disease of the recipient subject; (ii) increased survival of the recipient subject; (iii) reduced inflammation; or (iv) a combination thereof, compared to a control.

According to some embodiments, the administration of the allogeneic or autologous hematopoietic cell population comprising a genetically engineered T cell population to a recipient subject afflicted with a hematologic cancer results in a decreased risk of a graft versus host reaction in the recipient subject while preserving graft versus tumor immunity.

According to some embodiments, the administration of the allogeneic or autologous hematopoietic cell population comprising the population of genetically engineered T cells results in decreased clinical signs of graft-versus-host disease of the recipient subject compared to a control, where the control comprises non-genetically engineered T cells. According to some embodiments, clinical signs of graft-versus-host disease include, but are not limited to, rash, itchiness, blisters, diarrhea, abdominal pain, nausea, vomiting, loss of appetite, fever, jaundice, skin thickening, skin tightness, dry mouth, mouth sores, dry eyes, sensitivity to light, swelling, cough, difficulty breathing, weak muscles, stiff muscles, joint pain, and hair loss. According to some embodiments, the decrease in clinical signs is measurable, e.g., by at least 10%, at least 15%, at least 20%, 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a control.

According to some embodiments, the administration of the allogeneic or autologous hematopoietic cell population comprising the population of genetically engineered T cells results in increased survival of the recipient subject compared to a control. According to some embodiments, increased survival is measured using the Kaplan-Meier survival analysis.

According to some embodiments, the administration of the allogeneic or autologous hematopoietic cell population comprising the population of genetically engineered T cells results in reduced inflammation compared to a control subject in the recipient subject compared to a control.

According to some embodiments, reduced inflammation is measured by detecting inflammatory markers including, without limitation, C-reactive protein (CRP). CRP tests [mayoclinic.org/tests-procedures/c-reactive-protein-test/about/pac-20385228, ncbi.nlm.nih.gov/books/NBK441843] are highly sensitive and measure CRP levels in the blood. Laboratory values for CRP vary, with no standardized reporting method currently in use. Results are typically presented in either mg/dL or mg/L, with hsCRP commonly reported in mg/dL.

Conventional CRP assays typically are used to evaluate infection, inflammatory disorders and tissue injury. High sensitivity CRP (hsCRP) assays are indicated for identifying and stratifying individuals at risk of developing cardiovascular disease in the future.
Less than 0.3 mg/dL: Normal, typically observed in most healthy adults
0.3 to 1.0 mg/dL: Normal or minor elevation, often seen in conditions such as obesity, pregnancy, depression, diabetes, common cold, gingivitis, periodontitis, sedentary lifestyle, cigarette smoking, and genetic polymorphisms
1.0 to 10.0 mg/dL: Moderate elevation, commonly associated with systemic inflammation such as rheumatoid arthritis, systemic lupus erythematosus, other autoimmune diseases, malignancies, myocardial infarction, pancreatitis, and bronchitis
More than 10.0 mg/dL: Marked elevation, typically linked to acute bacterial infections, viral infections, systemic vasculitis, and major trauma
More than 50.0 mg/dL: Severe elevation, generally seen in acute bacterial infections.

According to some embodiments, inflammation is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or 100% compared to a control.

According to some embodiments, the administration of the allogeneic or autologous hematopoietic cell population comprising the population of genetically engineered T cells results in increased expression of one or more of IFN-stimulated genes, genes involved in cell migration, genes involved in tissue infiltration, genes encoding inhibitory receptors, genes encoding transcription factors, genes involved in inflammatory response, genes involved in INFα response, genes involved in IFN-γ response, compared to a control.

According to some embodiments, the IFN stimulated genes comprise IFI27, IFITM1, and ISG15. IFI27, or interferon alpha-inducible protein 27, is a protein encoded by an IFN stimulating gene that plays a complex role in the immune system, particularly in the response to viral infections [uniprot.org].

IFITM1, or interferon-induced transmembrane protein 1, is a protein encoded by an IFN stimulating gene that plays a dual role as an antiviral defense and a factor in cancer progression [uniprot.org].

ISG15, or interferon-stimulated gene 15, is a protein encoded by an IFN stimulating gene that is a ubiquitin-like protein that plays a critical role in the innate immune response [uniprot.org].

According to some embodiments, the genes involved in cell migration and tissue infiltration comprise ITGA1, ITGA3, CCR5, CCR9, α4β7, CXCR3, and CCR7.

ITGA1, or integrin alpha 1, is a protein involved in promoting cell migration, particularly tumor metastasis [uniprot.org].

ITGA3, or integrin alpha 3, is linked to cell migration and tissue infiltration by facilitating the signaling required for cytoskeletal rearrangement and movement [uniprot.org].

CCR7, a chemokine receptor, is a G-protein coupled receptor involved in regulating the movement of immune cells to lymph nodes and other lymphoid organs [uniprot.org].

α4β7 is an integrin on the surface of immune cells that functions as a homing receptor to direct them to mucosal tissues [uniprot.org].

CCR5 is a chemokine receptor that is involved in cell migration, particularly in immune cells such as T cells and monocytes. The migration is triggered by the presence of its ligand, CCL5, guiding cells to specific locations for functions such as fighting infection, initiating an immune response, or invading tissue [uniprot.org].

CCR9 is involved in directing immune cells, such as T cells and DCs, particularly to the small intestine and the thymus, by responding to chemokine CCL25 [uniprot.org].

CXCR3 is a chemokine receptor that plays a role in guiding the movement of immune cells to areas of inflammation. CXCR3 is activated by chemokines CXCL9 and CXCL10 [uniprot.org].

According to some embodiments, the genes encoding inhibitory receptors comprise HAVCR2, LAG3, ENTPD1, and TIGIT.

HAVCR2 is a gene that encodes the protein TIM3, which is a negative regulator of the immune system, helping control immune and inflammatory responses and plays a role in immune tolerance [uniprot.org].

LAG3 is a protein that acts as an immune checkpoint receptor on the surface of immune cells, such as T cells, to help regulate immune responses [uniprot.org].

LILRB4 is an immunoreceptor tyrosine-based inhibition motif-containing receptor and a marker of monocytic leukemia [Deng, M. et al. Nature (2018) 562:605-609].

ENTPD1 is a gene that encodes the protein CD39, which is a plasma membrane enzyme that breaks down extracellular ATP and ADP into AMP. It is a key regulator of immune responses and helps suppress unwanted immune activity [uniprot.org].

TIGIT, or T-cell immunoglobulin and ITIM domain, is an immune checkpoint protein found on the surface of T cells, which acts as an inhibitory receptor that helps regulate the immune system, often by dampening immune responses to prevent autoimmune disease [uniprot.org].

According to some embodiments, the genes encoding transcription factors comprise PRDM1, TOX, STAT1, and CEBPB.

PRDMI (also known as BLIMP-1), is a gene that encodes a transcription factor involved in a range of immune functions. It is a transcriptional repressor that plays a critical role in regulating cell fate, maintaining immune tolerance, and preventing cancer by silencing stem-cell related genes [uniprot.org].

TOX is a transcription factor that plays a role in the development and differentiation of T cells. It can promote long-term survival of T cells but also contributes to T cell exhaustion [uniprot.org].

STAT1 is a transcription factor that plays a crucial role in the immune system's response to infection. It is activated by cytokines and is involved in suppressing gene expression [uniprot.org].

CEBPB is a gene that encodes a transcription factor that plays a vital role in immune reactions to inflammation and infections. It influences inflammatory diseases, cancer progression, and the tumor microenvironment by controlling the expression of genes related to inflammation and immune checkpoints [uniprot.org].

According to some embodiments, the genes involved in inflammatory response, INFα response, and IFN-γ response comprise LAG3, HAVCR2, and TOX.

According to some embodiments, the administration of the allogeneic or autologous hematopoietic cell population comprising the population of genetically engineered T cells results in increased expression of p-SHP2 compared to a control.

p-SHP2 is the phosphorylated form of the SHP2 protein, which is a critical enzyme involved in cellular signaling pathways, particularly in regulating the response to growth factors and cytokines. The activated p-SHP2 is crucial for regulating many signaling pathways, including the Ras-MAPK pathway (see, FIG. 26), which is involved in cell growth and proliferation, and the JAK/STAT pathway (see, FIGS. 27A and 27B), which is involved in immune responses [Zhang J, et al. J Cell Mol Med. 2015 September; 19 (9): 2075-83].

According to some embodiments, gene expression is measured using RNA-seq. According to some embodiments, gene expression is analyzed using a gene set enrichment analysis (GSEA).

According to some embodiments, the result of administration of the allogeneic or autologous hematopoietic cell population comprising the subpopulation of genetically engineered T cells comprises an increased frequency of PD-1+CX3CR1+CD4+ conventional T cells in the recipient subject compared to a control, wherein the increased frequency of PD-1+CX3CR1+CD4+ conventional T cells in the recipient subject is detectable in peripheral blood, bone marrow, liver, spleen, or a combination thereof. According to some embodiments, the PD-1+CX3CR1+CD4+ conventional T cells are TOX+TCF1− T cells.

According to some embodiments, preserving graft versus tumor immunity in the recipient subject maintains the production of cytotoxic effector T cells in the recipient subject.

According to some embodiments, the administration of the genetically engineered allogeneic or autologous population of T cells is by intravenous infusion. According to some embodiments, the recipient subject is a mammal. According to some embodiments, the recipient subject is human.

### Additional Agents

In some aspects, the described methods include combination of the described cell therapy with other therapeutic agents.

According to some embodiments, the method includes administering an approved immune checkpoint inhibitor at a dose standard for the cancer indication [Wardill, H. et al. Cancers (Basel) (2023) 15 (16): 4137; McClanahan, F. et al. Blood (2015) 126 (2): 203-11 (CLL); Gomez-Llobell, M. et al. Front Oncol. (2022) 12:882531 (AML); Al-Hadidi, S A and Lee, H J. JCO Oncology Practice (2021) 17 (2): doi.org/10.1200/OP.20.00771 (cHL)].

Immune checkpoints engage when proteins on the surface of T cells recognize and bind to partner immune checkpoint proteins on other cells, such as some tumor cells. When the checkpoint and partner proteins bind together, they send an “off” signal to the T cells, which can prevent the immune system from destroying the cancer. Immune checkpoint inhibitors can block checkpoint proteins from binding with their partner proteins, which prevents the “off” signal from being sent, allowing the T cells to kill cancer cells. Checkpoint inhibitors are not effective in all cancers.

Examples of commercially available checkpoint inhibitors include anti-PD1 (e.g., lambrolizumab/pembrolizumab (KEYTRUDA®, or its biosimilars) and nivolumab (OPDIVO® or its biosimilars), anti-PD-L1 (Atezolizumab, TECENTRIQ®, MPDL3280A, or its biosimilars), and anti-CTLA4 (ipilimumab, YERVOY® or its biosimilars).

According to some embodiments, the additional agent comprises rituximab (anti-CD20). According to some embodiments, the additional agent comprises alemtuzumab (anti-CD52). According to some embodiments, the additional agent comprises epratuzumab (anti-CD22). Rituximab, alemtuzumab and epratuzumab have been incorporated into ALL or CLL regimens because of their activity against certain leukemia associated antigens.

According to some embodiments, the additional agent is a PD-1 agonist. For example, Curnock [JCI Insight (2021) 6 (20): e152468] identified several single variable domain on a heavy chain (VHH) PD-1 antibodies with low nanomolar affinities and fused these to the PPI15-24 TCR to produce a panel of PD-1 agonist ImmTAAI molecules that mimic the ability of PD-L1 to facilitate the colocalization of PD-1 with the TCR complex at the target cell-T cell interface and inhibit T cell function locally. For example, PD1 agonist antibody peresolimab (Eli Lilly) is in phase III. Another example, Rosnilimab (AnaptysBio), is also a PD1 agonist.

According to some embodiments, the additional agent is alpha-1-antitrypsin (AAT). It has been demonstrated that monotherapy with clinical-grade human AAT (hAAT) reduced circulating pro-inflammatory cytokines, diminished Graft vs Host Disease (GvHD) severity, and prolonged animal survival after experimental allogeneic bone marrow transfer (Tawara et al., Proc Natl Acad Sci USA. (2012) January 10; 109 (2): 564-9), incorporated herein by reference. AAT treatment reduced the expansion of alloreactive T effector cells but enhanced the recovery of T regulatory T-cells, (Tregs) thus altering the ratio of donor T effector to T regulatory cells in favor of reducing the pathological process. In vitro, AAT suppressed LPS-induced in vitro secretion of proinflammatory cytokines such as TNF-α and IL-1β, enhanced the production of the anti-inflammatory cytokine IL-10, and impaired NF-κB translocation in the host dendritic cells. Marcondes, Blood. 2014 (October 30; 124 (18): 2881-91) incorporated herein by reference show that treatment with AAT not only ameliorated GvHD but also preserved and perhaps even enhanced the graft vs leukemia (GVL) effect.

According to some embodiments, the additional agent is an immunomodulator. According to some embodiments, the immunomodulator comprises a corticosteroid. According to some embodiments, the corticosteroid comprises prednisolone or dexamethasone. According to some embodiments, the immunomodulator comprises an immunosuppressant. According to some embodiments, the immunosuppressant comprises azathioprine, cyclosporine, methotrexate, tacrolimus, sirolimus, or mycophenolate mofetil. According to some embodiments, the immunomodulator comprises a monoclonal antibody. According to some embodiments, the monoclonal antibody comprises adalimumab (Humira® or its biosimilars), infliximab (Remicade® or its biosimilars, anakinra (Kineret®, an interleukin-1 receptor antagonist used to reduce the signs or symptoms of rheumatoid arthritis or its biosimilars), or tocilizumab (brand name Actemr® for treatment of rheumatoid arthritis or its biosimilars). According to some embodiments, the immunomodulators comprise a Janus Kinase (JAK) inhibitor (e.g., Rinvoq® or its biosimilars (upadacitinib), Xeljianz® or its biosimilars (tofacitinib), filgotinib (Jyseleca® or its biosimilars). According to some embodiments, the JAK inhibitors comprise baricitinib (Olumiant® or its biosimilars) or ruxolitinib (Opzelura® or its biosimilars).

According to some embodiments, the additional agent is an anti-autoimmune agent to reduce inflammation. According to some embodiments, the anti-autoimmune agent comprises a TNF inhibitor. According to some embodiments, TNF inhibitors comprise adalimumab (Humira® or its biosimilars), certolizumab (Cimzia® or its biosimilars), etanercept (Enebrel® or its biosimilars), golimumab (Simponi® or its biosimilars), infliximab (Remicade® or its biosimilars).

According to some embodiments, the autoimmune agent comprises an interleukin inhibitor. According to some embodiments the interleukin inhibitor comprises anakinra (Kineret® or its biosimilars), canakinumab (Ilaris® or its biosimilars), tocilizumab (Actemra® or its biosimilars), sarilumab (Kevzara® or its biosimilars), secukinumab (Cosentyx® or its biosimilars), ustekinumab (Stelara® or its biosimilars).

According to some embodiments, the autoimmune agent comprises T cell and/or B cell inhibitors. According to some embodiments, T cell and B cell inhibitors include abatacept (Orencia® or its biosimilars), rituximab (Rituxan® or its biosimilars), or belimumab (Benlysta® or its biosimilars).

According to some embodiments, the additional agent is a chemotherapy. Nonlimiting examples of chemotherapies include alkylating agents (e.g., altretamine (Hexalen® or its biosimilars), bendamustine (Treanda® or its biosimilars), busulfan (Busulfex® or its biosimilars), carboplatin (Paraplatin® or its biosimilars), chlorambucil (Leukeran® or its biosimilars), cisplatin (Platinol® or its biosimilars), cyclophosphamide (Cytoxan® or its biosimilars), dacarbazine (DTIC-Dome® or its biosimilars), ifosfamide (Ifex® or its biosimilars), mechlorethamine (Mustargen® or its biosimilars), melphalan (Alkeran® or its biosimilars), oxaliplatin (Eloxatin® or its biosimilars), procarbazine (Matulane® or its biosimilars), temozolomide (Temodar® or its biosimilars), thiotepa (Tepadina® or its biosimilars), trabectedin (Yondelis® or its biosimilars), carmustine (BiCNU® or its biosimilars), lomustine (Gleostine® or its biosimilars), streptozocin (Zanosar® or its biosimilars), or their biosimilars), antimetabolites (e.g., 5-fluorouracil (Adrucil® or its biosimilars), 6-mercaptopurine (Purixan® or its biosimilars), azacitidine (Vidaza® or its biosimilars), capaecitabine (Xeloda® or its biosimilars), cladribine (Mavenclad® or its biosimilars), clofarabine (Clolar® or its biosimilars), cytarabine (Cytosar-U® or its biosimilars), decitabine (Dacogen® or its biosimilars), floxuridine (FUDR® or its biosimilars), fludarabine (Fludara® or its biosimilars), gemcitabine (Gemzar® or its biosimilars), hydroxyurea (Droxia® or its biosimilars), methotrexate (Maxtrex® or its biosimilars), nelarabine (Arranon® or its biosimilars), pemetrexed (Alimta® or its biosimilars), pentostatin (Nipent® or its biosimilars), pralatrexate (Folotyn® or its biosimilars), thioguanine (Tabloid® or its biosimilars), trifluridine (Viroptic® or its biosimilars), topoisomerase inhibitors (e.g., etoposide (VePesid® or its biosimilars), irinotecan (Camptosar® or its biosimilars), irinotecan liposomal (Onivyde® or its biosimilars), mitoxantrone (Novantrone® or its biosimilars), teniposide (Vumon® or its biosimilars), topotecan (Hycamtin® or its biosimilars), mitotic inhibitors (e.g., cabazitaxel (Jevtana® or its biosimilars), docetaxel (Taxotere® or its biosimilars), nab-paclitaxel (Abraxane® or its biosimilars), paclitaxel (Taxol® or its biosimilars), vinblastine (Velban® or its biosimilars), vincristine (Oncovin® or its biosimilars), vincristine liposomal (Marqibo® or its biosimilars), vinorelbine (Navelbine® or its biosimilars), antitumor antibiotics (e.g., daunorubicin (Cerubidine® or its biosimilars), doxorubicin (Adriamycin® or its biosimilars), doxorubicin liposomal (Doxil® or its biosimilars), epirubicin (Ellence® or its biosimilars), idarubicin (Idamycin® or its biosimilars), mutoxantrone (Novantrone® or its biosimilars), valrubicin (Valstar® or its biosimilars), bleomycin (Blenoxane® or its biosimilars), dactinomycin (Cosmegen® or its biosimilars), mitomycin-c (Mutamycin® or its biosimilars), and other chemotherapies (e.g., all-trans-retinoic acid (Vesanoid® or its biosimilars), arsenic trioxide (Trisenox® or its biosimilars), asparaginase (Rylaze® or its biosimilars), eribulin (Halaven® or its biosimilars), ixabepilone (Ixempra® or its biosimilars), mitotane (Lysodren® or its biosimilars), omacetaxine (Synribo® or its biosimilars), pegaspargase (Oncaspar® or its biosimilars), procarbazine (Matulane® or its biosimilars), romidepsin (Istodax® or its biosimilars), vorinostat (Zolinza® or its biosimilars).

According to some embodiments, the chemotherapy comprises fludarabine (Fludara® or its biosimilars), busulfan (Myleran® or its biosimilars), melphalan (Alkeran® or its biosimilars), cyclophosphamide (Neosar® or its biosimilars), doxorubicin (Adriamycin® or its biosimilars), cisplatin (Platinol® or its biosimilars), carboplatin (Paraplatin® or its biosimilars), 5-fluorouracil (Efudex® or its biosimilars), methotrexate (Maxtrex® or its biosimilars), paclitaxel (Taxol® or its biosimilars), gemcitabine (Gemzar® or its biosimilars), etoposide (VePesid® or its biosimilars), vincristine (Oncovin® or its biosimilars), temozolomide (Temodar® or its biosimilars), cytarabine (Cytosar-U® or its biosimilars), mercaptopurine (Purixan® or its biosimilars), daunorubicin (Cerubidine® or its biosimilars), carmustine (BiCNU® or its biosimilars), irinotecan (Camptosar® or its biosimilars), idarubicin (Idamycin® or its biosimilars), amsacrine (Amekrin® or its biosimilars), ifosfamide (Ifex® or its biosimilars), or methylprednisolone (Medrol® or its biosimilars).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates, which may need to be independently confirmed.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Engineered Suppressor T Cells Overexpressing IFN-α and PD-L1 Inhibit GVHD but Retain GVL Effects in Mice

### Introduction

Living cell therapy for immune regulation could potentially achieve long-lasting effects on inhibiting graft-versus-host disease (GVHD), a life-threatening complication after allogeneic hematopoietic cell transplantation (allo-HCT). However, novel immune regulatory cells with better persistence and sustained immune-modulatory function are needed.

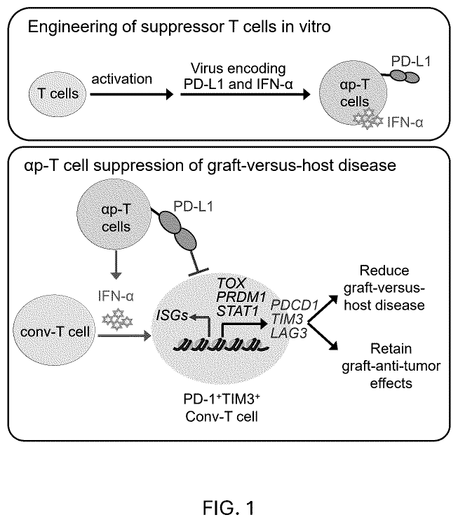

Here, engineering human T cells to co-express IFN-α2 and PD-L1 (named αρ-T cells) rendered them a potent capacity to inhibit xenogeneic GVHD without impairing anti-leukemia in immunodeficient mice are demonstrated. These αρ-T cells maintained stable expression of PD-L1 and IFN-α and potent immunosuppressive effects in peripheral tissues during GVHD control. CD4⁺αρ-T cells activated transcriptional programs that promoted effector differentiation, exhaustion and proliferation inhibition in both themselves and their treated conventional T (conv-T) cells, ultimately reducing GVHD. These CD4⁺αρ-T cells created a synergistic regulatory loop that modulated conv-T cell responses, in which IFN-α suppressed expansion and survival of activated conv-T cells, promoted their differentiation into PD-1⁺TIM3⁺exhaustion-like T cells, and sensitized them to PD-L1-mediated suppression. Further verified were these regulatory effects of murine CD4⁺αρ-T cells on reducing GVHD in immunocompetent mice of allo-HCT. Thus, αρ-T cells may represent a novel translational strategy to improve the safety and efficacy of allo-HCT and inhibit other inflammatory disorders in a broad context (FIG. 1).

FIG. 1 shows a schematic illustrating that engineering human T cells to co-express IFN-α2 and PD-L1 (i.e., αρ-T cells) renders them effective to inhibit xenogeneic graft-versus-host disease (x-GVHD) without impairing anti-leukemia in immunodeficient mice. These αρ-T cells maintained stable expression of PD-L1 and IFN-α and potent immunosuppressive effects in peripheral tissues during GVHD control. CD4⁺αρ-T cells activated transcriptional programs that promoted effector differentiation, exhaustion, and proliferation inhibition in both, themselves and their treated conventional T (conv-T) cells, ultimately reducing GVHD. These CD4⁺αρ-T cells created a synergistic regulatory loop that modulated conv-T cell responses, in which IFN-α suppressed expansion and survival of activated conv-T cells, promoted their differentiation into PD-1⁺TIM3⁺exhaustion-like T cells, and sensitized them to PD-L1-mediated suppression. Further verified were these regulatory effects of murine CD4⁺αρ-T cells on reducing GVHD in immunocompetent mice of allogeneic hematopoietic cell transplantation (allo-HCT). Thus, αρ-T cells may represent a novel translational strategy to improve the safety and efficacy of allo-HCT and inhibit other inflammatory disorders in a broad context.

### Materials and Methods

### Human Samples and T Cells

Peripheral blood (PB) samples were collected from G-CSF-mobilized PB stem cells (G-PBSCs) of allogeneic donors. Patients undergoing allo-HCT were diagnosed and treated at Penn State University College of Medicine. The characteristics of the allo-HCT recipients are summarized in Table 1. The study was approved by the Institutional Review Board of Penn State University College of Medicine. Human T cells were isolated from buffy coat of deidentified healthy donors (New York Blood Center, NY).

TABLE 1
Patient Characteristics

| | GVHD (0-I) | | GVHD (II-IV) | | |
|---|---|---|---|---|---|
| Patient Characteristics | (N = 11) | | (N = 13) | | P Value |
| Median Patient Age, years (range) | 55 | (25-68) | 53.1 | (24-71) | 0.7880 |

Disease, N (%)

| Acute Lymphoid Leukemia | 2 | (18.2) | 0 | | 0.2428 |
|---|---|---|---|---|---|
| Acute Myeloid Leukemia | 9 | (81.8) | 8 | (61.5) | |

| | | | |
|---|---|---|---|
| Chronic Myelomonocytic Leukemia | 0 | 1 | (7.7) |
| Myelodysplastic Syndrome | 0 | 1 | (7.7) |
| Non-Hodkins Lymphoma | 0 | 2 | (15.4) |
| Peripheral T Cell Lymphoma | 0 | 1 | (7.7) |

Disease Status at transplant, N (%)

| | | | | | |
|---|---|---|---|---|---|
| Complete Remission | 10 | (90.9) | 10 | (76.9) | >0.9999 |
| Partial Remission | 1 | (9.1) | 2 | (15.4) | |

| | | | |
|---|---|---|---|
| Relapsed/Refractory/Untreated | 0 | 1 | (7.7) |

Donor Type

| | | | | | |
|---|---|---|---|---|---|
| Matched Unrelated Donor | 9 | (81.8) | 11 | (84.6) | >0.9999 |
| Matched Related Donor | 1 | (9.1) | 1 | (7.7) | |

| | | | |
|---|---|---|---|
| Haploidentical Related Donor | 0 | 1 | (7.7) |

Conditioning Regimen, N (%)

| | | | | | |
|---|---|---|---|---|---|
| Myeloablative* | 2 | (18.2) | 2 | (15.4) | >0.9999 |
| Reduced Intensity Conditioning/Non- | 9 | (81.8) | 11 | (84.6) | |
| Myeloablative** | | | | | |

Transplant Cell Dose, Mean (range)

| | | | | | |
|---|---|---|---|---|---|
| CD34+ (10⁶/kg) | 3.8 | (1.83-5.0) | 4.4 | (4-5.24) | 0.1541 |
| CD3+ (10⁸/kg) | 1.39 | (0.9-2.85) | 1.2 | (0.82-2.08) | 0.5941 |

GVHD Prophylaxis

| | | | | | |
|---|---|---|---|---|---|
| PTcy, MMF, Tacrolimus | 1 | (9.1) | 2 | (15.4) | >0.9999 |
| MTX, Tacrolimus | 10 | (90.9) | 11 | (84.6) | |

*Myeloablative Regimens: Flu/Bu4, Flu/Bu3
**Non-Myeloablative Regimens: Flu/Bu2, Flu/Bu2/Total Body Irradiation, Flu/Mel100/Total Body Irradiation

Flu/Bu refers to allogeneic myeloablative conditioning with fludarabine and busulfan. Mel 100 refers to a reduced dose of melphalan (100 mg/m²).

### Mice

NSG mice, C57BL/6 (B6, H-2b, CD45.2+), B6.SJL (H-2b, CD45.1+) and BALB/c (H-2d) mice were purchased from The Jackson laboratory (Bar Harbor, ME). Experimental protocols were approved by Hackensack Meridian Health's Institutional Animal Care & Use Committee.

### Cell Lines

K562 cells, 293T cells, Platinum-E cells, Raji cells and MV4-11 cells were obtained from ATCC. K562 cells and MV4-11 cells were cultured in IMDM supplemented with 10% FBS, glutamine, and antibiotics. 293T cells and Platinum-E cells were cultured in DMEM supplemented with 10% FBS, glutamine, and antibiotics. Raji cells were cultured in RPMI-1640 supplemented with 10% FBS and antibiotics.

Isolation and Phenotypic Analysis of pDCs

pDCs were isolated by magnetically activated cell sorting using CD304 (BDCA-4/Neuropilin-1) microbeads kit (Miltenyi Biotec, Auburn, CA) from the buffy coat of deidentified healthy donors (New York Blood Center, New York, NY). MNCs from normal healthy donors were isolated by Ficoll® Hypaque density gradient centrifugation. Cells were then magnetically labeled with anti-BDCA-4 antibody coupled to colloidal paramagnetic microbeads and passed through a magnetic separation column twice (Miltenyi Biotec). Poly-chromatic staining of cells with CD123 PE, HLA-DR Percp-cy5.5, CD1c PE-cy7 and Lineage FITC confirmed the purity of Lin−HLA-DR+CD1c−CD123+pDCs (>90%). For phenotypic analysis, MNCs were stimulated with LPS (100 ng/ml)+Resiquimod (InvivoGen) R848 (100 ng/ml) overnight and stained with antibody cocktail for surface marker analysis. For cytokine assay, MNCs were stimulated with 5 μM CpG (ODN2216, InvivoGen, San Diego, CA) for 8 h and then add Golgistop for an additional 8 h. Cells were stained with an antibody cocktail for surface markers followed by the IFN-α2 V450 antibody. Flow cytometric analysis was performed on LSRFortessa X20 and data were analyzed with FloJo software (BD Biosciences, San Jose, CA).

### Human and Murine T Cells Preparation

T cell depleted bone marrow (TCD-BM) was prepared by depleting T cells with microbead-conjugated anti-CD4/CD8 antibodies. Naïve T cells were isolated from spleens and lymph nodes using microbead-conjugated antibodies (MiniMACS; Miltenyi Biotech). Purity was consistently >92%.

### Antibodies (Abs), Flow Cytometry Analysis and Cell Sorting

All antibodies used for immunofluorescence staining were purchased from eBioscience (San Diego, CA), BioLegend (San Diego, CA) or BD Biosciences (San Jose, CA). Magnetic microbead-conjugated Abs and streptavidin were purchased from Miltenyi-Biotech. The antibodies are listed in Table 2. Recombinant human IL-2 protein was purchased from R&D Systems (Minneapolis, MN). Cells were stained with appropriate concentrations of mAbs. Dead cells were excluded using Fixable Viability Dye from eBioscience (San Diego, CA). Flow cytometry analyses were performed using LSRFortessa™ X-20 (BD Bioscience). Cells were sorted on BD Melody (BD Bioscience).

TABLE 2
List of Antibodies

| | | | | | |
|---|---|---|---|---|---|
| | | | | | Catalog |
| Antigen | Clone | Fluorophore | Dilution | Source | No. |

Human

| | | | | | |
|---|---|---|---|---|---|
| FastImmune | SK7, 3G8, SJ25C1L27, | FITC | 1:20 | BD | 340546 |
| Lineage | MφP9, NCAM16.2 | | | | |
| Cocktail 1 (lin1) | | | | | |
| (CD3, CD14, | | | | | |
| CD16, CD19, | | | | | |
| CD20, CD56) | | | | | |
| HLA-DR | G46.6 (L243) | PerCP-Cy5.5 | 1:100 | BD | 552764 |
| CD123 | 9F5 | PE | 1:100 | BD | 555644 |
| CD1c | L161 | PE/Cy7 | 1:100 | Biolegend | 331515 |
| CD274 (PD-L1) | MIH1 | PE-CF594 | 1:100 | BD | 563742 |
| CD274 (PD-L1) | MIH1 | BUV395 | 1:100 | BD | 740320 |
| CD85k (ILT3) | ZM4.1 | APC | 1:100 | Biolegend | 333016 |
| (LILRB4) | | | | | |
| IFN-α[2b] | 7N4-1 | V450 | 1:50 | BD | 561382 |
| SHP2(pY542) | L99-921 | PE | 1:50 | BD | 560389 |
| LPAM-1 | Vedolizumab297.rMAb | Alexa | 1:100 | BD | 571601 |
| Integrin α4β7) | | Fluor ™ 647 | | | |
| CD195 (CCR5) | J418F1 | BV421 | 1:100 | Biolegend | 359117 |
| CXCR3 | 1C6/CXCR3 | BB700 | 1:100 | BD | 566533 |
| CCR9 | L053E8 | PE/CY7 | 1:100 | Biolegend | 358909 |
| CD45 | HI30 | APC | 1:200 | Biolegend | 304012 |
| CD4 | OKT4 | BV605 | 1:200 | Biolegend | 317438 |
| CD8 | SK1 | BV785 | 1:200 | Biolegend | 344740 |
| Ki-67 | Ki-67 | PerCP/Cy5.5 | 1:200 | Biolegend | 350520 |
| IFN-α | 4S.B3 | PE | 1:100 | Biolegend | 502509 |
| CD279 (PD-1) | EH12.2H7 | APC | 1:200 | Biolegend | 329908 |
| CD279 (PD-1) | NAT105 | BV421 | 1:200 | Biolegend | 367422 |
| CD366 (TIM-3) | F38-2E2 | PE/Cy7 | 1:200 | Biolegend | 345014 |
| EOMES | WD1928 | FITC | 1:200 | eBioscience | 11-4877-41 |
| TOX | REA473 | APC | 1:100 | Miltenyi | 130-118-335 |
| T-bet | 4B10 | PE | 1:100 | eBioscience | 12-5825-82 |
| Granzyme B | QA18A28 | BV421 | 1:100 | Biolegend | 396414 |

Murine

| | | | | | |
|---|---|---|---|---|---|
| CD4 | GK1.5 | BUV805 | 1:200 | BD | 612900 |
| CD45.1 | A20 | FITC | 1:100 | Biolegend | 110706 |
| CD45.2 | 104 | APC/Cy7 | 1:100 | Biolegend | 109824 |
| CD274 (PD-L1) | MIH6 | PE/Cy7 | 1:200 | Biolegend | 153613 |
| IFN-α | XMG1.2 | AF700 | 1:100 | Biolegend | 505824 |
| CD4 | GK1.5 | BUV805 | 1:200 | BD | 612900 |
| CD45.1 | A20 | FITC | 1:100 | Biolegend | 110706 |
| CD45.2 | 104 | APC/Cy7 | 1:100 | Biolegend | 109824 |
| CD274 (PD-L1) | MIH6 | PE/Cy7 | 1:200 | Biolegend | 153613 |
| IFN-α | XMG1.2 | AF700 | 1:100 | Biolegend | 505824 |
| CD4 | GK1.5 | BUV805 | 1:200 | BD | 612900 |
| CD45.1 | A20 | FITC | 1:100 | Biolegend | 110706 |
| CD45.2 | 104 | APC/Cy7 | 1:100 | Biolegend | 109824 |
| CD274 (PD-L1) | MIH6 | PE/Cy7 | 1:200 | Biolegend | 153613 |
| IFN-α | XMG1.2 | AF700 | 1:100 | Biolegend | 505824 |

Human DCs were characterized as negative for lineage markers (CD3 (for T cells), CD14 (for monocytes/macrophages), CD16 (for NK cells, activated monocytes/macrophages and neutrophils), CD19, CD20 (B cells) and CD56 (NK cells) and positive for HLA-DR as described in Reizis, B. (2019). Immunity 50, 37-50, Mochizuki, K., et al. (2013). Journal of Immunology 190, 3772-3782, and Mochizuki, K., et al. (2016). Blood 127, 3270-3280. Among these human DCs, CD123+ cells were defined as pDCs and CD1c+ and CD123-cells were defined as cDCs.

### Plasmid and Virus Preparation

The human PD-L1 Consensus Coding sequence (CCDS) with or IFN-α2a, was cloned into the pLU-CMV-puro vector and pLU-EF1α-puro vector to generate pLU-CMV (EF1a)-PDL1−P2A−IFNα2a-puro plasmids. The murine PD-L1 CCDS sequence, together with IFN-α2a, was cloned into the MigR1-IRES-puro vector to generate MigR1-PDL1-IFNα2a-IRES-puro plasmids. HEK293T cells were transfected with lentiviral plasmids and packaging plasmids using TransIT®-LT1 Transfection Reagent (Mirus Bio, Madison, WI), as described in TransIT®-LT1 Full Transfection Protocol (mirusbio.com/product/transit-lt1-transfection-reagent). Platinum-E cells were transfected with retroviral plasmids and packaging plasmids using TransIT®-LT1 Transfection Reagent to generate retrovirus. Virus supernatants were collected at 48- and 72-hours post-transfection and stored at −80° C. for future use.

In brief, the plasmid transfection of cells involves the following steps:

1) plate cells approximately 18-24 hours prior to transfection in complete growth medium. Ideally cells should be at least 80% confluent prior to transfection.

2) prepare the transfection reagent-DNA complex by adding plasmid DNA and the transfection reagent into reduced-serum growth medium and incubate for 15-30 minutes.

3) distribute the transfection reagent-DNA complexes to the cells and incubate for 24−72 hours.

### K562 and T Cell Engineering

The K562 cell line was infected with lentivirus encoding PD-L1 or LILRB4, followed by FACS sorting to isolate the PD-L1 or LILRB4-positive populations. For human αρ-T cell preparation, CD4+ and CD8+ T cells were mixed at 1:1 ratio and activated with Dynabeads Human T-Activator CD3/CD28 (Gibco, NY) at a 1:1 bead-to-cell ratio in RPMI-1640 supplemented with 10% FBS, Glutamine, antibiotics and recombinant human IL-2 (10.0 ng/ml). Twenty-four hours later, T cells were transduced at a multiplicity of infection (MOI) of 3 to 5. Cells were cultured in vitro for 7 days, generating human αρ-T cells. Cells infected with empty vector viruses are referred to as v-T cells. For murine αρ-T cell preparation, murine CD4+ T cells were activated with anti-CD3/CD28 antibody (1.0 μg/ml) for 36 hours following by retrovirus infection. Cells were cultured in vitro for 7 days and PD-L1+ cells were sorted as murine αρ-T cells.

### CD4+ T Cell Co-Culture Assay

Human CD4+ T cells were labeled with 5 μM Cell trace violet (CVT) for cell dividing/proliferation assay. CVT labeled CD4+ T cells were mixed with CpG stimulated pDCs, K562 cell lines, αρ-T cells, or Raji cells at various ratio in the presence of anti-CD3/28 or anti-CD3 antibody alone. Cells were counted on day 5 or day 7. Cell dividing/proliferation was examined on day 5 after culture. Live/dead dye was used for cell death analysis at day 5 and day 7.

### ELISA

Cell culture supernatants or serum were collected and analyzed using the Human IFN-α2/IFNλ2 DuoSet ELISA kit (R&D Systems, Minneapolis, MN, USA), as described at rndsystems.com/products/human-ifn-alpha-2-ifna2-duoset-elisa_dy9345-05.

In brief, the ELISA assay comprises the steps of:

1) Adding 100 μL of sample or standards in Reagent Diluent, or an appropriate diluent, per well. Cover with an adhesive strip and incubate 2 hours at room temperature.

2) Repeating the aspiration/wash as in step 2 of Plate Preparation.

3) Adding 100 μL of the Detection Antibody, diluted in Reagent Diluent, to each well. Cover with a new adhesive strip and incubate 2 hours at room temperature.

4) Repeating the aspiration/wash as in step 2 of Plate Preparation.

5) Adding 100 μL of the working dilution of Streptavidin-HRP to each well. Cover the plate and incubate for 20 minutes at room temperature. Avoid placing the plate in direct light.

6) Repeating the aspiration/wash as in step 2.

7) Adding 100 μL of Substrate Solution to each well. Incubate for 20 minutes at room temperature. Avoid placing the plate in direct light.

8) Adding 50 μL of Stop Solution to each well. Gently tap the plate to ensure thorough mixing.

9) Determining the optical density of each well immediately, using a microplate reader set to 450 nm. If wavelength correction is available, set to 540 nm or 570 nm. If wavelength correction is not available, readings at 540 nm or 570 nm are subtracted from the readings at 450 nm. This subtraction will correct for optical imperfections in the plate. Readings made directly at 450 nm without correction may be higher and less accurate.

### RNA Sequencing and Analysis

Total mRNA was isolated using TRIzol™ reagent (Invitrogen Corp., Carlsbad, CA) from the sorted cells that were activated by PMA+ionomycin for 2 hours (as described in Invitrogen's TRIzol™ reagent user guide, Pub. No. MAN001271), and was submitted to Admera Health (South Plainsfield, NJ) who assessed sample quality with the Agilent Bioanalyzer and prepared the library using the NEBnext Ultra RNA-Poly-A kit (as described at neb.com/en-us/products/e7770-nebnext-ultra-ii-rna-library-prep-kit-for-illumina, manual E7770_E7775). Libraries were analyzed using Illumina next generation sequencing and relative quantification was provided by Admera Health. Read counts were subjected to paired differential expression analysis using the R package DESeq2. Genes were considered significant for upregulation/downregulation if P<0.05 and q<0.05. q<0.01 and FC≥1.5 were used as cutoffs to identify significantly changed genes. RNA-seq data were deposited in the NCBI's Gene Expression Omnibus database with accession number GSE292397.

### Real-Time RT-PCR

Total mRNA was isolated using TRIzol reagent (Invitrogen Corp.) from the sorted cells. cDNA was synthesized with SuperScript™ IV VILO™ Master Mix with ezDNase™ (Thermo Fisher Scientific, Waltham, MA, USA). qPCR was performed using SYBR Green PCR Master Mix (Thermo Fisher Scientific) on a QuantStudio 5 real-time PCR system (Thermo Fisher Scientific) with the following cycling conditions: 95° C. for 2 min, followed by 40 cycles of 95° C. for 15 s and 60° C. for 1 min. Relative gene expression was calculated using the 2\hat{ }-ΔCt method, normalized to 18S. Primer sequences were:

18S (foraward: 5′-ACCCGTTGAACCCCATTCGTGA-3′
(SEQ ID NO: 1), reverse: 5′-GCCTCACTAAACCAT
CCAATCGG-3′ (SEQ ID NO: 2));
ISG15 (forward: 5′-CGCAGATCACCCAGAAGATCG-3′
(SEQ ID NO: 3), reverse: 5′-TTCGTCGCATTTGTC
CACCA-3′ (SEQ ID NO: 4));
and
IL2 (forward: 5′-AGCACTTCCTCCAGAGGTTTG-3′
(SEQ ID NO: 5), reverse: 5′-ACTCACCAGGATGCT
CACAT-3′ (SEQ ID NO: 6)).

Induction of x-GVHD and GVT Effects

To establish the x-GVHD model, 10×10⁶ TCD-PBMCs were injected into NSG mice by tail vein at day −7 to generate hu-NSG mice. On day 0, 10×10⁶ v-T cells or αρ-T cells were administered to the hu-NSG mice to induce x-GVHD. Body weight and clinical score were monitored as previous described in Zhang, Y., et al. (2005). Alloreactive Memory T Cells Are Responsible for the Persistence of Graft-versus-Host Disease. Journal of immunology 174, 3051-3058. To induce GVT effects, RajiTGL cells (10⁵ cells) or MV4-11TGL cells (10⁶ cells) were injected intravenously into 8- to 12-week-old NSG mice at day 0, followed by injection (i.v.) of 10×10⁶ v-T cells with or without 8×10⁶ αρ-T cells. Leukemic growth was monitored using bioluminescence imaging.

### Murine GVHD Models

Mice underwent bone marrow transplantation as described in Zhang, Y., et al. (2005). Journal of Immunology 174, 3051-3058. To induce GVHD, Balb/c mice were irradiated with 8.50 Gy irradiation from an X-Ray source followed by transplantation with donor B6 TCD-BM cells (5×10⁶), with or without adding naïve CD4+ T cells. Recipients were monitored for survival and clinical signs of GVHD using the scoring system that includes weight loss, posture, mobility, fur and skin impairment.

### Bioluminescent Imaging

Mice were injected intraperitoneally with 200 μL of firefly luciferin (Caliper Life Sciences, Hopkinton, MA). After anesthesia, bioluminescent signals were captured using an IVIS Lumina X5 Imaging System (PerkinElmer, Inc., Waltham, MA). Image analysis was performed using Aura software (Spectral Instruments Imaging, Tucson, AZ).

### Histopathology

Specimens of liver, intestine and skin were provided to 2 pathologists for histopathologic assessment of GVHD (Histopathology & Tissue Shared Resource, Lombardi Comprehensive Cancer Center, Georgetown University). They employed a semiquantitative scoring system for abnormalities known to be associated with GVHD as described in Huang, Q., et al. (2017). Blood, and Zaiken, M. C., et al. (2022). Blood 139, 2983-2997. In brief, liver GVHD was scored by the severity of lymphocytic infiltrate, number of involved tracts and severity of liver cell necrosis; the maximum score is 9. Lung GVHD was scored by periluminal infiltrates, pneumonitis, and the severity of lung tissue damage; the maximum score is 9. Images were obtained with a Leica DM1000 LED (10/0.25 HI PLAN lens, 100× magnification, Leica MC170 HD camera).

### Statistics

Data were statistically analyzed using the two-tailed Student's t test, one-way ANOVA and two-way ANOVA. All analyses were performed using GraphPad Prism 10.0 software (GraphPad, San Diego, CA). p<0.05 was set as the statistical significance.

Human pDCs and their Derived IFN-α and PD-L1 have Potent Immunosuppressive Effects

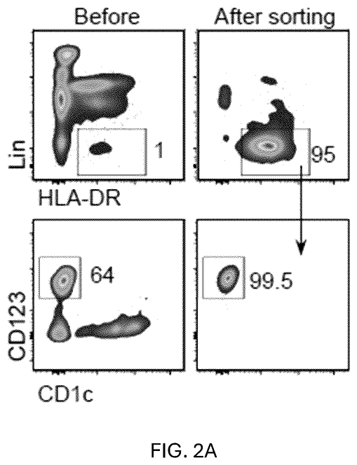

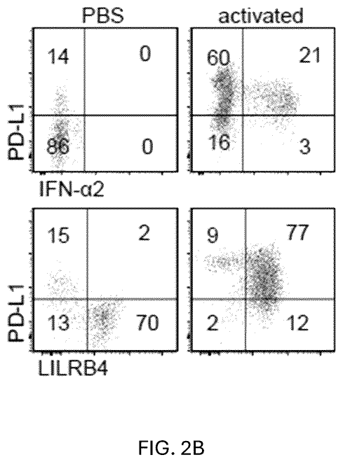

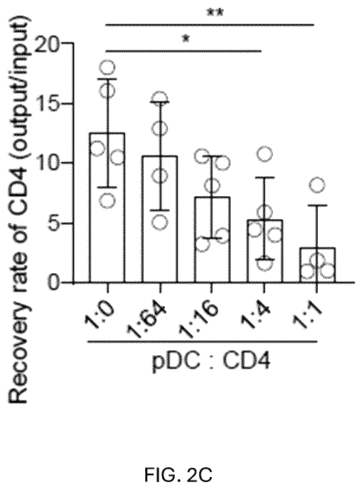

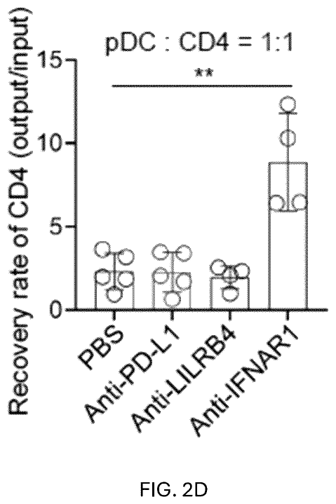

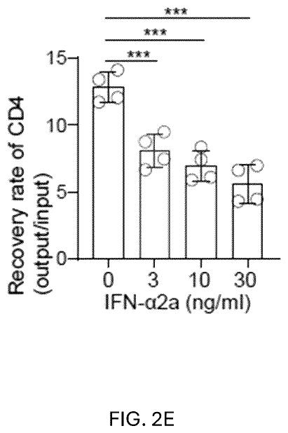

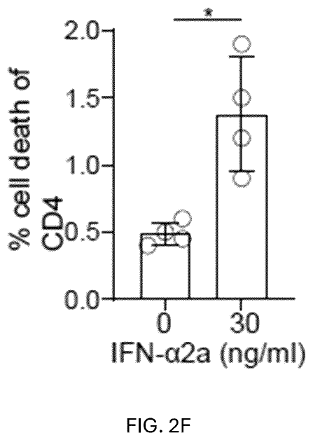

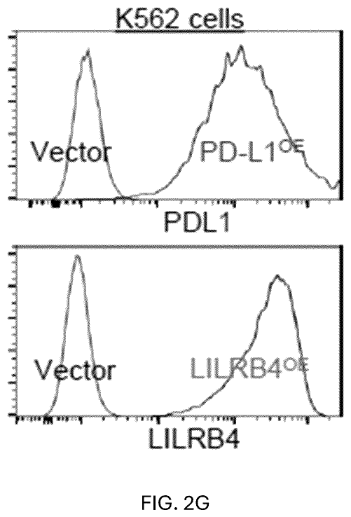

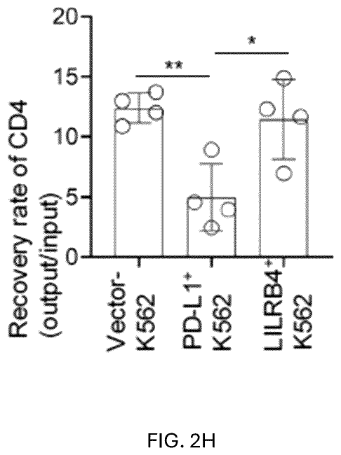

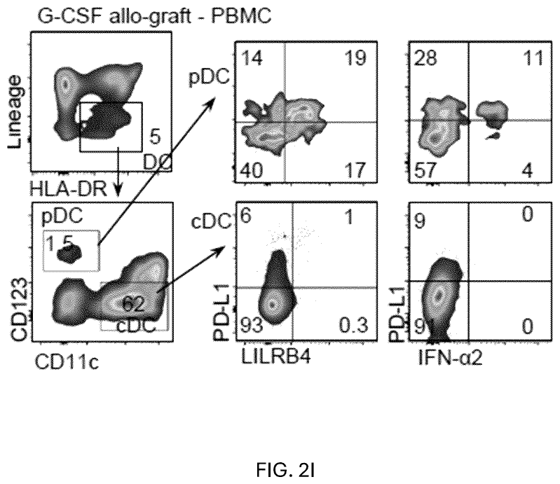

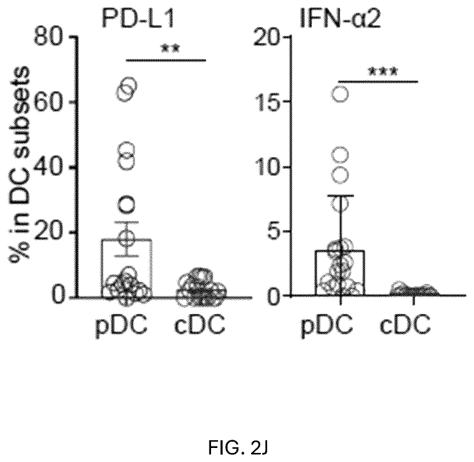

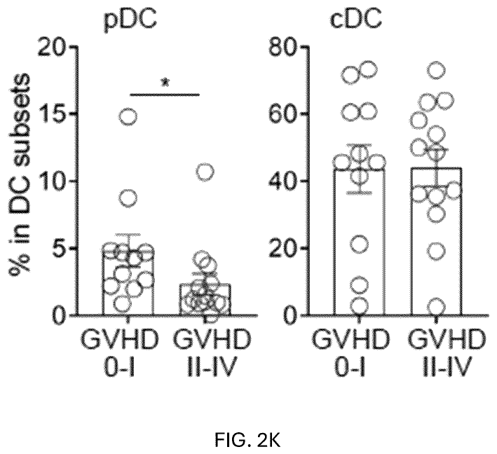

To test the impact of human pDCs on activated T cell proliferation and survival, lineage (Lin)−HLA-DR⁺CD123+CD1c−pDCs were FACs sorted from the peripheral blood (PB) of healthy donors (FIG. 2A), activated with a TLR agonist cocktail to induce IFN-α and PD-L1 (FIG. 2B), and added to TCR-activated autologous CD4+ T cells (FIG. 2C). pDCs suppressed expansion of TCR-activated CD4+ T cells in a cell dose-dependent manner (FIG. 2C). Blockade of IFNAR1 using its neutralizing antibody (Ab) abrogated pDCs' repressive effects (FIG. 2D), whereas adding IFN-α2a, a subtype of IFN-α family proteins [Sumida, T. S., et al. (2022). Nat Immunol 23, 632-642], to the culture of TCR-activated CD4+ T cells repressed their expansion and survival (FIGS. 2E-2F). These data suggest that IFN-α is important for mediating pDCs' suppressive effects in human T cells. Blockade of PD-L1 did not reduce the effect of pDCs on repressing expansion of TCR-activated CD4⁺ T cells (FIG. 2D). To rule out the possibility that pDC-derived IFN-α could mask the repressing effect of PD-L1, K562 cells were transduced with lentivirus encoding human PD-L1 to produce PD-L1+K562 cells, with K562 cells expressing immunoregulatory molecule LILRB4 as control (FIG. 2G). Addition of PD-L1+K562 cells rather than LILRB4+K562 cells dramatically reduced expansion of TCR-activated CD4+ T cells (FIG. 2H), corroborating the inhibitory role of PD-L1.

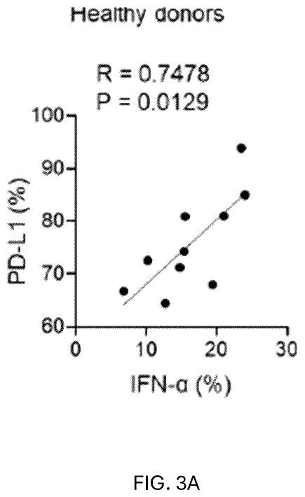

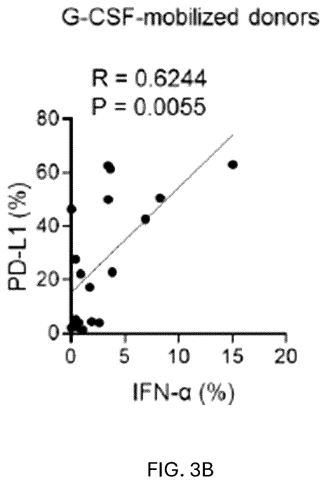

Next, the association of human pDCs in G-CSF mobilized peripheral blood stem cell (G-PBSC) allografts was assessed with the occurrence of severe GVHD in allo-HCT patients. Clinical studies suggested that a low level of pDCs in the PB at the time of engraftment predicted the occurrence of severe GVHD in patients [Li, B., et al. (2021). Transplant Cell Ther 27, 611 e611-611 e612]. Upon stimulation with a TLR agonist cocktail, pDCs derived from G-PBSC allografts produced high levels of IFN-α and PD-L1 proteins (FIGS. 2I-2J). Person correlation analysis revealed that the expression of IFN-α and PD-L1 was highly correlated in activated pDCs either from the PB of healthy donors (n=10) or G-PBSC grafts (n=18, in which IFN-α and PD-L1 were examined simultaneously) (FIGS. 3A-3B, Table 1). Patients who developed grade II-IV acute GVHD had an approximately 2-fold lower frequency of pDCs in the PBSC allograft than patients with grade 0-I GVHD (FIG. 2K), consistent with the suppressive effect of human pDCs. These data highlight the potential of utilizing hematopoietic cells that co-express PD-L1 and IFN-α as a novel strategy to prevent GVHD instead of purifying the rare and fragile human pDCs.

FIG. 2A, FIG. 2B, FIG. 2C, FIG. 2D, FIG. 2E, FIG. 2F, FIG. 2G, FIG. 2H, FIG. 2I, and FIG. 2K show immune modulatory roles of human pDCs. FIG. 2A shows pDCs that were FACS-sorted from healthy donors (HD) with anti-CD304 microbeads. Flow plots show cell purity before and after cell sorting. FIG. 2B shows sorted pDCs that were activated with TLR agonist cocktail (i.e., a mixture of different molecules that activate toll-like receptors to stimulate immune responses-TLR4 agonist LPS, 100.0 ng/ml; TLR7/8 agonist R848, 100.0 ng/ml; and TLR9 agonist CpG ODN2216, 5.0 μM) overnight. Dot plots show PD-L1, IFN-α2 and LILRB4 expression on pDCs. FIG. 2C shows human CD4⁺ T cells that were cocultured with autologous pDCs in the presence of anti-CD3/28 antibody (Ab) (1.0 μg/ml for each) as indicated. Graph shows expansion of CD4⁺ T cells 7 days after culture. FIG. 2D shows human CD4⁺ T cells that were cocultured with autologous pDCs at 1:1 ratio in the presence of anti-CD3/28 Ab, with or without anti-PD-L1, anti-LILRB4 or anti-IFNAR1 neutralizing Ab (10.0 μg/ml). Graph shows expansion of CD4⁺ T cells 7 days after culture. FIG. 2E shows CD4⁺ T cells that were activated with anti-CD3/28 Ab cultured in the presence of IFN-α2a for 7 days to test their expansion. FIG. 2F shows CD4⁺ T cells that were activated with anti-CD3/28 Ab cultured in the presence of IFN-α2a for 7 days to test their cell death (flow cytometric analysis). FIG. 2G shows K562 cells (i.e., a human cancer cell line derived from a person with chronic myeloid leukemia) that were transduced with lentivirus encoding human PD-L1 or LILRB4. Histograms display expressions of PD-L1 and LILRB4 on K562 cells. FIG. 2H shows CD4⁺ T cells that were cocultured with vector control or PD-L1- and LILRB4-overexpressing K562 cells at 1:1 ratio. Graphs show expansion of CD4⁺ T cells 7 days after culture. FIG. 2I shows PBMCs from G-CSF mobilized HD (G-CSF HD, n=24) that were stimulated with the TLR agonist cocktail overnight. Cells were harvested for flow cytometry analysis. Plots show Lineage (Lin)⁻HLA-DR⁺ cells DC, CD11c⁺CD123⁻cDCs (conventional dendritic cells) and CD11c-CD123+pDCs and their expression of PD-L1, LILRB4 and IFN-α2. FIG. 2J shows PBMCs from G-CSF mobilized HD (G-CSF HD, n=24) were stimulated with the TLR agonist cocktail overnight. Cells were harvested for flow cytometry analysis. Graphs show the fraction of PD-L1- and IFN-α2-expressing pDCs and cDCs. FIG. 2K shows PBMCs from G-CSF mobilized HD (G-CSF HD, n=24) were stimulated with the TLR agonist cocktail overnight. Cells were harvested for flow cytometry analysis pDC and cDC frequencies in grafts for patients grade 0-I GVHD (n=11) and patients with grade II-IV GVHD (n=13). *, P<0.05; **, P<0.01; ***, P<0.001.

CD304 (BDCA-4/Neuropilin-1) is specifically expressed in pDCs in human blood, bone marrow, and cord blood. Exclusive expression of CD304 on pDCs allows for their direct isolation. In blood and bone marrow, CD304 pDCs are CD4+, CD45RA+, CD303 (BDCA-2)+, CD141 (BDCA-3) dim, CD1c (BDCA-1)−, and CD2−. They lack expression of lineage markers (CD3, CD14, CD16, CD19, CD20, CD56) and neither express myeloid markers such as CD13 and CD33, nor Fc receptors such as CD32, CD64, or FcεRI. Their lymphoid origin is indicated by their plasmacytoid morphology and the expression of the pre-T cell receptor a-chain. Freshly isolated CD1c (BDCA-1)+ or CD141 (BDCA-3)++ myeloid blood dendritic cells and monocytes do not express CD304 (BDCA-4/Neuropilin-1), but expression of CD304 (BDCA-4/Neuropilin-1) is induced on myeloid blood dendritic cells and monocytes upon culturing [Jarrossay, D. et al. (2001) Eur. J. Immunol. 31:3388-3393, Dzionek, A. et al. (2000), J Immunol 165:6037-6046, Grabbe, S. et al. (2000) Immunol. Today 21:431-433].

Culturing cells in the presence of anti-CD3 and anti-CD28 antibodies is a method for activating and expanding T cells by using antibodies that bind to both CD3 and CD28 surface receptors on T cells. This process provides the T cells with both the primary signal (CD3) and the essential co-stimulatory signal (CD28) needed for robust proliferation, without triggering cell death or anergy that can occur with CD3 stimulation alone.

PD-L1, or programmed death-ligand 1, is a protein that can be found on both normal and cancer cells. It functions as an immune checkpoint by binding the PD-1 receptor on T cells, which prevents T cells from attaching and killing the cell expressing PD-L1. IFN-α2 is a protein belonging to the interferon alpha family, which plays a key role in the immune response to viruses and cancer. It is a type I interferon that acts as a cytokine, secreted by infected cells to trigger an antiviral state in other cells by activating signaling pathways like JAK-STAT. LILRB4, or leukocyte immunoglobulin-like receptor B4, is an immune checkpoint protein found on myeloid cells that helps regulate immune responses. It plays a role in both normal physiological processes, like immune cell differentiation, and various diseases, such as autoimmune diseases and cancers. [Deng, M. et al. Nature (2018) 562:605-609].

Different subsets of dendritic cells have different phenotypes. Lineage (Lin)−HLA-DR+ cells are used to identify the general population of dendritic cells. These cells are Lin−, i.e., negative for a panel of lineage markers (e.g., CD3, CD14, CD15, CD16, CD19, CD20, CD56), which helps distinguish DCs from lymphocytes, monocytes, granulocytes, etc., and they are HLA-DR+, meaning they express the MHC class II molecule HLA-DR, which is a key identifier for mature DCs. The CD11c+CD123-phenotype of DCs represents a more specific marker combination used to identify conventional DCs (cDCs) [Kang S J, et al. Front Immunol. 2021 Jul. 1; 12:700755].

The CD11c−CD123+ phenotype identifies plasmacytoid dendritic cells (pDCs), which is a type of immune cell defined by the absence of the CD11c marker and the presence of the CD123 marker on their surface [Kang S J, et al. Front Immunol. 2021 Jul. 1; 12:700755].

FIG. 3A and FIG. 3B show Pearson correlation analysis of PD-L1 and IFN-α expression in human pDCs. FIG. 3A shows the frequency of IFN-α2 on pDCs obtained from PB of healthy donors. FIG. 3B shows the frequence of IFN-α2 on pDCs obtained from G-CSF mobilized PBSC allo-grafts.

### IFN-α and PD-L1-Modified Human T Cells Prevent Xenogeneic GVHD.

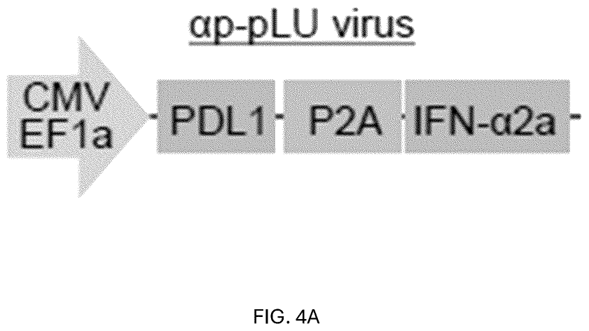

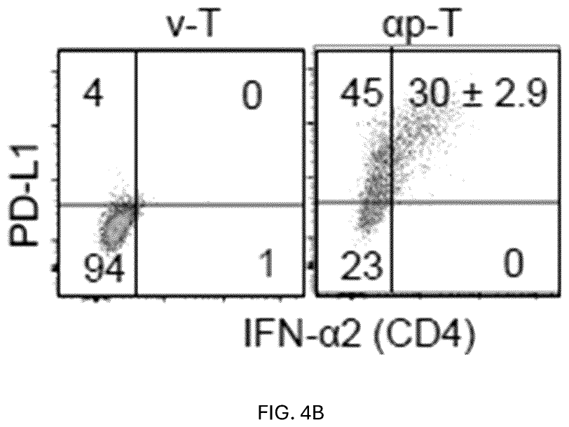

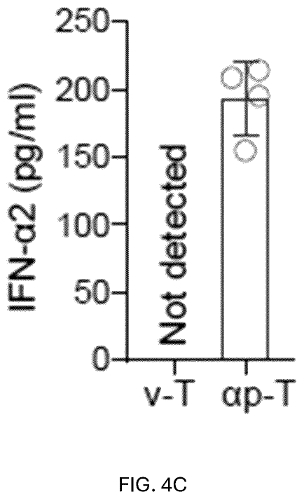

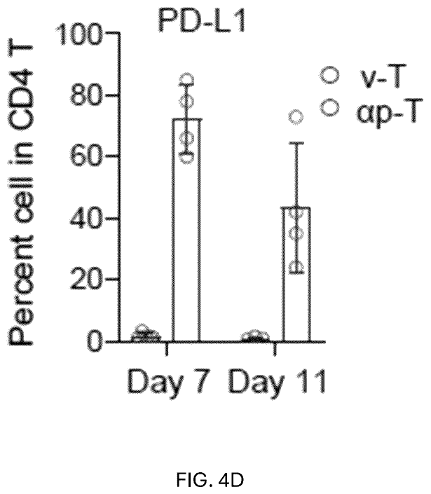

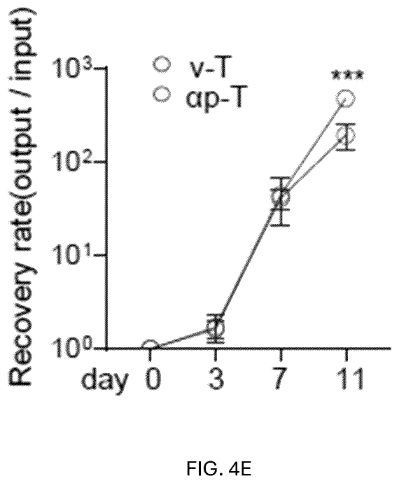

Genetic modifications of human T cells, such as chimeric antigen receptor T-cell therapy, have been developed to successfully treat cancer [June, C. H., et al. (2018). Science 359, 1361-1365]. To test whether PD-L1 and IFN-α can be delivered by T cells to prevent GVHD, lentivirus encoding PD-L1 and IFN-α2a (αρ-pLU virus, FIG. 4A) were generated and infected human CD4+ and CD8+ T cells to co-express PD-L1 and IFN-α2a (named αρ-T cells, FIG. 4B), with empty lentiviral vector transduced cells (named v-T cells) as control. These engineered αρ-T cells secreted high levels of soluble IFN-α2 (FIG. 4C), stably expressed PD-L1 (FIG. 4D) and showed inferior expansion capacity compared to empty vector control T cells (named v-T cells) (FIG. 4E).

FIG. 4A, FIG. 4B, FIG. 4C, FIG. 4D, and FIG. 4E shows generation of human αρ-T cells that can inhibit x-GVHD. FIG. 4A shows a schematic structure of the lentiviral vector encoding PD-L1 and IFN-α2a. FIG. 4B shows empty lentiviral vector transduced T cells (v-T) and αρ-pLU virus transduced human T cells (both CD4⁺ and CD8⁺) (αρ-T cells) that were expanded 7 days and stimulated with PMA and Ionomycin for 6 hours. Plots showed PD-L1 and IFN-α2 expression on CD4 v-T or αρ-T cells. FIG. 4C shows v-T or αρ-T cells that were cultured at 0.5×10⁶/ml overnight and supernatant were harvest for ELISA. Bar graph showed IFN-α2 level in the supernatant on day 7 from 4 healthy donors. FIG. 4D shows the percentage of PD-L1 expression in cultured v-T and αρ-T cells from 4 healthy donors. FIG. 4E shows the expansion of v-T and αρ-T cells cultured in medium containing human IL-2 (10.0 ng/ml). *, P<0.05; **, P<0.01; ***, P<0.001.

PMA (phorbol 12-myristate 13-acetate) and ionomycin are compounds used to stimulate immune cells, such as T cells, to produce cytokines and undergo other responses such as proliferation and apoptosis. PMA activates protein kinase C (PKC), which is crucial for cell activation and gene expression, while ionomycin, which is a calcium ionophore that transports calcium ions from outside the cell into the cytoplasm, increases intracellular calcium levels.

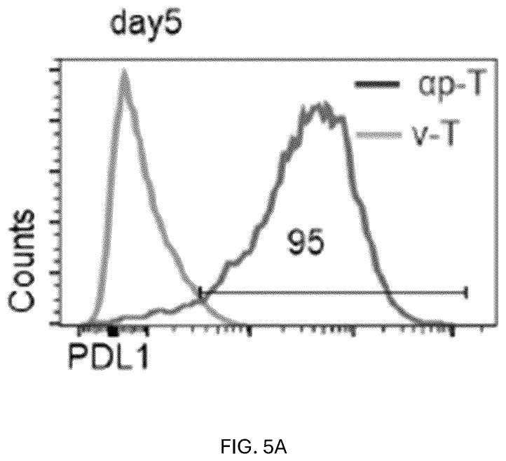

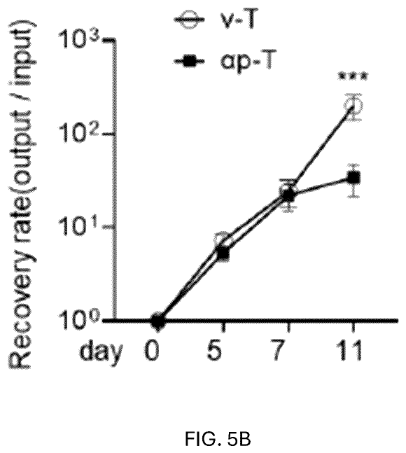

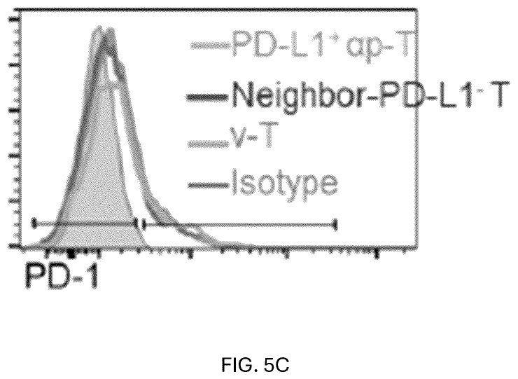

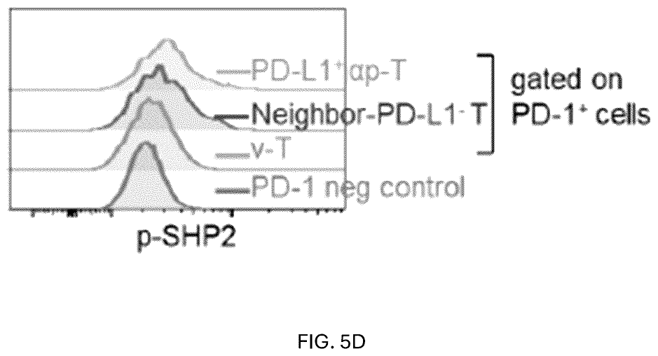

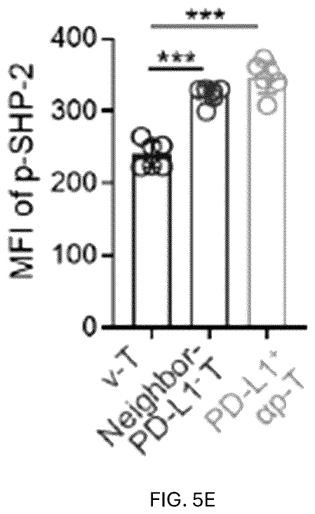

To rule out a possible effect from neighboring T cells on expansion of αρ-T cells, PD-L1⁺αρ-T cells (>95% purity) were FACS sorted at day 5 after activation (FIG. 5A). Six days later, these purified αρ-T cells expanded approximately 5.7-fold less than control v-T cells (FIG. 5B), suggesting that an intrinsic mechanism rather than neighboring cells may limit proliferative capacity of αρ-T cells. Despite this reduced expansion capacity, up to 350.0×10⁶ αρ-T cells could be readily generated from 10.0×10⁶ input cells over a period of 11 days culture (FIG. 5B), suggesting that an adequate number of αρ-T cells for clinical use can be achieved.

Binding of PD-L1 to PD-1 on T cells may suppress their proliferation. It was observed that like v-T cells, PD-L1+αρ-T cells and their neighboring PD-L1− T cells expressed low levels of PD-1 at day 7 of culture (FIG. 5C), consistent with the observation that PD-1 is transiently increased on activated T cells but declines to low levels as the TCR signaling decreases [Bally, A. P., et al. (2016). Journal of Immunology 196, 2431-2437, Ghoneim, H. E., et al. (2017). Cell 170, 142-157 e119]. The binding of PD-L1 to PD-1 recruits and activates SHP-2 phosphatase, which suppresses TCR and CD28 signaling [Patsoukis, N., et al. (2020). Sci Adv 6]. SHP-2 phosphatase is a protein tyrosine phosphatase crucial for regulating cell signaling; it plays a key role in normal development, cell growth, and various disease states. Despite being a phosphatase (i.e., an enzyme that removes phosphate groups), it often acts as a positive regulator by dephosphorylating specific proteins, which enhances signals such as those from cytokines [Chen X, et al. Front Immunol. 2024 Mar. 5; 15:1340726].

Compared to v-T cells, both PD-L1+αρ-T cells and neighboring PD-L1− T cells showed significantly higher phosphorylated SHP-2 (p-SHP2) expression (FIGS. 5D-5E), indicating activation of PD-1 signaling in these PD-1-expressing cells.

FIG. 5A, FIG. 5B, FIG. 5C, FIG. 5D, and FIG. 5E show purified PD-L1+αρ-T cell expansion in vitro and PD-1 signaling in cell subsets. PD-L1+αρ-T cells were sorted on day 5 and continued culturing until day 11 with v-T cells as a control. FIG. 5A shows a histogram depicting purity of PD-L1+αρ-T cells on day 5 after culture. FIG. 5B shows PD-L1+αρ-T cell expansion rate in vitro from day 0 to day 11. FIG. 5C shows a histogram depicting PD-1 expression on v-T cells or PD-L1 positive or negative components from αρ-T cells on day 7 after culture. FIG. 5D shows a histogram depicting pSHP2 expression on v-T cells or PD-L1 positive or negative components from αρ-T cells on day 7 after culture. FIG. 5E shows bar graphs of MFI of pSHP2 expression on T cell subsets. ***, P<0.001.

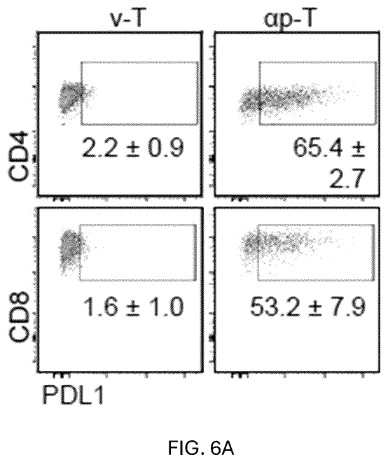

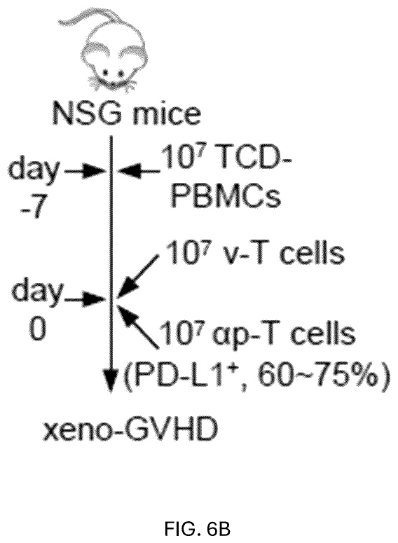

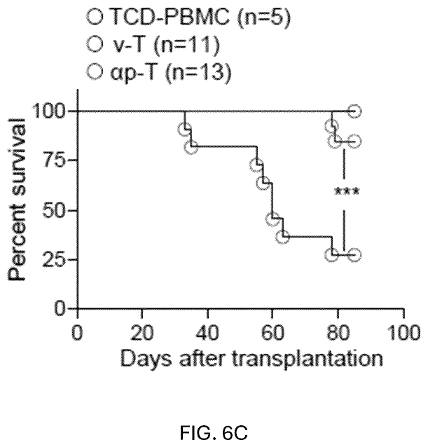

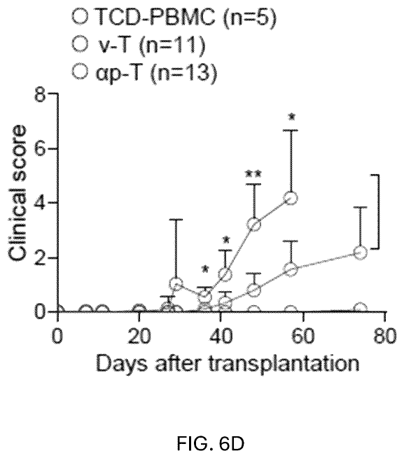

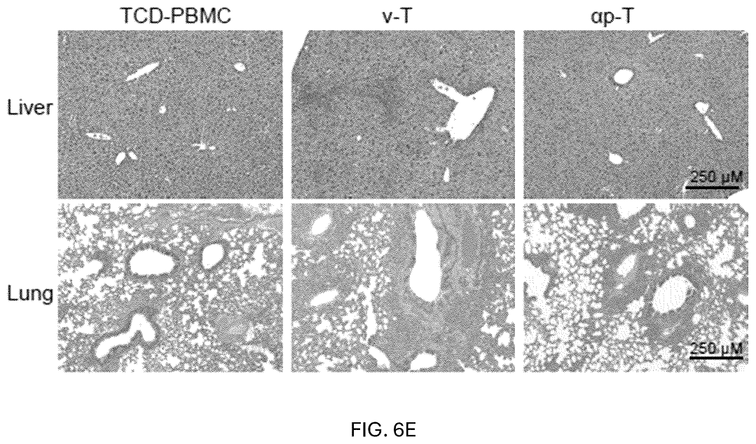

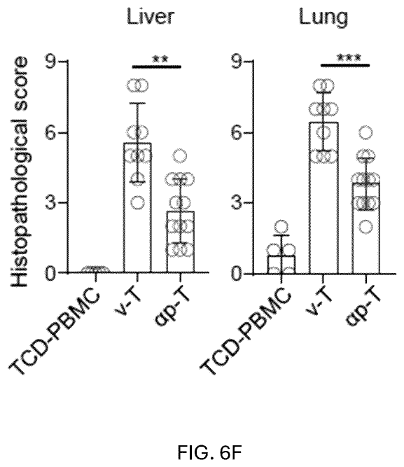

To examine the role of these αρ-T cells in repressing xenogeneic GVHD (x-GVHD), healthy human donor-derived T cell-depleted (TCD)-peripheral blood mononuclear cells (PBMCs) were transplanted into NSG mice (immunodeficient) to generate humanized NSG mice (hu-NSG mice) as described [Wang, Y., et al. (2023). Blood. 10.1182/blood.2023021126, Jia, B., et al. (2022). J Leukoc Biol 111, 427-437]. The same donor's T cells were used to generate v-T cells and αρ-T cells. Seven days later, v-T cells (equal ratio of CD4+ and CD8+ T cells) and unpurified αρ-T cell products (containing 60% to 75% of PD-L1+ T cells) were separately transferred into hu-NSG mice (FIGS. 6A-6B). Infusion of αρ-T cells prevented x-GVHD in these mice, with 84.6% of them surviving without clinical signs of severe GVHD over the observation period of 85 days (FIGS. 6C-6D). In contrast, 72.7% of hu-NSG mice given v-T cells died from severe GVHD (FIGS. 6C-6D). Histopathological examination showed that compared to v-T cell recipients, αρ-T cell recipients had significantly reduced inflammation in the liver and lung, two primary target organs in this model (FIGS. 6E-6F), indicating that αρ-T cells can prevent x-GVHD.

FIG. 6A, FIG. 6B, FIG. 6C, FIG. 6D, FIG. 6E, and FIG. 6F show NSG mice that were administered 10 million TCD-PBMCs at day −7, followed by injection of 10 million v-T or αρ-T cell products at day 0. FIG. 6A shows the PD-L1 expression in v-T and αρ-T cells (mean±SD) before transplantation. FIG. 6B shows the schematic experimental procedure. FIG. 6C shows the overall survival. FIG. 6D shows the clinical score of the animals. FIG. 6E shows tissues that were harvested at study endpoint. Images (H&E stained) were obtained at ×100 magnification. FIG. 6F show graphs that depict the histological score of inflammation in the liver and lung. *, P<0.05; **, P<0.01; ***, P<0.001.

αρ-T Cells Suppress Xeno-Reactive T Cell Responses During x-GVHD.

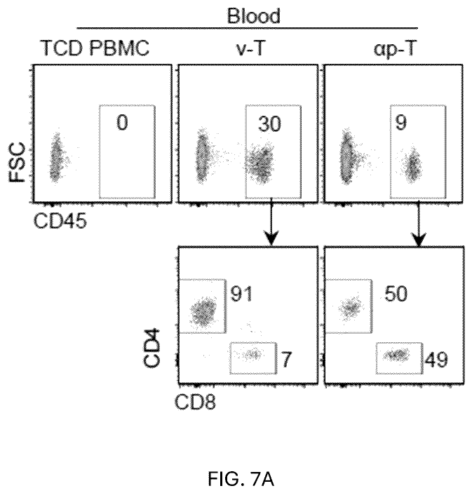

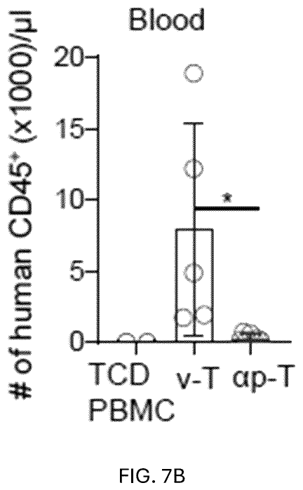

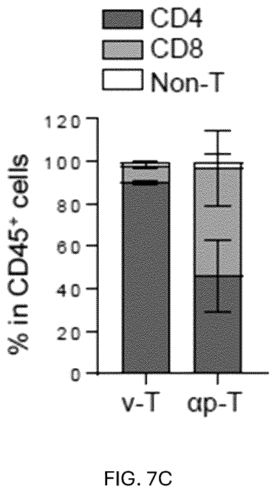

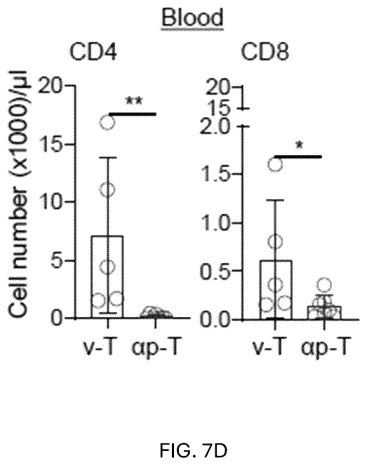

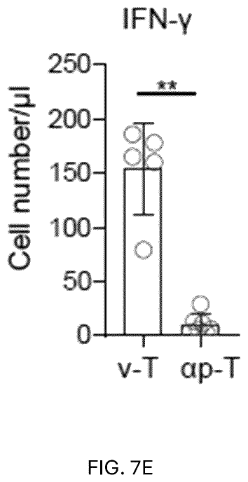

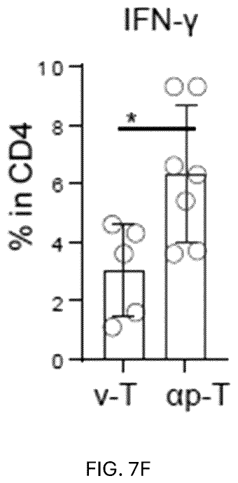

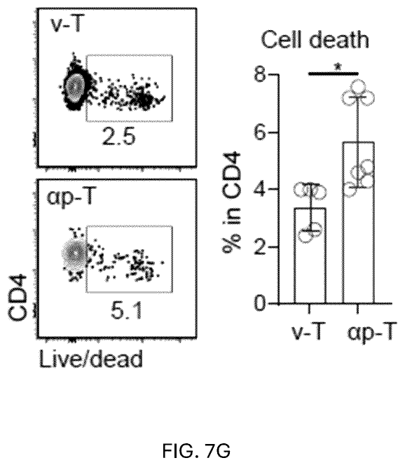

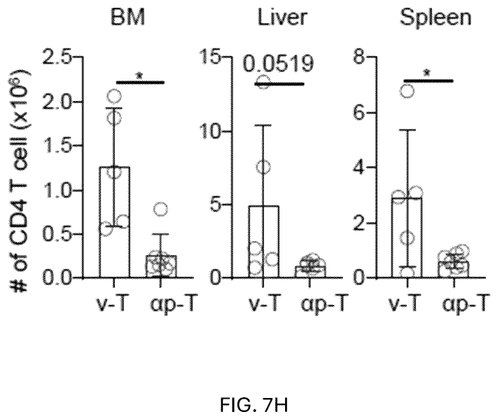

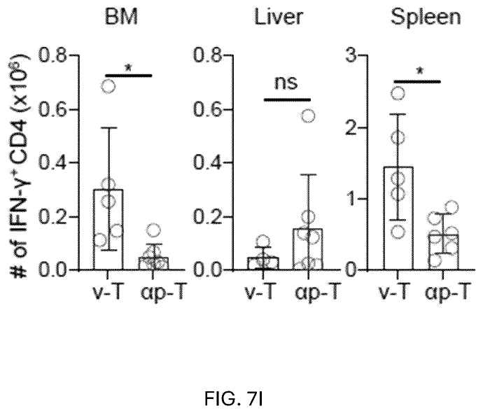

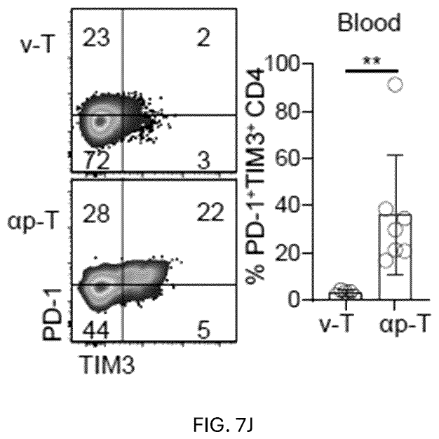

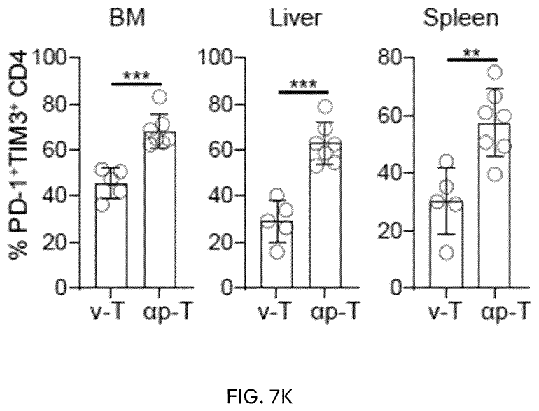

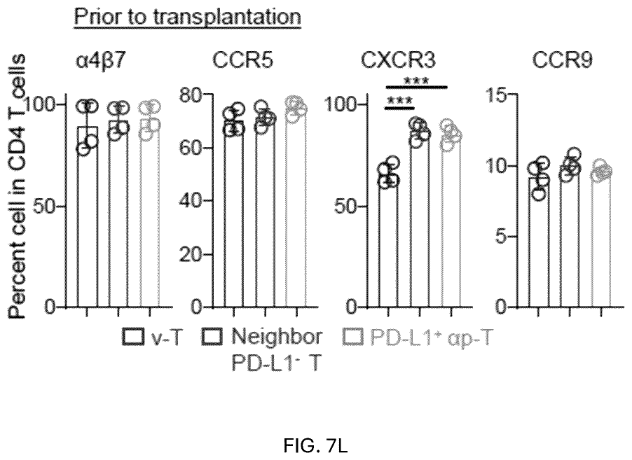

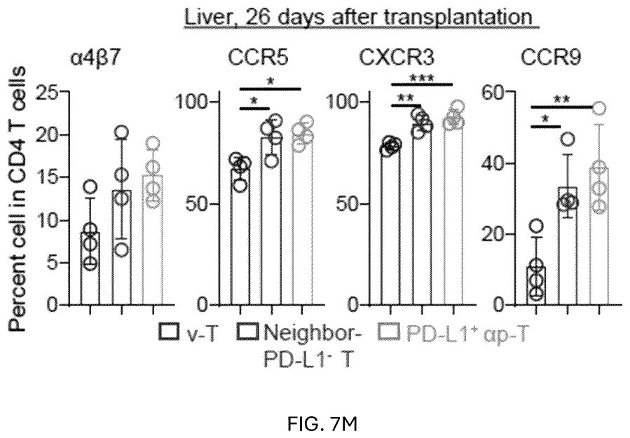

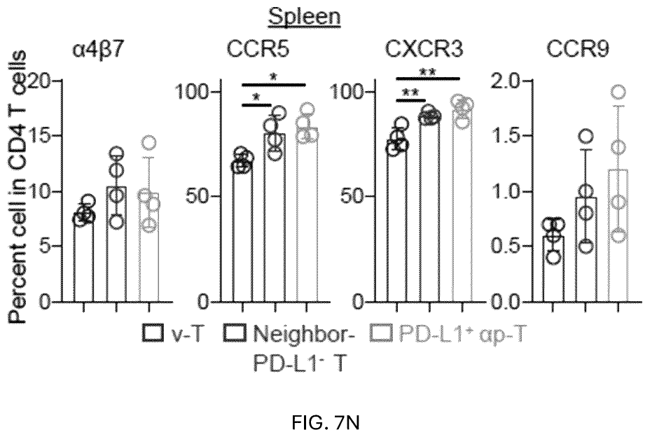

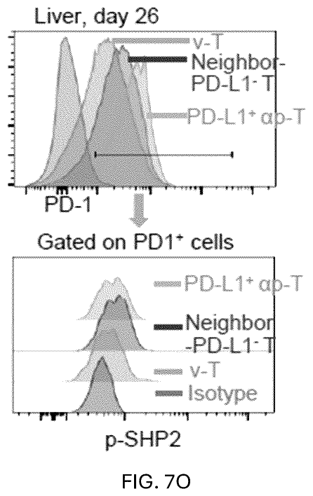

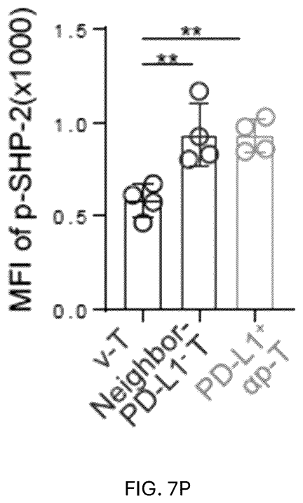

To understand the mechanisms that mediate αρ-T cell repression of x-GVHD, donor T cells from the circulating PB of NSG mice were collected at 46 days after transplantation when x-GVHD clearly occurred in hu-NSG mice given v-T cells (FIGS. 6C-6D). Compared to v-T cell recipients, αρ-T cell recipients showed a 26-fold decrease in total T cell numbers (FIGS. 7A-7B). Notably, in the PB of hu-NSG mice given v-T cells, the frequency and number of donor CD4+ T cells were approximately 10-fold higher than CD8+ T cells (FIGS. 7C-7D). This suggests a primary role for CD4+ T cells in mediating x-GVHD in this model, consistent with other observations [Ni, X., et al. (2017). J Clin Invest 127, 1960-1977]. To this end, the focus was on assessing the impact of αρ-T cell treatment on GVHD-causing CD4+ T cell responses.

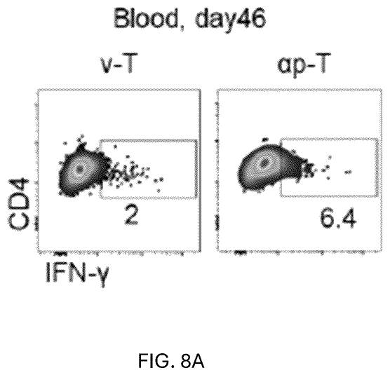

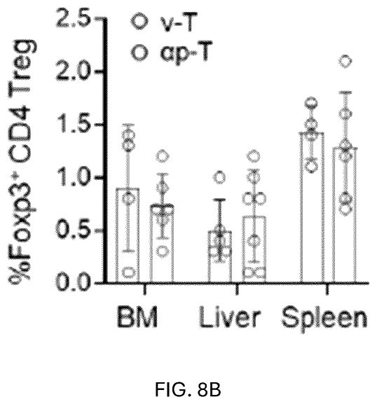

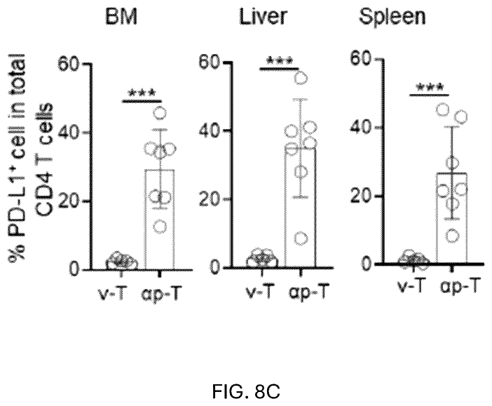

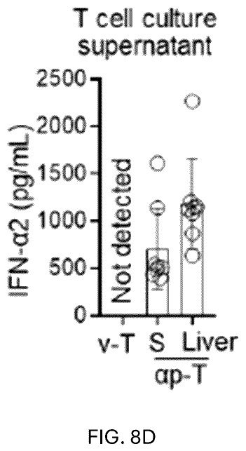

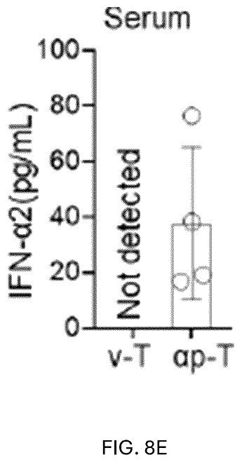

It was found that infusion of αρ-T cells resulted in significantly fewer numbers of total CD4+ T cells and IFN-γ⁺CD4+ effector T cells in the PB 46 days after transplantation (FIGS. 7D-7E). At the single cell level, αρ-T cells had higher frequencies of IFN-γ-producing effector cells and dead cells than v-T cells (FIGS. 7F-7G, FIG. 8A), suggesting that αρ-T cells may promote the generation of short-lived effector T cells. Furthermore, compared to NSG mice given v-T cells (CD4+ and CD8+ T cells), NSG mice receiving αρ-T cells had significantly fewer total numbers of donor CD4+ T cells in the BM, liver and spleen, along with a marked decrease in IFN-α-producing effector CD4+ T cells in the BM and spleen (FIGS. 7H-7I) but not changes in generation of Tregs (FIG. 8B). These findings corroborate the observations that IFN-α and PD-L1 can suppress in vivo expansion and survival of GVHD T cells [Tian, Y., et al. (2021). J Clin Invest 131, Ni, X., et al. (2017). J Clin Invest 127, 1960-1977, Robb, R. J., et al. (2011). Blood 118, 3399-3409, Song, Q., et al. (2021). Blood 137, 2243-2255, Deng, R., et al. (2015). Journal of immunology 194, 560-574].

Type I IFNs promote expression of coinhibitory receptors on human T cells, inducing PD-1 and TIM3 [Sumida, T. S., et al. (2022). Nat Immunol 23, 632-642]. At the end point of 85 days after transplantation, surviving CD4+αρ-T cells derived from the liver and spleen maintained high levels of PD-L1 (FIG. 8C) and secreted IFN-α2 (FIG. 8D). Furthermore, high levels of serum IFN-α2 were detected in NSG mice receiving CD4+αρ-T cells (FIG. 8E). These results indicate that αρ-T cells sustain their functionality during long-term disease control. It has been shown that PD-1/PD-L1 and TIM3/galectin-9 (TIM3 ligand) play important roles in repressing GVHD [Ni, X., et al. (2017). J Clin Invest 127, 1960-1977, Song, Q., et al. (2021). Blood 137, 2243-2255, Haspot, F., et al. (2008). Blood 112, 2149-2155, Li, X., et al. (2012). Journal of immunology 188, 724-734, Saha, A., et al. (2013). Blood 122, 3062-3073, Veenstra, R. G., et al. (2012). Blood 120, 682-690]. Thus, alloreactive T cells expressing high levels of PD-1 and TIM3 may have reduced capacity to mediate GVHD upon interaction with their ligands [Ni, X., et al. (2017). J Clin Invest 127, 1960-1977, Song, Q., et al. (2021). Blood 137, 2243-2255, Haspot, F., et al. (2008). Blood 112, 2149-2155, Li, X., et al. (2012). Journal of immunology 188, 724-734, Saha, A., et al. (2013). Blood 122, 3062-3073, Veenstra, R. G., et al. (2012). Blood 120, 682-690].

To assess whether αρ-T cell treatment may induce generation of PD1+TIM3+ T cells from αρ-T cells themselves, unpurified αρ-T cells (containing 60% of PD-L1+ cells) were transferred by infusion into hu-NSG mice. CD4+αρ-T cells stably produced significantly higher frequencies of PD-1+TIM3+ cells in the BM, spleen and liver than v-T cells 55 days after transfer (FIGS. 7J-7K). PD-1+TIM3+ T cells signify terminal exhausted T cells (term-TEX) that have reduced capacity to proliferate and survive [Jin, H. T., et al. (2010). Proc Natl Acad Sci USA 107, 14733-14738, Granier, C., et al. (2017). Cancer Res 77, 1075-1082, Petrie, J. C., and Scott, A. K. (1987). Br Med J (Clin Res Ed) 294, 919-920]. Thus, αρ-T cell-induced generation of PD-1+TIM3+ term-TEX-like cells may also be important for suppressing GVHD.

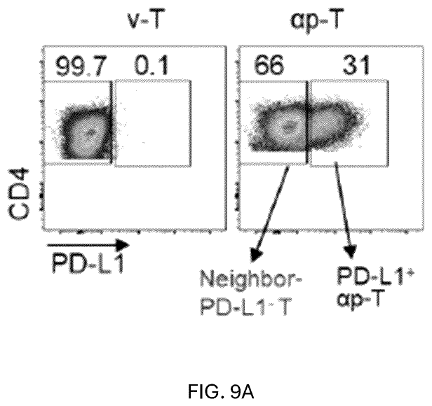

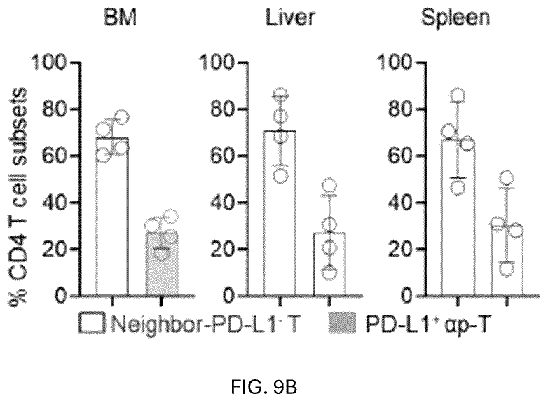

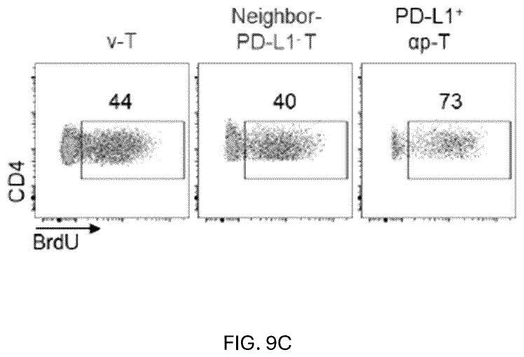

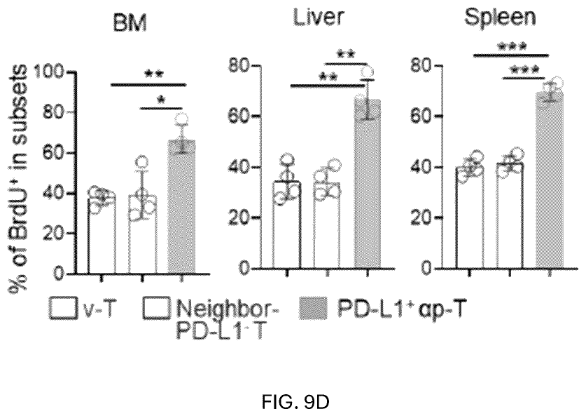

To assess whether αρ-T cells could have reduced migratory capacity, their expression of migration-related molecules (α4β7, CCR5, CCR9, CXCR3) was analyzed. Flow cytometric analysis showed that compared with CD4⁺v-T cells, CD4⁺PD-L1+αρ-T cells and neighboring PD-L1− T cells both expressed similar levels of α4β7, CCR5, and CCR9, but significantly higher CXCR3 prior to transplantation (FIG. 7L). Furthermore, tissue-infiltrating PD-L1+αρ-T cells and neighboring PD-L1− T cells (FIGS. 9A-9B) isolated from the GVHD target organ (liver and spleen) 26 days after transplantation prior to clinical signs of GVHD occurred, displayed significantly higher frequencies of CCR5 and CXCR3 than v-T cells (FIGS. 7M-7N). Importantly, like v-T cells, these tissue-infiltrating CD4⁺PD-L1+αρ-T cells retained proliferative capacity upon exposure to persistent xenogeneic antigens (FIGS. 9C-9D).

Consistent with reports that chronic antigen stimulation induces stable PD-1 expression [Bally, A. P., et al. (2016). Journal of immunology 196, 2431-2437, Ghoneim, H. E., et al. (2017). Cell 170, 142-157 e119], liver-infiltrating PD-L1⁺αρ-T cells and neighboring PD-L1− T cells showed high PD-1 expression (FIG. 7O). These PD-L1⁺αρ-T cells and PD-L1− T cells had significantly higher levels of p-SHP2 than v-T cells (FIGS. 7O-7P), indicating in vivo activation of PD-1 signaling. These data indicate that GVHD inhibition likely results from decreased survival capability of PD-L1+αρ-T cells and their neighboring PD-L1− T cells.

FIG. 7A, FIG. 7B, FIG. 7C, FIG. 7D, FIG. 7E, FIG. 7F, FIG. 7G, FIG. 7H, FIG. 7I, FIG. 7J, FIG. 7K, FIG. 7L, FIG. 7M, FIG. 7N, FIG. 7O, and FIG. 7P show that αρ-T cell treatment reduces xeno-reactive T cell responses in x-GVHD mice. Circulating peripheral blood (PB) was collected on day 46 after transplantation for flow cytometric analysis. FIG. 7A shows dot plots of circulating PB that was collected on day 46 after transplantation for flow cytometric analysis, depicting human CD45⁺ cells in the PB (upper panel) and frequencies of CD4⁺ and CD8⁺ T cells within gated CD45⁺ cells (lower panel). FIG. 7B shows cell number of human CD45⁺ cells in the PB. FIG. 7C shows graphs depicting the frequencies of donor cells. FIG. 7D shows total cell number of circulating PB. FIG. 7E shows IFN-γ⁺CD4⁺ T cells of circulating PB. FIG. 7F shows the frequency of IFN-γ⁺CD4⁺ T cells in circulating PB. FIG. 7G shows the viability of donor CD4⁺ T cells. FIG. 7H shows the total number of CD4⁺ T cells in bone marrow (BM), liver and spleen tissues that were harvested at study end time point. FIG. 7I shows IFN-γ⁺CD4⁺ T cells in BM, liver, and spleen tissues that were harvested at study end time point. FIG. 7J shows plots depicting PD-1⁺TIM3⁺expression of CD4 T cells in the PB, BM, liver and spleen. FIG. 7K shows bar graphs depicting PD-1⁺TIM3⁺expression of CD4 T cells in the PB, BM, liver and spleen. FIG. 7L shows bar graphs depicting percent cell of adhesion molecular expression on CD4⁺ T cells in vitro prior to transplantation. FIG. 7M shows bar graphs show frequency of adhesion molecule-expressing CD4⁺ T cells derived from GVHD livers of mice received v-T or αρ-T cells 26 days after transplantation. FIG. 7N shows bar graphs show frequency of adhesion molecule-expressing CD4⁺ T cells derived from GVHD spleens of mice received v-T or αρ-T cells 26 days after transplantation. FIG. 7O shows histograms depicting PD-1 (upper panel) and p-SHP2 (lower panel) expression on CD4⁺ T cell subsets. FIG. 7P shows bar graphs depicting pSHP2 expression on CD4⁺ T cell subset derived from liver. *, P<0.05; **, P<0.01; ***, P<0.001.

FIG. 8A, FIG. 8B, FIG. 8C, FIG. 8D, and FIG. 8E show the characterization of human αρ-T cells in x-GVHD mice. Blood was collected on day 46. Tissues were collected at study endpoints. FIG. 8A shows representative dot plots showing IFN-g production in CD4+ T cells derived from v-T or αρ-T cell recipients. FIG. 8B shows graphs depicting percentage of Foxp3+CD4+ Tregs in total CD4+ T cells derived from v-T or αρ-T cell recipients. FIG. 8C shows graphs depicting PD-L1 percentage in CD4+ T cells derived from v-T or αρ-T cell recipients. FIG. 8D shows mononuclear cells that were collected and stimulated with PMA and ionomycin for 8 hours from liver and spleen on day 85 after transplantation. FIG. 8E shows a graph depicting IFN-α2 expression in serum on day 85 after transplantation. *, P<0.05; **, P<0.01; ***, P<0.001.

FIG. 9A, FIG. 9B, FIG. 9C and FIG. 9D show PD-L1+αρ-T cells displaying increased dividing capacity in vivo. NSG mice were administered with 10 million TCD PBMCs on day −7, followed by injection of 10 million αρ-T cell products on day 0. Mice were sacrificed for analysis on day 26 after transplantation. FIG. 9A shows representative plots depicting PD-L1 expression from liver. FIG. 9B shows bar graphs depicting percent cell of PD-L1 positive CD4+ T cells in tissues. FIG. 9C shows representative plots depicting BrdU+ cells in CD4+ T cell subsets from liver. BrdU, or 5-bromo-2′-deoxyuridine, is a thymidine analog used to study cell proliferation. It is incorporated into newly synthesized DNA during S-phase of the cell cycle and is subsequently detected using antibodies to identify which cells are dividing. FIG. 9D shows bar graphs depicting percent cell of BrdU incorporation in CD4+ T cell subsets. *, P<0.05; **, P<0.01; ***, P<0.001.

αρ-T Cell-Derived IFN-α Activates Gene Programs that Promote T Cell Death and Exhaustion.

Type I IFNs (including IFN-α and IFN-β) have long been recognized for their ability to inhibit T cell proliferation, promote effector differentiation and sensitize T cells to activation-induced cell death [Sumida, T. S., et al. (2022). Nat Immunol 23, 632-642, Robb, R. J., et al. (2011). Blood 118, 3399-3409, Curtsinger, J. M., et al. (2005). Journal of Immunology 174, 4465-4469, Fradelizi, D., and Gresser, I. (1982). J Exp Med 155, 1610-1622, Kuka, M., et al. (2019). Immunol Lett 215, 19-23, Chen, W., et al. (2022). Cell Rep 41, 111647].

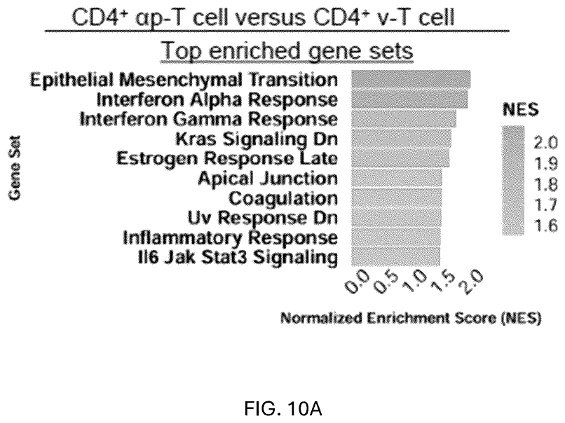

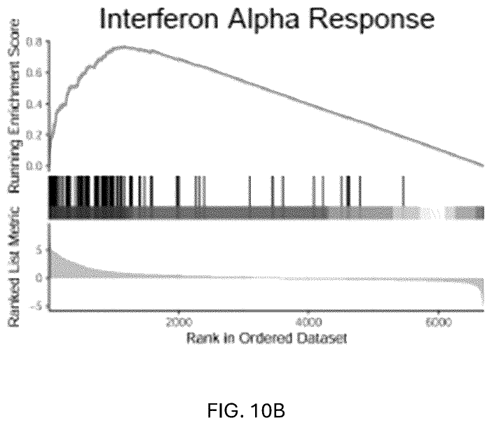

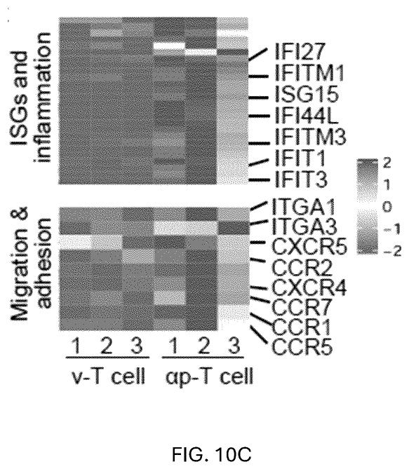

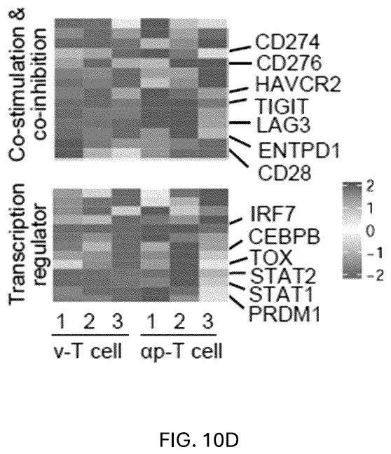

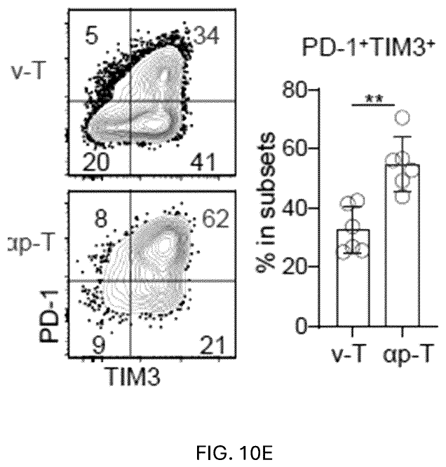

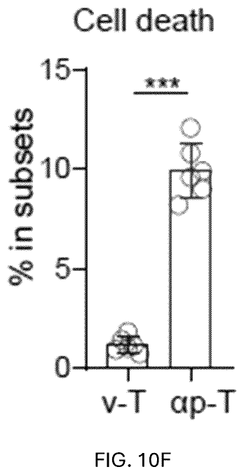

To understand how IFN-α-derived from human CD4+αρ-T cells influences the gene programs critical for survival, expansion and differentiation in themselves, RNA-seq analysis was performed using highly purified PD-L1+CD4+αρ-T cells and control CD4+ v-T cells. Compared to CD4+v-T cells, CD4+αρ-T cells upregulated 1381 genes (>1.5-fold changes, p-adjusted value <0.05) and down-regulated 656 genes. GSEA analysis revealed that these upregulated differentially expressed genes (DEGs) in CD4+αρ-T cells were associated with IFN response and inflammatory response (FIGS. 10A-10B), indicative of activated IFN-α signaling in these αρ-T cells. Indeed, CD4+αρ-T cells increased expression of IFN-Stimulated Genes (ISGs, e.g., IFI27, IFITM1, and ISG15) and genes encoding cell migration and tissue infiltration (ITGA1, ITGA3, CCR5, and CCR7) (FIG. 10C). They also upregulated expression of genes encoding inhibitory receptors (HAVCR2 (encoding TIM3), LAG3, ENTPD1 (encoding CD39) and TIGIT) and transcription factors (PRDM1, TOX, STAT1, and CEBPB) critical for promoting generation of short-lived effector cells and TEX cells (FIG. 10D). Flow cytometry analysis showed the CD4+αρ-T cells produced significantly higher frequencies of PD-1+TIM3+ cells than their counterpart v-T cells (FIG. 10E), which was associated with increased cell death (FIG. 10F).

FIG. 10A, FIG. 10B, FIG. 10C, FIG. 10D, FIG. 10E and FIG. 10F show the characteristics of CD4⁺αρ-T cells suppressive function. CD4⁺PD-L1⁺αρ-T cells and v-T cells were produced and FACS-sorted at day 12 of culture for bulk RNA-seq analysis after being stimulated with PMA and ionomycin for 2 hours. FIG. 10A shows bar plots depicting GSEA enrichment analysis. FIG. 10B shows GSEA analysis depicting featured pathways. FIG. 10C shows a heatmap depicting the expression of featured differential expressed genes (DEGs). FIG. 10D shows a heatmap depicting the expression of featured differential expressed genes (DEGs). FIG. 10E shows FACS-sorted day 12 PD-L1⁺αρ-T cells and v-T cells that were re-cultured in the presence of anti-CD3/28 Ab for an additional 10 days. Plots and graphs show the percentage of PD1⁺TIM3⁺ cells. FIG. 10F shows FACS-sorted day 12 PD-L1⁺αρ-T cells and v-T cells that were re-cultured in the presence of anti-CD3/28 Ab for an additional 10 days. Graph shows cell viability. *, P<0.05; **, P<0.01; ***, P<0.001.

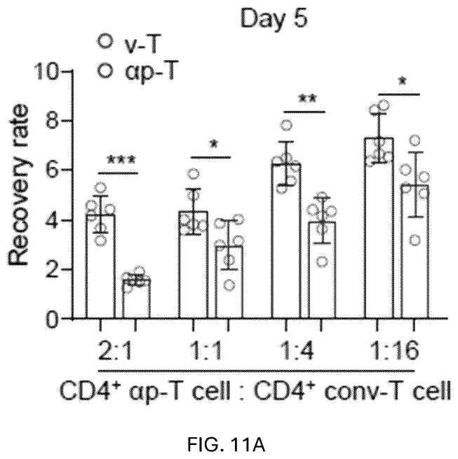

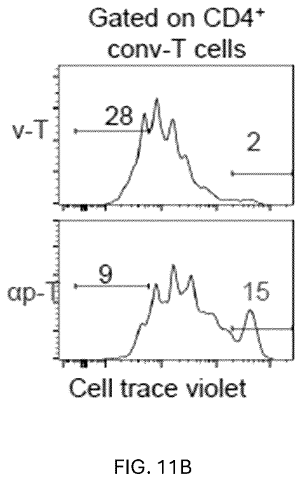

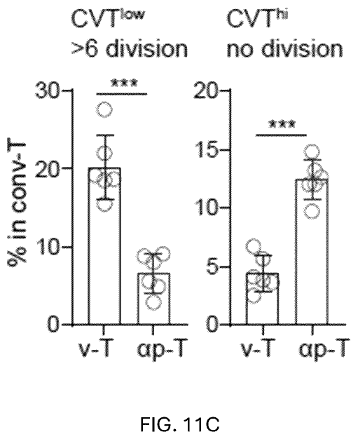

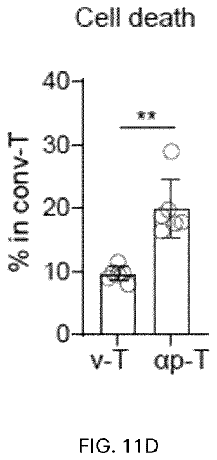

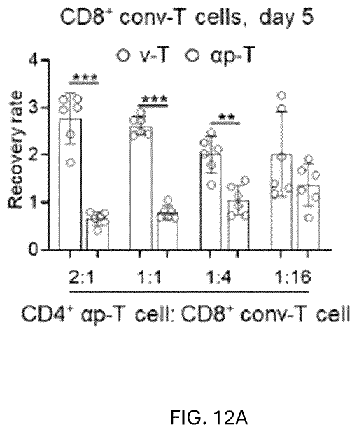

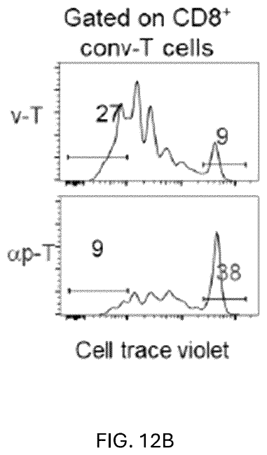

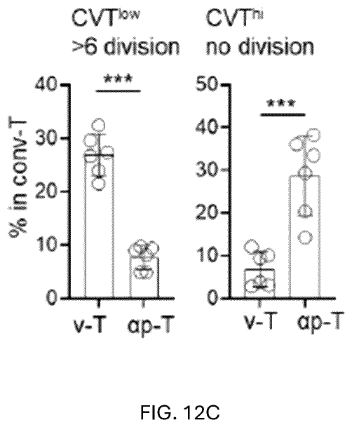

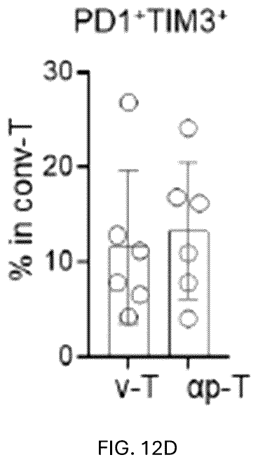

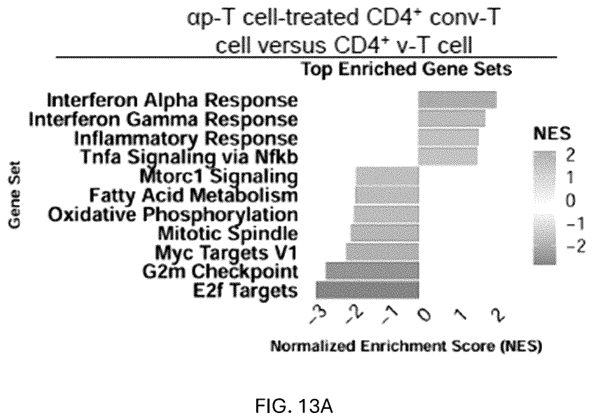

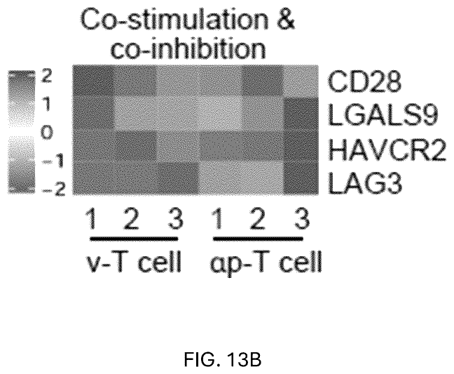

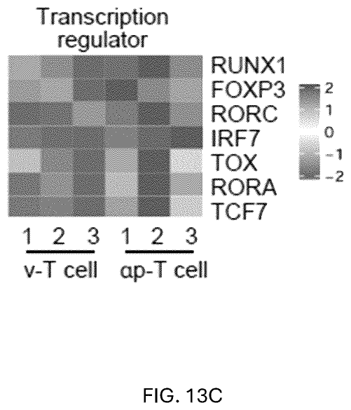

To assess the direct effect of αρ-T cells on conventional T (conv-T) cells, green fluorescent protein (GFP)-expressing (GFP+) CD4+αρ-T cells (suppressors) were FACS sorted and added to cultures of TCR-activated conv-T cells (both CD4 and CD8) at various ratios of suppressors to conv-T cells. Compared to GFP+CD4+v-T cells, suppressor CD4+αρ-T cells dose-dependently suppressed expansion of CD4+ conv-T cells (FIG. 11A), which was associated with reduced cell proliferation (FIGS. 11B-11C) and increased cell death of these conv-T cell (FIG. 11D). CD4+αρ-T cell treatment also suppressed proliferation of CD8+ conv-T cells (FIGS. 12A-12C) but had no significant effects on their generation of PD-1+TIM3+ cells under this culture condition (FIG. 12D). RNA-seq analysis revealed that compared to CD4+v-T cells, suppressor CD4+αρ-T cell-treated CD4+ conv-T cells upregulated DEGs associated with IFN-α response, IFN-gamma response and inflammatory response (FIG. 13A). Among these DEGs, increased expression of LAG3, HAVCR2 and TOX transcripts but decreased CD28 were indicative of T cell exhaustion (FIGS. 13B-13C). Those down-regulated DEGs were associated with oxidative phosphorylation and Mtorc1 signaling, as well as mitotic nuclear division and DNA replication (FIG. 13A), which are critical for T cell proliferation and survival [MacIver, N.J., et al. (2013). Annu Rev Immunol 31, 259-283].

FIG. 11A, FIG. 11B, FIG. 11C and FIG. 11D show GFP-expressing CD4⁺αρ-T cells or GFP-expressing CD4⁺v-T cells that were FACS-sorted 12 days after production, stimulated with PMA+ionomycin for additional 2 hrs, added to autologous non-transduced CD4⁺ conv-T cells labeled with CellTrace violet (CTV) at varying ratios, activated with anti-CD3 Ab (1.0 μg/ml) and anti-CD28 Ab (50.0 ng/ml) and cultured for additional 5 days. FIG. 11A shows a graph depicting the expansion of CD4⁺ conv-T cells. FIG. 11B shows plots depicting CTV dilution. FIG. 11C shows graphs depicting CTV dilution. FIG. 11D shows CD4+ conv-T cells that were collected 8 days after cultured with CD4⁺αρ-T cells or GFP-expressing CD4⁺v-T cells, restimulated with anti-CD3 (1.0 μg/ml) for additional 6 hrs to measure their viability. *, P<0.05; **, P<0.01; ***, P<0.001.

FIG. 12A, FIG. 12B, FIG. 12C and FIG. 12D show αρ-T cells suppress autologous primary CD8+ conv-T cells. GFP-expressing CD4+αρ-T cells or GFP-expressing CD8+v-T cells were FACS-sorted 12 days after production, stimulated with PMA+Ionomycin for additional 2 hours, added to autologous CD8+ conv-T cell labeled with CellTrace™ violet (CTV) at varying ratios, activated with anti-CD3 Ab (1 μg/mL) and anti-CD28 Ab (50 ng/mL) and cultured for an additional 5 days. FIG. 12A shows the recovery rates of cultured Cd8+ conv-T cells on day 5 of culture. FIG. 12B shows a plot depicting CellTrace™ dilution on day 5 of culture. FIG. 12C shows a graph depicting CellTrace™ dilution on day 5 of culture. FIG. 12D shows the fraction of PD-1+TIM3+ cells in CD8+ conv-T cells. **, P<0.01; ***, P<0.001.

FIG. 13A, FIG. 13B, and FIG. 13C show PD-L1⁻CD4⁺ conv-T cells that were FACS-sorted 5 days after cultured with CD4⁺αρ-T cells and CD4+v-T cells, respectively, and stimulated with PMA and ionomycin for 2 hours for bulk RNA-seq analysis. FIG. 13A shows a GSEA bar plot for Hallmark pathways. FIG. 13B shows a heatmap depicting expression of representative DEGs. FIG. 13C shows a heatmap depicting expression of additional representative DEGs. *, P<0.05; **, P<0.01; ***, P<0.001.

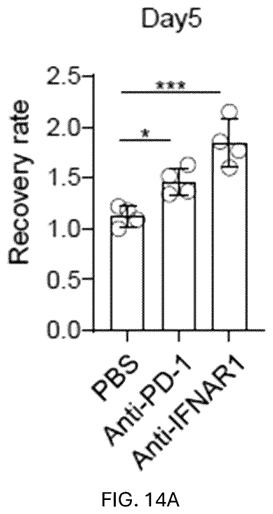

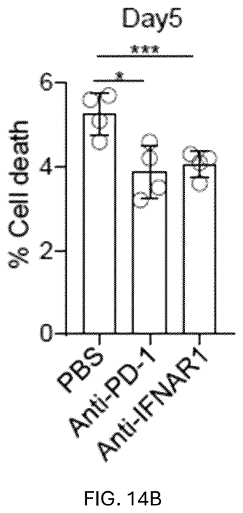

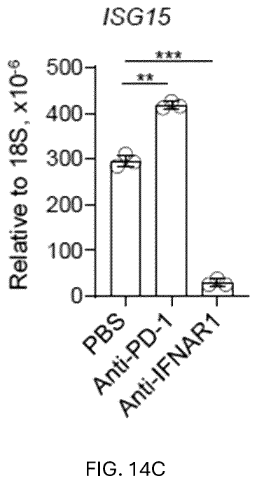

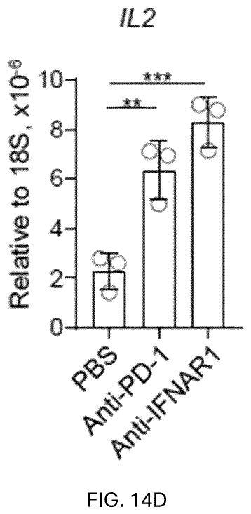

It was assessed whether the observed changes in conv-T cell proliferation and gene expression are due to receptor-mediated stimulation by IFN-α and PD-L1. PD-1− or IFNAR1-specific neutralizing Ab were added to TCR-activated conv-T cells (i.e., non-transfected cells) that were cocultured with suppressor CD4+αρ-T cells. Blockade of either PD-1 or IFNAR1 significantly improved the expansion and survival capacity of conv-T cells (FIGS. 14A-14B). IFN-α signaling induces a broad range of ISGs and active PD-1 signaling represses IL-2 expression [Patsoukis, N., et al. (2020). Sci Adv 6, Riley, J. L. (2009). Immunol Rev 229, 114-125]. It was found that blockade of IFNAR1 and PD-1 resulted in dramatically decreased expression of ISG15 and increased IL2 genes, respectively (FIGS. 14C-14D). These data indicate that both IFNAR1 and PD-1 signaling mediate important roles in αρ-T cell modulation of conv-T cell proliferation and gene expression.

FIG. 14A, FIG. 14B, FIG. 14C and FIG. 14D show the effects of a neutralizing Ab against PD-1 or IFNAR1 that was added into the CD4⁺αρ-T cells and CVT labeled CD4⁺ conv-T cell coculture system from day 0-day 5. FIG. 14A shows recovery rate of conv-T cells in each group. FIG. 14B shows cell death of conv-T cells in each group. FIG. 14C shows bar graphs depicting RNA levels of ISG15 expression on conv-T cells. Dividing conv-T cells were sorted on day 5 after coculture. FIG. 14D shows IL-2 expression on conv-T cells. Dividing conv-T cells were sorted on day 5 after coculture. *, P<0.05; **, P<0.01; ***, P<0.001.

Collectively, these data suggest that enforced expression of IFN-α in T cells activates transcription programs critical for promoting effector differentiation, exhaustion and cell death, thereby reducing expansion and persistence of either αρ-T cells and their treated conv-T cells.

### IFN-α and PD-L1 Form a Synergistic Regulatory Loop to Modulate Conv-T Cell Responses.

During GVHD, alloreactive T cells upregulate expression of both PD-1 and TIM3, thereby becoming sensitized to suppressive effects of their cognate ligands [Ni, X., et al. (2017). J Clin Invest 127, 1960-1977, Song, Q., et al. (2021). Blood 137, 2243-2255, Haspot, F., et al. (2008). Blood 112, 2149-2155, Li, X., et al. (2012). Journal of immunology 188, 724-734, Saha, A., et al. (2013). Blood 122, 3062-3073, Veenstra, R. G., et al. (2012). Blood 120, 682-690].

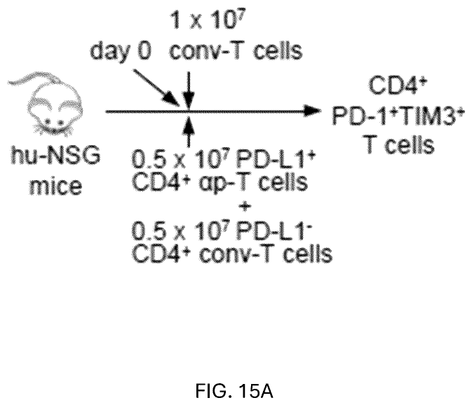

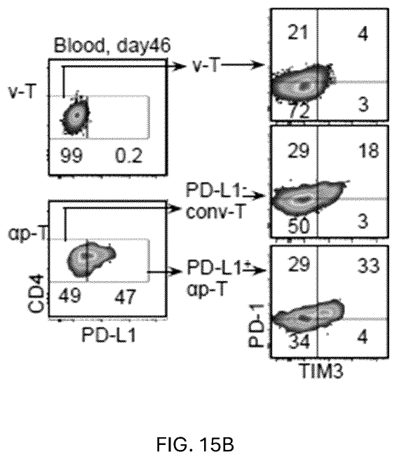

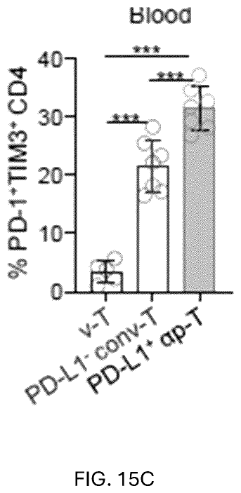

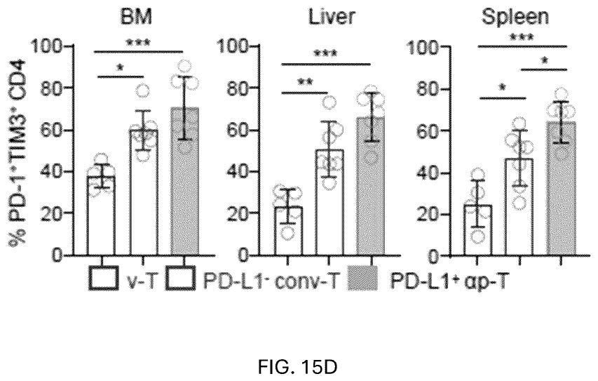

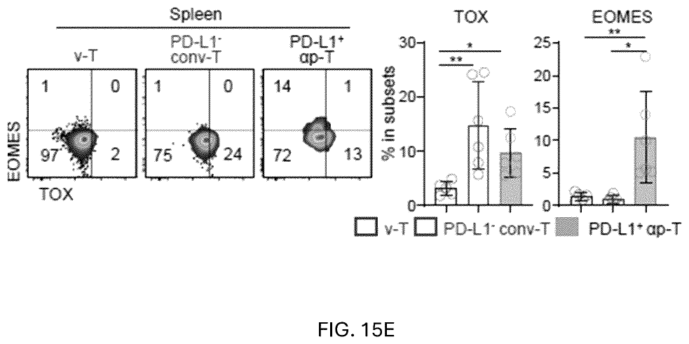

In this regard, it was reasoned that αρ-T cell induction of PD-1+TIM3+ cells rendered them of enhanced sensitivity to PD-L1-mediated suppression. To test this hypothesis, two strategies were used. First, the precise effect of αρ-T cell treatment on promoting the in vivo generation of PD-1+TIM3+ cells from conv-T cells during GVHD was examined. Equal numbers of PD-L1+CD4+αρ-T cells and CD4+ conv-T cells were mixed and transferred into hu-NSG mice by infusion, with transfer of CD4+ conv-T cells alone as control (FIG. 15A). Compared to control CD4+ conv-T cells, PD-L1+CD4+αρ-T cells and neighboring PD-L1−CD4+ conv-T cells in hu-NSG receiving the cell mixture each produced significantly higher frequencies of PD-1+TIM3+ cells in the PB, BM, liver and spleen 46 days after transplantation (FIGS. 15B-15D). Transcription factors such as EOMES and TOX are important for T cell exhaustion, with EOMES linked to terminally exhausted T cells and TOX promoting the expression of PD-1 [Seo, H., et al. (2019). Proc Natl Acad Sci USA 116, 12410-12415. 10.1073/pnas. 1905675116, Jenkins, E., et al. (2023). Oxf Open Immunol 4, iqad006. 10.1093/oxfimm/iqad006].

Flow cytometric analysis revealed that CD4+αρ-T cell treatment resulted in marked increase in expression of TOX by PD-L1−CD4+ conv-T cells (FIG. 15E). This suggests that CD4+αρ-T cells can promote CD4+ conv-T cells to differentiate into PD-1+TIM3+ term-TEX-like cells during GVHD.

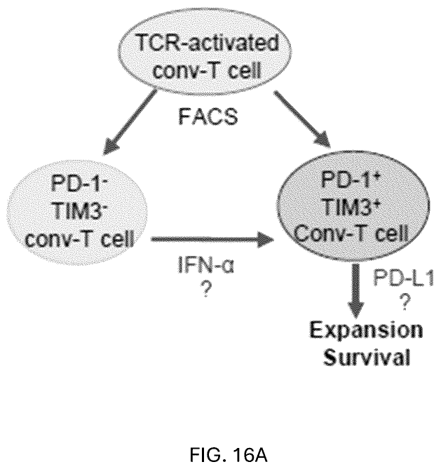

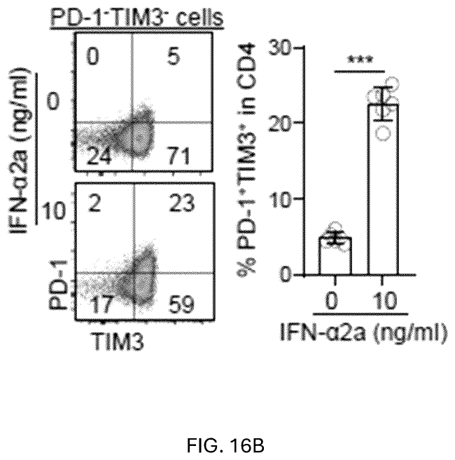

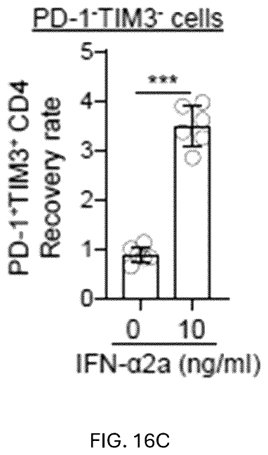

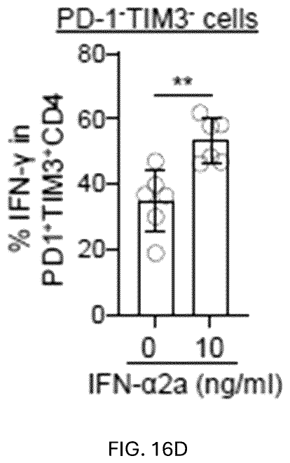

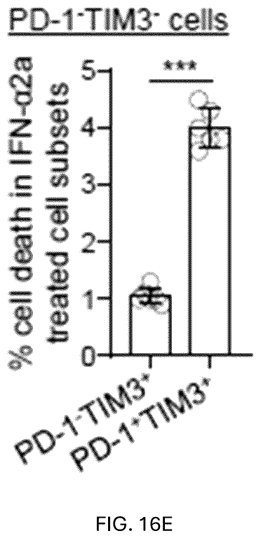

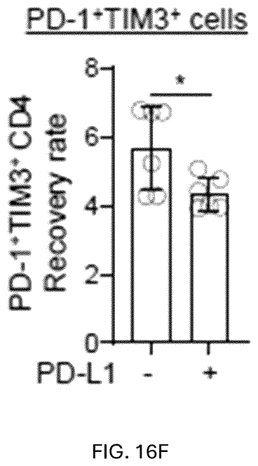

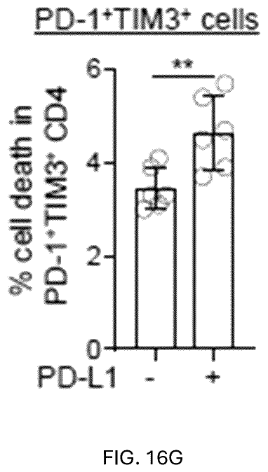

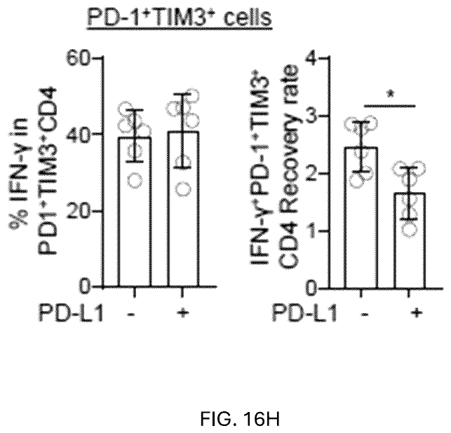

Second, individual roles of IFN-α and PD-L1 in CD4+ conv-T cells were tested. PD-1−TIM3− and PD-1+TIM3+ cells from primary cultured TCR-activated CD4+ conv-T cells were FACS sorted, which had never been treated with αρ-T cells, and separately cultured them in the presence of PBS, IFN-α2a or PD-L1+K562 cells, respectively (FIG. 16A). Primary PD-1−TIM3− cells stimulated with IFN-α produced 3-4-fold higher in frequency and numbers of secondary PD-1+TIM3+ cells (FIGS. 16B and 16C), consistent with the observations in animals (FIGS. 15B, FIG. 15C, FIG. 15D).

These IFN-α-induced PD-1+TIM3+ cells produced higher levels of IFN-γ than those derived from PBS-treated control (FIG. 16D), suggesting a pro-effector differentiation role of IFN-α. Furthermore, in cultures treated with IFN-α2a, PD-1+TIM3+ T cells experienced 4-fold higher frequency of death than T cells lacking both markers (PD-1−TIM3−) (FIG. 16E). Introduction of PD-L1+K562 cells to the culture of primary PD-1+TIM3+ cells resulted in a moderate but statistically significant reduction in the expansion and survival of these PD-1+TIM3+ cells (FIGS. 16E-16G), which was associated with markedly reduced numbers of IFN-γ-producing PD-1+TIM3+ T cells (FIG. 16H). Altogether, IFN-α and PD-L1 form a synergistic regulatory loop to suppress TCR-driven responses of conv-T cells. IFN-α promoted the generation of PD-1+TIM3+ term-TEX-like cells, whereas PD-L1 induces exhaustion and death of these PD-1+TIM3+ T cells.

FIG. 15A, FIG. 15B, FIG. 15C, FIG. 15D, and FIG. 15E show that αρ-T cells produce a synergistic reinforcing regulatory loop to repress activated conv-T cell responses. FIG. 15A shows equal numbers of CD4⁺PD-L1⁺αρ-T cells plus CD4⁺ conv-T cells were injected into hu-NSG mice to induce GVH reaction, with transfer of CD4⁺ conv-T cells as control. FIG. 15B shows flow cytometric plots of donor T cells in the PB from NSG mice 46 days after transplantation. FIG. 15C shows flow cytometric graphs of donor T cells in the PB from NSG mice 46 days after transplantation. FIG. 15D shows expression of PD-1 and TIM3 on Donor T cells that were isolated from BM, liver and spleen. FIG. 15E shows expression of TOX and EOMES in T cell subsets. *, P<0.05; **, P<0.01; ***, P<0.001.

FIG. 16A, FIG. 16B, FIG. 16C, FIG. 16D, FIG. 16E, FIG. 16F, FIG. 16G, and FIG. 16H show PD-1⁻TIM3⁻ cells and PD-1⁺TIM3⁺ cells that were FACS-sorted from v-T cells at day 12 of culture, and re-cultured in the presence of anti-CD3 Ab with or without adding IFN-α2a (10.0 ng/ml), K562 or PD-L1⁺K562 for additional 7 days. FIG. 16A shows the graphic summary of the experiment design. FIG. 16B shows frequencies of PD-1⁺TIM3⁺ cells. FIG. 16C shows the recovery rate of PD-1⁺TIM3⁺ cells. FIG. 16D shows IFN-γ expression percentage of PD-1⁺TIM3⁺ cells. FIG. 16E shows frequencies of cell death in IFN-α2a treated cell subsets generated from PD-1⁻TIM3⁻ cells. FIG. 16F shows the recovery rate of PD-1⁺TIM3⁺ cells. FIG. 16G shows the frequency of cell death of PD-1⁺TIM3⁺ cells. FIG. 16H shows the IFN-γ expression percentage of PD1+TIM3+CD4 and the recovery rate of IFN-γ⁺PD-1⁺TIM3⁺. *, P<0.05; **, P<0.01; ***, P<0.001.

### Murine CD4⁺αρ-T Cell Treatment Ameliorates GVHD in Immunocompetent Mice.

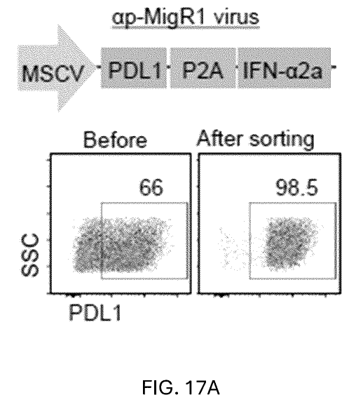

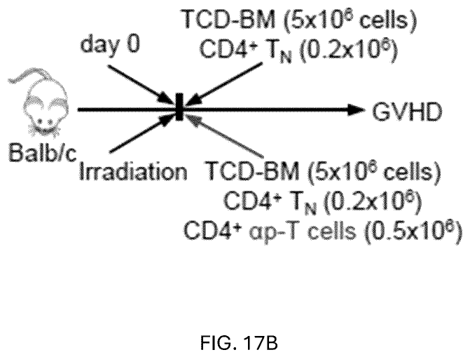

To understand murine CD4+αρ-T cell effects on repressing GVHD in immunocompetent mice of allo-HCT, retrovirus encoding murine IFN-α and PD-L1 (αρ-MigR1 virus) were produced (FIG. 17A). Murine CD4+ T cells of B6 mouse origin (C57BL/6 mice, commonly used as genetic backgrounds for genetically modified mice) were transduced with αρ-MigR1 virus to generate suppressor CD4+αρ-T cells. PD-L1+CD4+αρ-T cells were FACS sorted, mixed with or without B6 CD4+ T_{N} cells, and transplanted together with TCD-BM into lethally irradiated Balb/c mice (FIG. 17A and FIG. 17B).

FIG. 17A and FIG. 17B show that murine CD4⁺αρ-T cells reduced GVHD and T cell response in vivo. FIG. 17A shows the retroviral vector structure encoding murine PD-L1 and IFN-α (αρ-MigR1, upper panel). Murine CD4+ T cells were activated with anti-CD3/28 Ab and infected with αρ-MigR1 retrovirus. PD-L1⁺αρ-T cells were sorted by FACS (lower panel). FIG. 17B shows a schematic of the experimental procedure. *, P<0.05; **, P<0.01; ***, P<0.001.

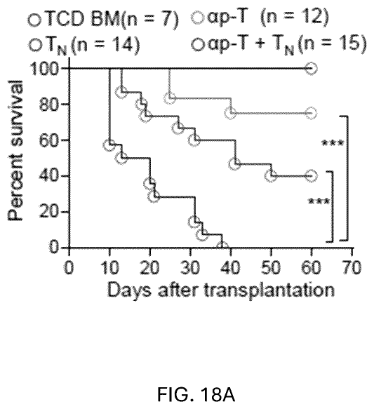

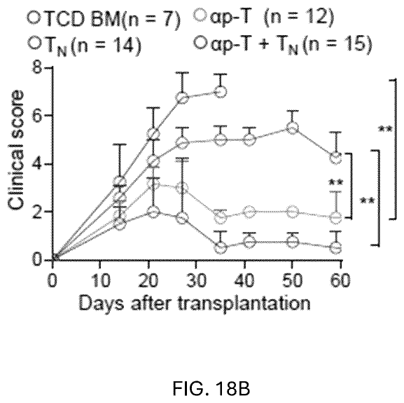

Infusion of CD4+αρ-T cells alone did not induce severe GVHD. Addition of CD4+αρ-T cells to donor CD4+ T_{N} cells significantly ameliorated severity of acute GVHD in Balb/c recipients compared to Balb/c recipients given CD4+ T_{N} cells (FIG. 18A and FIG. 18B). These data suggest that CD4+αρ-T cells possess potent capacity to reduce GVHD in immunocompetent mice.

FIG. 18A and FIG. 18B show Balb/c recipients that were irradiated with 8.5 Gy and transplanted with 5×10⁶ B6xB6/SJL F1 (CD45.1+CD45.2+) TCD BM (TCD-BM, n=7), together with 0.5×10⁶ FACS-sorted B6/SJL (CD45.1+) PD-L1⁺CD4⁺αρ-T cells (αρ-T, n=12), 0.2×10⁶ B6 (CD45.2+) CD4⁺ T_{N} cells (T_{N}, n=14), or 0.2×10⁶ CD4⁺ T_{N} cells+0.5×10⁶ PDL1+CD4⁺αρ-T cells (αρ-T+T_{N}, n=15). FIG. 18A shows survival. FIG. 18B shows clinical score. *, P<0.05; **, P<0.01; ***, P<0.001.

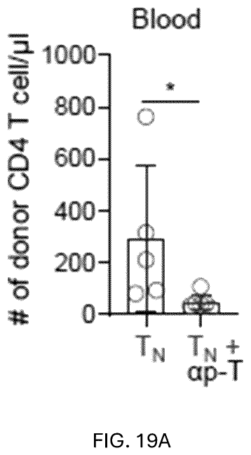

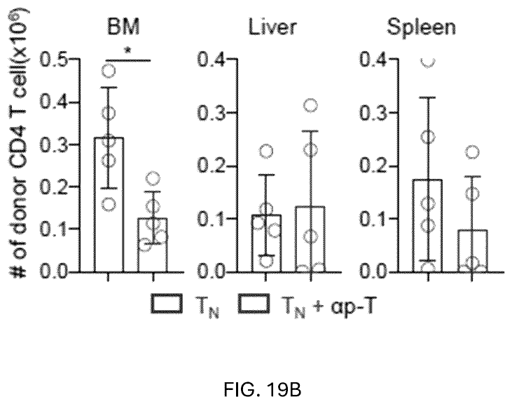

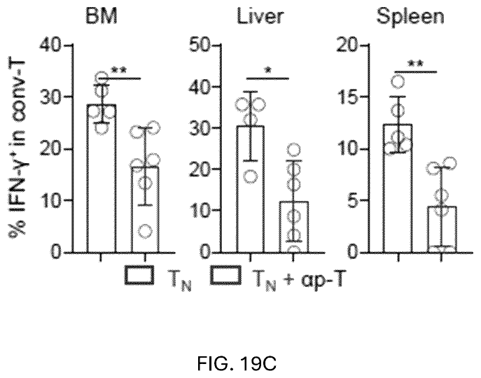

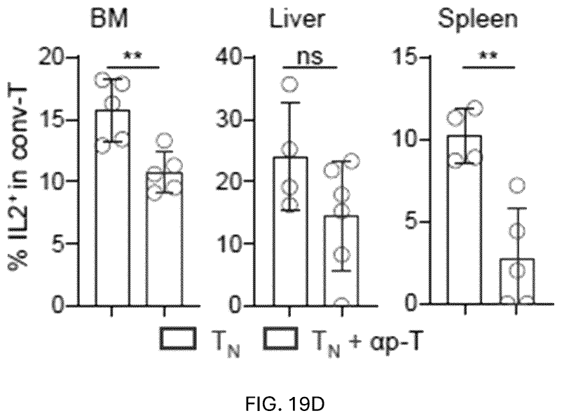

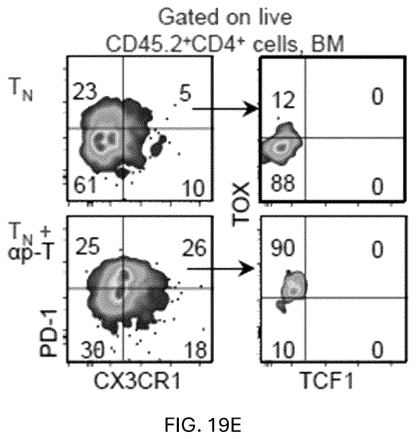

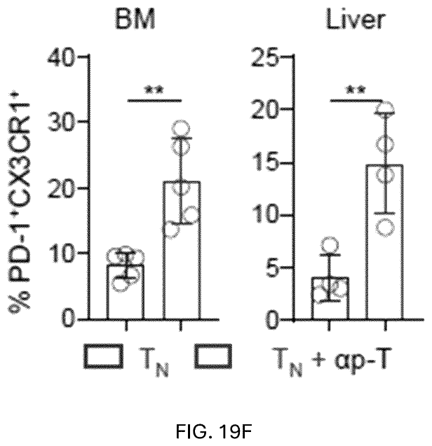

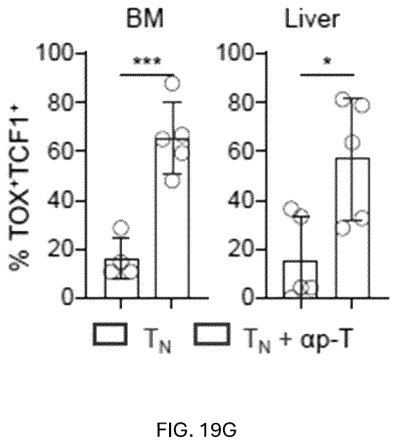

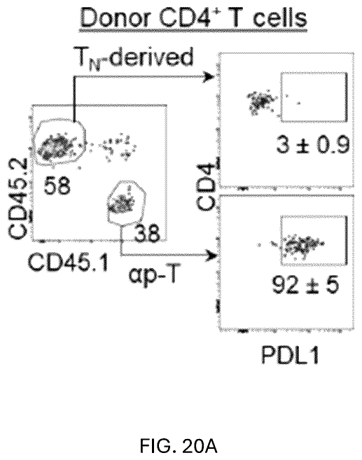

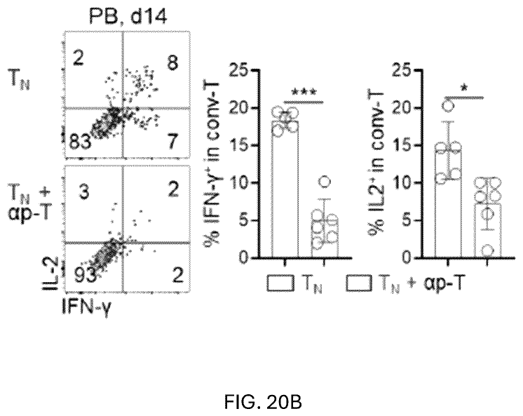

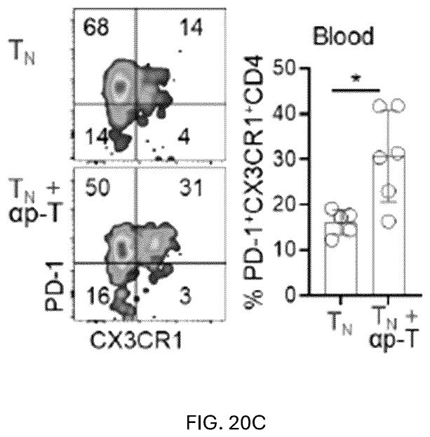

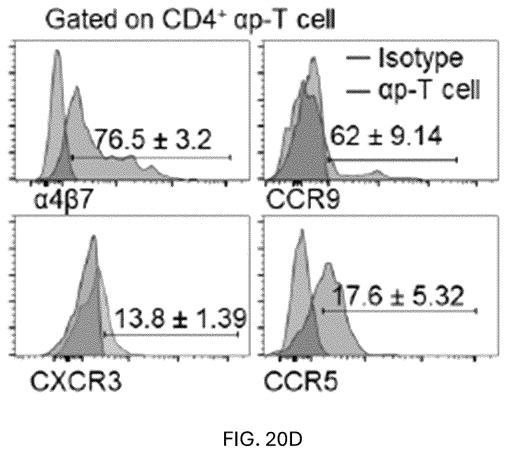

To examine the underlying mechanism, donor B6 CD4+ T_{N} cells (CD45.2+) and B6xB6/SJL TCD-BM (CD45.2+CD45.1+) were transplanted, mixed with or without purified PD-L1+CD4+αρ-T cells (CD45.1+), into lethally irradiated Balb/c mice. Suppressor CD4+αρ-T cell treatment markedly reduced total number of alloreactive T cells in the PB and BM (FIGS. 19A, FIG. 19B, FIG. 20A), and decreased frequencies of IFN-γ-producing alloreactive effector T cells in the PB (FIG. 20B) and GVHD target tissues (BM, spleen and liver) (FIG. 19C). IL-2-producing T cells were also significantly reduced in the PB, BM and spleen of recipients treated with CD4+αρ-T cells compared to untreated control recipients (FIG. 20B and FIG. 19D), consistent with the effect of the PD-L1/PD-1 pathway on repressing IL-2 production [Chemnitz, J. M., et al. (2004). Journal of Immunology 173, 945-954, Sharpe, A. H., and Pauken, K. E. (2018). Nat Rev Immunol 18, 153-167].

In GVHD mice, T cells with high levels of PD-1, CX3CR1 and TOX but low levels of TCF1 are term-TEX cells [Wang, Y., et al. (2023). Blood. 10.1182/blood.2023021126, Sacirbegovic, F., et al. (2023). Immunity. 10.1016/j.immuni.2023.01.003, Senjo, H., et al. 2024. J Clin Invest 134].

Compared to PBS control, suppressor CD4+αρ-T cell treatment resulted in a markedly increased frequency of PD-1+CX3CR1+CD4+ conv-T cells in the PB, BM, spleen and liver (FIG. 20C, FIG. 19E, FIG. 19F). These PD-1+CX3CR1+CD4+ T cells derived from recipients given CD4+αρ-T cells were TOX+TCF1− cells (FIG. 19G), indicative of term-TEX-like cells.

Chemokine receptors CCR9 and CCR5 and adhesion molecule α4β7 are required for T cell migration into GVHD tissues [Wysocki, C. A., et al. (2005). Blood 105, 4191-4199, Reshef, R., et al. (2012). N Engl J Med 367, 135-145. 10.1056/NEJMoa1201248]. Suppressor CD4+αρ-T cells derived from the spleen expressed high levels of CCR5, CCR9 and α4β7 (FIG. 20D). These results suggest CD4+αρ-T cells retain the capacity to traffic into GVHD target tissues where they promote the generation term-TEX cells that may have reduced capacity to mediate GVHD.

FIG. 19A, FIG. 19B, FIG. 19C, FIG. 19D, FIG. 19E, FIG. 19F, and FIG. 19G show an analysis of blood collected on day 14 and tissues collected on day 21 after transplantation. FIG. 19A shows the number of donor CD4+ T cells in the peripheral blood (PB). FIG. 19B shows GVHD target tissues. FIG. 19C shows IFN-γ+ cytokine production in donor CD4+ T cells. FIG. 19D shows IL2+ cytokine production in donor CD4+ T cells. FIG. 19E depicts a plot showing the frequency of PD-1⁺CX3CR1⁺CD4⁺ conv-T cells and frequency of TOX⁺TCF1⁻cells within the PD-1⁺CX3CR1⁺CD4⁺ conv-T cell population. FIG. 19F shows a graph showing the frequency of PD-1⁺CX3CR1⁺CD4⁺ conv-T cells within the PD-1⁺CX3CR1⁺CD4⁺ conv-T cell population. FIG. 19G shows a graph showing the frequency of TOX⁺TCF1⁻ cells within the PD-1⁺CX3CR1⁺CD4⁺ conv-T cell population. *, P<0.05; **, P<0.01; ***, P<0.001.

FIG. 20A, FIG. 20B, FIG. 20C, and FIG. 20D show murine CD4+αρ-T cells reduced GVHD and T cell response in vivo. Balb/c recipients were irradiated with 8.5Gy and transplanted with 5×10⁶ B6xB6/SJL F1 (CD45.1+CD45.2+) TCD BM (TCD-BM, n=7), together with 0.5×10⁶ FACS-sorted B6/SJL (CD45.1+) PD-L1+CD4+αρ-T cells plus 0.2×10⁶ B6 (CD45.2+) CD4+ T_{N} cells (αρ-T+T_{N}, n=6), or (0.2×10⁶ CD4+ T_{N} cells (T_{N}, n=6). FIG. 20A shows cytokine expression of blood collected on day 14. FIG. 20B shows cytokine expression of blood collected on day 14. FIG. 20C shows PD-1+CX3CR1+ cell percentage on CD45.2+ T_{N} derived conv-T cells. FIG. 20D shows histograms depicting the fraction of molecules critical for T cell migration on CD4+αρ-T cells derived from spleen. *, P<0.05; **, P<0.01; ***, P<0.001.

### Delayed Infusion of Donor αρ-T Cells Arrests Ongoing GVHD.

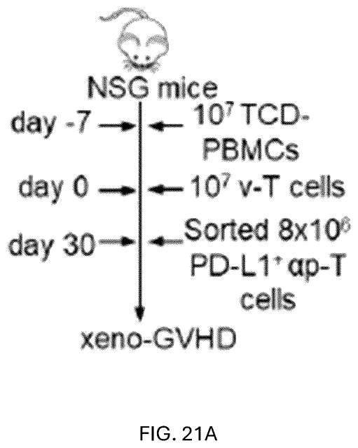

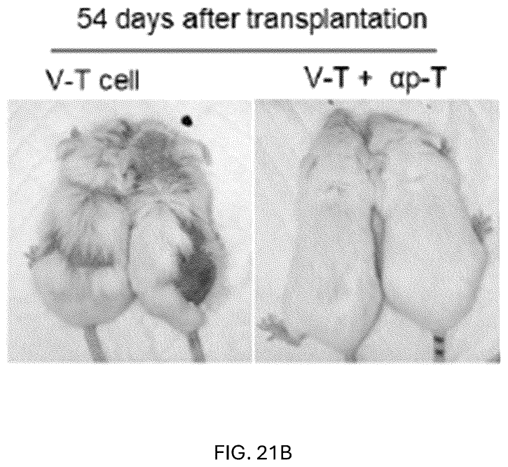

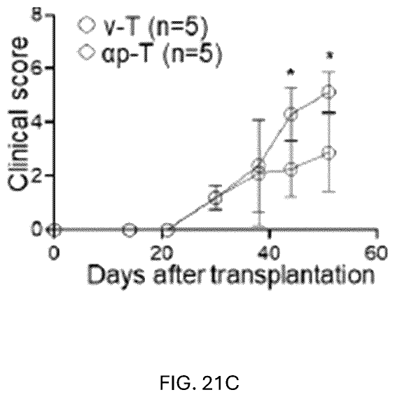

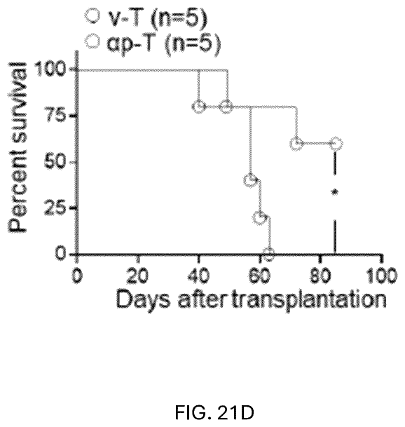

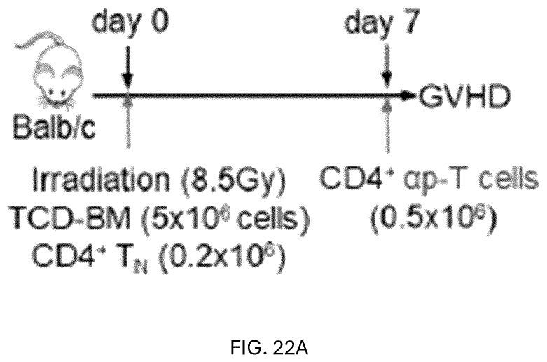

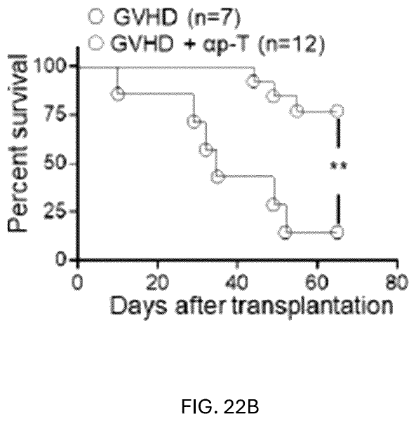

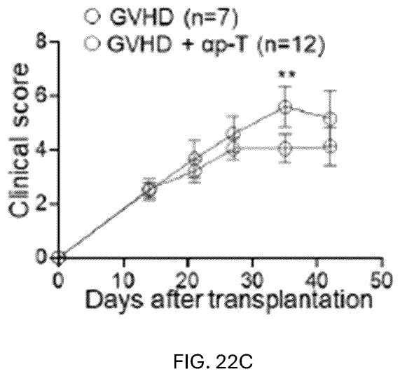

The therapeutic potential of αρ-T cells on reducing GVHD was evaluated in both immunodeficient NSG and immunocompetent Balb/c mouse models. Transfer of purified human PD-L1⁺αρ-T cells (8×10⁶) to NSG mice 30 days after receiving human TCD-PBMCs and v-T cells, when clinical signs of x-GVHD occurred, reduced disease progression and prolonged survival (FIG. 21A, FIG. 21B, FIG. 21C, FIG. 21D). Similarly, in the immunocompetent B6 to Balb/c GVHD model, transfer of murine PD-L1⁺αρ-T cells into Balb/c recipients 7 days after infusion of B6 T cells plus TCD-BM, when GVHD onset occurred, effectively reduced the disease severity and improved the survival (FIG. 22A, FIG. 22B, FIG. 22C). These data suggest that PD-L1⁺αρ-T cells possess potent immunoregulatory activity capable of suppressing ongoing GVHD.

FIG. 21A, FIG. 21B, FIG. 21C, FIG. 21D show αρ-T cell administration suppressed the onset of GVHD. FIG. 21A shows a schematic depicting the experimental procedure. NSG mice were administered with 10 million TCD PBMCs at day −7, followed by injection of 10 million v-T cell products on day 0. Sorted PD-L1+αρ-T cells were administered on day 30. FIG. 21B shows photos of mice with or without αρ-T cell treatment. FIG. 21C shows clinical scores over time after transplantation. FIG. 21D shows overall survival of animals. *, P<0.05; **, P<0.01.

FIG. 22A, FIG. 22B, FIG. 22C shows Balb/c recipients that were irradiated with 8.5 Gy and transplanted with 5×10⁶ B6 TCD-BM together with 0.2×10⁶ B6 CD4+ T_{N} cells to induce GVHD. FACS sorted 0.5×10⁶ PD-L1+CD4+αρ-T cells were administered on day 7. FIG. 22A shows a schematic depicting the experimental procedure. FIG. 22B shows percent survival over time after transplantation. FIG. 22C shows clinical score over time after transplantation. *, P<0.05; **, P<0.01.

### αρ-T Cell Treatment Preserves Anti-Leukemia Activity.

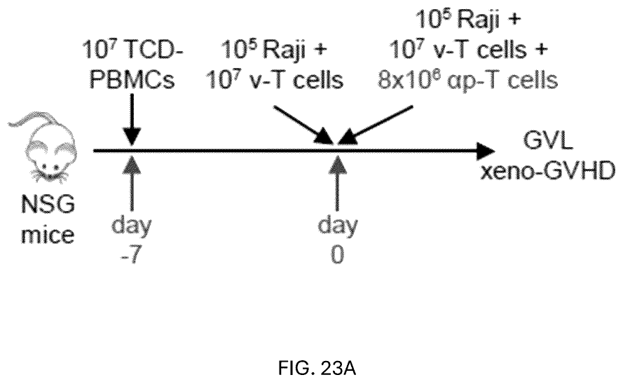

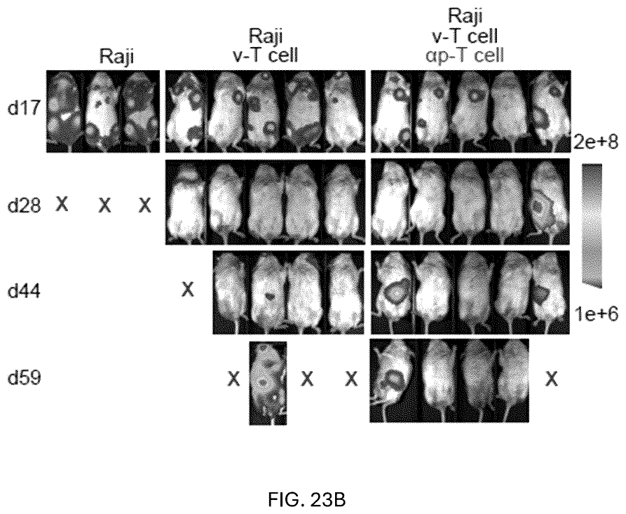

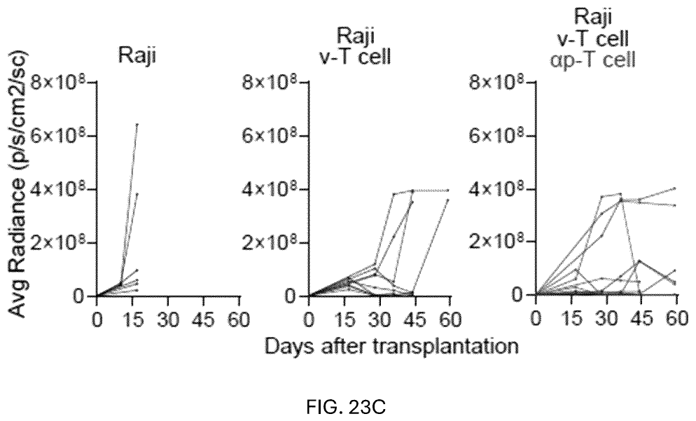

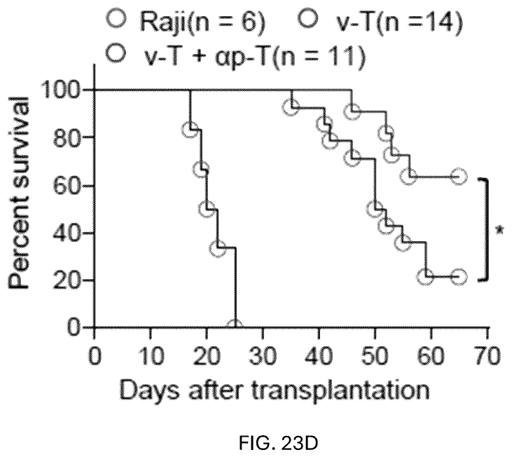

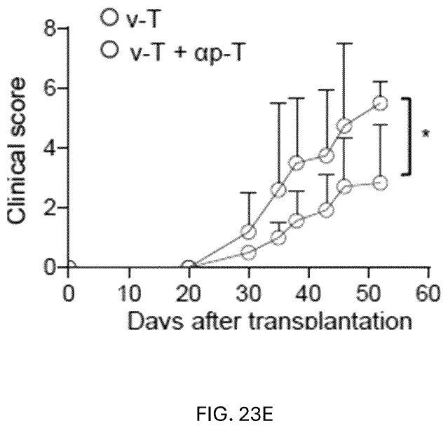

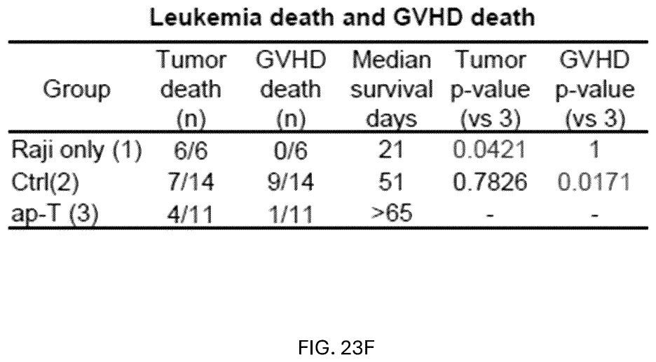

Retaining GVL effects is highly desirable after allo-HCT [Blazar, B. R., et al. (2020). Nat Rev Clin Oncol 17, 475-492, Hefazi, M., et al. (2021). Int J Mol Sci 22]. Therefore, the impact of αρ-T cell treatment on the GVL activity was examined using human xenograft leukemia-bearing hu-NSG mice. Human Raji lymphoma cells (10⁵ cells) were injected (i.v.) at day 0 into hu-NSG mice that were given TCD-PBMCs 7 days earlier, followed by transfer of (i.v.) v-T cells of healthy donor origin (10⁷ cells) that were mixed with or without αρ-T cells (8.0×10⁶ cells) at day 0 (FIG. 23A). αρ-T cell treatment did not impair the capacity of v-T cells to inhibit leukemia growth (FIGS. 23B-23C) but significantly increased overall survival rates of leukemia-bearing mice (7/11, 63.6%) compared to the control group (3/14, 21.4%) (FIG. 23D), which was associated with reduced GVHD clinical score (FIG. 23E) and GVHD-associated death (FIG. 23F).

FIG. 23A, FIG. 23B, FIG. 23C, FIG. 23D, FIG. 23E, FIG. 23F show that human αρ-T cell treatment preserves GVT effects in human xenograft leukemia-bearing NSG mice. FIG. 23A shows the schematic experimental procedure. NSG mice were administrated of 10×10⁶ TCD-PBMCs at day −7, followed by injection of 1.0×10⁵ Raji^{TGL} cells together with 10×10⁶ autologous v-T cells, or 10×10⁶ autologous v-T cells+8×10⁶ αρ-T cells at day 0. FIG. 23B shows representative in vivo imaging at different time points. FIG. 23C shows quantification of photon flux over time. FIG. 23D shows Kaplan-Meier survival analysis. FIG. 23E shows GVHD clinical scores. FIG. 23F shows a summary table of leukemia- or GVHD-related mortality. *, P<0.05.

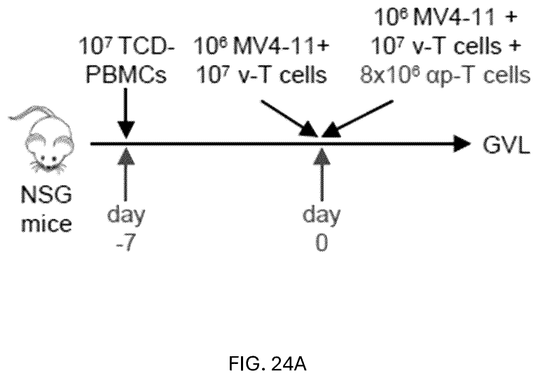

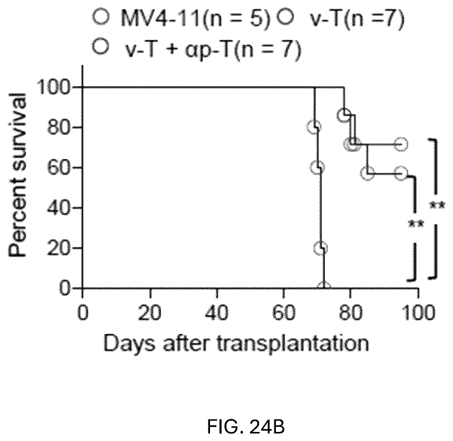

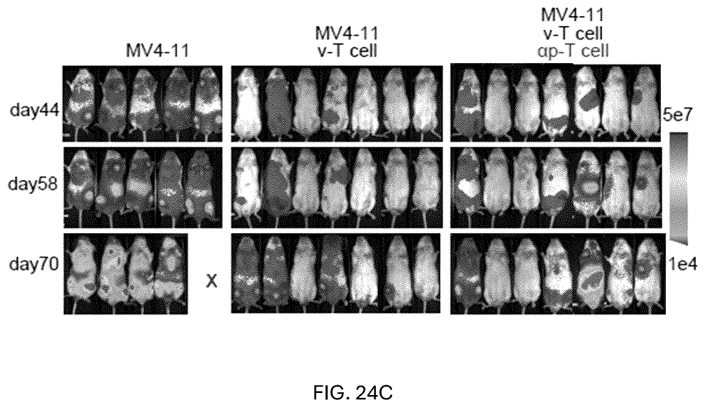

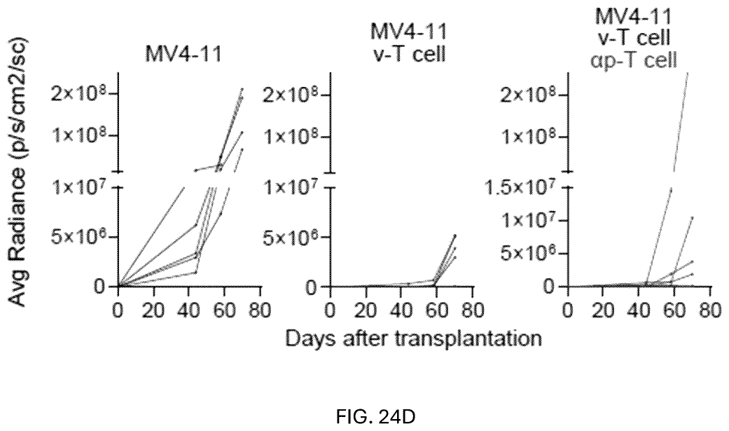

Using a second GVL model, it was further demonstrated that the transfer of human αρ-T cells did not impair the antitumor activity against MV4-11 leukemia (a human acute myeloid leukemia cell line), ruling out a model-specific effect (FIG. 24A, FIG. 24B, FIG. 24C, FIG. 24D). Thus, αρ-T cell treatment preserves anti-leukemia activity via a mechanism of maintaining the production of cytotoxic effector T cells.

FIG. 24A, FIG. 24B, FIG. 24C, FIG. 24D show NSG mice that were administered 10×10⁶ TCD-PBMCs at day −7, followed by injection of 106 MV4-11^{TGL} cells together with 10×10⁶ autologous v-T cells, or 10×10⁶ autologous v-T cells+8×10⁶ αρ-T cells at day 0. FIG. 24A shows a schematic of the experimental procedure. FIG. 24B shows Kaplan-Meier survival analysis. FIG. 24C shows representative in vivo imaging at different time points. FIG. 24D shows quantification of photon flux over time. *, P<0.05.

αρ-T Cells Resist Immunosuppressant Tacrolimus and have No Inhibitory Effects on CMV-Specific T Cells in Cultures.

In clinical practice, immunosuppressive agents, such as calcineurin inhibitors (e.g., cyclosporin A and tacrolimus) are routinely administered early during the allo-HSCT period [Blazar, B. R., et al. (2020). Nat Rev Clin Oncol 17, 475-492, Zeiser, R., and Blazar, B. R. (2017). N Engl J Med 377, 2565-2579, Zeiser, R., and Blazar, B. R. (2017). N Engl J Med 377, 2167-2179].

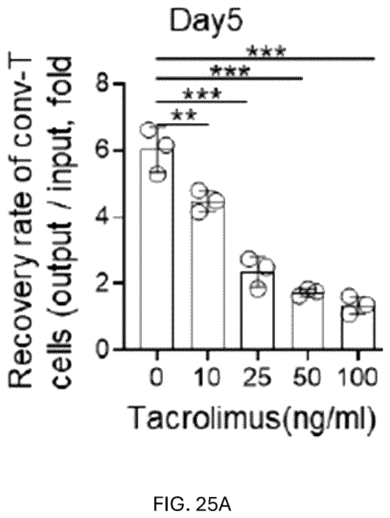

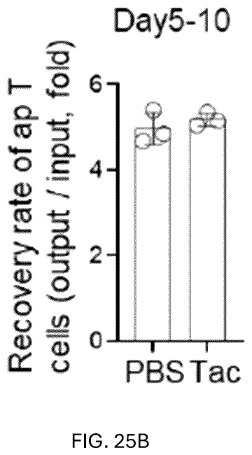

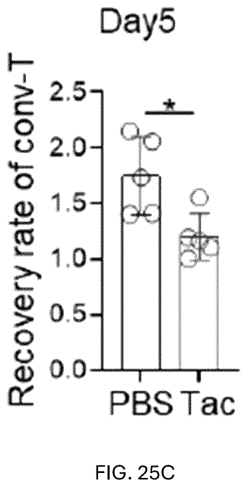

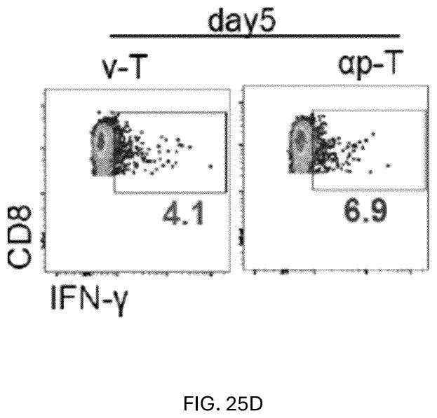

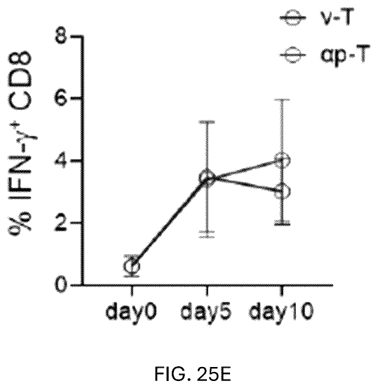

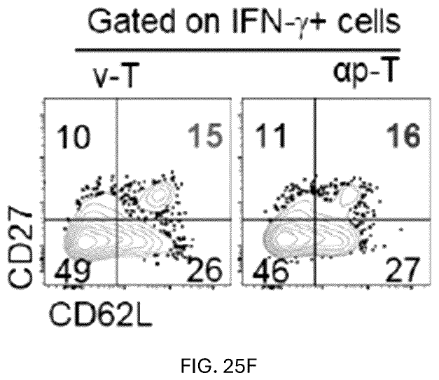

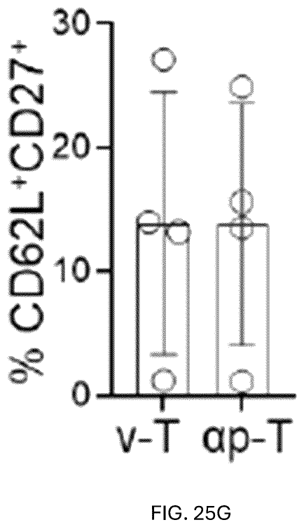

It was observed that tacrolimus (a potent immunosuppressant drug primarily used to prevent organ rejection after a transplant) effectively reduced proliferation of TCR-activated conv-T cell expansion in vitro (FIG. 25A). In contrast, tacrolimus neither reduced expansion nor decreased suppressive function of αρ-T cells in cultures (FIGS. 25B-25C).

In addition, using an immunomodulatory approach to suppress GVHD remains a major concern because it increases the risk of viral infection, such as cytomegalovirus (CMV) infection [Jakharia, N., et al. (2021). Curr Treat Options Infect Dis 13, 123-140]. The impact of αρ-T cells on proliferation and memory phenotype of CMV-specific CD8⁺ T cells in cultures was evaluated. Unlike their effects on repressing TCR-activated T cells, αρ-T cells neither impaired the survival of CMV-specific IFN-γ-producing CD8⁺ T cells (FIGS. 25D-25E), which were activated by hCMV peptides as described in Hanley, P. J., et al. (2015). Sci Transl Med 7, 285ra263, nor altered the frequency of memory-phenotype CMV-specific T cells (FIGS. 25F-25G). Altogether, these data indicate that αρ-T cell treatment can potentially preserve antiviral immunity.

FIG. 25A, FIG. 25B, FIG. 25C, FIG. 25D, FIG. 25E, FIG. 25F, and FIG. 25G show αρ-T cells are not affected by immunosuppressive agents and retain antiviral function in vitro. FIG. 25A shows conv-T cells that were activated with plate-bound anti-CD3 (1.0 μg/mL) and anti-CD28 (1.0 μg/mL). Bar graphs show recovery rate of conv-T cells treated with various doses of Tacrolimus (Tac). FIG. 25B shows PD-L1+αρ-T cells that were sorted on day 5 and treated with Tac (25 ng/mL) from day 5 to day 10. Bar graphs show recovery of αρ-T cells. FIG. 25C shows αρ-T cell that were treated with or without Tac and were co-cultured with conv-T cells, activated with anti-CD3 (1 μg/mL) and anti-CD28 (50 ng/ml), and cultured for 5 additional days. Bar graphs show recovery of conv-T cells. FIG. 25D shows representative plots depicting IFN-γ expression in CD8 T cells. FIG. 25E shows representative graphs depicting IFN-γ expression in CD8 T cells. FIG. 25F shows representative plots showing CD62L+CD27+ memory subsets among IFN-γ+CD8 T cells. FIG. 25G shows representative graphs showing CD62L+CD27+ memory subsets among IFN-γ+CD8 T cells. *, P<0.05; **, P<0.01; ***, P<0.001.

### DISCUSSION

Novel approaches that produce immune regulatory cells, which can robustly expand in culture, persist in vivo after infusion, migrate into GVHD target tissues and sustain immune modulatory function in vivo, remain an unmet need to improve efficacy of living cell therapy for GVHD inhibition [Doglio, M., et al. (2022). Front Immunol 13, 1045168, Voermans, C., and Hazenberg, M. D. (2020). Blood 136, 410-417, Blazar, B. R., et al. (2018 Blood 131, 2651-2660].

It was found that while human pDCs possessed strong immunosuppressive effects on antigen-driven T cell responses, they were a rare and fragile cell population which limited their clinical application. Instead, it was demonstrated herein that suppressor αρ-T cells met these requirements. In culture, αρ-T cells robustly expanded and maintained stable expression of PD-L1 and IFN-α during expansion. This allows the generation of a sufficient number of immune suppressor cells for therapeutic purposes. Following infusion into the allogeneic recipient, suppressor αρ-T cells survived, persisted and infiltrated into peripheral tissues, where they repressed GVHD. Furthermore, this αρ-T cell treatment preserved the GVL effect, significantly improving the survival of human xenograft leukemia-bearing NSG mice. Thus, suppressor αρ-T cells may represent a new and translational strategy to improve the safety and efficacy of allo-HCT.

A unique feature of αρ-T cells is that they retained the capacity to persist and infiltrate GVHD target tissues, which distinguish them from other types of immunoregulatory cells like Tregs and MSCs [Doglio, M., et al. (2022). Front Immunol 13, 1045168, Voermans, C., and Hazenberg, M. D. (2020). Blood 136, 410-417, Blazar, B. R., et al. (2018). Blood 131, 2651-2660, Mamo, T., et al. (2022). Transfusion 62, 904-915].

MSCs are generated from BM cells, have great variability and treatment outcomes, and cannot be detected in vivo after infusion. This makes it difficult to understand at the tissue level why MSCs fail to suppress GVHD [Blazar, B. R., et al. (2018). Blood 131, 2651-2660].

Tregs have shown promising results in preventing GVHD in patients [Hefazi, M., et al. (2021). Int J Mol Sci 22, Blazar, B. R., et al. (2018). Blood 131, 2651-2660]. However, inadequate purification and ex vivo expansion of Tregs may be a major limitation for their application. Furthermore, the limited capacity of Tregs to persist in vivo after infusion has caused loss of their suppressive effects [Mamo, T., et al. (2022). Transfusion 62, 904-915].

As described herein, following infusion, CD4+αρ-T cells persisted and infiltrated GVHD target tissues in both immunodeficient and immunocompetent mice of GVHD. Antigen stimulation is essential for maintaining expansion and persistence of T cells in vivo [Blank, C. U., et al. (2019). Nat Rev Immunol 19, 665-674]. Without being limited by theory, further studies will investigate the possible role of host antigen stimulation in sustaining αρ-T cells in GVHD target tissues, which is important for understanding their biological properties.

αρ-T cells suppression of GVHD may involve multiple mechanisms. Previous studies demonstrate that IFN-α promotes expression of genes encoding TRAIL and FasL critical for activation of caspase-3 and caspase-8 to induce cell death [Bahl, K., et al. (2006). Journal of immunology 176, 4284-4295, Malireddi, R. K., and Kanneganti, T. D. (2013). Front Cell Infect Microbiol 3, 77, Shi, W., et al. (2022). Oncol Lett 24, 235].

IFN-α also activates the cell death process through the JAK1-STAT signaling pathway [Shi, W., et al. (2022). Oncol Lett 24, 235]. In mice, administration of IFN-α attenuates donor CD4+ T cell-mediated GVHD [Tian, Y., et al. (2021). J Clin Invest 131, Robb, R. J., et al. (2011). Blood 118, 3399-3409].

It was observed that CD4+αρ-T cell treatment upregulated expression of IFN-α-induced gene programs in conv-CD4+ T cells, including STAT1, IRF7 and a group of ISGs, and impaired expansion and survival capabilities of these conv-CD4+ T cells. Thus, IFN-α derived from CD4+αρ-T cells may play a crucial role in preventing GVHD.

Without being limited by theory, as disclosed herein, IFN-α and PD-L1 derived from CD4+αρ-T cells could synergistically suppress GVHD reactions. PD-L1/PD-1 interactions are critical for dampening the immune response to both self-antigen and foreign antigens. It has been shown that PD-L1 ligation of PD-1 induces apoptosis, exhaustion and anergy in antigen-driven T cells, resulting in inhibition of GVHD [Ni, X., et al. (2017). J Clin Invest 127, 1960-1977, Song, Q., et al. (2021). Blood 137, 2243-2255, Haspot, F., et al. (2008). Blood 112, 2149-2155, Li, X., et al. (2012). Journal of Immunology 188, 724-734, Saha, A., et al. (2013). Blood 122, 3062-3073].

Previous studies suggested that IFN-α induces expression of inhibitory receptors (PD-1, TIM3 and LAG3) and T cell exhaustion [Chen, W., et al. (2022). Cell Rep 41, 111647]. As disclosed herein, CD4+αρ-T cells promoted the generation of PD-1+TIM3+ term-TEX-like cells from CD4+ conv-T cells both in cultures and in x-GVHD model. IFN-α treatment recapitulated this effect of CD4+αρ-T cells in cultures. Incubation of PD-1+TIM3+ T cells in the presence of PD-L1-expressing K562 cells impaired expansion and survival capabilities of PD-1+TIM3+ T cells. Thus, CD4+αρ-T cell treatment creates a reinforcing regulatory loop to effectively inhibit GVHD, in which IFN-α impairs expansion and survival of host-reactive T cells, promotes generation of PD-1+TIM3+ term-TEX-like cells, thereby enhancing their sensitivity to PD-L1-mediate immune suppression.

Despite the decreased expansion capacity of PD-L1⁺αρ-T cells, it appeared that tissue infiltrating PD-L1⁺αρ-T cells had potent ability to suppress ongoing GVHD. In both the immunodeficient NSG and immunocompetent Balb/c mouse models, delayed infusion of donor αρ-T cells after the onset of GVHD significantly attenuated the disease severity and improved the overall survival of recipients. These findings strongly argue the notion that αρ-T cells have great capability to actively suppress pathogenic GVH reaction.

The mechanisms by which αρ-T cells preserved GVL activity while preventing GVHD may be complex. First, αρ-T cell-derived IFN-α can act directly or indirectly on preserving the GVL effect. IFN-α has been shown to provoke anti-leukemia activity. In mice, administration of IFN-α attenuates donor CD4+ T cell-mediated GVHD but retains CD8+ T cell-mediated GVL effect [Tian, Y., et al. (2021). J Clin Invest 131, Robb, R. J., et al. (2011). Blood 118, 3399-3409]. Infusion of IFN-α to patients undergoing allo-HCT resulted in potent GVL effects without inducing severe GVHD [Henden, A. S., et al. (2019). Blood Adv 3, 3013-3019, Magenau, J. M., et al. (2021). Blood Adv 5, 5047-5056]. IFN-α can also act on innate immune cells to secrete IL-15 [Huber, J. P., and Farrar, J. D. (2011). Immunology 132, 466-474], which plays important roles in augmenting GVL effects [Mathew, N. R., et al. (2018). Nat Med 24, 282-291].

Second, it is suggested that the alloreactivity threshold for eliminating leukemia in hematopoietic and lymphoid tissues (HLTs) appears to be lower than that required for mediating severe GVHD in non-HLTs [He, S., et al. (2013). Blood 122, 4119-4128. blood-2013-05-50518, Huang, Q., et al. (2017). Blood, Valenzuela, J. O., et al. (2009). J Clin Invest 119, 3774-3786].

While αρ-T cell treatment impaired expansion and caused exhaustion of host-reactive conv-T cells, it promoted differentiation of host-reactive T cells into IFN-γ-producing effector cells during GVHD. Therefore, αρ-T cell treatment could reduce alloreactivity to the threshold insufficient to mediate GVHD but capable of eliminating leukemia.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A cell therapy method comprising:
(a) genetically engineering a population of T cells derived from a healthy donor to overexpress PD-L1 and IFN-α2, thereby generating a population of αρ-T cells,
wherein the genetically engineered population of αρ-T cells is characterized by expansion in vitro, persistence in vivo, capacity to infiltrate target tissues, and sustained immune modulatory function; and
(b) administering a hematopoietic cell transplant comprising the genetically engineered population of αρ-T cells to a recipient subject in need thereof, wherein the recipient subject in need thereof is afflicted with an autoimmune or alloimmune disorder,
wherein the administering of the population of T cells derived from a healthy donor genetically engineered to overexpress PD-L1 and IFN-α2 (αρ-T cells) is by intravenous infusion.

2. The cell therapy method of claim 1, wherein the genetic engineering comprises:
(a) transducing T cells from a healthy donor with a lentivirus vector encoding PD-L1 and IFN-α2;
(b) isolating PD-L1 and IFN-α2-expressing T cells using fluorescence activated cell sorting or magnetic beads to form a purified population of PD-L1, IFN-α2 expressing αρ-T cells;
(c) expanding the purified population of αρ-T cells by culturing; and
(d) activating the expanded purified population of αρ-T cells.

3. The cell therapy method of claim 1, wherein the population of αρ-T cells is allogeneic or autologous to the recipient patient.

4. The cell therapy method according to claim 1, wherein:
(a) the recipient subject is a mammal;
(b) the mammal is a human; or
(c) when the mammal is a human, the human is a child, a young adult, or an adult.

5. The cell therapy method according to claim 1, wherein the genetically engineered T cell population comprises genetically engineered CD4+ T cells, genetically engineered CD8+ T cells, or both genetically engineered CD4+ T cells and genetically engineered CD8+ T cells.

6. The cell therapy method of claim 1, wherein upregulating expression of IFN-α induced gene programs includes increased expression of IFN-stimulated genes, genes involved in cell migration, genes involved in tissue infiltration, genes encoding inhibitory receptors, genes encoding transcription factors, genes involved in inflammatory response, genes involved in INFα response, genes involved in IFN-γ response, or a combination thereof.

7. The cell therapy method according to claim 1, wherein:
(i) the IFN stimulated genes comprise IFI27, IFITM1, and ISG15;
(ii) the genes involved in cell migration and tissue infiltration comprise ITGA1, ITGA3, CCR5, CCR9, α4β7, CXCR3, and CCR7;
(iii) the genes encoding inhibitory receptors comprise HAVCR2, LAG3, ENTPD1, and TIGIT;
(iv) the genes encoding transcription factors comprise PRDM1, TOX, STAT1, and CEBPB;
(v) the genes involved in inflammatory response, INFα response, and IFN-γ response comprise LAG3, HAVCR2, and TOX.

8. The cell therapy method according to claim 3, wherein the transplantation of the allogeneic or autologous hematopoietic cells comprising the population of genetically engineered T cells results in increased expression of Src homology 2-containing protein tyrosine phosphatase 2 (pSHP2) compared to a control subject.

9. The cell therapy method according to claim 1, wherein the administered hematopoietic cell transplant comprising the population of genetically engineered T cells increases frequency of PD-1+CX3CR1+CD4+ conventional T cells in the recipient subject compared to a control subject, and
wherein the increased frequency of PD-1+CX3CR1+CD4+ conventional T cells in the recipient subject is detectable in one or more of peripheral blood, bone marrow, liver, or spleen.

10. The cell therapy method according to claim 8, wherein the PD-1+CX3CR1+CD4+ conventional T cells are TOX+TCF1− T cells.

11. The cell therapy method according to claim 1, wherein the recipient subject in need thereof is afflicted with a hematologic cancer, and
wherein the method suppresses graft versus host disease (GVHD), thereby decreasing risk of a graft versus host reaction in the recipient subject while preserving graft versus tumor (GVT) immunity compared to a non-genetically engineered control.

12. The cell therapy method according to claim 11, wherein the hematologic cancer is a leukemia, a myeloma, or a lymphoma.

13. The cell therapy method according to claim 12, wherein the leukemia is an acute myeloid leukemia, acute lymphoid leukemia, chronic myelomonocytic leukemia, chronic myeloid leukemia, myelodysplastic syndrome (MDS), or a myeloproliferative neoplasm, or
wherein the lymphoma is a non-Hodgkin's lymphoma.

14. The cell therapy method according to claim 13, wherein the non-Hodgkin's lymphoma is a B cell lymphoma or a T cell lymphoma.

15. The cell therapy method according to claim 11, wherein the population of genetically engineered αρ-T cells suppresses GVHD by:
(a) upregulating expression of IFN-α induced gene programs in alloreactive non-genetically engineered CD4+ T cells in the administered hematopoietic cell transplant;
(b) impairing expansion and survival of alloreactive T cell populations in the administered hematopoietic cell transplant;
(c) inducing apoptosis, T cell exhaustion, and T cell anergy of the non-genetically engineered alloreactive T cells in the transplant by PD-L1 ligation of PD-1;
(d) promoting differentiation of host-reactive T cells into IFN-γ producing effector cells; or
(e) a combination thereof.

16. The cell therapy method according to claim 15, wherein the exhaustion-like T cells are characterized by a PD-1+TIM3+ phenotype.

17. The cell therapy method according to claim 11, wherein the administered hematopoietic cell transplant comprising the population of genetically engineered αρ-T cells:
(i) decreases clinical signs of ongoing graft-versus-host disease in the recipient subject compared to a control subject;
(ii) increases survival of the recipient subject compared to a control subject;
(iii) reduces inflammation compared to a control subject; or
(iv) a combination thereof.

18. The cell therapy method according to claim 11, wherein preserving GVT immunity in the recipient subject comprises maintaining the production of cytotoxic effector T cells in the recipient subject.

19. The cell therapy method according to claim 11, further comprising administering an additional therapeutic agent.

20. The cell therapy method according to claim 19, wherein the additional therapeutic agent comprises an immunomodulator, a chemotherapy agent, or a combination thereof.

21. The cell therapy method according to claim 20, wherein the immunomodulator is ipilimumab (YERVOY®).

22. The cell therapy method of claim 1, wherein the recipient subject in need thereof is afflicted with or at risk for an autoimmune disorder or disorder with an autoimmune component; and
wherein the administering of hematopoietic cells comprising the population of genetically engineered T cells:
(i) suppresses expansion of autoreactive conventional T cells in the recipient subject compared to a control subject;
(ii) decreases survival of autoreactive conventional T cells in the recipient subject compared to a control subject;
(iii) increases autoreactive conventional T cell differentiation into exhaustion-like T cells in the recipient subject compared to a control subject; or
(v) a combination thereof.

23. The cell therapy method according to claim 22, wherein the method reduces expansion of autoreactive populations of T cells by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% compared to a control subject.

24. The cell therapy method according to claim 22, wherein the survival of the autoreactive populations of conventional T cells in the recipient subject is decreased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% compared to a control subject.

25. The cell therapy method according to claim 22, further comprising administering an additional therapeutic agent.

26. The cell therapy method according to claim 25, wherein the additional therapeutic agent comprises alpha-1-antitrypsin, prednisolone, dexamethasone, azathioprine, cyclosporine, tacrolimus, sirolimus, methotrexate, mycophenolate mofetil, adalimumab, infliximab, anakinra, tocilizumab, baricitinib, ruxolitinib, certolizumab, etanercept, golimumab, canakinumab, sarilumab, secukinumab, ustekinumab, abatacept, rituximab, fludarabine, busulfan, melphalan, or belimumab.
